(19) United States
(12) Patent Application Publication
(10) Pub. No.: US 2013/0252950 A1
(43) Pub. Date: Sep. 26, 2013

(54) TARGETING MTOR SUBSTRATES IN TREATING PROLIFERATIVE DISEASES

(71) Applicants: John Blenis, Newtonville, MA (US); Steven P. Gygi, Foxborough, MA (US); Yonghao Yu, Roxbury, MA (US)

(72) Inventors: John Blenis, Newtonville, MA (US); Steven P. Gygi, Foxborough, MA (US); Yonghao Yu, Roxbury, MA (US)

(73) Assignee: President and Fellows of Harvard College, Cambridge, MA (US)

(21) Appl. No.: 13/825,723

(22) PCT Filed: Sep. 23, 2011
(86) PCT No.: PCT/US11/53035

§ 371 Date: Jun. 11, 2013

Related U.S. Application Data
(60) Provisional application No. 61/403,932, filed on Sep. 23, 2010.

(51) Int. Cl. C12Q 1/48; G01N 33/68

(52) U.S. Cl. CPC C12Q 1/485; G01N 33/68
USPC 514/232.5; 435/15; 514/291; 435/7.1; 506/9

(57) ABSTRACT

Provided are over 300 mTOR kinase targets identified by a comprehensive phosphoproteomics assay. Methods of targeting mTOR kinase targets, methods to determine the level of mTOR activity by measuring the level of phosphorylation of an mTOR targeted phosphorylation site, methods for distinguishing different classes of mTOR activity in a cell based on phosphoproteomic analysis of mTOR-targeted proteins are also provided. Also provided is the classification of a hyperproliferative disease based on phosphoproteomic analysis of mTOR-targeted proteins, as well as the personalization of therapeutic methods for the treatment of hyperproliferative disease based on phosphoproteomics. Also provided are therapeutic methods including administering to a subject an mTOR inhibitor, an mTOR inhibitor and an additional kinase inhibitor, or a dual inhibitor of mTOR and an additional kinase based on the phosphorylation levels of mTOR targets determined in the subject. Some aspects of this invention relate to the discovery that GrblO is an mTOR-targeted tumor suppressor gene.

### RELATED APPLICATIONS

This application claims priority under 35 U.S.C. §119(e) to U.S. provisional patent application, U.S. Ser. No. 61/403,932, filed Sep. 23, 2010, which is incorporated herein by reference.

### GOVERNMENT SUPPORT

This invention was made with U.S. Government support under grant numbers GM051405 and HG3456 awarded by the National Institutes of Health. The U.S. Government has certain rights in the invention.

### BACKGROUND

mTOR is an evolutionarily conserved ser/thr protein kinase that controls many critical cellular processes including growth, protein translation, metabolic flux, and cell survival. mTOR functions as the core catalytic component of two structurally and functionally distinct signaling complexes. mTOR complex 1 (mTORC1) regulates cell growth and is responsible for the well-characterized role of mTOR in controlling protein translation whereas mTOR complex 2 (mTORC2) regulates cell survival and the actin cytoskeleton (1-3). The mechanisms responsible for modulating mTORC1 and mTORC2 activity in response to upstream inputs such as growth factors, energetic status, and amino acid levels have been well studied (1). In contrast, relatively few direct substrates of mTOR have been identified and in many cases the mechanisms underlying mTOR's ability to regulate important aspects of cell biology are not known.

Misregulated mTOR activity is a common feature of most cancers (2). Despite great interest, clinical trials for evaluating the selective mTORC1 inhibitor rapamycin as an anti-cancer agent have met with limited success (3). Rapamycin resistance has emerged as a major challenge to its clinical use (4) and is caused in part by feedback loops that activate the PI3K and MAPK signaling pathways in rapamycin-treated cells through poorly understood mechanisms (5, 6).

### SUMMARY OF THE INVENTION

Functional characterization of the mTOR signaling pathways has been hampered by the paucity of substrates that have been identified to date. Identifying novel substrates of mTORC1 and mTORC2 is important for making progress toward our general understanding how mTOR signals to downstream effectors and to specifically define components of the feedback loops involved in rapamycin resistance. The best-characterized mTORC1 substrates include p70S6K and 4EBP, whereas mTORC2 phosphorylates several members of the AGC kinase family, including Akt, SGK, and PKC.

The present invention is based in part on large-scale quantitative phospho-proteomics experiments that were performed to define the entire signaling networks downstream of both mTORC1 and mTORC2. Novel mTORC1 substrates identified herein include, but are not limited to Grb10, FOXK1, ZEB2, NDRG3, LARP1, SRPK2, CDK12, MIB1, and IBTK.

Extensive characterization of a novel mTORC1 substrate, the growth factor receptor-bound protein 10 (Grb10), shows that mTORC1-mediated phosphorylation stabilizes Grb10, leading to feedback inhibition of the PI3K and MAPK pathways. In addition, Grb10 expression was shown to be frequently downregulated in a variety of cancers. Interestingly, loss of Grb10 and the well-established tumor suppressor PTEN are mutually exclusive events. Grb10 has been found to be both a novel mTORC1 substrate and a tumor suppressor with relevance across a broad spectrum of cancer subtypes.

In some aspects, the invention provides methods for determining mTOR kinase activity in a cell, the method comprising obtaining a cancer cell from a subject diagnosed to have a cancer, determining the level of Grb10 phosphorylation in the cell, and comparing the level of Grb10 phosphorylation to a reference level, wherein if the level of Grb10 phosphorylation in the cell is higher than the reference level, then the cell is determined to exhibit an elevated level of mTOR kinase activity. In some embodiments, the cell is a normal or healthy cell. In some embodiments, the cell is a cell obtained from a subject not diagnosed with a neoplastic disease. In some embodiments, the cell is a cell obtained from a subject not diagnosed with a cancer. In some embodiments, the cell is a neoplastic cell. In some embodiments, the cell is a cancer cell. In some embodiments, the cell is a cell obtained from a subject diagnosed with a neoplastic disease, for example, with a cancer.

In some aspects, the invention provides methods for selecting a treatment of a neoplastic disease, for example, of a cancer, in a subject, the method comprising obtaining a cell from a subject diagnosed to have a neoplastic disease, for example, a cancer, exhibiting an elevated level of mTOR activity; determining the level of Grb10 expression in the cell; and comparing the level of Grb10 expression to a reference level, wherein if the level of Grb10 expression in the cell is higher than the reference level, then the cell is determined to exhibit a high likelihood of expressing an elevated level of PI3K, Akt, and/or MAPK activity upon being contacted with an mTORC1 inhibitor.

Other aspects of this invention provide methods for determining mTOR kinase activity in a cell by determining the level of phosphorylation of a phosphorylation site disclosed in Table 1, 2, 3, 7, 8, or 11 in the cell, and comparing the level of phosphorylation to a reference level, wherein if the level of phosphorylation is higher than the reference level, then the cell is determined to exhibit an elevated level of mTOR kinase activity, or if the level of phosphorylation is equal or lower than the reference level, then the cell is determined to not exhibit an elevated level of mTOR kinase activity. In certain embodiments, if a phosphorylation site determined to exhibit a higher level of phosphorylation in the cell as compared to the reference level is a class I phosphorylation site, then the cell is determined to exhibit an elevated level of rapamycin-sensitive mTORC1 activity. In certain embodiments, if a phosphorylation site determined to exhibit a higher level of phosphorylation in the cell as compared to the reference level is a class II phosphorylation site, then the cell is determined to exhibit an elevated level of rapamycin-insensitive mTORC1 and/or mTORC2 activity. In some embodiments, if a phosphorylation site determined to exhibit a higher level of phosphorylation in the cell as compared to the reference level is a class III phosphorylation site, then the cell is determined to exhibit an elevated level of rapamycin-sensitive mTOR activity, or an elevated level of rapamycin-insensitive mTOR activity, or both.

In some embodiments, the methods provided herein further comprise selecting a method of treatment of the subject based on the level of phosphorylation of the phosphorylation site. In some embodiments, if the cell is determined to exhibit an elevated level of mTOR kinase activity, then a method of treatment is selected that includes administering an effective amount of an mTOR kinase inhibitor to the subject. In some embodiments, if the cell is determined to not exhibit an elevated level of mTOR kinase activity, then a method of treatment is selected that does not include administering an mTOR kinase inhibitor. In some embodiments, if the cell is determined to exhibit an elevated level of rapamycin-sensitive mTORC1 activity, then a method of treatment is selected that comprises administering an effective amount of an inhibitor of rapamycin-sensitive mTORC1 kinase activity (e.g., rapamycin or a rapamycin analog) to the subject. In some embodiments, if the cell is determined to exhibit an elevated level of rapamycin-insensitive mTORC1 and/or mTORC2 activity, then a method of treatment is selected that comprises administering an effective amount of an inhibitor of rapamycin-insensitive mTORC1 kinase activity to the subject. In some embodiments, if the cell is determined to exhibit an elevated level of rapamycin-sensitive mTOR kinase activity, or an elevated level of rapamycin-insensitive mTOR kinase activity, or both, then a method of treatment is selected that comprises administering an effective amount of an inhibitor of rapamycin-sensitive and rapamycin-insensitive mTOR kinase activity or a combination of an inhibitor of rapamycin-sensitive mTOR kinase activity and an inhibitor of rapamycin-insensitive mTOR kinase activity to the subject.

In some embodiments, the method of treatment further comprises administering to the subject an effective amount of a compound that stabilizes Grb10 or that inhibits the degradation of Grb10. In some embodiments, the method of treatment further comprises administering an effective amount of an inhibitor of PI3K, Akt, or MAPK to the subject.

Some aspects of this invention provide a phosphoproteomics array that includes a plurality of phosphosensitive antibodies or antibody fragments each of which specifically binds to phosphorylation site disclosed in Table 1, 2, 3, 7, 8, or 11. In some aspects, the invention also provides a method of using a phosphoproteomics array to determine mTOR activity in a cell by contacting a proteinaceous sample derived from a cell, for example, from a cancer cell, with a phosphoproteomics array as described herein under conditions suitable for a protein expressed in the cell to bind to an antibody or antibody fragment of the array, determining the level of phosphorylated protein bound to an antibody or antibody fragment of the array, and comparing the level of phosphorylated protein bound to an antibody or antibody fragment of the array to a reference level. If the level phosphorylated protein in the sample derived from the cell is higher than the reference level, then the cell is determined to exhibit an elevated level of mTOR kinase activity.

In other aspects, the invention provides methods of identifying an mTOR kinase inhibitor by contacting an mTOR kinase with a polypeptide with a phosphorylation site disclosed in Table 1, 2, 3, 7, 8, or 11 under conditions suitable for the mTOR kinase to phosphorylate the phosphorylation site in the presence of a candidate agent, determining the level of phosphorylation at the phosphorylation site, and comparing the level of phosphorylation to a reference level. If the level of phosphorylation is lower than the reference level, then the candidate agent is identified as an mTOR kinase inhibitor.

In some embodiments involving a phosphorylation site disclosed in Table 1, 2, 3, 7, 8, or 11, the phosphorylation site is not a phosphorylation site previously known to be an mTOR target.

The subject matter of this application may involve, in some cases, interrelated products, alternative solutions to a particular problem, and/or a plurality of different uses of a single method or agent. Other advantages, features, and uses of the invention will become apparent from the following detailed description of non-limiting embodiments of the invention when considered in conjunction with the accompanying drawings.

### BRIEF DESCRIPTION OF THE DRAWINGS

FIG. 1. Sample preparation and data analysis for quantitative phosphorylation profiling of mTOR signaling. (A) Schematics of the two quantitative mass spectrometry experiments are shown with a plot highlighting the distribution of phosphopeptides identified in each screen. See data summary in Table 5. Note that most of the phosphopeptides have a ratio of 1:1 between the light and heavy populations and hence have a value close to 0 on a Log₂ axis. Proteins with downregulated phosphorylation in each screen are highlighted in the red box. (B) Typical quantitative MS and MS/MS spectra in which LS*SLRAS*TSKSESSQK (SEQ ID NO: 1) from ribosomal protein S6 (S235 and S240) are identified as a rapamycin-sensitive phosphopeptide. Note the light and heavy peptides differ by 26 Da, corresponding to 2 labeled Lys and 1 labeled Arg in this particular peptide. Sequence in lower panel: SEQ ID NO: 1722. (C) Quantitative differences between the rapamycin-sensitive and -insensitive mTOR downstream phosphorylation events. Phosphopeptides identified in both screens were extracted and their corresponding Log₂ ratios (fold-changes) were plotted. (D) The top ten pathways enriched in the down-regulated phospho-proteins identified in the rapamycin (Rapa) screen.

FIG. 2. Phosphorylation of Grb10 at S501/S503 is sensitive to rapamycin inhibition. (A) Identification of a doubly-phosphorylated rapamycin-sensitive Grb10 peptide (MNILSS*QS*PLHPSTLNAVIHR, SEQ ID NO: 2, * indicates phosphorylation sites) corresponding to S501/S503. (B) Evolutionary conservation of Grb10 S501/S503 among vertebrate species. (C) Phosphorylation of Grb10 at S501/S503 shows rapamycin sensitivity in vivo. TSC2−/− cells were starved for serum and treated with 20 nM rapamycin for the indicated times. Sequences, from top to bottom: SEQ ID NO: 1723 to SEQ ID NO: 1727, respectively. (D) Phosphorylation of Grb10 at S501/501 is sensitive to amino acids withdrawal. TSC2−/− cells were starved in DMEM overnight and then transferred to D-PBS for the indicated times. (E) Phosphorylation of Grb10 at S501/S503 is not affected by the pan-kinase inhibitor, staurosporine. TSC2−/− cells were starved for serum and treated with either 100 nM staurosporine or Ku-0063794 at the indicated concentrations for two hours. (F) Grb10 phosphorylation is increased upon growth factor stimulation. Wild type (WT) mouse embryonic fibroblasts (MEFs) were starved for serum overnight and then were stimulated with insulin (100 nM) or serum (10%) for 15 min. The cells were preincubated with the indicated compounds for two hours. AktVIII (1 μM) is a specific inhibitor of Akt whereas AZD (AZD6244, 5 μM) specifically inhibits MEK. Rapamycin (rapa) was used at 20 nM. (G) Grb10 phosphorylation at S501/S503 is sensitive to various mTOR kinase inhibitors. TSC2−/− cells were serum-starved and treated with the indicated compounds for two hours. The concentrations of the compounds were rapamycin 20 nM, LY (LY294002) 20 μM, BEZ235 (NVP-BEZ235) 500 nM, torin 100 nM, and pp 242 1 μM.

FIG. 3. mTOR-mediated Grb10 phosphorylation is important for Grb10 stability. (A) Grb10 interacts with raptor, but not rictor. HA-tagged Grb10 was co-transfected with Myc-raptor or Myc-rictor in HEK293T cells. Cells were lysed in lysis buffer A, and the lysates were subjected to immunoprecipitation using anti-HA antibody conjugated beads. Raptor and rictor were probed with an antibody against the Myc-tag. (B) Grb10 is phosphorylated by mTOR in vitro. Recombinant Grb10 was prepared from bacteria (the GST-fused Grb10 shows a molecular weight of 80 kDa) and was incubated with recombinant mTOR in vitro. Phosphorylation of Grb10 at S501/S503 was detected by using the phospho-specific antibody against these two sites. (C) Long-term rapamycin treatment leads to Grb10 degradation in TSC2−/− cells. Note that Grb10 protein expression levels inversely correlated with Akt activity. Grb10 is highly overexpressed in TSC2−/− cells. mRNA level was determined using quantitative RT-PCR based on three biological replicate experiments. (D) Knockdown of raptor in TSC2−/− cells decreased Grb10 protein level. Cells were starved overnight and the lysates were probed with the antibodies indicated. (E) Grb10 is highly overexpressed in TSC2−/− cells. (F) S501A/S503A mutant is unstable compared with the wild-type or the S501D/S503D mutant. The same amount of DNA was transfected into HEK293T cells. (G) Rapamycin failed to induce degradation of the S501D/S503D mutant. S501D/S503D mutant (DD) was stably expressed in TSC2−/− cells and cells were treated with 20 nM rapamycin for the indicated times. Endogenous Grb10 was detected using an antibody that preferentially recognizes mouse Grb10 whereas the Grb10 DD mutant (of human origin) was detected using an anti-HA antibody.

FIG. 4. Grb10 is involved in the feedback inhibition loop from mTORC1 to PI3K and MAPK and GRB10 mRNA expression is significantly down-regulated in many cancers and is negatively correlated with PTEN expression. (A) Knockdown of Grb10 in TSC2−/− cells resulted in PI3K and MAPK hyperactivation after insulin or IGF stimulation. (B) Knockdown of Grb10 in TSC2−/− cells protected cells against apoptosis. Grb10 knockdown and control cells were starved overnight and then treated with 100 nM staurosporine for 5 hrs to induce apoptosis. (C) Box plots indicating that GRB10 expression is significantly lower in many tumor types compared to their corresponding normal tissues. (Only the tumor types that showed significantly lower GRB10 expression in cancer vs. normal in at least three independent microarray datasets are included). (D) Heat maps indicating a strong negative correlation between GRB10 and PTEN expression in breast carcinomas and myelomas. Low levels of GRB10 expression rarely occurred in tumors that also showed low levels of PTEN expression. (E) Scatter plots comparing the expression levels of GRB10 and PTEN levels in the normal and tumor samples, collected overall from 6 different tissue types, where GRB10 was shown to be significantly down-regulated in cancer vs. normal. The negative correlation between GRB10 and PTEN expression is evident in the tumor (P<0.001) but not in the corresponding normal samples.

FIG. 5. Data analysis for the rapamycin screen and Ku-0063794 screen. (A) Phosphopeptides enrichment in the second biological replicate experiment of the rapamycin screen. Number of phospho- and nonphospho-peptides, in each SCX fraction was plotted. (B) Number of downregulated proteins in the two biological replicates of the rapamycin screen. (C) Ku-0063794 inhibits insulin-induced Akt phosphorylation. (D) Pathways enriched in the downregulated phospho-proteins identified in the Ku-0063794 screen. The top ten overrepresented pathways were shown. Analysis was performed using DAVID. See Huang D W, Sherman B T, Lempicki R A. Systematic and integrative analysis of large gene lists using DAVID Bioinformatics Resources. Nature Protoc. 2009; 4(1):44-57; and Dennis G Jr, Sherman B T, Hosack D A, Yang J, Gao W, Lane H C, Lempicki R A. DAVID: Database for Annotation, Visualization, and Integrated Discovery. Genome Biol. 2003; 4(5):P3; both incorporated herein by reference for disclosure of integrative analysis methods for large datasets.

FIG. 6. Examples of protein phosphorylation changes identified in the rapamycin and Ku-0063794 screens. Note that the light cells were treated with rapamycin and the heavy cells were treated with DMSO in the rapamycin screen (2^{nd} biological replicate). For the Ku-0063794 screen, light cells were treated with rapamycin whereas the heavy cells were treated with a combination of rapamycin and Ku-0063794. (A) Phosphorylation changes of different sites on 4EBP in the rapamycin screen and Ku-0063794 screen. Phosphorylation of T36/T45 was partially responsive to rapamycin and was completely abolished as a result of Ku-0063794 treatment. In contrast, T70 phosphorylation is mTOR-independent (B) NRDG1 phosphorylation at S330/S333 was sensitive to Ku-0063794 but not rapamycin inhibition. (C) GSK3β phosphorylation at S9 decreased in both rapamycin and Ku-0063794 screen.

FIG. 7. Identification of the rapamycin-sensitive phosphorylation sites on mTOR (A) Intensities of an mTOR peptide ²⁴⁷¹AGTTVPES*HIS*FIGDGLVKPEALNKK²⁴⁹⁶ (SEQ ID NO: 3, * indicates phosphorylation sites) from the rapamycin-treated (Light) and control (Heavy) TSC2−/− cells (B) Domain structure of mTOR and conservation of S2478 and S2481 across different species. Sequences, from top to bottom: SEQ ID NO: 1728 to SEQ ID NO: 1735, respectively. (C) Immunoblot experiments showing phospho-mTOR at S2481 is inhibited by acute rapamycin treatment. TSC2−/− cells were starved and then treated with 20 nM rapamycin for the indicated times. (D) mTOR kinase inhibitor pp 242 completely ablated mTOR phosphorylation at S2481. TSC2−/− cells were starved and were then treated with 1 pp 242 for two hours.

FIG. 8. Identification of rapamycin-sensitive phosphorylation sites on Grb10. (A) MS/MS experiments identified that phosphorylation of residues S501/S503 of Grb10 is strongly inhibited by rapamycin. (B) Phosphorylation at S455/S458 is rapamycin-insensitive. Sequences, from top to bottom: SEQ ID NO: 1736 and SEQ ID NO: 1737, respectively.

FIG. 9. Phosphorylation of Grb10 at S501/S503 regulates its stability. (A) An antibody showed specificity towards Grb10 phosphorylation at S501/S503. Preincubation of the antibody with the blocking antibody completely eliminated the immunoreactivity. (B) Further validation of the phospho-specific antibody raised against the Grb10 S501/S503 sites. Grb10 WT, S501A/S503A (AA) and S501D/S503D (DD) were transfected into HEK239T cells and were probed with the antibody. Note that this antibody detected wild type (WT) Grb10, but neither of the mutant proteins. (C) Treatment of TSC2−/− cells with various mTOR kinase inhibitors led to Grb10 degradation. (D) Both rapamycin and NVP-BEZ235 treatment led to a decreased level of Grb10 in TSC1−/− cells. (E) Grb10 is highly overexpressed in TSC1−/− cells compared with their wild-type counterparts. (F) rapamycin treatment of TSC2−/− cells stably expressing wild type (WT) Grb10 led to lower levels of both endogenous and exogenous Grb10.

FIG. 10. Grb10 is involved in the feedback loop from mTORC1 to PI3K. (A) Overexpression of Grb10 in HEK293 cells inhibited PI3K activation. W, wild type (WT) Grb10, A, AA mutant and D, DD mutant. Knockdown of Grb10 in TSC2−/− cells led to IRS hyperphosphorylation after insulin stimulation. (B) Overexpression of Grb10 in HEK239 cells suppressed IRS tyrosine phosphorylation and PI3K recruitment after insulin stimulation. HA-tagged Grb10 was transfected and IRS2 immunoprecipitates were analyzed. Phosphorylation at Y612 of IRS was detected. (C) HA-tagged Grb10 was transfected and p85 immunoprecipitates were analyzed. (D) Knockdown of Grb10 in TSC2−/− cells led to increased IRS Y612 phosphorylation upon insulin stimulation. (E) Knockdown of Grb10 in TSC2−/− cells protected these cells against etoposide-induced apoptosis. Cells were starved overnight and were treated with 100 μM etoposide for the indicated times.

### DEFINITIONS

In the tables and the database, *, @, ̂ and # denote phosphorylation (Ser, Thr and Tyr), heavy Lys, heavy Arg and oxidation (Met), respectively. International Protein Index (IPI) reference numbers are given in some of the tables and the database provided herein. The IPI database can be accessed at the European Bioinformatics Institute homepage (www.ebi.ac.uk/), for example, at (www.ebi.ac.uk/IPI/IPIhelp.html). Each IPI database entry provided by accession number is incorporated herein by reference for disclosure of the respective proteins amino acid sequence and accompanying protein information.

The term “antibody,” as used herein, refers to an immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes:IgG, IgM, IgA, IgD, and IgE. Derivatives of the IgG class, however, are preferred in the present invention.

The term “antibody fragment,” as used herein, refers to any derivative of an antibody which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv, diabody, and Fd fragments. The antibody fragment may be produced by any means. For instance, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

Single-chain Fvs (scFvs) are recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility.

Diabodies are dimeric scFvs. The components of diabodies typically have shorter peptide linkers than most scFvs, and they show a preference for associating as dimers.

An Fv fragment is an antibody fragment which consists of one V_{H} and one V_{L} domain held together by noncovalent interactions. The term dsFv is used herein to refer to an Fv with an engineered intermolecular disulfide bond to stabilize the V_{H}-V_{L} pair.

A F(ab′)₂ fragment is an antibody fragment essentially equivalent to that obtained from immunoglobulins (typically IgG) by digestion with an enzyme pepsin at pH 4.0-4.5. The fragment may be recombinantly produced.

A Fab fragment is an antibody fragment essentially equivalent to that obtained by reduction of the disulfide bridge or bridges joining the two heavy chain pieces in the F(ab′)₂ fragment. The Fab′ fragment may be recombinantly produced.

A Fab fragment is an antibody fragment essentially equivalent to that obtained by digestion of immunoglobulins (typically IgG) with the enzyme papain. The Fab fragment may be recombinantly produced. The heavy chain segment of the Fab fragment is the Fd piece.

The term “binding agent”, as used herein, refers to an agent binding a target molecule, for example, a polypeptide comprising a phosphorylation site provided herein, with high specificity. Examples of binding agents are antibodies, antibody fragments, aptamers, and adnectins.

The term “phosphosensitive”, as used herein in the context of a binding agent, refers to a binding agent that specifically binds to a phosphorylation site, for example, a phosphorylation site provided herein, in either the phosphorylated or non-phosphorylated state. In some embodiments, a phosphosensitive binding agent provided herein binds to the phosphorylation site in its phosphorylated state, but does not significantly bind the phosphorylation site in a non-phosphorylated state. Phosphosensitive binding agents, for example, phosphosensitive antibodies or antibody fragments, accordingly, allow for the detection of phosphorylation at a specific phosphorylation site.

The term “cancer”, as used herein, refers to a malignant neoplastic disease. Most cancers are characterized by hyperproliferation of a cell population. In some embodiments, a cancer manifests as a solid tumor. In some embodiments, a cancer manifests as a liquid tumor. Non-limiting examples of cancers include carcinomas (derived from epithelial cells, e.g., some forms of breast, prostate, lung and colon cancer), sarcomas (derived from connective tissue or mesenchymal cells), lymphoma and leukemia (derived from hematopoietic cells) and seminomas (derived from germ cells).

The term “cancer cell”, as used herein, refers to a malignant neoplastic cell. In some embodiments, a cancer cell is part of a neoplastic cell population. In some embodiments, a cancer cell is a cell of a solid tumor. In some embodiments, a cancer cell is a cell of a liquid tumor. In some embodiments, a cancer cell carries a mutation that affects regulation of cell cycle control. In some embodiments, a cancer cell is a cell obtained from a tumor in a subject.

The term “candidate agent”, as used herein refers to a molecule to be tested for a specific property, for example, for its ability to inhibit mTOR kinase activity. In some embodiments, a candidate agent is a small molecule. In some embodiments, a candidate agent is a polypeptide or protein. In some embodiments, a candidate agent is a binding agent. In some embodiments, a candidate agent is a nucleic acid.

The term “determining a level of expression”, as used herein, refers to performing an assay to determine the level of a gene product expressed in a cell or tissue, for example, in a cancer cell or tumor tissue. In some embodiments, the assay includes obtaining a cell from a subject, for example, by biopsy. In some embodiments, the gene product is a transcript, for example, an mRNA. In other embodiments, the gene product is a protein, for example, an mTOR substrate disclosed herein, or a protein comprising a phosphorylation site disclosed herein. In some embodiments, the gene product is a protein that is phosphorylated at a specific phosphorylation site. In some embodiments, the gene product is a protein that is not phosphorylated at a specific phosphorylation site. Methods, assays, and reagents to determine the level of a gene product in a cell or tissue are described herein and are well known to those of skill in the art. See, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Methods to determine transcript levels include, for example, RT-PCR, northern blot, in situ hybridization, microarray assays, and massive parallel sequencing assays. Methods to determine protein levels include, for example, western blot, immunohistochemistry, ELISA, protein array assays, and mass spectrometry.

The term “high risk of expressing an elevated level of PI3K, Akt, and/or MAPK activity”, as used herein in the context of cells, refers to a condition in a cell that is likely to result in upregulation of PI3K, Akt, and/or MAPK kinase activity, when the cell is contacted with a therapeutic agent, for example, with an mTOR inhibitor. The term “risk” is used interchangeably with the term “likelihood” in this context. Such risk can be conferred through feedback-relief triggered by the therapeutic target. For example, in some embodiments, a cell, for example, a cell derived from a tumor in a subject, is determined to have a high likelihood of expressing an elevated level of PI3K, Akt, and/or MAPK, if it is established that the cell highly expresses a feedback-mediator, for example, phosphorylated Grb10, that limits PI3K, Akt, and/or MAPK expression. If the feedback-mediator is a known substrate of an oncogenic kinase, for example, if the feedback-mediator is the mTOR kinase substrate Grb10, then the cell is likely to exhibit feedback relief upon administration of an inhibitor of the kinase, for example, an mTOR kinase inhibitor, which, in turn, may increase the cell's proliferation and/or survival capacity.

The term “inhibitor” as used herein in the context of kinases, refers to a molecule that inhibits the activity of a kinase. In some embodiments, an inhibitor diminishes the catalytic activity of a kinase. In some embodiments, an inhibitor abolishes the catalytic activity of a kinase. In some embodiments, the inhibitor is a small molecule. In some embodiments, the inhibitor is a nucleic acid or a polypeptide. In some embodiments, the inhibitor is a binding agent. In some embodiments, a kinase inhibitor effects inhibition by down-regulating expression of the kinase. In some embodiments, a kinase inhibitor effects inhibition by binding the kinase and interfering with the kinase reaction. In some embodiments, a kinase inhibitor is an allosteric kinase inhibitor. The term “allosteric kinase inhibitor” refers to a kinase inhibitor, for example, a small molecule, that binds its target kinase, wherein the binding of the inhibitor results in an allosteric change in the kinase molecule, leading to diminished kinase activity. Allosteric changes leading to diminished kinase activity can be changes resulting in a reduction of the kinase's ability to bind a substrate molecule, or changes resulting in a reduction of the kinases ability to transfer a phosphate group to a substrate molecule. In some embodiments, the kinase inhibitor is a catalytic kinase inhibitor. In some embodiments, a catalytic inhibitor, which itself cannot partake in a kinase reaction, competes with a kinase substrate for binding. In some embodiments, a catalytic kinase inhibitor is an ATP-competitive inhibitor.

The term “effective amount”, as used herein in the context of kinase inhibitors, refers to an amount of kinase inhibitor sufficient to achieve a measurable inhibition of kinase activity, or an amount of kinase inhibitor sufficient to achieve a clinically desirable outcome. For example, an effective amount of the mTOR inhibitor rapamycin is, in some embodiments, an amount of rapamycin that, when administered to a cell or tissue, results in a measurable decrease in mTOR kinase activity in the cell or tissue. In some embodiments, an effective amount of a kinase inhibitor is an amount that is effective to reduce the activity of the target kinase in a cell or tissue to less than 75%, less than 50%, less than 40%, less than 30%, less than 25%, less than 20%, less than 10%, less than 5%, less than 2.5%, less than 2%, or less than 1% of the activity in the cell or tissue not treated with the inhibitor. Assays for measuring kinase activity are well known in the art and described in more detail elsewhere herein. In some embodiments, an effective amount of a kinase inhibitor, for example, of rapamycin, is an amount that, when administered to a subject, for example, a subject having a cancer with elevated mTOR activity, results in a clinically desirable outcome. In some embodiments, a clinically desirable outcome is reversal of a disease, for example, a decrease in neoplastic or malignant cell number, tumor size, or cell proliferation rate, for example, in a subject having a proliferative disease, e.g., a neoplastic disease or a cancer, or a delay in the progression of a disease, for example a progression from one tumor stage to the next, or from a benign to a malignant neoplastic disease.

The term “mTOR kinase inhibitor” as used herein, refers to a molecule inhibiting a kinase activity of the mTOR kinase. Examples of mTOR kinase inhibitors are mTORC1 kinase inhibitors and mTORC1/2 inhibitors. Examples of mTORC1 kinase inhibitors are rapamycin and rapamycin analogs (e.g., Ridaforolimus, Sirolimus or Everolimus). Examples of mTORC1/2 inhibitors are PP242, PP30, AZD8055, OSI-027, WYE354, INK-128, XL388, and torin1. Further examples of mTOR inhibitors are inhibitors that target mTOR and an additional kinase or additional kinases, for example, dual PI3K/mTOR kinase inhibitors (e.g., NVP-BEZ235, BGT-226, XL-765, GSK2126458, or SF1126).

The term “PI3K kinase inhibitor”, as used herein, refers to a molecule that inhibits PI3K kinase activity. Examples of PI3K inhibitors are ZSTK474, TGX221, GDC0941, LY294002, XL147, PX147, BKM120, GSK 615, CAL101, and PX-866.

The term “mutation”, as used herein, refers to a change in a gene sequence, for example, a deletion, insertion, inversion, transposition, or substitution. In some embodiments, the mutation results in a change of the expression level of the gene product encoded by the respective gene. In some embodiments, a mutation is a mutation in a gene involved in an mTOR signaling pathway, for example, a gene encoding a protein that regulates, directly or indirectly, mTOR kinase activity. Examples of genes involved in an mTOR signaling pathway are Ras, Raf, MAPK, RSK, receptor tyrosine kinases, PI3K (Phosphoinositide 3-kinase), PTEN (phosphatase and tensin homolog), Akt (Protein Kinase B), TSC1/2 (Tuberous sclerosis protein 1, Tuberous sclerosis protein 2, respectively), MEK (Dual specificity mitogen-activated protein kinase kinase 1, MAPK21), LKB (, and NF2 (Neurofibromatosis 2). Methods to determine whether a cell carries a mutation in a gene are well known to those of skill in the related arts.

The term “phosphoproteomic profile”, as used herein, refers to a dataset comprising information regarding the level of phosphorylation of a plurality of phosphorylation sites in a biological sample, for example, a proteinaceous sample derived from a cell or tissue sample. Phosphoproteomic profiles of multiple samples can be compared and similarities and dissimilarities in such profiles can be detected and quantified by methods well known to those of skill in the art, including, but not limited to, supervised and non-supervised learning, hierarchical clustering, nearest neighbor analysis. In some embodiments, a phosphoproteomics profile of a clinical sample, for example, of a sample derived from a malignant cell or tissue sample of a subject is compared to a reference sample from healthy cells or tissue, for example, to determine aberrations in protein phosphorylation in the malignant cell or tissue sample. In some embodiments, a phosphoproteomics profile of a clinical sample at issue is compared to phosphoproteomics profiles of clinical samples of known character, for example, to classify the clinical sample at issue. Phosphoproteomic profiles can be classified by methods well known in the art, including the building and application of predictors for the classification.

The term “phosphorylation level”, as used herein, refers to the proportion of phosphorylated polypeptides carrying a certain phosphorylation site in a sample relation to all polypeptides carrying the phosphorylation site in the sample. For example, if the total number of polypeptides carrying a specific phosphorylation site in a sample is 10, and 3 of these proteins are phosphorylated at that site, while 7 are not, then the phosphorylation level of this phosphorylation site in the sample would be 30%. Phosphorylation levels can be detected and quantified by methods known to those in the art, for example, by protein detection using phosphosensitive binding agents, e.g., phosphosensitive antibodies or antibody fragments. Most protein quantitation methods known to those of skill in the art do not allow for precise molecule counting, so phosphorylation levels are often approximated based on estimations of the number or fractions of phosphorylated proteins in a sample, for example, after western blot or protein microarray analysis using phosphosensitive antibodies or antibody fragments.

The term “phosphorylation site”, as used herein, refers to an amino acid residue within an amino acid sequence, or motif, that can be phosphorylated, for example, by a kinase targeting the respective site. A phosphorylation site is a substrate of a kinase if it can be phosphorylated by that kinase. For example, 5421 and S432 are phosphorylation sites in Grb10 isoform 3 within the motif MSNILSS*QS*PLHPSTLNAVIHR (SEQ ID NO: 4), and are both mTOR substrates, as described elsewhere herein. mTOR phosphorylation sites can further be classified into three classes, as used herein. The term “class I phosphorylation site”, as used herein, refers to a phosphorylation site that is targeted by rapamycin-sensitive mTORC1 activity. The term “class II phosphorylation site,” as used herein, refers to a phosphorylation site that is targeted by rapamycin-insensitive mTORC1 and/or mTORC2 activity. The term “class III phosphorylation site,” as used herein, refers to a phosphorylation site that is targeted by rapamycin-sensitive mTOR activity, rapamycin-insensitive mTOR activity, or both.

The term “proliferative disease,” as used herein, refers to any disease in which cell or tissue homeostasis is disturbed in that a cell or cell population exhibits an abnormally elevated proliferation rate. Proliferative diseases include hyperproliferative diseases, such as pre-neoplastic hyperplastic conditions and neoplastic diseases. Neoplastic diseases are characterized by an abnormal proliferation of cells and include both benign and malignant neoplasias. Malignant neoplasia is also referred to a s cancer.

The term “reference”, interchangeably used with the term “control” herein, refers to a value, sample, or parameter that serves as a baseline for comparing a value, sample, or parameter of interest to. The use of a reference can be of value in many methods that allow for the detection of absolute levels, for example, of expression, phosphorylation, or protein binding, and is essential in methods that yield semi-quantitative or relative results. Comparing an assay result obtained for a cell or tissue in question, for example, a cell or tissue obtained from a tumor in a subject, to a reference result allows a determination whether the result is abnormal. Depending on their nature, abnormal results can support the inference of specific molecular or cellular aberrations and, in some embodiments, a selection of a course of treatment over another. In some embodiments, a reference value is obtained from cells of the same cell type or the same tissue of origin as the cell in question. For example, in some embodiments, a diseased and a healthy cell is obtained from a subject, and the cells are assayed by methods provided herein in parallel. The value observed in the healthy cell, for example, a level of phosphorylation of a protein then typically serves as the reference level to which the level observed in the cell in question is compared. In other embodiments, the reference level is an average level observed or expected in normal cells. In some embodiments, the reference level is a range typically observed in healthy cells. A suitable reference depends, of course, on the type of assay and sample involved. A suitable reference for a given assay or sample will readily be apparent to those of skill in the art. The following list of exemplary references is for illustration only, since the invention is not limited in this respect.

In some embodiments, a suitable reference level, for example, a suitable reference phosphorylation or expression level, is a level observed or expected in a healthy cell or tissue of the same type as the cell or tissue in question. In some embodiments, for example, in some embodiments where a tumor biopsy is assayed, a suitable reference level is a level observed or expected in healthy cells or tissue of the same tissue the tumor originated from, or in cells or tissue adjacent to the tumor. In some embodiments, for example, in some embodiments where a cell is assayed, a reference cell is a healthy cell that is of the same cell type or tissue of origin as the cell in question. In some embodiments, a reference cell is a cell exhibiting normal mTOR kinase activity. In some embodiments, a reference cell is a malignant cell of known phenotype, for example, a malignant cell known to exhibit elevated mTOR kinase activity, a cell known to exhibit elevated mTORC1 kinase activity, or a cell known to exhibit elevated mTORC1/2 kinase activity. In some embodiments, a reference cell is a malignant cell of known phenotype, for example, a rapamycin-sensitive cancer cell or a rapamycin-sensitive cancer cell.

In some embodiments, a suitable reference level is an average level calculated or approximated from historic data. In some embodiments, a reference level is a level obtained from a reference cell or tissue assayed in parallel to the cell or tissue in question. In some embodiments, a level obtained from a cell or tissue sample is determined to be different (higher or lower) than the reference level, if it is statistically significantly different (higher or lower) than the reference level. In some embodiments, a level obtained from a cell or tissue sample is determined to be higher than the reference level, if the level is at least about 1.25-fold, at least about 1.5-fold, at least about 2-fold, at least about 3-fold, at least about 5-fold, or at least about 10-fold the reference level. In some embodiments, a level obtained from a cell or tissue sample is determined to be lower than the reference level, if it is less than about 0.75-fold, less than about 0.70-fold, less than about 0.60-fold, less than about 0.50-fold, less than about 0.40-fold, less than about 0.30-fold, less than about 0.25-fold, less than about 0.10-fold, less than about 0.05-fold, or less than about 0.01-fold the reference level. In some embodiments, a level obtained from a cell or tissue is determined to be substantially similar to the reference level, if it is not statistically significantly different from the reference level. In some embodiments, a level obtained from a cell or tissue is determined to be substantially similar to a reference level, if it is within the range of 0.75-fold to 1.25-fold of the reference level. In some embodiments, a profile comprising a plurality of levels obtained from a cell or tissue sample is determined to be substantially similar to a reference profile, if the profiles cluster together in a clustering analysis. In some embodiments, a multi-value profile obtained from a cell or tissue is determined to be substantially similar to a reference profile, if the correlation coefficient between the profiles is at least 0.75, at least 0.8, at least 0.85, at least 0.9, at least 0.91, at least 0.92, at least 0.93, at least 0.94, at least 0.95, at least 0.96, at least 0.97, at least 0.98, or at least 0.99.

The term “sample”, as used herein, refers to a biological sample. A biological sample typically comprises a cell or tissue, or biological material derived from a cell or tissue. Examples of biological samples are cell samples and tissue samples obtained from a subject, for example, from a subject having a cancer, cell or tissue cultures, and extracts or preparations obtained from any such samples, for example, protein extracts, isolated proteins or isolated nucleic acids obtained from such samples.

A “proteinaceous” sample is a sample comprising proteins or fragments of proteins. In some embodiments, a proteinaceous sample is a sample derived from a cell that includes proteins or fragments of proteins expressed in the cell. In some embodiments, the sample includes a lysed cell. In some embodiments, the sample includes proteins isolated from a cell. In some embodiments, the sample includes an isolated, enriched, or purified protein fraction from a cell, for example, a protein fraction obtained by methods described herein or known to those of skill in the art to separate nucleic acids, carbohydrates, and/or lipids from proteins or fractions of proteins. In some embodiments, the sample is prepared under conditions suitable for obtaining native proteins from a cell. In some embodiments, the sample is prepared under conditions that do not significantly affect native protein phosphorylation patterns. In some embodiments, the sample is prepared under conditions that do not significantly affect the antigenic structure of a phosphorylation site identified herein, for example, a phosphorylation site identified herein to be a target of mTOR kinase activity. In some embodiments, the sample is prepared under conditions suitable for obtaining denatured or fragmented proteins.

The term “subject,” as used herein, refers to an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a subject diagnosed to have a cancer. In some embodiments, the subject is a subject diagnosed to have a cancer that exhibits an elevated level of mTOR kinase activity. In some embodiments, the subject is a subject not diagnosed with a cancer.

The term “target,” as used herein in the context of kinases, refers to a substrate of a kinase. The term can, accordingly, refer to a protein (e.g., Grb10 is a target of mTOR kinase), to an amino acid sequence comprising a phosphorylation site (e.g., MNILSS*QS*PLHPSTLNAVIHR (SEQ ID NO: 5) or MNILGS*QS*PLHPSTLSTVIHR (SEQ ID NO: 6) are target sequences of mTOR), or to an amino acid residue (e.g. S421 and S423 of mouse Grb10, isoform 3, are targets of mTOR).

The term “tumor,” as used herein, refers to a neoplasm or a solid lesion formed by neoplastic cells. A tumor can be benign, pre-malignant, or malignant. In some embodiments, the tumor is a malignant tumor.

The term “treatment”, interchangeably used herein with the term “therapy,” as used herein, refers to a clinical intervention aimed to prevent or to ameliorate a disease or condition in a subject. In some embodiments, a treatment is aimed to ameliorate an existing condition, for example, a cancer in a subject. In some embodiments, a treatment is aimed to prevent a condition from occurring or from recurring. For example, in some embodiments, an mTOR kinase inhibitor is administered to a subject having a cancer exhibiting an elevated level of mTOR activity in order to inhibit cell proliferation in the malignant cells. For another example, in some embodiments, an mTOR kinase inhibitor is administered to a subject having a cancer exhibiting an elevated level of mTOR activity after a malignant tumor has been removed from the subject, in order to prevent or delay recurrence of the disease. The foregoing examples are non-limiting, and those of skill in the art will readily envision further scenarios of treatment as the invention is not limited in this respect. Some embodiments include a choice of treatment, referring to a selection of a clinical intervention from a number of alternatives, or to a design of a clinical intervention to meet the specific needs of an individual subject. In some embodiments, a choice of treatment involves the design of a personalized therapeutic approach for a subject having a cancer exhibiting elevated mTOR activity based on the results from diagnostic methods provided herein. For example, in some embodiments, a choice of treatment includes administering to a subject having cancer a specific mTOR inhibitor in combination with an additional kinase inhibitor targeting an mTOR feedback-inhibited pathway based on a determination that cancer cells in the subject exhibit high mTOR activity and elevated levels of a feedback-inhibitor targeted by mTOR in order to avoid feedback relief. In some embodiments, a choice of treatment includes the determination of an appropriate treatment. Some embodiments further include carrying out the selected treatment.

### DETAILED DESCRIPTION OF CERTAIN EMBODIMENTS

This invention relates to the identification of proteins and sites on those proteins that are phosphorylated by mTOR kinase. Some aspects of this invention relate to the surprising discovery that some proteins that are substrates of mTOR kinase regulate mTOR upstream signaling pathways, thus providing feedback regulatory circuits. Some aspects of this invention relate to methods of classifying cells, for example, neoplastic cells or cancer cells, based on the phosphorylation status of mTOR targets. Some aspects of this invention relate to methods of personalized treatment of subjects having a proliferative disease, for example, a neoplastic disease or a cancer, exhibiting elevated mTOR signaling based on the phosphorylation status of mTOR targets in the diseased cells. Other aspects of this invention relate to methods and materials for the generation of phosphoproteomics profiles that include phosphorylation data for a plurality of mTOR targets. Some aspects of this invention relate to classification of cells or tissues, for example, tumor cells or tissues, based on phosphoproteomic data, for example, based on phosphoproteomic profiles of the cells or tissues. Some aspects of this invention relate to methods of identifying mTOR kinase inhibitors by identifying compounds able to interfere with mTOR kinase activity towards one or more of the mTOR targets provided herein. Some aspects of this invention relate to the identification of Grb10 as a target of mTOR kinase activity, and to methods for determining the level of mTOR activity in a cell based on Grb10 phosphorylation status. Some aspects of this invention relate to the surprising discovery that Grb10 is not only a target of mTOR kinase activity, but also an inhibitor of PI3K kinase activity. Since PI3K is a positive regulator of mTOR kinase activity, Grb10 has been identified as a mediator of mTOR feedback inhibition herein. Other aspects of this invention relate to the surprising discovery that Grb10 and PTEN mutations are mutually exclusive in certain types of tumor cells. Accordingly, some aspects of this invention relate to the surprising discovery that Grb10 is a tumor suppressor and that Grb10 phosphorylation is a useful biomarker for determining mTOR kinase activity in cells, and particularly in tumor cells. Other aspects of this invention relate to methods and reagents useful for determining the phosphorylation status of a protein or phosphorylation site identified to be an mTOR target herein. For example, some aspects of this invention provide phosphosensitive antibodies or antibody fragments specifically binding an mTOR kinase target site identified herein in either the phosphorylated or the non-phosphorylated state, thus allowing for a determination of the phosphorylation state of such a site in a cell.

mTOR Target Proteins and Sites
Identification of mTOR Target Sites

Some aspects of this invention relate to the identification of proteins and phosphorylation sites thereon as downstream targets of mTOR kinase activity. Targets of mTOR kinase activity are provided in Tables 1 and 2.

Classification of mTOR Targets

Some aspects of this invention relate to the classification of mTOR target phosphorylation sites provided herein as class I (targets of rapamycin-sensitive mTORC1 kinase signaling), class II (targets of rapamycin-insensitive mTORC1 or mTORC2 signaling), and class III (targets of both rapamycin-sensitive and rapamycin-insensitive mTOR signaling) phosphorylation sites.

This classification is useful to determine the type(s) of mTOR signaling exhibited by a cell or tissue and can support a classification of cells that are targeted for therapy, for example, of cancer cells. For example, in some embodiments, the phosphorylation status of an mTOR target is determined in a cancer cell obtained from a subject with a cancer exhibiting elevated mTOR kinase activity. Classification of the type of mTOR signaling can, in some embodiments, be a basis for the selection of an appropriate therapeutic approach. For example, if it is determined that a class I target of mTOR signaling is phosphorylated, then the elevated mTOR kinase activity is rapamycin-sensitive mTORC1 kinase activity. In some such embodiments, a method of treatment is then typically selected that includes administration of an inhibitor of rapamycin-sensitive mTORC1 signaling, for example, rapamycin or a rapamycin analog.

To give but another example: if it is determined that a class II target of mTOR signaling is phosphorylated, then the elevated mTOR kinase activity is rapamycin-insensitive mTORC1 or mTORC2 kinase activity. In some such embodiments, a method of treatment is then typically selected that includes administration of an mTORC1/2 inhibitor as provided herein. In some embodiments where class I targets of mTOR signaling are found to not be phosphorylated, a method of treatment is typically selected that does not include administration of an mTORC1 inhibitor.

To give a third example: if it is determined that a class III target of mTOR signaling is phosphorylated, then a method of treatment is selected that includes administration of an mTORC1 inhibitor and an mTORC1/2 inhibitor as provided herein. In some embodiments, a method of treatment that includes administering an mTORC1 inhibitor or an mTORC1/2 inhibitor alone may be inappropriate.

Further, some aspects of this invention provide methods to analyze gene ontology distributions in sets of mTOR targets, for example, in order to determine whether a specific signaling pathway is targeted by mTOR in a given cell, or a given therapeutic or experimental scenario.

Exemplary Proteins Identified in the Rapamycin Phosphoproteomic Screen as Targets of mTORC1 Signaling.

The phosphoproteomic screen described herein identified numerous proteins as mTOR targets, for example, as mTORC1 targets. As described in more detail elsewhere herein, the expression, expression level, phosphorylation, or phosphorylation level of one or more of such mTOR target proteins, for example, the proteins described in any of tables 1-3, or the level of phosphorylation of their respective phosphorylation sites, is employed, in some embodiments, as a biomarker for monitoring or diagnosing disease. In some embodiments, the mTOR target proteins disclosed herein, for example, the mTORC1 target proteins described in any of tables 1-3, are used in pharmaceutical screens as drug targets for the development of drugs modulating mTOR pathway downstream effects. Some exemplary mTORC1 target proteins identified herein are described in more detail below. These descriptions of exemplary mTOR target proteins are for illustration of some aspects of this disclosure and are not meant to limit the scope of the invention. Grb10, one of the mTORC1 protein targets identified in the phosphoproteomics screen, is described in more detail elsewhere herein. Briefly, Grb10, also known as growth factor receptor-bound protein 10, is stabilized by mTORC1-mediated phosphorylation, which, in turn, results in feedback inhibition of the PI3K and MAPK pathways. This is consistent with Grb10 expression being frequently downregulated in a variety of cancers. Grb10 is a tumor suppressor with relevance across a broad spectrum of cancer subtypes, and the identification of Grb10 as an mTORC1 substrate links mTORC1 activity to cancers with aberrantly low Grb10 expression or stability. Accordingly, some embodiments provide Grb10 phosphorylation as a biomarker for mTORC1 activity in a cell, tissue, or sample of interest. Methods of using Grb10 expression or phosphorylation assays to monitor mTORC1 activity in a cell, tissue, or sample of interest are also provided. For example, some embodiments provide a method of monitoring the efficacy of a drug on mTORC1 activity in a cell, tissue, or sample, by measuring Grb10 expression or phosphorylation levels. In some embodiments, such assessments are used to diagnose or monitor a disease associated with aberrant levels of mTORC1 activity, for example, cancer, or to adjust a drug dosage, for example, to the minimal dosage necessary to achieve a target mTORC1 activity level, or to choose a suitable drug modulating mTORC1 activity from a plurality of drugs, for example, the drug that shows the highest efficacy at a given dose.

Some of the mTORC1 protein targets identified herein are potential regulators of gene expression. For example, FOXK1 (Forkhead box protein K1), also known to those of skill in the art as MNF, or MNF (myocyte nuclear factor)-beta, is a winged-helix protein expressed selectively and transiently in myogenic precursor cells of the heart and skeletal muscles, and collaborates with proteins of the mammalian Sin3 (mSin3) family to repress transcription. Mutated forms of MNF-beta that fail to bind mSin3 are defective in transcriptional repression and in negative growth regulation, an overexpression phenotype revealed in oncogenic transformation assays. These phenotypic traits associated with MNF-beta dysfunction are consistent with the mTORC1 phenotype.

For another example, ZEB2 (Zinc finger E-box-binding homeobox 2) belongs to the ZEB family of zinc finger transcription factors, which are essential regulators of gene expression during normal embryonic development. ZEB proteins induce epithelial to mesenchymal transition (EMT), a process in which epithelial cells become migratory mesenchymal cells. E-cadherin is a major target gene of ZEB transcriptional repressors, and e-cadherin downregulation is considered a hallmark of EMT. In recent years, the involvement of the ZEB proteins in pathological contexts has been documented as well. For example, ZEB proteins play an important role in mediating Ras-induced EMT in breast epithelial cells. Mutations in ZEB encoding genes cause severe syndromic malformations, and are implicated in malignant tumor progression. Without wishing to be bound by theory, ZEB2 is believed to be a critical target in lymphangioleiomyomatosis (LAM) that represents the point of convergence of the mTORC1 and ERK-MAP kinase pathways that are critical to this disease.

For another example, NDRG3 is the downstream target of N-Myc. Phosphorylation of NDRG3 links mTOR signaling pathway to Myc activity as an oncogenic transcription factor.

Accordingly, some embodiments provide FOXK1, ZEB2, and/or NDRG3 phosphorylation as a biomarker for mTORC1 activity in a cell, tissue, or sample of interest. Methods of using FOXK1, ZEB2, and/or NDRG3 expression or phosphorylation assays and levels useful for monitoring mTORC1 activity in a cell, tissue, or sample of interest are also provided. For example, some embodiments provide a method of monitoring the efficacy of a drug on mTORC1 activity in a cell, tissue, or sample, by measuring FOXK1, ZEB2, and/or NDRG3 expression or phosphorylation levels. In some embodiments, such assessments are used to monitor or diagnose a disease associated with aberrant mTORC1 activity, as manifest by aberrant FOXK1, ZEB2, and/or NDRG3 phosphorylation level(s), and/or to adjust a drug dosage, for example, to the minimal dosage necessary to achieve a target mTORC I activity level, or to choose a suitable drug modulating mTORC1 activity from a plurality of drugs, for example, the drug that exhibits the highest efficacy at a given dose.

Some of the mTORC1 protein targets identified herein are regulators of mRNA synthesis, mRNA processing, and protein synthesis. This is consistent with the role mTORC1 activity plays in the control of cell growth, which, if improperly regulated, can contribute to tumor genesis and/or growth. For example, LARP1 binds mRNA in vitro via both the La motif and the LARP1 domain. LARP-1 also down-regulates the Ras-MAPK pathway. Accordingly, without wishing to be bound by any particular theory, LARP1 phosphorylation represents an mTORC1-dependent regulation of this feedback loop. The LARP-1 protein colocalizes with P bodies, which function in RNA degradation, and it is believed that the cluster of LARP-1 homologs functions to control the expression of key developmental regulators. Without wishing to be bound by any particular theory, some aspects of this invention provide that LARP-1 functions in P-bodies to attenuate the abundance of Ras-MAPK pathway-regulated mRNAs.

For another example, EDC3 is associated with an mRNA-decapping complex required for removal of the 5′-cap from mRNA prior to its degradation from the 5″-end.

For another example, SRPK2 is involved in SR protein phosphorylation, which influences other aspects of mRNA metabolism, such as splice site selection (alternative splicing), mRNA export, nonsense-mediated decay (NMD), and translation efficiency. Accordingly, some aspects of this disclosure provide that SRPK2 links mTOR/S6K signaling to SR protein activity.

Another example of an MTORC1 target identified herein that regulates mRNA synthesis and processing is CDK12-(CDC2-related kinase, arginine/serine-rich). CDK12 is involved in the regulation of alternative mRNA splicing. Without wishing to be bound by any particular theory, it is believed that, similar to SRPK2, CDK12 is also involved in regulating mRNA splicing by mTOR signaling. A CDK12-binding partner, cyclin-L1 (CCNL1), is also identified as an mTORC1 target protein herein. Without wishing to be bound by any particular theory, similar to SRPK and CDK12, CCNL1 may contribute to mRNA biogenesis and cell proliferation.

Accordingly, some embodiments provide LARP1, SRPK2, and/or CDK12 phosphorylation as a biomarker for mTORC1 activity in a cell, tissue, or sample of interest. Methods of using LARP1, SRPK2, and/or CDK12 expression or phosphorylation levels to monitor mTORC1 activity in a cell, tissue, or sample of interest are also provided. For example, some embodiments provide a method of monitoring the efficacy of a drug on mTORC1 activity in a cell, tissue, or sample, by measuring LARP1, SRPK2, and/or CDK12 expression or phosphorylation levels. In some embodiments, such assessments are used to monitor or diagnose a disease associated with aberrant mTORC1 activity, as manifest by aberrant LARP1, SRPK2, and/or CDK12 phosphorylation level(s), and/or to adjust a drug dosage, for example, to the minimal dosage necessary to achieve a target mTORC1 activity level, or to choose a suitable drug modulating mTORC1 activity from a plurality of drugs, for example, the drug that exhibits the highest efficacy at a given dose.

Another example of an mTORC1 target protein identified herein is MIB1, an E3 ubiquitin-protein ligase that has been reported to ubiquinate Notch, which leads to the degradation of Notch. Accordingly, some aspects of this disclosure provide that mTORC1 activity is linked to Notch signaling via phosphorylation of MIB1. Yet another example of an mTORC1 target protein identified herein is IBTK (Isoform 2 of Inhibitor of Bruton tyrosine kinase), which is a protein tyrosine kinase implicated in the primary immunodeficiency disease X-linked agammaglobulinemia (Bruton agammaglobulinemia). IBTK is activated upon binding to PIP3 generated as a result of PI3K activation. Without wishing to be bound by any particular theory, phosphorylation of IBTK affects IBTK's ability to inhibit PI3K kinase activity. Accordingly, some embodiments of this invention are based on the recognition that there is a link between the mTORC1 signaling system and the regulation of tyrosine phosphorylation.

Accordingly, some embodiments provide MIB1 and/or IBTK phosphorylation as a biomarker for mTORC1 activity in a cell, tissue, or sample of interest. Methods of using MIB1 and/or IBTK expression or phosphorylation assays to monitor mTORC1 activity in a cell, tissue, or sample of interest are also provided. For example, some embodiments provide a method of monitoring the efficacy of a drug on mTORC1 activity in a cell, tissue, or sample, by measuring MIB1 and/or IBTK expression or phosphorylation levels. In some embodiments, such assessments are used to monitor or diagnose a disease associated with aberrant mTORC1 activity, as manifested by aberrant MIB1 and/or IBTK phosphorylation level(s), and/or to adjust a drug dosage, for example, to the minimal dosage necessary to achieve a target mTORC1 activity level, or to choose a suitable drug modulating mTORC1 activity from a plurality of drugs, for example, the drug that exhibits the highest efficacy at a given dose.

Representative protein sequences of Grb10, FOXK1, ZEB2, NDRG3, LARP1, SRPK2, CDK12, MIB1, IBTK, and other proteins identified herein to be mTORC1 substrates, can be found under the respective database entries provided in the tables and the database provided herein, and, for example, in the NCBI database under accession numbers NP_{—}001171100.1 (growth factor receptor-bound protein 10 (Grb10) isoform 2, Mus musculus, SEQ ID NO: 1720), NP_{—}034475.2 (growth factor receptor-bound protein 10 (Grb10) isoform 1, Mus musculus, SEQ ID NO: 1721), NP_{—}001001550.1 (growth factor receptor-bound protein 10 (Grb10) isoform c, SEQ ID NO: 7), NP_{—}005302.3 (Grb10 isoform a, SEQ ID NO: 8) and NP_{—}001001549.1 (Grb10 isoform b, SEQ ID NO: 9); NP_{—}001032242.1 (FOXK1, SEQ ID NO: 10); NP_{—}001165124.1 and NP_{—}055610.1 (ZEB2, SEQ ID NO: 11 and SEQ ID NO: 12, respectively); NP_{—}071922.2 and NP_{—}114402.1 (NDRG3, SEQ ID NO: 13 and SEQ ID NO: 14, respectively); NP_{—}056130.2 (LARP1, SEQ ID NO: 15); NP_{—}872633.1 and NP_{—}872634.1 (SRPK2, SEQ ID NO: 16 and SEQ ID NO: 17, respectively); NP_{—}055898.1 and NP_{—}057591.2 (CDK12, SEQ ID NO: 18 and SEQ ID NO: 19, respectively); NP_{—}065825.1 (MIB1, SEQ ID NO: 20); and NP_{—}056340.2 (IBTK, SEQ ID NO: 21). These database entries are incorporated herein by reference for disclosure of representative sequences of the respective proteins.

Feedback Circuitry Involving mTOR Targets

Some aspects of this invention relate to the surprising discovery that some mTOR target proteins are involved in feedback loops of mTOR signaling, for example, by inhibiting upstream modulators of mTOR signaling, such as PI3K, Akt, and MAPK, after being phosphorylated as a result of mTOR kinase activity. Some aspects of this invention relate to the surprising discovery that relief of feedback inhibition of cellular pro-survival, pro-proliferation signaling by mTOR downstream targets, for example, by Grb10, can contribute to rapamycin resistance or even clinically detrimental outcome in the treatment of neoplastic disease (e.g., cancer) with mTOR inhibitors. Some aspects of this invention relate to the identification, for the first time, of Grb10 as a target of mTOR kinase activity. Grb10, growth factor receptor-bound protein 10, is a protein well known to those of skill in the art. Representative protein sequences of Grb10 can be found under the respective database entries provided in the tables and the database provided herein, and, for example, in the NCBI database under accession numbers NP_{—}001001550.1 (growth factor receptor-bound protein 10 isoform c), NP_{—}005302.3 (isoform a) and NP_{—}001001549.1 (isoform b). These database entries are incorporated by reference for disclosure of representative Grb10 sequences.

### Feedback Inhibition of PI3K by Grb10

Some aspects of this invention relate to the surprising discovery that Grb10 is not only a target of mTOR kinase activity, but also a mediator of mTOR feedback inhibition and, thus, a tumor suppressor gene. As described in more detail elsewhere herein, mTOR-mediated phosphorylation of Grb10 results in stabilization and/or prevention of degradation of Grb10. Grb10, in turn, inhibits PI3K kinase activity. PI3K kinase activity targets mTOR and activates mTOR signaling. Accordingly, as provided by some aspects of this invention, treatment of a cancer exhibiting an elevated level of Grb10 phosphorylation can lead to relief of the Grb10-mediated feedback inhibition of PI3K. PI3K signaling, however, promotes proliferation and survival, and aberrant PI3K activity is known to contribute to carcinogenesis. The discovery that some mTOR targets identified herein, for example, Grb10, mediate a feedback inhibition of pro-survival and pro-proliferation signaling (e.g. PI3K, Akt, and MAPK signaling) in cancer cells, could explain why some types of cancer exhibiting elevated levels of mTOR activity do not respond favorably to treatment with an mTORC1 inhibitor (e.g., rapamycin) alone. The mTORC1 inhibitor may cause relief of the feedback inhibition which may, in turn result in sustained survival and proliferation, and even an increase or acceleration in disease progression or recurrence, as observed in some clinical trials of cancer treatment with mTOR inhibitors.

Also provided herein are methods of treating a cancer determined to exhibit an elevated level of Grb10 phosphorylation. In some embodiments, the method comprises administering to the subject an mTOR inhibitor and, additionally, an agent that stabilizes Grb10 or that inhibits the degradation of Grb10. In some embodiments, the method comprises effecting an inhibition of Grb10 degradation by administering a compound that inhibits Grb10 degradation. In some embodiments, the compound that inhibits Grb10 degradation is a ubiquitin ligase inhibitor, for example, a ubiquitin E3 ligase inhibitor. In some embodiments, the method comprises administering to the subject an mTOR inhibitor and, additionally, a PI3K inhibitor. PI3K inhibitors are known to those of skill in the art and described in more detail elsewhere herein.

### Diagnostic Methods

### Classification of Cancer Cells Based on Grb10 Phosphorylation

Some aspects of this invention provide methods for the classification of a neoplastic disease, for example, a cancer, based on the phosphorylation status of Grb10. In some embodiments, a method is provided that includes obtaining a neoplastic cell from a subject diagnosed to have a cancer, determining the level of Grb10 phosphorylation in the cell, and comparing the level of Grb10 phosphorylation to a reference level. In some embodiments, if the level of Grb10 phosphorylation in the cell is higher than the reference level, then the cell is determined to exhibit an elevated level of mTOR kinase activity. In some embodiments, the cell is determined to exhibit an elevated level of mTORC1 kinase activity based on an elevated level of Grb10 phosphorylation.

Methods of determining a level of protein phosphorylation, for example, of Grb10 phosphorylation, are well known to those of skill in the art. Exemplary methods as well as materials useful for such methods are described, for example, in Marjo de Graauw (Editor), Phospho-Proteomics: Methods and Protocols (Methods in Molecular Biology), Humana Press; 1 edition (2009), ISBN-10: 1603278338. Phosphosensitive binding agents, for example, phosphosensitive antibodies, are provided herein, are commercially available, or can be obtained by those of skill in the art with no more than routine experimentation.

Such methods include, for example, detection of phosphorylated protein with a phosphosensitive antibody and comparison of the amount of phosphorylated protein detected to the total amount of the protein in a sample. Other methods for quantitative or semi-quantitative detection of phosphorylated protein will be apparent to those of skill in the art and the invention is not limited in this respect.

In some embodiments, a method is provided that includes selecting a method of treatment of a neoplastic disease, for example, a cancer based on a cell from a subject having the disease exhibiting an elevated level of Grb10 phosphorylation. In some embodiments, an elevated level of Grb10 phosphorylation is indicative of an elevated level of rapamycin-sensitive mTORC1 kinase activity in the cell. In some embodiments, a method of treatment is chosen based on the cell exhibiting an elevated level of Grb10 phosphorylation that includes administration of an mTORC1 inhibitor, for example, a rapamycin or a rapamycin analog.

Classification of Cancer Cells Known to Exhibit Elevated Levels of mTOR Activity

Some aspects of this invention relate to the discovery that the mTOR target Grb10 is an inhibitor of the mTOR upstream regulators, PI3K, Akt, and MAPK, and, thus, can be characterized as a tumor suppressor gene and an mTOR feedback inhibitor. Some aspects of this invention provide a method for selecting a treatment of a cancer known to exhibit an elevated mTOR kinase activity based on the expression level of the mTORC1 target Grb10. In some embodiments, the method comprises obtaining a cancer cell from a subject diagnosed to have a cancer exhibiting an elevated level of mTOR activity, determining the level of Grb10 expression in the cell, and comparing the level of Grb10 expression to a reference level. In some embodiments, if the level of Grb10 expression in the cancer cell is higher than the reference level, then the cell is determined to exhibit a high likelihood of expressing an elevated level of PI3K, Akt, and/or MAPK activity upon being contacted with an mTORC1 inhibitor. In some embodiments, elevated expression of Grb10 supports the conclusion that upon treatment of the cell or a population of cells of the same type, mTORC1-dependent phosphorylation of Grb10 will be decreased or abolished, resulting in rapid ubiquitination and degradation of Grb10 protein, and, subsequently, in a relief of the Grb10-mediated feedback inhibition of PI3K, as described herein. This relief of feedback inhibition may, in some embodiments, result in elevated PI3K signaling. In some embodiments, elevated PI3K in a cancer cell is an undesirable response to treatment of a subject because it can lead to increased cell proliferation and/or survival, thus offsetting or even outweighing the beneficial effect of the administered mTORC1 inhibitor.

Measuring the expression level of an mTOR target protein can be achieved by using methods well known to those of skill in the art, including, but not limited to, protein expression assays, for example, immunostaining methods (e.g., western blot, protein microarray, immunohistochemistry, phosphoproteomic assays using phosphosensitive binding agents), ELISA, transcript expression assays, for example, RT-PCR, massive parallel sequencing assays, microarray assays, northern blot, or in situ hybridization. Other suitable methods will be apparent to those of skill in the art and the invention is not limited in this respect. See, for example, Sambrook et al., Molecular Cloning, second edition, Cold Spring Harbor Laboratory, Plainview, N.Y.; (1989); Ausubel et al., Current Protocols in Molecular Biology, Current Protocols (1989), and DNA Cloning: A Practical Approach, Volumes I and II (ed. D. N. Glover) IREL Press, Oxford, (1985); and Current Protocols in Molecular Biology, Wiley Publishing, accessible at www.currentprotocols.com; each of which is incorporated herein by reference.

In some embodiments, after a cancer cell is classified, a method of treatment of the subject from which the cell was obtained is selected based on the cancer cell exhibiting a high likelihood of expressing an elevated level of PI3K, Akt, and/or MAPK activity upon being contacted with an mTORC1 inhibitor. In some embodiments, the method of treatment comprises administering an effective amount of an mTOR kinase inhibitor and, additionally, an effective amount of an IGF1R, EGFR, PI3K, Akt, MEK, or RSK inhibitor, or a combination thereof. In some embodiments, the method of treatment comprises administering an effective amount of an mTOR kinase inhibitor and, additionally, of a compound stabilizing Grb10 to the subject. In some embodiments, the mTOR kinase inhibitor is an mTORC1 inhibitor, for example, rapamycin or a rapamycin analog. In some embodiments, the selected method of treatment is communicated to the subject, to a physician or other health care professional treating the subject. In some embodiments, the selected method of treatment is carried out.

Determining and Classifying mTOR Activity in a Cell Based on Phosphoproteomics

Some aspects of this invention provide methods for determining the level of mTOR activity in a cell based on analysis of the phosphorylation state of one or more mTOR targeted phosphorylation sites as provided herein. Some aspects of this invention provide methods to determine a class of mTOR signaling, for example, rapamycin-sensitive mTORC1 signaling, rapamycin-insensitive mTORC1 or mTORC2 signaling, or rapamycin-sensitive and/or rapamycin-insensitive mTOR signaling, in a cell based on an analysis of the phosphorylation state of a phosphorylation site as provided herein.

Methods useful for the determination of the phosphorylation level of a phosphorylation site in a cell are known to those of skill in the art. In some embodiments, such methods comprise obtaining a proteinaceous extract from the cell under conditions that allow for protein phosphorylation to be preserved with high fidelity. In some embodiments, such methods comprise contacting the cell extract with a phosphosensitive binding agent or with a plurality of phosphosensitive binding agents, for example, phosphosensitive antibodies or antibody fragments.

The invention provides phosphosensitive antibodies and antibody fragments to the phosphorylation sites described in the tables and the database. For example, phosphosensitive mTORC1 downstream effector antibodies, including, but not limited to, anti-phospho-NDRG3 (Ser331), anti-phospho-S501/503-Grb10, Anti-CDC2-related Kinase, Arg/Ser-Rich (Ser437), In some embodiments, antibodies provided herein are developed in rabbits. Other phosphosensitive antibodies are described elsewhere herein.

Phosphosensitive antibodies are useful in some embodiments to determine the phosphorylation level of one or more phosphorylation sites disclosed herein. One non-limiting example of such an embodiment is a phosphosensitive protein microarray assay. In some embodiments, the cell extract is contacted with a single phosphosensitive binding agent. In some embodiments, the cell extract is contacted with a plurality of binding agents in parallel. In some such embodiments, the cell is contacted with a microarray comprising a plurality of phosphosensitive binding antibodies or antibody fragments immobilized on a solid surface, for example, a glass surface. Phosphoproteomic assays, arrays, binding agents, and methods for sample preparation and analysis are well known in the art, and exemplary methods are described, for example, in Marjo de Graauw (Editor), Phospho-Proteomics: Methods and Protocols (Methods in Molecular Biology), Humana Press; 1 edition (2009), ISBN-10: 1603278338. The foregoing reference is incorporated herein by reference in its entirety for disclosure of methods and materials useful for the determination of the phosphorylation state of a phosphorylation site on a protein in a cell, tissue, or biological sample.

In some embodiments, the method comprises determining the level of phosphorylation of a phosphorylation site of a protein disclosed in Tables 1 or 2, and comparing the level of phosphorylation to a reference level. If the level of phosphorylation in the cell is higher than the reference level, then the cell is determined to exhibit an elevated level of mTOR kinase activity. If the level of phosphorylation in the cell is equal or lower than the reference level, then the cell is determined to not exhibit an elevated level of mTOR kinase activity. Methods of determining a level of phosphorylation are provided herein, and additional suitable methods will be apparent to those of skill in the art. The invention is not limited in this respect.

Suitable reference levels and methods of determining a reference level will be apparent to those of skill in the art. For example, if the cell is a neoplastic cell or cancer cell obtained from a subject, for example form a biopsy of a solid tumor in the subject, a suitable reference level may be obtained, in some embodiments, from a cell obtained from healthy or non-malignant tissue adjacent to the solid tumor, or a healthy cell of the same tissue of origin as the tumor cells from the same subject or from a different subject. If the cell is obtained from a healthy subject, a suitable reference level may be obtained from a cell of the same cell type obtained from another healthy subject. In some embodiments, a suitable reference level may be an average level or a range of levels observed or expected in cells obtained from healthy subjects that are of the same cell type of the same tissue of origin as the cell in question. In some embodiments, a reference level is a historical level, based on experience or prior experiments, or a level published or otherwise known in the art. Other suitable reference levels are described elsewhere herein and additional reference levels and methods to obtain such levels will be apparent to those of skill in the art.

In some embodiments, a plurality of phosphorylation sites are assayed including a Grb10 phosphorylation site, a pNDRG3 phosphorylation site, a CDK12 phosphorylation site, a FOXK1 phosphorylation site, a ZEB2 phosphorylation site, a LARP I phosphorylation site, an MIB1 phosphorylation site, an IBTK phosphorylation site, and/or a SRPK2 phosphorylation site. In some embodiments, a group of phosphorylation sites of proteins involved in a specific biological pathway are assayed, for example, of proteins known to those of skill in the art to be involved in a biological pathway disclosed in Table 10. In some embodiments, the plurality of phosphorylation sites includes mTOR target sites that were previously known. In some embodiments, the plurality of phosphorylation sites comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, at least 70, at least 80, at least 90, or at least 100 phosphorylation sites disclosed in Table 1. In some embodiments, the plurality of phosphorylation sites comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, at least 70, at least 80, at least 90, or at least 100 phosphorylation sites disclosed in Table 2. In some embodiments, the plurality of phosphorylation sites comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, at least 70, at least 80, at least 90, or at least 100 phosphorylation sites disclosed in Table 3 or in replicate described therein. In some embodiments, the plurality of phosphorylation sites comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, at least 70, at least 80, at least 90, or at least 100 phosphorylation sites disclosed in Table 7 or in replicate described therein. In some embodiments, the plurality of phosphorylation sites comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, at least 70, at least 80, at least 90, or at least 100 phosphorylation sites disclosed in Table 8. In some embodiments, the plurality of phosphorylation sites comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, at least 70, at least 80, at least 90, or at least 100 phosphorylation sites disclosed in Table 11, or in any Rapa or Ku replicate therein

In some embodiments, if a phosphorylation site determined to exhibit a higher level of phosphorylation in a cell in question, for example, a cancer cell obtained from a subject, as compared to a reference level is a class I phosphorylation site, then the cell is determined to exhibit an elevated level of rapamycin-sensitive mTORC1 activity. In some embodiments, if a phosphorylation site determined to exhibit a higher level of phosphorylation in the cell as compared to the reference level is a class II phosphorylation site, then the cell is determined to exhibit an elevated level of rapamycin-insensitive mTORC1 and/or mTORC2 activity. In some embodiments, if a phosphorylation site determined to exhibit a higher level of phosphorylation in the cell as compared to the reference level is a class III phosphorylation site, then the cell is determined to exhibit an elevated level of rapamycin-sensitive mTOR activity, or an elevated level of rapamycin-insensitive mTOR activity, or both. In some embodiments, if a cell is determined to exhibit a higher level of phosphorylation at phosphorylation sites of more than one class, then the cell is determined to exhibit a combination of elevated levels of mTOR signaling of the respective type.

In some embodiments, a method is provided comprising selecting a method of treatment based on the level of phosphorylation of the phosphorylation site(s) assayed. In some embodiments, if the cell is determined to exhibit an elevated level of mTOR kinase activity, then a method of treatment is selected that comprises administering an effective amount of an mTOR kinase inhibitor to the subject. In some embodiments, if the cell is determined to not exhibit an elevated level of mTOR kinase activity, then a method of treatment is selected that does not include administering an mTOR kinase inhibitor. In some embodiments, a method is provided that comprises selecting a method of treatment based on the classification of mTOR target sites that are determined to exhibit an elevated level of phosphorylation in the cell. For example, in some embodiments, if the cell is determined to exhibit an elevated level of rapamycin-sensitive mTORC1 activity, then a method of treatment is selected that comprises administering an effective amount of an inhibitor of rapamycin-sensitive mTORC1 kinase activity to the subject. In some embodiments, if the cell is determined to exhibit an elevated level of rapamycin-insensitive mTORC1 and/or mTORC2 activity, then a method of treatment is selected that comprises administering an effective amount of an inhibitor of rapamycin-insensitive mTORC1 kinase activity to the subject. In some embodiments, if the cell is determined to exhibit an elevated level of rapamycin-sensitive mTOR kinase activity, or an elevated level of rapamycin-insensitive mTOR kinase activity, or both, then a method of treatment is selected that comprises administering an effective amount of an inhibitor of rapamycin-sensitive and rapamycin-insensitive mTOR kinase activity or a combination of an inhibitor of rapamycin-sensitive mTOR kinase activity and an inhibitor of rapamycin-insensitive mTOR kinase activity to the subject.

Classification of a Cancer Based on Grb10 and/or PTEN Expression Levels

Some aspects of this invention relate to the surprising discovery that mutations leading to loss of function of the tumor suppressor Grb10 and mutations leading to loss of function of the tumor suppressor PTEN, are mutually exclusive. Loss of function of either tumor suppressor leads to elevated PI3K activity, which is thought to significantly contribute to carcinogenesis in certain types of cancer. In some embodiments, a method is provided that allows for the classification of a cancer based on Grb10 or PTEN expression levels. In some embodiments, the method comprises determining the level of expression of Grb10 in a cancer cell. In some embodiments, the method comprises determining the level of expression of PTEN in a cancer cell. In some embodiments, the method comprises determining the level of expression of Grb10 and PTEN in a cancer cell.

Expression levels of Grb10 and PTEN can be determined by various methods known to those of skill in the art including, for example methods for determining a level of protein, methods for determining a level of mRNA. Since phosphorylation affects protein stability, for example, stability of Grb10, methods for determining a level of protein phosphorylation may also be useful in assessing protein expression levels. Some such methods for expression analysis are provided herein, and additional methods will be apparent to those of skill in the art. The invention is not limited in this respect.

In some embodiments, the level of expression determined for Grb10 or PTEN is compared to a reference level, for example, to a level observed or expected in a healthy cell of the same cell type or of the same tissue of origin. In some embodiments, if the level of expression of Grb10 or of PTEN in the cell is lower than the reference level, then the cell is likely to exhibit an elevated level of PI3K activity. In some embodiments, if the cell is a neoplastic cell in a subject, a method of treatment with a combination of an mTOR inhibitor and a PI3K inhibitor or with a dual mTOR/PI3K inhibitor is indicated.

### Phosphoproteomics Binding Agents and Arrays

Phosphosensitive binding agents that specifically bind mTOR targets are also provided by the present invention. For example, some aspects of this invention provide antibodies and antibody fragments to each of the mTOR-targeted phosphorylation site disclosed in any of the Tables provided herein, for example, in any of Tables 1, 2, 3, 7, 8, or 11, and each such antibody is within the scope of the present invention. For example, some aspects of this invention provide aptamers and/or adnectins that specifically bind mTOR-targeted phosphorylation sites disclosed in Table 1, 2, 3, 7, 8, or 11, and each such aptamer or adnectin is within the scope of the present invention. In some embodiments, a kit is provided that comprises such phosphosensitive binding agents. A kit may also include a buffer, a container, control samples, or instructions.

Some embodiments provide a phosphoproteomics array that includes a plurality of phosphosensitive binding agents, for example, antibodies or antibody fragments, aptamers or adnectins, each of which specifically bind to a phosphorylation site disclosed in Table 1, 2, 3, 7, 8, or 11. In some embodiments the phosphosensitive binding agents are immobilized on a solid substrate, for example, on the surface of a glass slide, a bead, or a microtiter plate. In some embodiments, the plurality of phosphosensitive binding agents comprises binding agents that specifically bind to at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, or at least 100 phosphorylation sites disclosed in Table 1. In some embodiments, the plurality of phosphosensitive binding agents comprises binding agents that specifically bind to at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, or at least 100 phosphorylation sites disclosed in Table 2. In some embodiments, the plurality of phosphosensitive binding agents comprises binding agents that specifically bind to at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, or at least 100 phosphorylation sites disclosed in Table 3. In some embodiments, the plurality of phosphosensitive binding agents comprises binding agents that specifically bind to at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, or at least 100 phosphorylation sites disclosed in Table 7. In some embodiments, the plurality of phosphosensitive binding agents comprises binding agents that specifically bind to at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, or at least 100 phosphorylation sites disclosed in Table 8. In some embodiments, the plurality of phosphosensitive binding agents comprises binding agents that specifically bind to at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, or at least 100 phosphorylation sites disclosed in Table 11.

In some embodiments, an anti-phospho-S501/503-Grb10, anti-mTOR, anti-phospho-mTOR(S2481), anti-Grb10 (human), anti-phospho-Akt (Ser473), anti-Akt, anti-S6K, anti-phospho-S6K (T389), anti-IRS2, anti-PARP, anti-caspase 3, anti-4EBP, anti-4EBP (Ser37/46), anti-phospho-ribosomal protein S6 (Ser235/236), anti-ribosomal protein S6, anti-phospho-ERK1/2, anti-phospho-IRS (Y612), anti-p85 and anti-p110 of PI3K, and/or an anti ERK1/2 antibody or antibody fragment is provided. In some embodiments, phosphosensitive antibodies or antibody fragments are provided for mTOR upstream regulators, including, but not limited to anti-phospho-Akt (Ser473), anti-phospho-ERK1/2, anti-phospho-IRS (Y612), anti-p85, and anti-p110 of PI3K. In some embodiments, phosphosensitive antibodies or antibody fragments are provided for mTOR downstream effectors including, but not limited to anti-phospho-mTOR(S2481), anti-phospho-Akt (Ser473), anti-phospho-S6K (T389), anti-4EBP (Ser37/46), and anti-phospho-ribosomal protein S6 (Ser235/236).

In some embodiments, antibodies against mTORC1 downstream effectors identified for the first time herein are provided, including, anti-phospho-NDRG3 (Ser331), anti-phospho-S5011503-Grb10, and anti-CDC2-related kinase, Arg/Ser-Rich (Ser437).

Phosphosensitive binding agents, their generation and purification, and their use in assays, arrays, and methods for phosphoproteomics analyses of biological samples are well known in the art, and exemplary methods are described, for example, in Marjo de Graauw (Editor), Phospho-Proteomics: Methods and Protocols (Methods in Molecular Biology), Humana Press; 1 edition (2009), ISBN-10: 1603278338, incorporated herein by reference in its entirety. The foregoing reference is incorporated herein by reference in its entirety for disclosure of phosphosensitive binding agents, their generation and purification, and their use in assays, arrays, and methods for phosphoproteomics analyses of biological samples.

Methods of using a microarray comprising a plurality of phosphosensitive binding agents that specifically bind mTOR targets are also provided. In some embodiments, methods of using a phosphoproteomics array to determine mTOR activity in a cell, for example, in a cancer cell, are provided. In some embodiments, the method comprises contacting a proteinaceous sample derived from the cell with the phosphoproteomics array under conditions suitable for a protein expressed in the cell to bind to an antibody or antibody fragment of the array. Such conditions are well known to those of skill in the art and exemplary protocols for phosphoprotein microarrays are described in Marjo de Graauw (Editor), Phospho-Proteomics: Methods and Protocols (Methods in Molecular Biology), Humana Press; 1 edition (2009), ISBN-10: 1603278338, incorporated herein by reference for the disclosure of such methods and conditions. In some embodiments, the method further comprises determining a level of phosphorylated protein bound to a binding agent, for example, an antibody or antibody fragment of the array. In some embodiments, this step includes quantification, absolute or relative to a reference level, of the amount of protein bound to a specific binding agent. In some embodiments, the method includes comparing the level of phosphorylated protein bound to an antibody or antibody fragment of the array to a reference level, wherein if the level phosphorylated protein in the sample derived from the cell is higher than the reference level, then the cell is determined to exhibit an elevated level of mTOR kinase activity.

In some embodiments, methods are provided that include generating a phosphoproteomic profile of the cell. In some embodiments, the profile includes phosphorylation levels of a plurality of proteins, for example, mTOR targets expressed in the cell. In some embodiments, the method further includes comparing the phosphoproteomic profile of the cell with a phosphoproteomic profile of a control cell. In some embodiments, if the phosphoproteomic profile of the cell is similar to that of the control cell, then the cell is determined to exhibit a level of mTOR kinase activity similar to that of the control cell. Methods for comparing phosphoprotein profiles are well known in the art and include, for example, hierarchical clustering methods, supervised and unsupervised learning methods, classification methods, for example class predictor building methods based on phosphoproteomics profiles from cells of known character, and calculation of correlation parameters, such as distance analysis or correlation coefficient calculations. Useful similarity ranges are also known to those of skill in the art and the invention is not limited in this respect. For non-limiting examples of disclosures of such methods and algorithms, see Lim, Y. (2005) Mining the tumor phosphoproteome for cancer markers. Clin Cancer Res 11(9): 3163-3169; Kalume, D. et al. (2003) Tackling the phosphoproteome: tools and strategies Current Opinion in Chemical Biology 7: 64-69; Schmelzle, K. & White, F. (2006) Phosphoproteomic approaches to elucidate cellular signaling networks. Current Opinion in Chemical Biology 17: 406-414; Olsen J V et al. Global, in vivo, and site-specific phosphorylation dynamics in signaling networks. Cell. 2006 Nov. 3; 127(3):635-48; Mumby, M. & Brekken, D. (2005) Phosphoproteomics: new insights into cellular signaling. Genome Biology 6: 230.1-230.7; Zhang et al. J. Proteome Res. vol. 5 pp. 581-8 2006; Hoffert J D, et al. Quantitative phosphoproteomics of vasopressin-sensitive renal cells: regulation of aquaporin-2 phosphorylation at two sites. Proc Natl Acad Sci USA. 2006 May 2; 103(18):7159-64; Johnson, S & Hunter, T. (2004) Phosphoproteomics finds its timing. Nature Biotech 22(9): 1093-1094; and Marjo de Graauw (Editor), Phospho-Proteomics: Methods and Protocols (Methods in Molecular Biology), Humana Press; 1 edition (2009), ISBN-10: 1603278338; all incorporated herein by reference in their entirety

In some embodiments, the control cell is a healthy cell. In some embodiments, the control cell is a cell exhibiting normal mTOR kinase activity. In some embodiments, the control cell is a cancer cell. In some embodiments, the control cell is a rapamycin-sensitive cancer cell. In some embodiments, the control cell is a rapamycin insensitive cancer cell.

Identification of mTOR Kinase Inhibitors Based on mTOR Target Analysis

In some aspects, the invention provides methods of identifying an mTOR kinase inhibitors by analyzing the phosphorylation state of an mTOR target as provided herein after contacting a cell or test sample with a candidate agent. In some embodiments, mTOR phosphorylation efficiency directed towards a target disclosed in Table 1, 2, 3, 7, 8, or 11 is measured in the presence of a candidate agent. In some embodiments, the method includes contacting an mTOR kinase molecule with a polypeptide comprising a phosphorylation site disclosed in Table 1, 2, 3, 7, 8, or 11 under conditions suitable for the mTOR kinase to phosphorylate the phosphorylation site in the presence of a candidate agent. In some embodiments, the level of phosphorylation of the phosphorylation site is then determined and compared to a reference level. In some embodiments, if the level obtained in the presence of the candidate agent is lower than the reference level, then the candidate agent is identified as an mTOR kinase inhibitor.

In some embodiments, the phosphorylation site is a Grb10 phosphorylation site, a pNDRG3 phosphorylation site, a CDK12 phosphorylation site, a FOXK1 phosphorylation site, a ZEB2 phosphorylation site, a LARP1 phosphorylation site, an MIB1 phosphorylation site, an IBTK phosphorylation site, and/or a SRPK2 phosphorylation site. In some embodiments, the candidate agent is a polypeptide, an aptamer, an adnectin, or a small molecule. In some embodiments, the reference level is the level of phosphorylation of the phosphorylation site determined in the absence of the candidate agent. In some embodiments, the level determined in the presence of the candidate agent is lower than the reference level, if the level determined in the presence of the candidate agent is less than 75%, less than 50%, less than 40%, less than 30%, less than 25%, less than 20%, less than 10%, less than 5%, less than 2.5%, or less than 1% of the reference level.

In some embodiments, the contacting is performed in vitro. In some embodiments, the contacting is performed in vivo. In some embodiments, the reference level is a level observed or expected in the absence of the candidate agent. In some embodiments, the reference level is a level observed or expected in the absence of any candidate agent. In some embodiments, the reference level is a level observed or expected in the presence of a known agent. In some embodiments, the reference level is a level observed or expected in the presence of a control agent.

### Subjects and Cells

In some embodiments, the subject is an animal. In some embodiments, the subject is a domesticated animal. In some embodiments, the subject is a mammal. In some embodiments, the subject is a non-human mammal. In some embodiments, the subject is a non-human primate. In some embodiments, the subject is a mouse, rat, or rabbit. In some embodiments, the subject is a sheep, goat, cattle, pig, horse, dog, or cat. In some embodiments, the subject is a human.

In some embodiments, the subject is a healthy subject. In some embodiments, the subject is a subject having a hyperproliferative disease. In some embodiments, the subject is a subject having a neoplastic disease. In some embodiments, the subject is a subject having a cancer. In some embodiments, the subject is a subject having a cancer characterized and/or diagnosed to exhibit an elevated level of mTOR activity. In some embodiments, the subject is a subject who had a tumor removed.

In some embodiments, the cell is a healthy cell. In some embodiments, the cell is any cell. In some embodiments, the cell is a bacterial cell. In some embodiments, the cell is an animal cell. In some embodiments, the cell is a mammalian cell. In some embodiments, the cell is a human cell. In some embodiments, the cell is a cell of a cell line. In some embodiments, the cell is a transformed or immortalized cell. In some embodiments, the cell is a neoplastic cell. In some embodiments, the cell is a tumor cell. In some embodiments, the cell is a cancer cell. In some embodiments, the cell is obtained from a tumor in a subject, for example, by tumor biopsy. In some embodiments, the cell is a cell obtained from a tumor that has been removed from a subject.

In some embodiments, the cell is a cell known to exhibit an elevated level of mTOR activity. In some embodiments, the cell is a cell carrying a mutation in a gene involved in an mTOR signaling pathway. In some embodiments, the gene involved in an mTOR signaling pathway is a gene involved in IGF signaling, EGFR signaling, GF signaling, PI3K signaling, AKT signaling, MAPK signaling, Ras signaling, Raf signaling, or Rb signaling. In some embodiments, the gene is TSC1/2, a receptor tyrosine kinases (RTK), PI3K, PTEN, Akt, Ras, Raf, MEK, LKB, or NF2.

### Kinase Inhibitors

Kinase inhibitors useful for some aspects of this invention include, but are not limited to, mTOR inhibitors, PI3K inhibitors, Akt inhibitors, and MAPK inhibitors. Further, inhibitors useful for some methods provided herein include, for example, inhibitors of mTOR target degradation, for example, inhibitors of Grb10 degradation. In some embodiments, a kinase inhibitor, for example, an mTOR inhibitor, as provided herein, is an allosteric mTOR kinase inhibitor or a catalytic mTOR kinase inhibitor. In some embodiments, the allosteric mTOR kinase inhibitor is rapamycin or a rapamycin analog. In some embodiments, the catalytic mTOR kinase inhibitor is an ATP-competitive mTOR kinase inhibitor. Other allosteric and catalytic mTOR kinase inhibitors are well known to those of skill in the art, and the invention is not limited in this respect.

In some embodiments, an mTOR kinase inhibitor as provided herein is an mTORC1 inhibitor. In some embodiments, an mTOR kinase inhibitor as provided herein is an mTORC1/2 inhibitor. In some embodiments, an mTOR kinase inhibitor as provided herein is rapamycin or a rapamycin analog. In some embodiments, the rapamycin analog is Ridaforolimus, Sirolimus or Everolimus. In some embodiments, an mTOR kinase inhibitor as provided herein is PP242, PP30, AZD8055, OSI-027, WYE354, INK-128, XL388, torin1, rapamycin (sirolimus), FK506 (tacrolimus), CCI779 (temsirolimus), RAD001 (everolimus), AP23573 (deforolimus, ridaforolimus), S-trans,trans-farnesyl thiosalicylic acid (FTS), FKBP38, PX-866, Theophylline, Caffeine, LY303511, PI-103, 2-(morpholin-1-yl)pyrimido[2,1-aplpha]isoquinolin-4-one, or BEZ235 (NVP-BEZ235)

In some embodiments, an mTOR kinase inhibitor as provided herein is a dual PI3K/mTOR kinase inhibitor. In some embodiments, the dual PI3K/mTOR kinase inhibitor is NVP-BEZ235, BGT-226, XL-765, GSK2126458 or SF1126. Some of the mTOR kinase inhibitors listed immediately above are in various stages of clinical trials. Additional mTOR inhibitors will be apparent to the skilled artisan as they are well known in the art, and it should be appreciated that the invention is not limited in this respect.

In some embodiments, a PI3K inhibitor is provided that is useful in a therapeutic method provided by aspects of this invention. In some embodiments, a PI3K inhibitor as provided herein is ZSTK474, TGX221, GDC0941, or LY294002, XL147, PX147, BKM120, GSK 615, CAL101, PX-866, Quercetin, Tetrodotoxin citrate, Thioperamide maleate, IC87114, PI-103, BEZ235 (NVP-BEZ235), TGX-115, (−)-Deguelin, NU 7026, Myricetin, Tandutinib, SF1126, XL765, D-87503, D-106669, or GSK615. In some embodiments, a PI3K inhibitor provided herein is a dual PI3K/mTOR kinase inhibitor, for example, NVP-BEZ235, BGT-226, XL-765, GSK2126458 or SF1126. In some embodiments, an Akt inhibitor is provided that is useful in a therapeutic method provided by aspects of this invention. In some embodiments, the Akt inhibitor is perifosine, GSK690693, A443654 or MK2206.

In some embodiments, a compound is provided that inhibits the degradation of an mTOR target, for example, of Grb10, NDRG3, CDK12, FOXK1, ZEB2, LARP1, MIB1, IBTK, and/or SRPK2. In some embodiments, the compound that inhibits the degradation of an mTOR target is a ubiquitin ligase inhibitor. In some embodiments, the ubiquitin ligase inhibitor is an E3 ubiquitin ligase inhibitor.

### Compositions

In some embodiments, pharmaceutical compositions are provided that comprise an mTOR kinase inhibitor, a PI3K, Akt, or MAPK inhibitor, and/or an agent that inhibits the degradation of an mTOR target, for example, Grb10, NDRG3, CDK12, FOXK1, ZEB2, LARP1, MIB1, IBTK, and/or SRPK2. Pharmaceutical compositions provided herein preferably are sterile and contain an effective amount of one or more therapeutic agents as described herein for producing the desired response in a unit of weight or volume suitable for administration to a patient. If the desired response is amelioration of a hyperproliferative disease, neoplastic disease, or cancer, then the response can, for example, be measured by determining the proliferation of neoplastic or cancer cells in a subject after treatment by, for example, measuring tumor volume, evaluating regression, relapse, or disease symptoms, or by obtaining a cell sample and perform cell counting, flow cytometry, FACS, and other methods well known in the art to be suitable to determine cell proliferation.

In some embodiments, the pharmaceutical compositions as described herein may contain suitable buffering agents, for example, acetic acid in a salt form, citric acid in a salt form, boric acid in a salt form, and/or phosphoric acid in a salt form. The pharmaceutical compositions also may contain, optionally, suitable preservatives, such as ascorbic acid, benzalkonium chloride, benzyl alcohol, m-cresol, chlorobutanol, parabens, EDTA, EGTA, and/or thimerosal. The pharmaceutical compositions may conveniently be presented in unit dosage form and may be prepared by any of the methods well-known in the art of pharmacy.

In some embodiments, a therapeutic method or a method of formulating a kinase inhibitor into a medicament for therapeutic use may include the step of bringing the active agent, for example, a kinase inhibitor as described herein, into association with a carrier which constitutes one or more accessory ingredients. In general, compositions are prepared by uniformly and intimately bringing the active compound(s) into association with a liquid carrier, a finely divided solid carrier, or both, and then, if necessary, shaping the product.

In some therapeutic embodiments, a composition or pharmaceutic preparation provided herein is administered orally to a subject having a cancer. In some embodiments, compositions as described herein that are suitable for oral administration may be presented as discrete units, such as capsules, tablets, lozenges, each containing a predetermined amount of the active compound. Other examples of compositions include suspensions in aqueous liquids or non-aqueous liquids, such as a syrup, elixir, or an emulsion. Examples of compositions for parenteral administration include, without being limited to, sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Examples of aqueous carriers are water, alcoholic/aqueous solutions, emulsions or suspensions, for example, saline and buffered media. Examples of parenteral vehicles are sodium chloride solution, Ringer's dextrose, dextrose and sodium chloride, and lactated Ringer's or fixed oils. Examples for intravenous vehicles are fluid and nutrient replenishers, electrolyte replenishers (such as those based on Ringer's dextrose), and the like. Preservatives and other additives may also be present such as, for example, antimicrobials, anti-oxidants, chelating agents, and inert gases, and the like.

In some embodiments, a composition comprising a compound or a combination of compounds, useful in this invention, may further comprise an antioxidant to retard oxidation of one or more component. Additionally, the prevention of the action of microorganisms can be brought about by a preservative such as an antibacterial and antifungal agent, including but not limited to parabens (e.g., methylparabens, propylparabens), chlorobutanol, phenol, sorbic acid, thimerosal or combinations thereof.

The compounds useful in the invention, for example the mTOR, PI3K, Akt or MAPK inhibitors provided herein, may be derivatized in various ways. As used herein “derivatives” of the compounds (e.g., small molecule JAK2 and other kinase inhibitors) include salts (e.g., pharmaceutically acceptable salts), any complexes (e.g., inclusion complexes or clathrates with compounds such as cyclodextrins, or coordination complexes with metal ions such as Mn²+ and Zn²+), esters such as in vivo hydrolysable esters, free acids or bases, polymorphic forms of the compounds, solvates (e.g., hydrates), prodrugs or lipids, coupling partners and protecting groups. By “prodrugs” is meant for example any compound that is converted in vivo into a biologically active compound.

The term “pharmaceutically acceptable salt” in this respect refers to the relatively non-toxic, inorganic or organic acid addition salts of agents of the present invention. These salts can be prepared in situ in the administration vehicle or the dosage form manufacturing process, or by separately reacting a purified agent of the invention with a suitable organic or inorganic acid, and isolating the salt thus formed during subsequent purification. Representative salts include the bromide, chloride, sulfate, bisulfate, phosphate, phosphonate, nitrate, acetate, valerate, oleate, palmitate, stearate, laurate, benzoate, lactate, tosylate, citrate, maleate, fumarate, succinate, tartrate, napthylate, mesylate, glucoheptonate, lactobionate, laurylsulphonate salts, and the like. See, for example, Berge et al. (1977) J. Pharm. Sci. 66:1-19.

The pharmaceutically acceptable salts of the subject agents include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

As set out herein, certain compounds may contain a basic functional group, such as amino or alkylamino, and are, thus, capable of forming pharmaceutically acceptable salts with pharmaceutically acceptable acids. The term “pharmaceutically acceptable salts” in this respect refers to the relatively non-toxic, inorganic or organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, phosphonate, nitrate, acetate, valerate, oleate, palmitate, stearate, laurate, benzoate, lactate, tosylate, citrate, maleate, fumarate, succinate, tartrate, napthylate, mesylate, glucoheptonate, lactobionate, and laurylsulphonate salts and the like. See, for example, Berge et al. (1977) J. Pharm. Sci. 66:1-19.

The pharmaceutically acceptable salts of the compounds useful in the present invention include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine, and the like. See, for example, Berge et al. (1977) J. Pharm. Sci. 66:1-19.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes), and combinations thereof. The proper fluidity can be maintained, for example, by the use of a coating, such as lecithin; by the maintenance of the required particle size by dispersion in carriers such as, for example liquid polyol or lipids; by the use of surfactants such as, for example, hydroxypropylcellulose; or combinations thereof such methods. In many cases, it will be advisable to include isotonic agents, such as, for example, sugars, sodium chloride, or combinations thereof.

### Therapeutic Methods

Some aspects of this invention provide therapeutic methods, for example, methods for the treatment of subjects having a hyperproliferative disease, a neoplastic disease, or a cancer. In some embodiments, a method of treatment is personalized to a specific subject by taking into account the phosphorylation level of at least one an mTOR target site in the subject. In some embodiments, therapeutic methods disclosed herein include administration of an mTOR inhibitor, for example, of an mTORC1 or an mTORC1/2 inhibitor. In some embodiments, therapeutic methods described herein include administration of an mTOR inhibitor and an additional kinase inhibitor, for example, a PI3K inhibitor, a MAPK inhibitor, MEKIERK inhibitor, or an AKT/PKB inhibitor. In some embodiments, therapeutic methods provided herein include administration of a dual mTOR/PI3K inhibitor, or of a combination of an mTOR inhibitor and a PI3K inhibitor.

In some therapeutic embodiments, a composition disclosed herein, for example, a composition comprising an mTOR inhibitor or a composition comprising an mTOR inhibitor and a PI3K inhibitor is administered to a subject having a cancer in an effective amount. An effective amount, in some embodiments, is an amount sufficient to elicit a desired clinical response in the subject. In some embodiments, the desired response is a slowing or inhibiting of the progression of a disorder, for example, of a malignant neoplastic disorder. In some embodiments, this involves slowing the progression of the disease temporarily, although, in more preferable embodiments, it involves halting the progression of the disease permanently.

In some embodiments, the desired response is a permanent reduction of cancer cell proliferation, for example, to a level comparable to a level found in healthy individuals. In some embodiments, a desired response is the induction of cell death in a cancer cell, in a population of cancer cells, or in all cancer cells in a subject. In some embodiments, the desired response is delaying or preventing the manifestation of clinical symptoms characteristic of the disease or condition.

The effect of administering an mTOR inhibitor, either alone or in combination with an additional compound, for example, a PI3K inhibitor, as provided herein, can be monitored by routine methods well known to those of skill in the related medical arts, for example, by methods involving assessment of cancer cell proliferation.

What constitutes an effective amount will depend on the particular condition being treated, the severity of the condition, the individual patient parameters including age, physical condition, size, and weight, the duration of the treatment, the nature of concurrent therapy (if any), the specific route of administration and like factors within the knowledge and expertise of the health care professional treating the subject. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a lower dose or tolerable dose may be used for medical reasons.

In general, an effective amount of a therapeutic agent, for example, an mTOR inhibitor or a combination of an mTOR inhibitor with a PI3K, Akt, or MAPK inhibitor, as provided herein, for the treatment of a cancer exhibiting an elevated level of mTOR kinase activity is a dose that achieves an alleviation of the specific neoplastic disease or disorder being treated, for example, by prevention, inhibition, amelioration, delay, or elimination of a symptom of such a disease or disorder.

Some embodiments provide a method of inducing cell death and/or inhibiting proliferation in a neoplastic cell exhibiting elevated mTOR kinase activity by contacting the cell with an mTOR kinase inhibitor or a combination of an mTOR kinase inhibitor and another kinase inhibitor, for example, a PI3K, Akt, or MAPK inhibitor, chosen based on the determination of the type of mTOR kinase signaling active in the cell. In some embodiments, the neoplastic cell is contacted in vivo by administering a composition disclosed herein to a subject carrying the cell. In some embodiments, the neoplastic cell is contacted ex vivo. In some embodiments, the cell is contacted in vitro.

In some embodiments, a neoplastic cell is contacted in vivo, ex vivo, or in vitro, with an effective amount of an mTOR inhibitor or a combination of an mTOR inhibitor and an additional kinase inhibitor, for example, a PI3K, Akt, or MAPK inhibitor, as provided herein. An effective amount, in some embodiments, is an amount sufficient to elicit a desired response in the contacted cell. In some embodiments involving contacting a neoplastic cell exhibiting elevated mTOR activity, for example, because of a mutation in an mTOR upstream pathway, the desired response is a slowing or inhibiting of the proliferation of the cell. In some embodiments, this decreases the proliferation rate and/or cell viability and/or life span, although, in more preferable embodiments, it involves the induction of cell death in the contacted cell or cells.

In some embodiments, the therapeutic methods provided herein further involve the administration of an additional antiproliferative agent to a cancer cell or to a subject carrying a cancer cell, for example, as part of a malignant tumor. Additional antiproliferative agents useful in the methods described herein are well known in the art and include, but are not limited to chemotherapeutic agents (e.g., cytostatic, and cytotoxic agents). Cytotoxic and cytostatic drugs are drugs that kill malignant cells, or inhibit their proliferation, respectively. Examples of cytotoxic and cytostatic drugs include, for example, alkylating agents, antimetabolites, antitumor antibiotics, vinca alkaloids, taxanes, topoisomerase-I compounds, anthrapyrazoles, and epidophylotoxins. In addition, angiogenesis inhibiting drugs, including, for example, compounds that block growth promoting receptors (e.g., PDGF-R and VEGF-R) such as sunitinib (Sutent®) may be used as additional antiproliferative agents. Non-limiting examples of additional antiproliferative agents include Cytoxan® (Cyclophosphamide), Methotrexate, 5-Fluorouracil (5-FU), Adriamycin® (Doxorubicin), Prednisone, Nolvadex® (Tamoxifen), Taxol® (Paclitaxel), Leucovorin, Oncovin® (Vincristine), Thioplex® (Thiotepa), Arimidex® (Anastrozole), Taxotere® (Docetaxel), Navelbine®, (Vinorelbine), Gemzar® (Gemcitabine), Ifex® (Ifosfamide), Pemetrexed, Topotecan, Melphalan (L-Pam®), Cisplatin (Cisplatinum®, Platinol®), Carboplatin (Paraplatin®), Carmustine (BCNU; BiCNU®), Methotrexate, Edatrexate, Mitomycin C (Mutamycin®), Mitoxantrone (Novantrone®), Vincristine (Oncovin®), Vinblastine (Velban®), Vinorelbine (Navelbine®), Fenretinide, Topotecan, Irinotecan, 9-amino-camptothecin (9-AC); Biantrazole, Losoxantrone, Etoposide, and Teniposide.

Administration schedules, formulations, dosages, and administration routes of antiproliferative agents and compositions are well known to those in of skill in the art. Exemplary administration routes, schedules, and dosages of commonly used chemotherapeutic drugs are described in Perry, The Chemotherapy Source Book, 4^{th} Edition, Lippinkott Williams & Wilkins, 2008, incorporated herein by reference. Such administration schedules may comprise the administration of a single antiproliferative drug or the administration of a combination of such drugs, for example, one of the following, commonly administered combinations: CMF (cyclophosphamide, methotrexate, and 5-fluorouracil); classic CMF (oral cyclophosphamide plus methotrexate and 5-fluorouracil); CAF or FAC (cyclophosphamide, Adriamycin® (doxorubicin), and 5-fluorouracil); AC (Adriamycin® and cyclophosphamide); ACT (Adriamycin® plus cyclophosphamide and tamoxifen); AC taxol (Adriamycin® plus cyclophosphamide and paclitaxel (Taxol®); FACT (5-fluorouracil plus Adriamycin®, cyclophosphamide, and tamoxifen); A-CMF or Adria/CMF (4 cycles of Adriamycin® followed by 8 cycles of CMF); CMFP (CMF plus prednisone); CMFVP (CMF plus vincristine and prednisone); CAFMV (CAF plus methotrexate and vincristine); CMFVATN (CMF plus vincristine, Adriamycin®, thiotepa, and tamoxifen); MF (methotrexate plus 5-fluorouracil and leucovorin). The administration of such combinations of antiproliferative drugs and agents in addition to the administration of an mTOR kinase inhibitor with or without an additional kinase inhibitor, for example, a PI3K, Akt, or MAPK inhibitor, is also envisioned to be embraced by some embodiments.

The therapeutic inhibitors and compositions can be administered in a single dose comprising an effective amount of the individual agents. Multiple doses of the compounds of the invention are also contemplated. When a plurality of inhibitors are used together, they may be administered individually or sequentially, either in a single medicament or in separate units to provide therapeutic doses of the individual compounds. Many mTOR inhibitors, PI3K inhibitors, Akt inhibitors and MAPK inhibitors described herein are in clinical studies or even in clinical use. Therapeutic doses of such compounds are, accordingly, well known in the field of medicine. Dosages of compounds in clinical use are described in references such as Remington's Pharmaceutical Sciences, 18th ed., 1990; as well as many other medical references relied upon by the medical profession as guidance for the treatment of proliferation disorders.

### Administration Routes and Methods

A variety of administration routes are available for the kinase inhibitors and other therapeutic agents described herein. The particular mode selected will depend, of course, upon the particular compound selected, the particular condition being treated and the dosage required for therapeutic efficacy. The methods of this invention may be practiced using any mode of administration that is medically acceptable, meaning any mode that produces effective levels of compounds without causing clinically unacceptable adverse effects. Examples of modes of administration are parenteral routes. The term “parenteral” includes subcutaneous, intravenous, intramuscular, intraperitoneal, and intrasternal injection, or infusion techniques. Other routes include, but are not limited to, oral, nasal, dermal, sublingual, and local.

The formulations of the invention are administered in pharmaceutically acceptable solutions, which may routinely contain pharmaceutically acceptable concentrations of salts, buffering agents, preservatives, compatible carriers, adjuvants, and optionally other therapeutic ingredients.

According to the methods provided by aspects of the invention, the compounds described herein may be administered in a pharmaceutical composition. In some embodiments, a pharmaceutical composition comprises a compound provided by aspects of the invention and a pharmaceutically acceptable carrier. As used herein, a pharmaceutically acceptable carrier refers to a non-toxic material that does not interfere with the effectiveness of the biological activity of the active ingredients.

Pharmaceutically acceptable carriers include diluents, fillers, salts, buffers, stabilizers, solubilizers, and other materials which are well known in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

The compounds used in the invention may be formulated into preparations in solid, semi-solid, liquid or gaseous forms such as tablets, capsules, powders, granules, ointments, solutions, depositories, inhalants and injections, and usual ways for oral, parenteral or surgical administration. Some aspects of the invention also embrace pharmaceutical compositions which are formulated for local administration, such as by implants.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion. When the compounds described herein are used therapeutically, in certain embodiments a desirable route of administration may be by pulmonary aerosol.

In some embodiments, a compound provided by some aspects of the invention may be administered directly to a tissue. Direct tissue administration may be achieved by direct injection. A compound may be administered once or alternatively may be administered in a plurality of administrations. If administered multiple times, a compound may be administered via different routes. For example, the first (or the first few) administrations may be made directly into the affected tissue while later administrations may be systemic.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

Other delivery systems can include time-release, delayed release or sustained release delivery systems. Such systems can avoid repeated administrations of the compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Therapeutic formulations useful in the invention may be prepared for storage by mixing a kinase inhibitor having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

### EXAMPLES

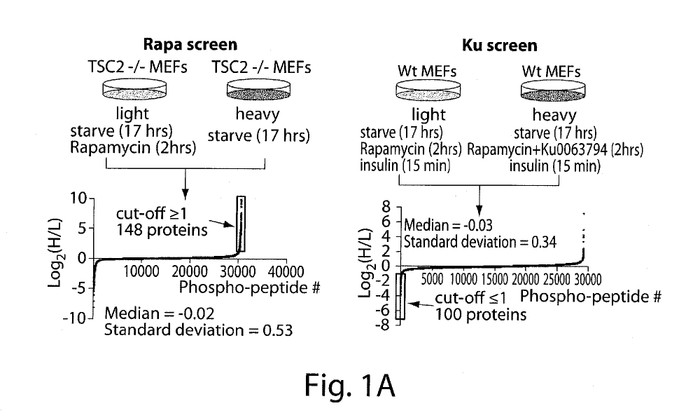

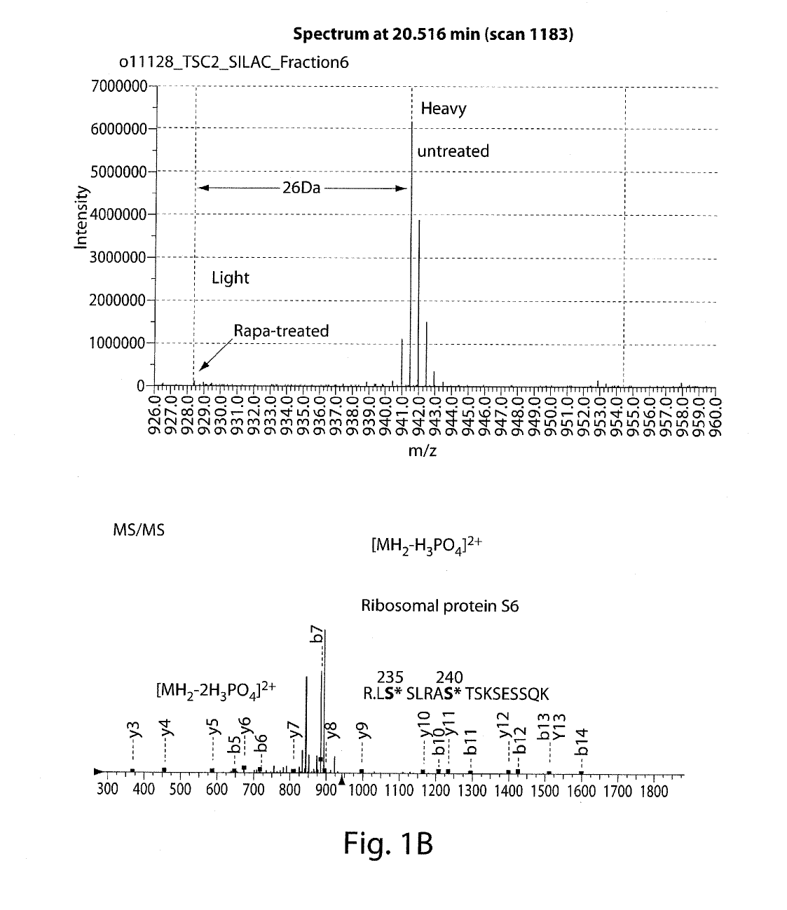

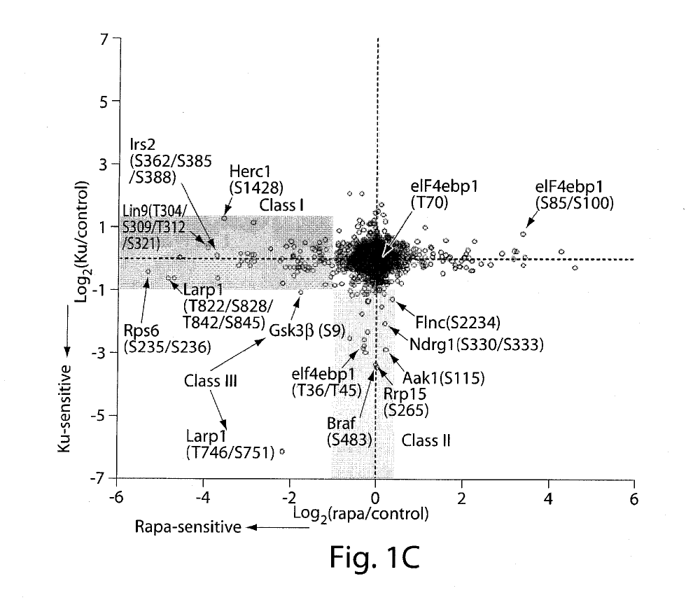

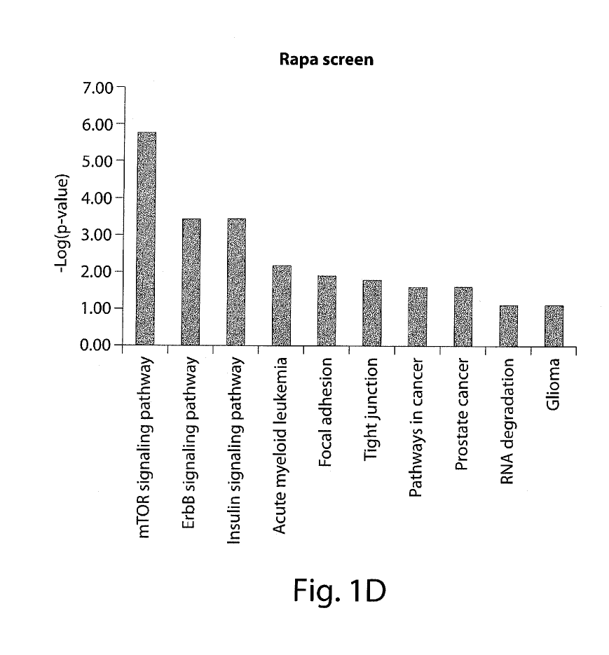

Two sets of large-scale, quantitative phospho-proteomics experiments were performed to fully define how mTOR-containing complexes signal to downstream effectors (FIG. 1A). The first SILAC experiment (rapamycin screen) was performed using growth factor-deprived TSC2−/− mouse embryonic fibroblasts (MEFs). Deletion of the TSC2 tumor suppressor gene decouples mTORC1 from many upstream inputs, leading to constitutive hyper-activation of mTORC1 signaling (7). mTORC1 is still potently and specifically inhibited by rapamycin in these cells, which provides a sensitized genetic background for the study of mTORC1 signaling in the absence of other mitogen-regulated phosphorylation cascades.

Global Quantitative Phosphoproteomics Defines the Signaling Networks Downstream of both mTORC1 and mTORC2

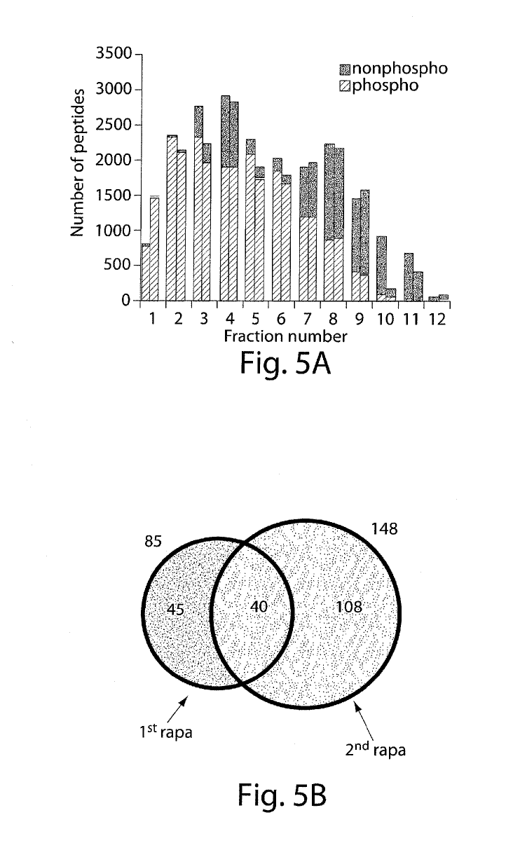

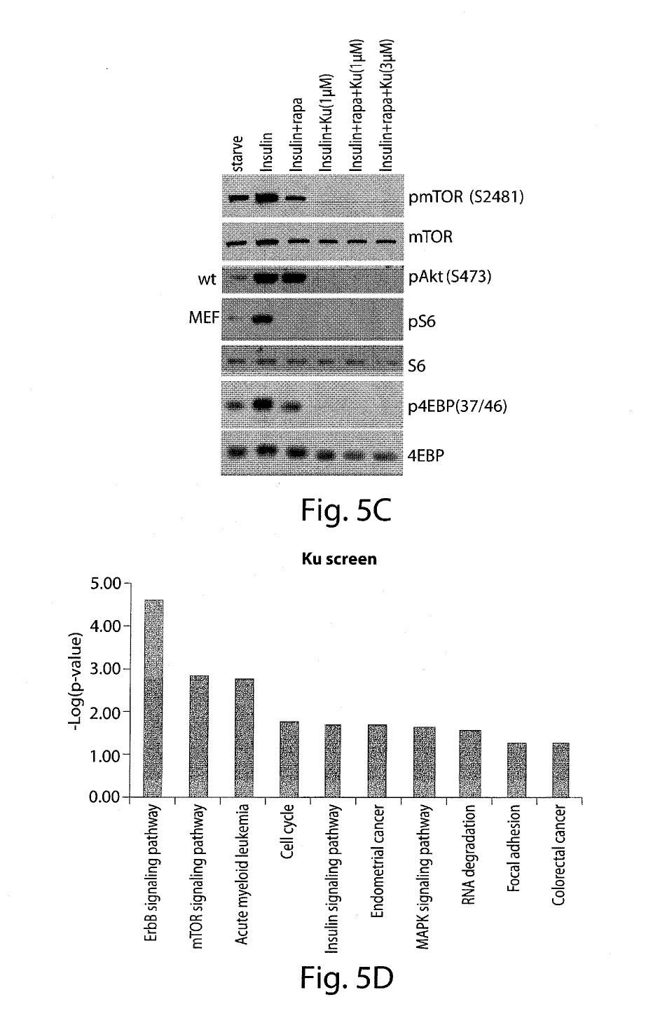

Two populations of TSC2−/− cells were grown in parallel, one in conventional media (“light”) and the other in media containing [¹³C₆¹⁵N₂]lysine and [¹³C₆¹⁵N₄]arginine (“heavy”) (FIG. 1A and Table 5). As shown in FIG. 5A, phosphopeptides were successfully enriched using a two-step SCX-IMAC procedure with the phosphopeptides representing 67.8% of the peptides identified. We performed two biological replicates of this experimental design with cross-labeling (swapping the labeled state of the rapamycin-treated cells, Table 5), from which a total of 14,635 (FDR=0.25%) and 32,500 (FDR=0.28%) phosphopeptides were identified, respectively. These corresponded to identification of at least 4,484 and 6,832 unique phosphorylation sites on 1,615 and 1,866 proteins, respectively. The quantitation was highly accurate (Table 5) with 93.7% of the phosphoproteome changing less than 50% in abundance between the rapamycin-treated (light) and control (heavy) cells (second biological replicate experiment, Median Log₂(H/L)=−0.02 and standard deviation Log₂(H/L)=0.53, FIG. 1A). Gene names and sequences of the phosphopeptides whose intensities decrease after rapamycin treatment (Rapamycin screen) are identified in Table 7. Note that the Rapamycin screen includes two biological replicates. For the first replicate (Table 7 Rapa Replicate 1), the light cells were controls whereas the heavy cells were treated with rapamycin. For the second replicate experiment (Table 7 Rapa Replicate 2), the light cells were treated with rapamycin and heavy cells were controls.

Based on the tight distribution of the quantified phosphopeptides, we considered phosphorylated peptides whose relative abundance decreased ≧2-fold as regulated in a rapamycin-sensitive fashion. Using this criteria, several hundred peptides (corresponding to 148 and 85 proteins respectively, in the two biological replicates, Table 11) were determined to contain rapamycin-sensitive phosphorylation sites. Table 11 identifies proteins with downregulated phosphorylation identified in the rapamycin and Ku-0063794 screen. Note that there are two biological replicate experiments for the rapamycin screen (“Table 11 Rapa Replicate 1,” and “Table 11 Rapa Replicate 2,” respectively) and one Ku-0063749 replicate (“Table 11 Ku”). Treatment for the light and heavy cells in each experiment is described in FIG. 1C. We observed a similar distribution for the phosphopeptides in the first biological replicate experiment and found 85 proteins (Table 11) that carried rapamycin-sensitive phosphorylation sites. In addition, there was a substantial overlap in the down-regulated proteins between replicates and 40 of these proteins were found in both experiments (FIG. 5B).

For the Ku-screen, wild-type MEFs were grown in light and heavy SILAC media and starved overnight for serum. The light cells were treated with 20 nM rapamycin for 2 hrs, while the heavy cells were treated in parallel with a combination of 20 nM rapamycin and 2 μM of Ku-0063794 for 2 hrs. Both the light and heavy cell were subsequently stimulated with insulin for 15 minutes and samples were pooled and analyzed by quantitative mass spectrometry. Ku-0063794 is a recently identified compound that competitively inhibits mTOR kinase activity by occupying the ATP-binding pocket (FIG. 5C) (4, 11). For the Ku-screen, a total of 34,642 phosphopeptides were identified with a false discovery rate of 0.28%, which corresponded to identification of 6,220 unique phosphorylation sites on 1,867 proteins (Table 5). Gene names and sequences of the phosphopeptides whose intensities decrease after Ku-0063794 treatment are identified in Table 8. Note that the light cells were treated with rapamycin and serve as controls whereas the heavy cells were treated with a combination of rapamycin and Ku-0063794. A very tight distribution of phosphopeptides was observed (Median Log₂(H/L)=−0.03 and standard deviation Log₂(H/L)=0.34) and phosphorylated peptides whose relative abundance decreased ≧2-fold were considered regulated by Ku-sensitive, but rapamycin-insensitive mTOR signaling (FIG. 1A). Using these criteria, 100 proteins were determined to contain downregulated phosphorylation after Ku-0063794 treatment (Table 11). The identified phopho-proteins included many known downstream targets of mTORC2 (Table 6), including GSK3β, Braf, Akt1s1 (Akt substrate) and NDRG1 (SGK substrate). Combining the data from both the Rapa-screen and the Ku-screen, we identified a total of 81,777 phosphopeptides, and more than 11,271 unique phosphorylations sites on 2,778 proteins, with 9,694 of the sites confidently localized (A score ≧13) (Table 5).

Phosphorylation Specificity of the Downstream Effectors in the mTORC1 and mTORC2 Signaling Networks

Table 9 describes the classification of mTOR targets identified in the rapamycin and Ku-0063794 screens. Class I includes downstream effectors of rapamycin-sensitive mTORC1. Class II includes downstream effectors of rapamycin-insensitive mTORC1 or mTORC2. Class II includes the proteins downstream of both mTORC1 and mTORC2. Names of the genes and the sequences of the phosphopeptides are shown.

Class I sites represent phosphorylation events that are mediated by rapamycin-sensitive mTORC1/S6K signaling, (e.g. phospho-rpS6 S235/S236 phosphorylation).

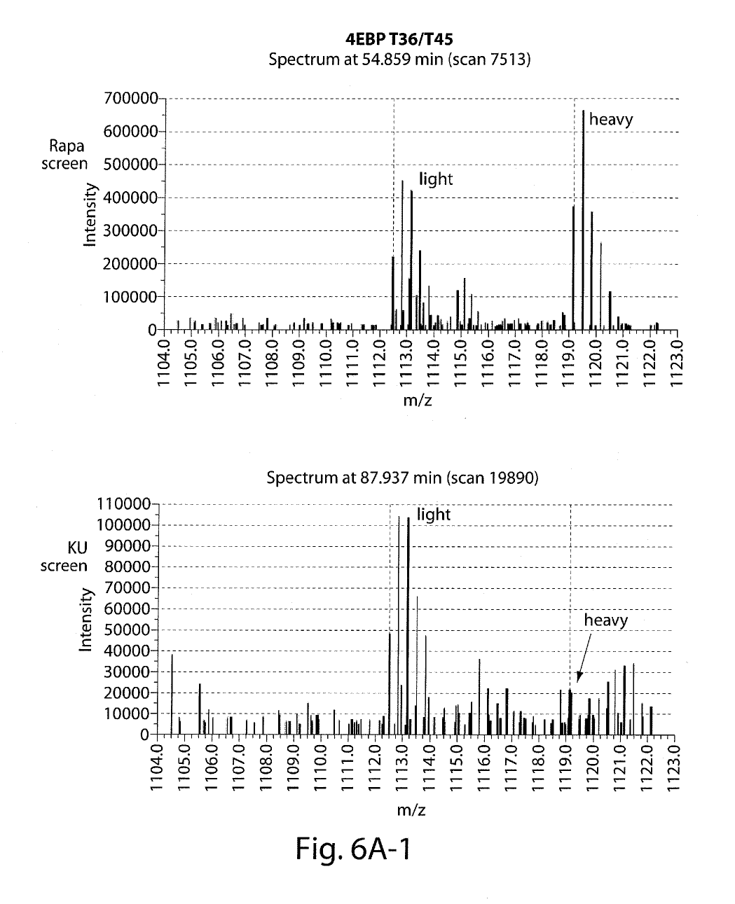

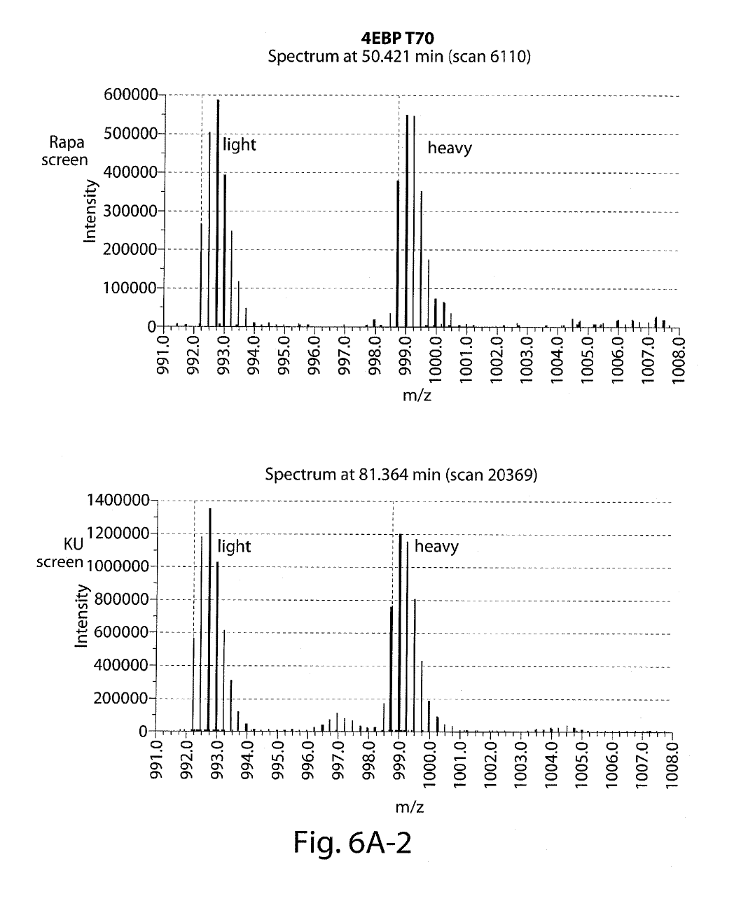

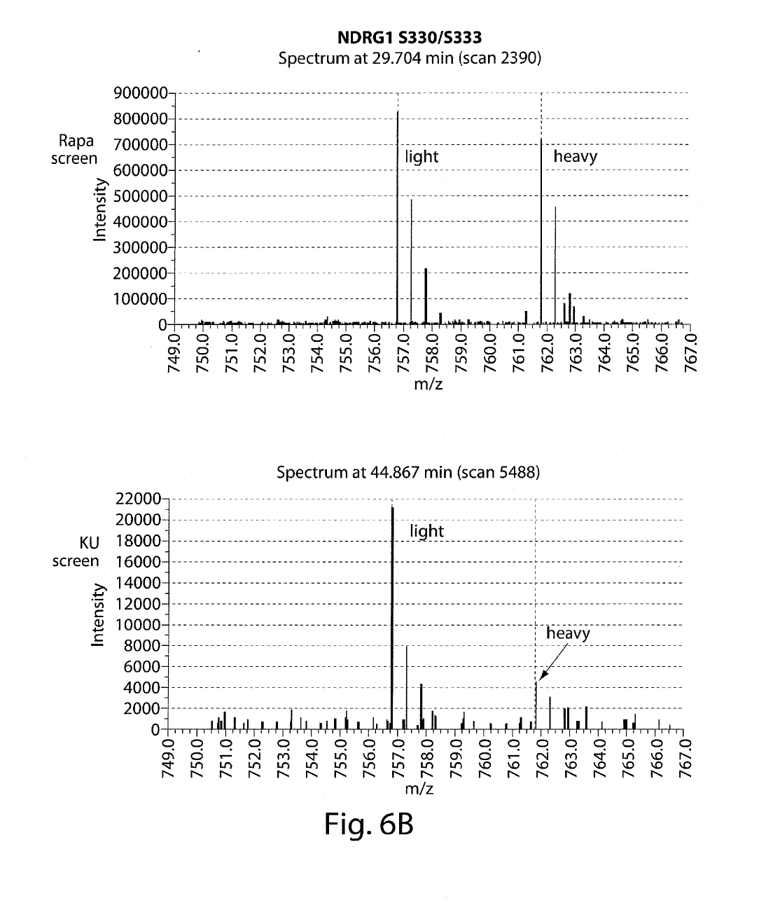

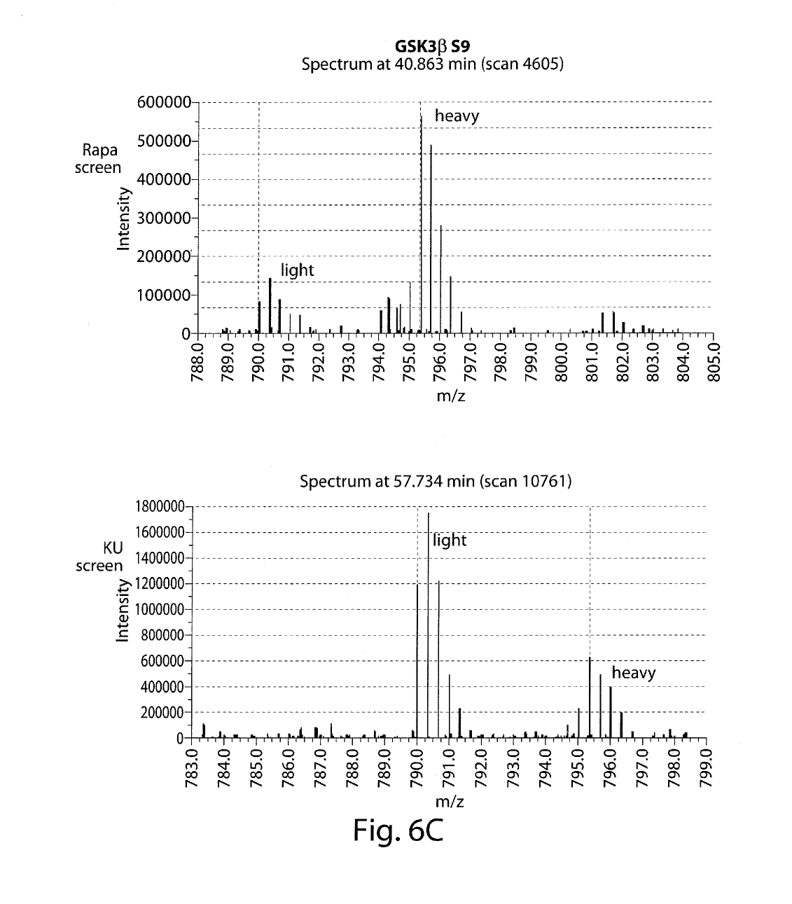

Class II sites represent phosphorylation events mediated by rapamycin-insensitive mTORC1 or mTORC2 function. For example, 4EBP1 T36/T45 phosphorylation, previously characterized as a rapamycin insensitive mTORC1 substrate (12), only decreased slightly (20%) after rapamycin treatment, while phosphorylation at these sites decreased dramatically (7.2-fold) in the Ku-0063794 screen (FIGS. 1C, 5C and 6A). As another example, NDRG1 was also identified to contain Class II phosphorylation sites at S330/S333 (FIGS. 1C and 6B) and was recently shown to be a substrate of SGK (8), whose activation is under the control of mTORC2.

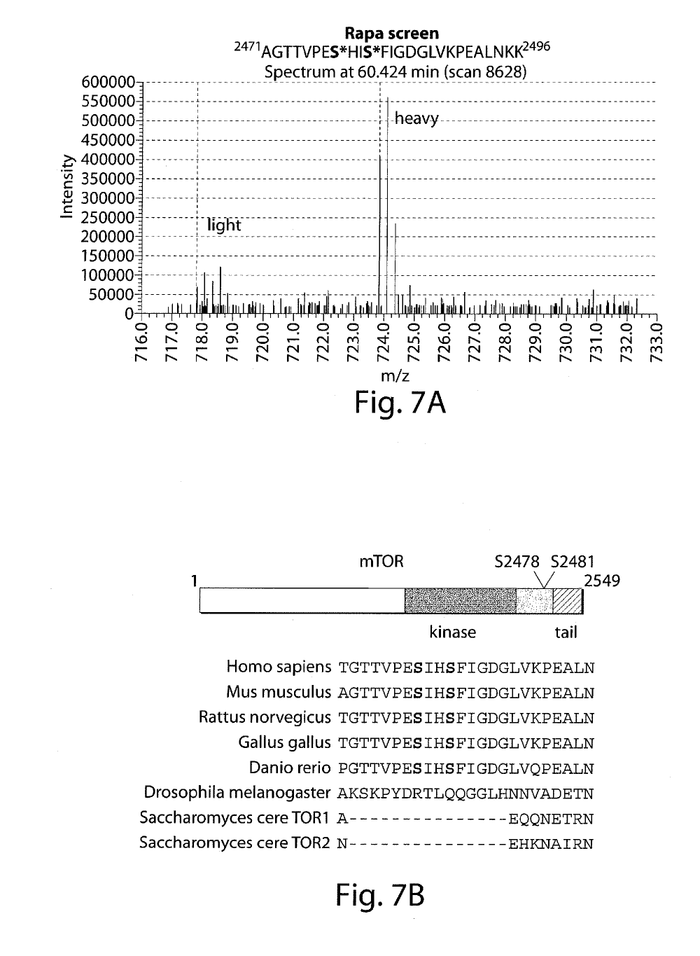

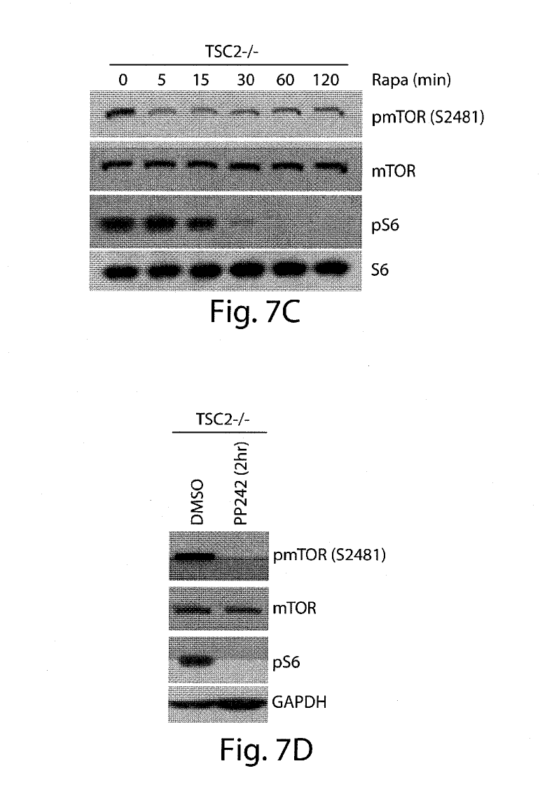

Class III represents phosphorylation sites that are both the rapamycin-sensitive and Ku-sensitive mTOR substrates. For example, GSK3β S9 phosphorylation is down-regulated by approximately 3.3-fold and 2.2-fold in the rapamycin and Ku-0063794 screens, respectively (FIGS. 1C and 6C), consistent with the previous observation that it can be a substrate of both Akt and S6K (9). As another example for the Class III phosphorylation, the abundance of mTOR autophosphorylation at S2478/S2481 decreased 3.6-fold in the rapamycin screen (FIG. 7A). Surprisingly, it was previously shown that S2481 is a conserved, rapamycin-insensitive, autophosphorylation site of mTOR (FIG. 7B) (20). Immunoblot analysis confirmed that site was indeed rapamycin-sensitive (FIGS. 5C and 7C). Intriguingly, acute rapamycin treatment only led to partial dephosphorylation of this site. In contrast, mTOR S2481 phosphorylation was completely abolished as a result of mTOR kinase inhibitor treatment (FIGS. 5C and 7D). Taken together, the data demonstrate that mTOR S2481 phosphorylation is regulated in both a rapamycin sensitive- and insensitive-manner.

There were several surprising examples of phosphopeptides that are insensitive to both rapamycin and Ku-0063794 as identified by the two phosphoproteomics screens. The intensity of a T70 singly phosphorylated 4EBP 1 peptide did not change in either the rapamycin or the Ku-0063794 screen (FIGS. 1C and 6A), indicating the 4EBP T70 is not phosphorylated by mTOR in this context. Indeed, several reports suggested that this site might be targeted by ERK2, another proline-directed kinase (21). In addition, we found that the 4EBP-1 S85/S 100 phosphorylation was also mTOR-independent (FIG. 1C).

Linking Phosphorylation Events to the Biological Processes Regulated by mTORC1 and mTORC2

To assess the predicated effects of mTOR inhibition in cells as a result of either rapamycin or Ku-0063794 treatment, we mapped the downregulated proteins in the two screens to Gene Ontology (GO) terms with respect to Biological Process (BP) and also performed gene functional classification analysis of these proteins. Table 10 describes the results of GO analysis of the hits identified in the rapamycin and Ku-0063794 screens. Pathways that the rapamycin-sensitive and Ku-0063794-sensitive hits overrepresented are shown in “Table 10 Rapa pathways” and “Table 10 Ku pathways”, respectively. Also shown is the biological process that the rapamycin-sensitive and Ku-0063794-sensitive hits overrepresented (Table 10 Rapa BP GO, and Table 10 Ku BP GO, respectively). Interestingly, for the rapamycin screen, the top enriched BPs included negative regulation of macromolecule metabolic process (P=4.2×10⁻⁷), negative regulation of cellular biosynthetic process (P=4.2×10⁻⁶), response to insulin stimulus (P=2.6×10⁻⁴), negative regulation of transcription (P=2.7×10⁻⁴) and vesicle-mediated transport (P=6.0×10⁻⁴). By grouping the hits based on functional similarities, we also found that the downregulated proteins in the rapamycin screen were enriched for kinases (P=2.1×10⁻²⁰), WD40-repeat containing proteins (P=8.9×10⁻¹²), proteins involved in transcription regulation (P=3.1×10⁻⁹) and proteins involved in RNA processing (P=3.3×10⁻⁵). The identification of many kinases in the downregulated proteins provides interesting points for potential signal integration and crosstalk. One example is the Ser/Thr kinase, unc-51-like kinase 1 (ULK1). Previously published research has demonstrated that ULK1 is positively involved in autophagy response (22). ULK1 has also been shown to interact with mTORC1 through binding to raptor (23). We identified three phosphorylation sites on ULK1 (S747/S757/T763) that showed a 4-fold downregulation after rapamycin treatment (Table 1), suggesting that ULK1 is likely a bona fide mTORC1 substrate. As mTORC1 is known to antagonize macroautophagy, it is tempting to speculate that mTORC1 negative regulates ULK1 either directly or indirectly via inhibitory phosphorylation events. Interestingly, in mammalian cells, knockdown of the related kinase ULK2 had no effect on the autophagic response (22). Interestingly, all three of the rapamycin-sensitive ULK1 phosphorylation sites are proline-directed (SP or TP), whereas two of the three comparable sites in ULK2 possess a C-terminal alanine, making it unlikely that these residues are similarly phosphorylated by mTORC1 (FIG. 7E).

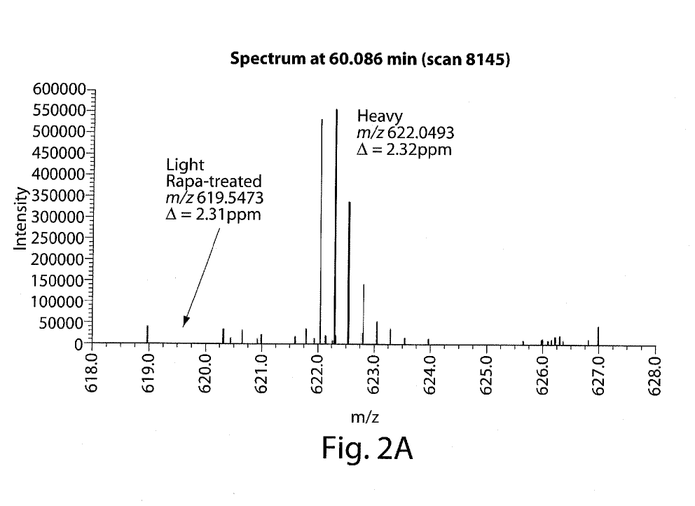

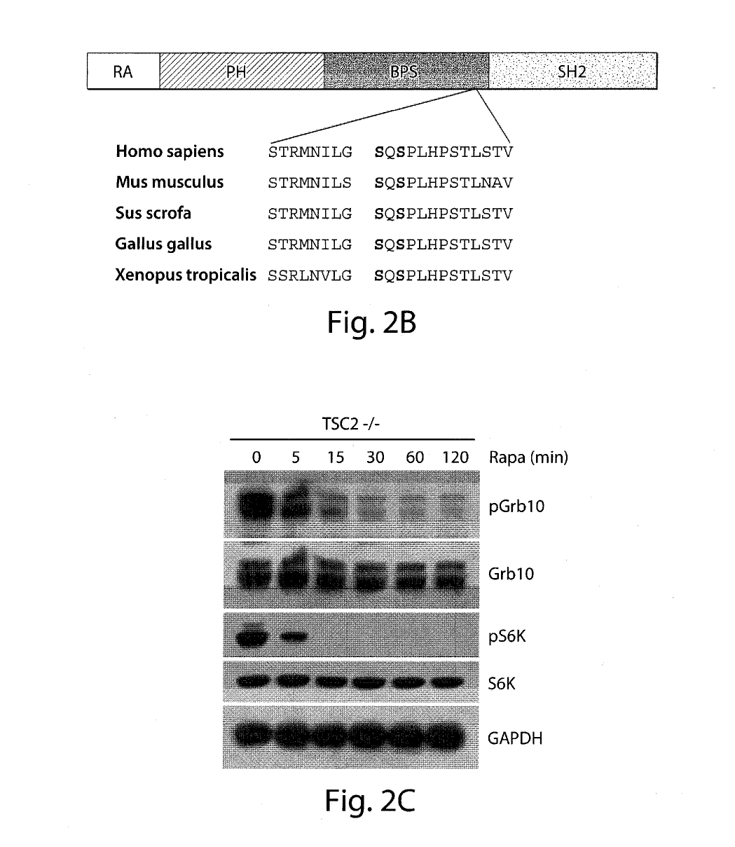

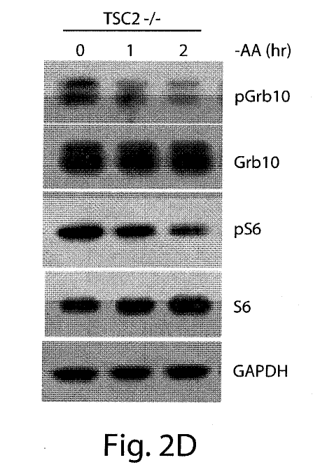

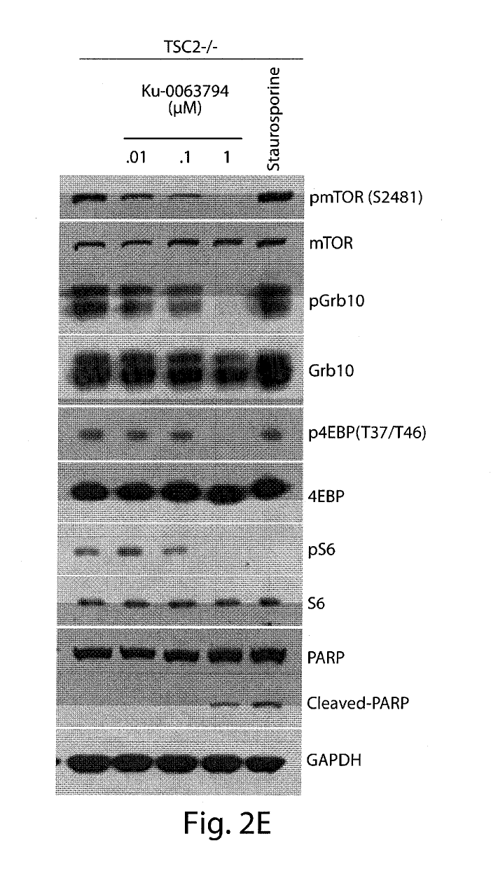

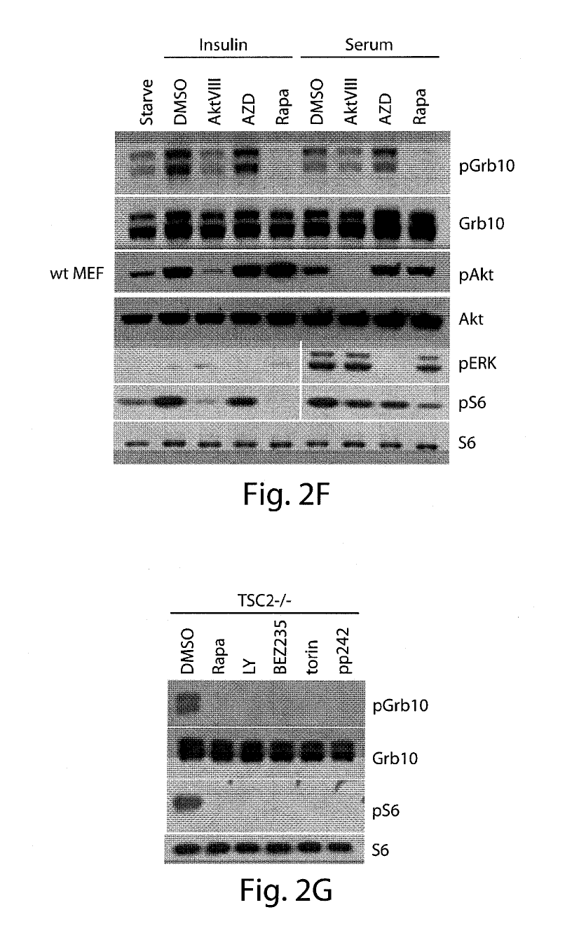

For the downregulated proteins identified in the Ku-0063794 screen, the top enriched BPs included lamellipodium assembly (P=0.003), protein amino acid phosphorylation (P=0.007), the insulin receptor signaling pathway (P=0.01), actin filament-based process (P=0.015) and nucleocytoplasmic transport (P=0.015). Gene functional classification analysis revealed that the downregulated proteins were enriched for ATP-binding proteins (P=2.4×10⁻¹¹) and zinc-finger domain containing proteins (P=2.1×10⁻⁴). Intriguingly, it has been suggested that mTORC2 promotes organization of the actin cytoskeleton upon growth factor stimulation through a mechanism that is poorly defined. The identification of a total of 10 proteins (CCDC88A, ARHGEF17, NCK1, myo9b, Mtap1b, Epb4.113, vel, Spnb2, FLNC, Npm1) in the Ku-0063794 screen that are related to cytoskeleton regulation provides an opportunity for the discovery of novel points of regulation. For example, we identified a peptide from filamin-C (FLNC) in the Ku-0063794 screen that was singly phosphorylated at S2234 (FIG. 2). S2234 is localized in an AGC kinase substrate motif (RERLGS*F, SEQ ID NO: 22) and has previously been shown to be phosphorylated by Akt in vitro (24). We found S2234 could be categorized as a Class II phosphorylation site (rapamycin-insensitive and Ku0063794-sensitive, FIG. 2 and Table 1), suggesting that it is not an S6K substrate but rather the substrate of Akt or SGK. FLNC crosslinks actin filaments into a three-dimensional network and is involved in assembling signaling complexes near the cell membrane (25). Interestingly, filamin-A (FLNA) has been shown to be phosphorylated at a similar site (S2152, RRRAPS*V, SEQ ID NO: 23) by RSK and Pak protein kinases which regulates FLNA function in cell migration (26). It will clearly be of great interest to investigate whether mTORC2 regulates actin assembly and cell migration through Akt- or SGK-mediated phosphorylation of FLNC and whether this phosphorylation coordinates regulation of these processes with Ras-, Cdc42- and Rac-mediated phosphorylation of FLNA.

The analysis identified 4,484 and 6,832 unique phosphorylation sites on 1,615 and 1,866 proteins from two biological replicate experiments, respectively, and achieved a high level of accuracy in quantitation (Table 5). Several hundred peptides corresponding to 148 and 85 proteins in the two replicates (Table 11) were determined to contain rapamycin-sensitive phosphorylation sites (defined as phosphorylated peptides whose relative abundance decreased ≧2-fold in response to rapamycin treatment). There was a substantial overlap in the downregulated proteins between the replicates, as 40 proteins were found in both experiments (FIG. 5B). Supporting the validity of the approach, many known effectors of the mTORC1 signaling pathway were identified in the downregulated population (Table 6), including p70S6K, 4EBP1/2, Akt1s1 (PRAS40), rpS6, eIF4B, eIF4G1 and GSK3β. A representative identification of the known rapamycin-sensitive phosphorylation sites on ribosomal protein S6 is shown in FIG. 1B.

Rapamycin is an allosteric inhibitor that only partially inhibits mTORC1 signaling and has no effect on the activity of mTORC2 under short-term treatment conditions (3). In contrast, newly discovered ATP-competitive mTOR inhibitors block the activity of both mTORC1 and mTORC2 (2). To identify rapamycin-insensitive mTORC1, and mTORC2 substrates, we used the mTOR kinase inhibitor Ku-0063794 and performed a second SILAC experiment (Ku-0063794 screen) (FIG. 1A). The light cells were treated with 20 nM rapamycin for 2 hrs, while the heavy cells were treated in parallel with a combination of 20 nM rapamycin and 2 μM of Ku-0063794 for 2 hrs. Both the light and heavy cells were subsequently stimulated with insulin for 15 minutes and samples were pooled and analyzed by quantitative mass spectrometry. By creating a rapamycin-inhibited background in both the light and heavy cells, the combination of insulin stimulation and treatment with the mTOR kinase inhibitor leads to the identification of proteins specficially phosphorylated by rapamycin-insensitive mTOR signaling and kinases activated downstream of mTORC2 such as Akt and SGK.

In this experiment, one hundred proteins were determined to contain down-regulated phosphorylation after Ku-0063794 treatment (Table 11). The identified phospho-proteins included many known downstream targets of mTORC2 (Table 6), including GSK313, Braf, Akt1s1 (Akt substrates) and NDRG1 (SGK substrate).

To identify the specific downstream effectors of the two mTOR complexes, we compared the fold-change in phospho-peptides identified in both the rapamycin and Ku-0063794 screens (FIG. 1C). As expected, the majority of phospho-peptides in the cell are not affected by either rapamycin or Ku-0063794 treatment and show a heavy:light ratio close to 1:1 in both screens. However, there are clearly phosphopeptides that are mediated by mTORC1 and/or mTORC2 signaling, which can further be divided into three categories. Class I sites include rapamycin-sensitive events that are not further decreased by Ku-0063794 treatment, and represent canonical mTORC1 downstream effectors, such as rpS6 S235/236 phosphorylation (FIG. 1C). Class II sites include Ku-0063794-sensitive events that are not affected by rapamycin treatment and represent either rapamycin-resistant mTORC1 downstream effectors, such as 4EBP1 T36/T45 (FIGS. 1C, 5C and 6A), or sites downstream of the mTORC2 signaling, such as the recently described SGK-mediated NDRG1 S330/S333 phosphorylation events (FIGS. 1C and 6B) (8). Finally, Class III sites are sensitive to both compounds. For example, GSK3β S9 phosphorylation is downregulated in both the rapamycin and Ku-0063794 screens (FIGS. 1C and 6C), consistent with the previous observation that GSK313 can be a substrate of both Akt and S6K (9). In addition, we identified mTOR autophosphorylation at S2481 as a Class III site that is regulated in both a rapamycin sensitive- and insensitive-manner (FIG. 7) (these target classes are discussed in more detail elsewhere herein).

We performed pathway analysis using DAVID (10), to determine the signaling networks that are statistically overrepresented by the hits in the rapamycin and Ku-0063794 screens (FIGS. 1D and 5D). Not surprisingly, proteins with downregulated phosphorylation in both screens were highly enriched for the canonical mTOR pathway (P=1.6×10⁻⁶, ranked first, for the rapamycin screen and P=0.0014, ranked second, for the Ku-0063794 screen).

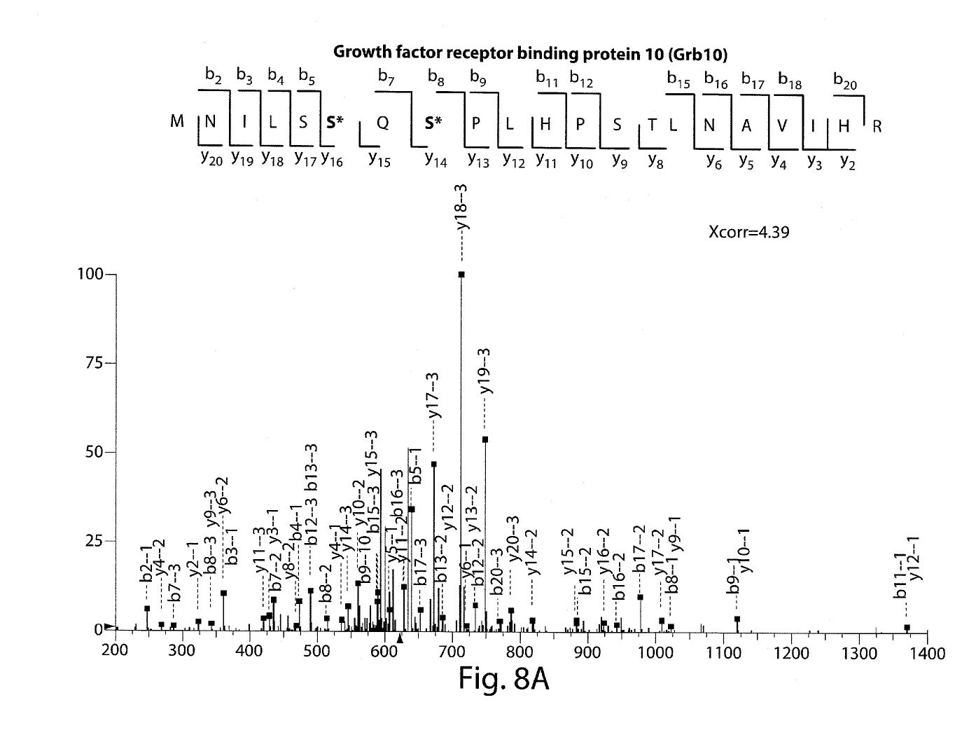

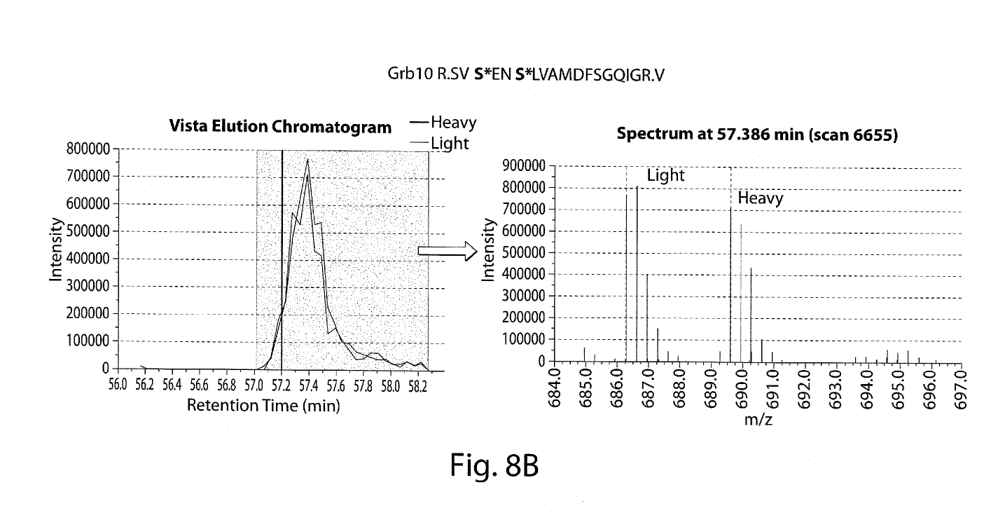

Interestingly, one of the enriched GO classes in the rapamycin screen is the transmembrane receptor protein tyrosine kinase (RTK) signaling pathway (P=0.01), suggesting that mTORC1 might inhibit proteins in the upstream PI3K and MAPK pathways through modulating the activities of these RTKs. In particular, we observed that phosphorylation of two sites on the growth factor receptor-bound protein 10 (Grb10, S501/S503, ⁴⁹⁶MNILSS*QS*PLHPSTLNAVIHR⁵¹⁶ (SEQ ID NO: 24), mass error=2.32 ppm, Xcorr=4.39) was strongly inhibited by rapamycin (FIGS. 2A and 8A). The level of phosphorylation decreased by more than 27-fold after a 2 h rapamycin treatment (Table 6). The intensity of a triply phosphorylated Grb10 peptide (T76/S96/S104, insufficient MS/MS fragment ions to localize T76) also decreased by about five-fold after rapamycin treatment (Table 6). In contrast, phosphorylation of two other Grb10 sites, 5455 and 5458, did not change after a 2-hr rapamycin treatment (FIG. 8B), suggesting that decreased S501/S503 phosphorylation was not the result of a change in Grb10 protein abundance.

Grb10 belongs to the growth factor receptor-bound (Grb) protein family, which contains Grb7, Grb10 and Grb14. Members of this protein family serve as cellular adaptor proteins that bind to activated receptor tyrosine kinases (11). Grb10 has an N-terminal Ras-associating (RA) domain, a PH domain, a C-terminal SH2 domain and a BPS (between PH and SH2) domain, in which the two rapamycin-sensitive phosphorylation sites reside (FIG. 2B). The S501/S503 sites, and their flanking sequences, are highly conserved in vertebrates (FIG. 2B), suggesting that they could be functionally relevant and phosphorylated by a common evolutionarily conserved kinase, such as mTOR.

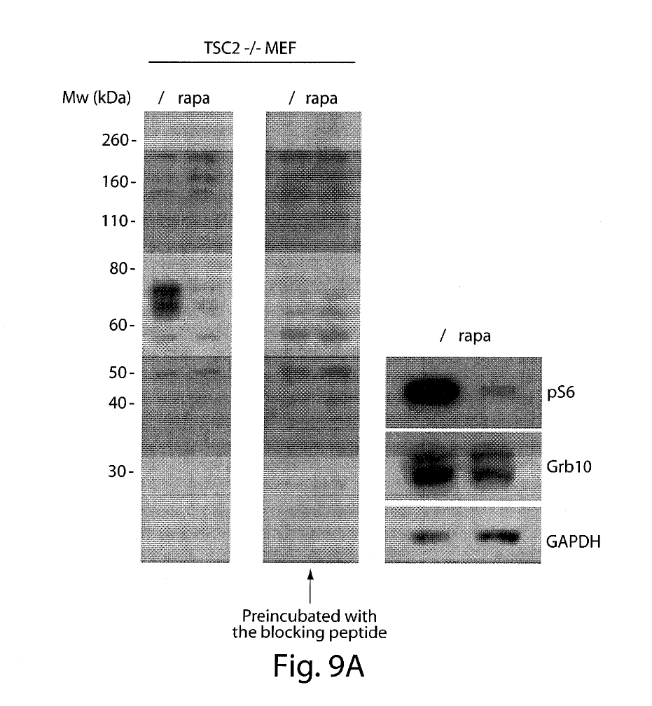

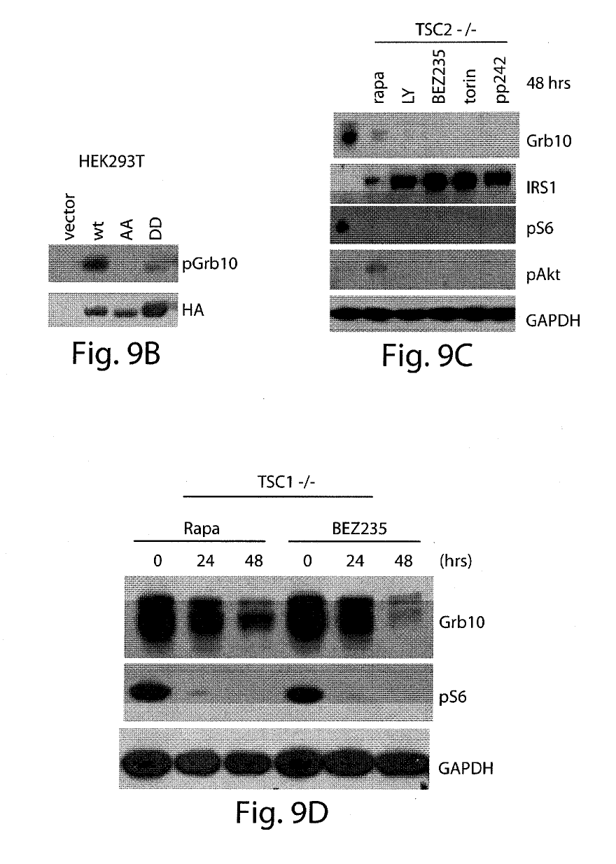

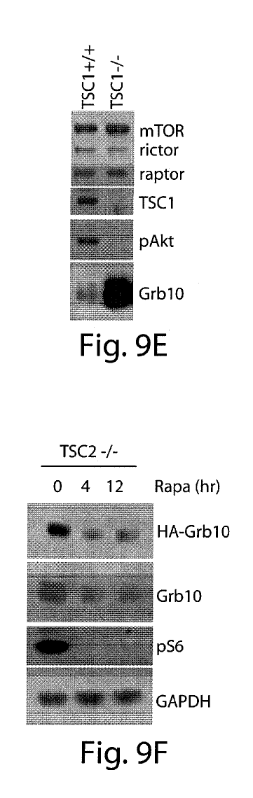

We developed a phosphospecific antibody (FIGS. 9A and 9B) to further characterize these two Grb10 phosphorylation sites. Treatment of TSC2−/− MEFs with 20 nM rapamycin induced rapid dephosphorylation of Grb10, within 15 min, and remained completely inhibited for the remainder of the time course examined (FIG. 2C). The dephosphorylation kinetics correlated well with that of S6K. We also found that Grb10 phosphorylation at S501/S503 is sensitive to amino acid availability in TSC2−/− MEFs (FIG. 2D).

To determine whether the Grb10 S501/S503 sites can be phosphorylated by other kinases, we treated TSC2−/− cells with staurosporine, a broad-spectrum kinase inhibitor that does not suppress mTOR activity (12). No change in the phosphorylation level of Grb10 S501/S503 phosphorylation was observed in response to staurosporine treatment (FIG. 2E). In contrast, treatment of Ku-0063794 led to a dose-dependent dephosphorylation of Grb10, correlating with the dephosphorylation of mTOR, 4EBP and rpS6. Interestingly, S6K activity was inhibited by staurosporine treatment, as shown by a complete loss of rpS6 phosphorylation, suggesting that Grb10 S501/S503 was directly phosphorylated by mTORC1 rather than by S6K.

We further investigated the effects of growth factor stimulation and rapamycin-mediated inhibition on Grb10 phosphorylation in other cell types. Wild-type MEFs were serum-starved and then stimulated with either insulin or 10% serum, both of which led to a robust increase in Grb10 phosphorylation (FIG. 2F). The insulin- or serum-induced increase in Grb10 phosphorylation was completely blocked by rapamycin pre-treatment. It was previously reported that Grb10 S503 (S476 in human Grb10 isoform 3) was phosphorylated by ERK1/2 in vitro (13). We found that inhibiting MEK by using AZD6244 completely abolished the activities of ERK but had no effect on Grb10 S501/S503 phosphorylation (FIG. 2F), indicating that phosphorylation at these two sites on Grb10 is not mediated by ERK in vivo. We also tested other mTOR catalytic site inhibitors, including LY294002, NVP-BEZ235, torin and pp 242 (14), all of which completely abolished Grb10 S501/S503 phosphorylation (FIG. 2G).

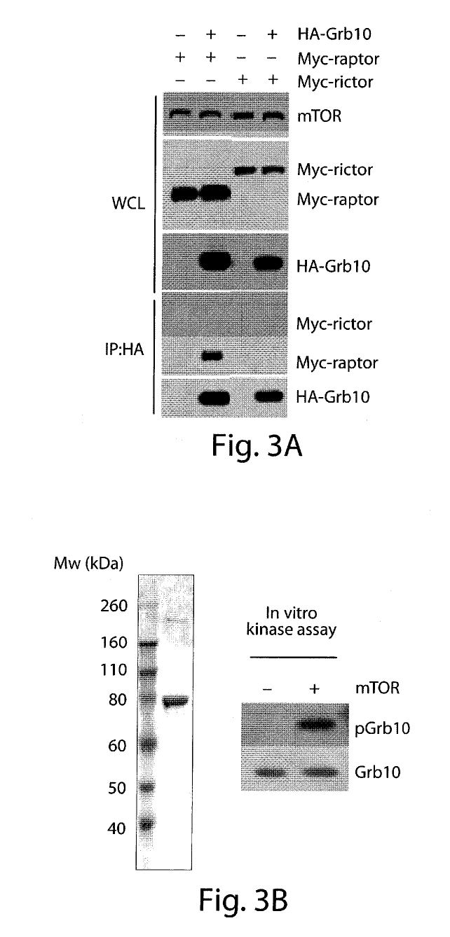

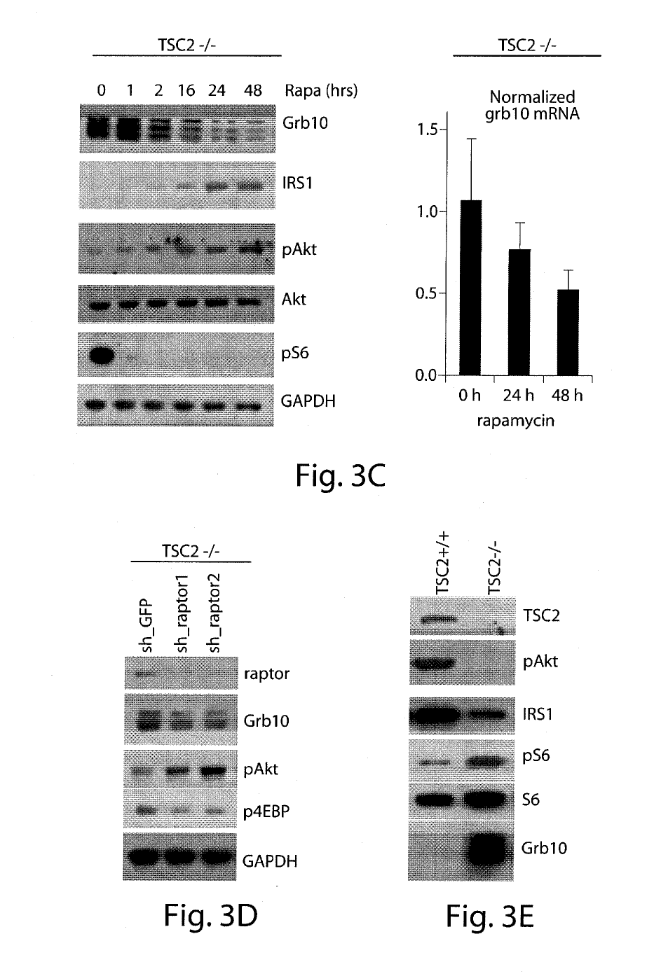

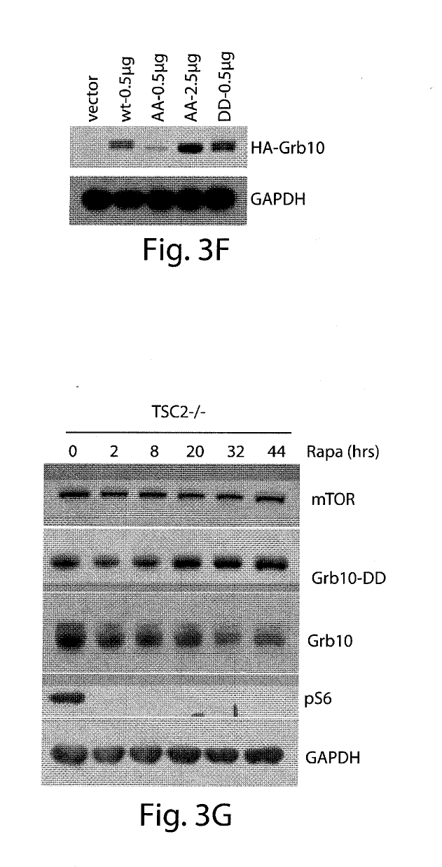

We next examined the in vivo interaction between Grb10 and the components of the mTOR complexes. We co-overexpressed HA-tagged Grb10 with Myc-tagged raptor or rictor in HEK293T cells. Grb10 was found to interact with raptor, but not rictor, suggesting Grb10 is a binding partner of mTORC1, but not mTORC2 (FIG. 3A). To further evaluate the possibility that Grb10 is a substrate of mTORC1, we prepared recombinant Grb10 from bacteria and subjected it to an in vitro kinase assay using recombinant mTOR. As shown in FIG. 3B, Grb10 was robustly phosphorylated by recombinant mTOR at S501/S503.

We found long-term inhibition of mTORC1 by rapamycin led to a significant decrease in Grb10 protein levels (FIG. 3C) while Grb10 mRNA levels showed a modest (approximately 2-fold) decrease, suggesting mTORC1 activity positively regulates Grb10 expression at both the transcriptional and post-translational levels. Similarly, knockdown of the mTORC1 component raptor led to a decreased level of Grb10 protein, correlating with diminished mTORC1 activity, as shown by phospho-4EBP levels (FIG. 3D). In addition, long-term treatment with mTOR kinase inhibitors, including LY-294002, NVP-BEZ235, torin and pp 242 all led to reduced Grb10 protein expression (FIG. 9C). Similar results were obtained in TSC1−/− MEFs (FIG. 9D). In contrast, we found Grb10 was highly overexpressed in TSC2−/− and TSC1−/− MEFs compared with their wild-type counterparts (FIGS. 3E and 9E), correlating with elevated mTORC1 activity in these cells.

To explore whether mTORC1-mediated Grb10 stabilization depends on phosphorylation on S501/S503, we transfected WT-Grb10, Grb10-S501A/S503A (AA) mutant and Grb10-S501D/S503D (DD) mutant into HEK293T cells. Exogenous wild type (WT) and DD mutant Grb10 proteins were expressed at equal levels, while expression of the AA mutant was markedly reduced, suggesting lower protein stability for the unphosphoryated form of Grb10 (FIG. 3F). To confirm this result, we generated TSC2−/− MEFs stably expressing the HA-tagged Grb10-DD mutant and treated these cells with rapamycin. Long-term rapamycin treatment decreased endogenous, wild-type Grb10 levels but had no effect on the DD mutant protein levels (FIG. 3G). To rule out the possibility that this result was an artifact based on protein overexpression, we repeated the experiment on TSC2−/− cells expressing HA-tagged wild-type Grb10, in which rapamycin treatment decreased both the endogenous and ectopically expressed Grb10 (FIG. 9F). These data support a critical role for mTORC1 in stabilizing Grb10 through phosphorylation of the S5011S503 residues.

Grb10 has been suggested to function as a negative regulator of the insulin signaling pathway. In mice with genetically disrupted Grb10 function, overgrowth of both the embryo and placenta was observed, and these mice are approximately 30% larger than normal at birth (15). In addition, enhanced activity of the insulin-stimulated PI3K/Akt pathway was observed in insulin target tissues, including skeletal muscle and adipose tissue (16). We examined the role of mTORC1-mediated Grb10 protein stabilization and accumulation in mTORC1 mediated negative feedback on the PI3K/Akt pathway.

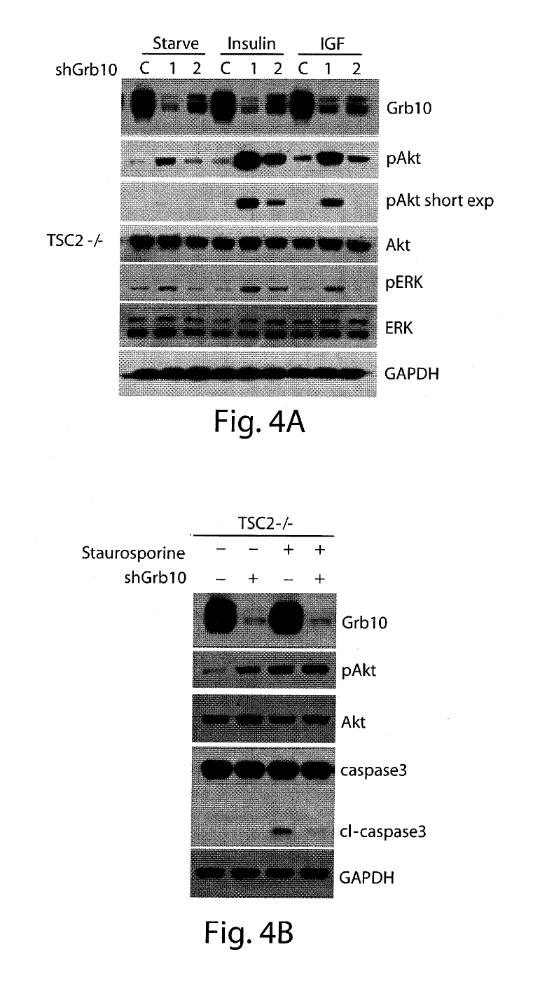

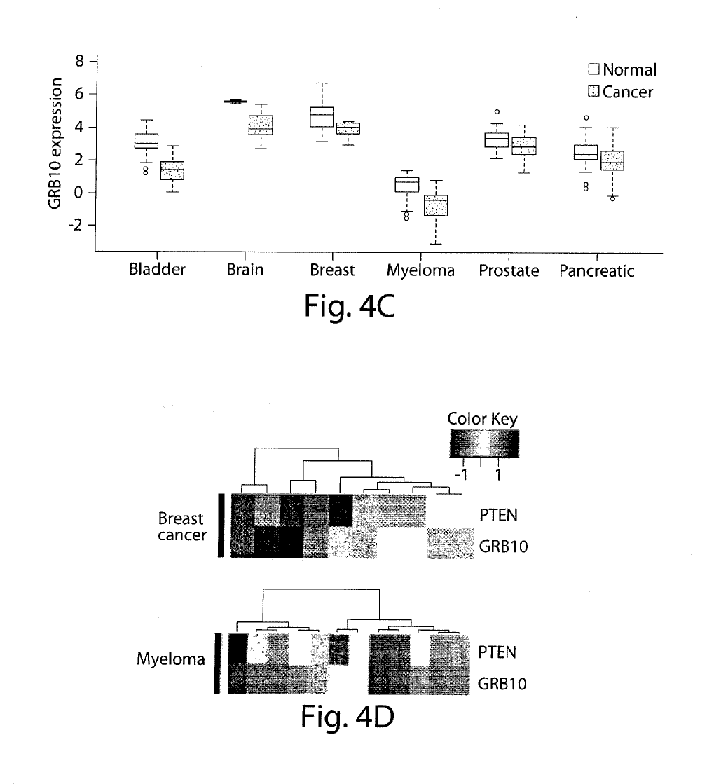

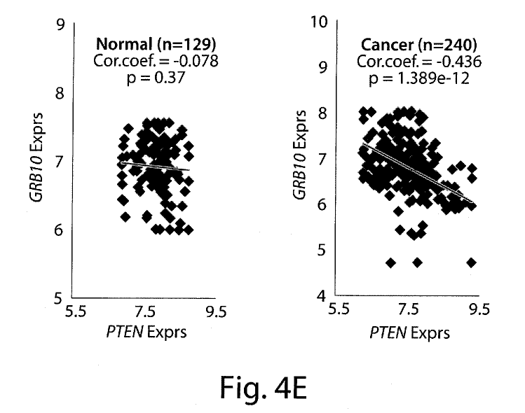

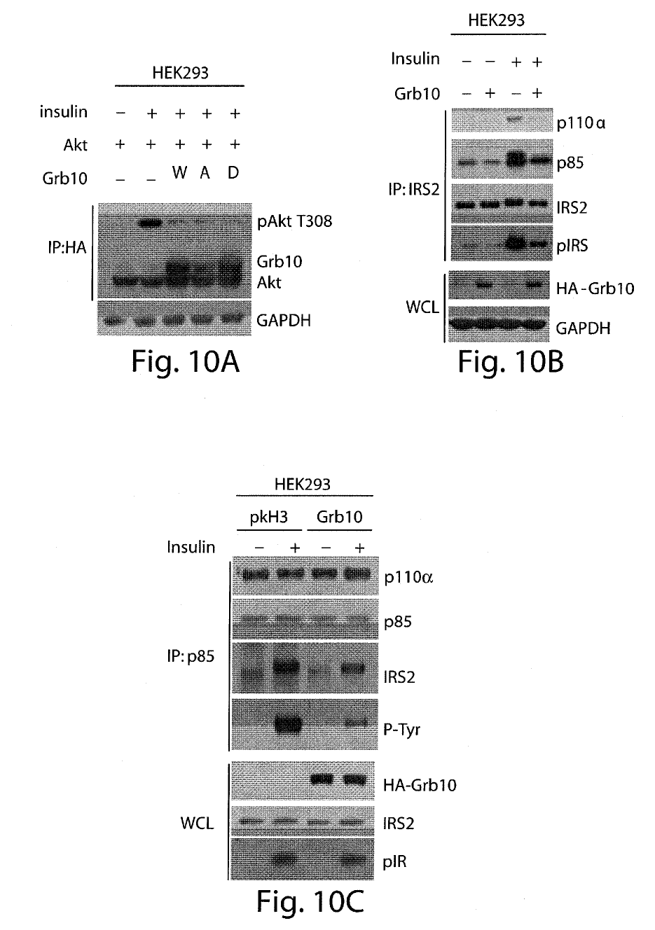

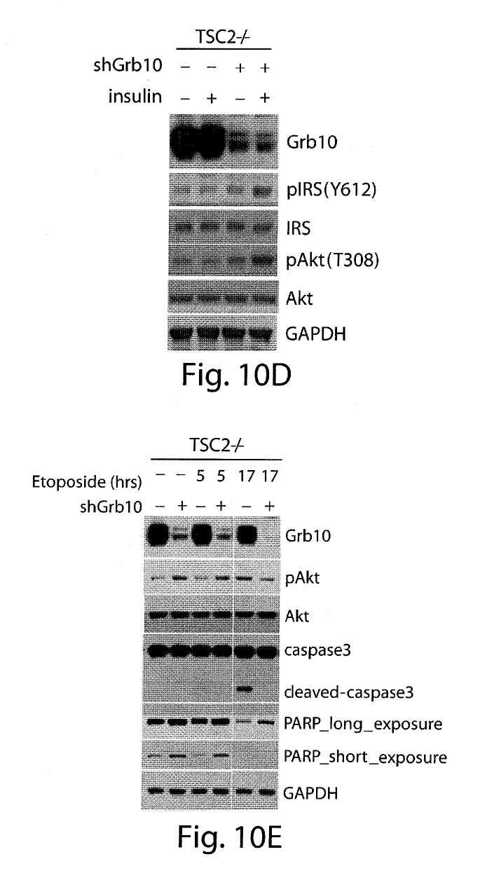

As expected from previous reports (5, 6), both the PI3K/Akt and MAPK pathways were highly suppressed in TSC2−/− cells due to constitutively elevated mTORC1 activity. In contrast, Akt was strongly activated in Grb10 knockdown cells under both serum-starved and insulin/IGF stimulation conditions (FIG. 4A). In addition, Grb10 knockdown also led to ERK hyperactivation upon insulin/IGF stimulation. Conversely, overexpression of Grb10 in HEK293 cells substantially interfered with activation of PI3K, as judged by decreased Akt T308 phosphorylation (FIG. 10A). The observed inhibition was not affected by the phosphorylation status of S501/S503. Previous work demonstrated that Grb10 interferes with insulin-mediated PI3K activation by binding to, and inhibiting, the insulin receptor (11). We confirmed this observation and demonstrated that Grb10 overexpression interferes with insulin receptor-dependent IRS phosphorylation and subsequent PI3K recruitment (FIGS. 10B, 10C and 10D).

The current model of the negative feedback loop from mTORC1 to PI3K involves S6K-mediated phosphorylation and subsequent degradation of IRS 1/2 (6). However, it has also been shown that overexpression of a dominant-negative, kinase-deficient S6K1 failed to completely recapitulate the hyperactivation of Akt induced by overexpression of kinase-dead mTOR, suggesting mTORC1 is directly involved in modulating this feedback inhibition loop (17). Our data clearly show that hyperactivation of mTORC1/S6K promotes feedback loop inhibition of PI3K through a two-prong mechanism: (1) mTORC1/S6K-mediated phosphorylation and degradation of a positive regulator of PI3K signaling, IRS, and (2) mTORC1-mediated phosphorylation and accumulation of a negative regulator of PI3K signaling, Grb10.

Numerous genetic alterations in cancers result in hyperactivation of the mTOR complexes. Based on these observations, rapamycin analogues are currently approved, or being assessed in various clinical trials, as targeted therapeutics against several cancer subtypes. However, with a few exceptions, the current results have been disappointing, as the clinical outcome of rapamycin treatment is unpredictable and rapamycin is largely ineffective as a monotherapy (3). In particular, it has been shown that post-surgical, maintenance rapamycin treatment led to PI3K/Akt activation in glioblastoma patients, and this activation was associated with shorter time-to-progression (4). We asked whether PI3K hyperactivation induced by Grb10 knockdown (which phenotypically mimics rapamycin-induced Grb10 depletion) would contribute to the survival of cells undergoing stress-induced apoptosis. In response to either staurosporine or etoposide, reduced caspase 3 cleavage was observed in Grb10 knockdown cells compared to control cells. These results indicate that Grb10 depletion is sufficient to protect cells from apoptosis (FIGS. 4B and 10E). Combined with the recent finding that rapamycin can protect cells from energy stress-induced death (18), these results provide a plausible mechanism as to why the effect of rapamycin therapy is cytostatic rather than cytotoxic in some types of cancers and suggest a complete understanding of the feedback inhibition control will be critical in designing combination therapy involving rapamycin and DNA-damaging reagents or reagents targeting metabolic pathways.

To explore the role of Grb10 in cancer progression, we performed a comprehensive meta-analysis of GRB10 expression based on published microarray data. GRB10 expression is significantly down-regulated in many tumor types compared to their normal tissue counterparts (FIG. 4C). The most profound down-regulation was observed in infiltrating bladder urothelial carcinoma, glioblastoma, breast, myeloma, prostate and pancreatic cancers. Given that loss of Grb10 results in a dramatic activation of the PI3K/Akt pathway (FIG. 4A), we performed correlation analysis between variations in the expression of GRB10 and PTEN, a known negative regulator of the PI3K/Akt pathway and tumor suppressor gene. While both GRB10 and PTEN are ubiquitously down-regulated, in all cases there was a significantly (p<0.05) negative correlation between GRB10 and PTEN expression. The most striking differences were observed in breast carcinoma (Pearson correlation coefficient=−0.7, p=0.02) and myeloma (Pearson correlation coefficient=−0.82, p=0.001) (FIG. 4D). The data suggest that GRB10 loss provides an alternative mechanism of PI3K/Akt activation when PTEN expression is retained. It is particularly compelling that this correlation is only observed in tumor samples but not the normal tissue controls (FIG. 4E). It has been previously reported that PIK3CA mutations and PTEN loss are mutually exclusive in breast cancer (19), suggesting that an increased level of PIP₃ resulting from of genetic alteration of either PIK3CA or PTEN relieves selective pressure targeting the other gene. Similarly, Grb10 loss, which results in PI3K hyperactivation, might provide the cells with growth and survival advantages that are redundant with respect to PTEN loss-of-function, suggesting that Grb10 might be a novel tumor suppressor that is regulated by mTORC1. These data point to the exciting therapeutic prospects of targeting Grb10 stability in cancer therapy.

### Materials and Methods

### Cells and Reagents

Human embryonic kidney (HEK) 293E cells, immortalized wild-type mouse embryonic fibroblast (MEF) cells and TSC2−/− MEFs (a kind gift from David Kwiatkowski, Brigham and Women's Hospital) were maintained in Dulbecco's modified Eagle's medium (DMEM) supplemented with 10% fetal bovine serum. In collaboration with Millipore Inc., we generated anti-phospho-5501/S503-Grb10 antibodies. Anti-mTOR, anti-phospho-mTOR (S2481), anti-Grb10 (human), anti-phospho-Akt (S473), anti-phospho-Akt (T308), anti-Akt, anti-S6K, anti-phospho-S6K (T389), anti-IRS2, anti-PARP, anti-caspase 3, anti-4EBP, anti-4EBP (T37/T46), anti-phospho-ribosomal protein S6 (S235/S236), and anti-ribosomal protein S6 antibodies were obtained from Cell Signaling Technology. Anti-phospho-ERK1/2 antibody, insulin, Phorbol Myristate Acetate (PMA), Epidermal Growth Factor (EGF) and polybrene were purchased from Sigma. Anti-Grb10 (mouse), anti-phospho-IRS (Y612), anti-p85 and anti-p110 of PI3K antibodies were purchased from Santa Cruz, Invitrogen, Millipore and BD, respectively. ERK1/2 antibody and anti-HA antibody were prepared in the lab. LY294002 and AktVIII inhibitor were purchased from Calbiochem. Lipofectamine 2000 was purchased from Invitrogen. Torin was kindly provided by Nathanael Gray (Dana Farber Cancer Institute).

### SILAC Cell Culture

TSC2−/− MEFs were used in the rapamycin screen due to constitutive hyperactivation of mTORC1 signaling in this cell line. Cells were grown in light ([¹²C₆¹⁴N₂]Lys, [¹²C₆¹⁴N₄]Arg) and heavy ([¹³C₆¹⁵N₂]Lys, [¹³C₆¹⁵N₄]Arg) DMEM (Cambridge Isotope Labs), respectively. Both light and heavy DMEM were supplemented with 10% dialyzed FBS (Invitrogen). Cells were serum-deprived for 17 hours and cells were cultured inheavy media were treated with 20 nM rapamycin for two hours. We performed two biological replicates of this experimental design with cross-labeling (swapping the labeled state of the rapamycin-treated cells). For the purposes of illustration, the data for biological replicate #2 is presented in FIGS. 1A and 1D.

The Ku-0063794 screen was performed using wild-type (WT) MEFs. Cells were grown in the aforementioned SILAC media. Both the light and heavy cells were starved of serum for 17 hrs. The light cells were treated with 20 nM rapamycin for 2 hrs, while the heavy cells were treated with a combination of 20 nM rapamycin and 2 μM Ku-0063794 for 2 hrs. Both the light and heavy cells were then stimulated with 100 nM insulin for 15 min.

### Sample Preparation for Mass Spectrometric Analysis

The heavy and light cells were lysed in urea buffer (8 M urea, 20 mM HEPES pH 7.0, 75 mM β-glycerolphosphate, 1 mM sodium vanadate, 1 mM DTT and 1.5 mM EGTA) and the lysates were combined at a 1:1 ratio. Lysates were reduced by adding DTT to a final concentration of 3 mM, followed by incubation at room temperature for 20 min. Cysteines were alkylated by adding iodoacetamide to a final concentration of 50 mM, followed by incubation in the dark for 20 min. The lysates were diluted to a final concentration of 2 M urea by addition of 100 mM NH₄OAC and were digested overnight with sequencing-grade trypsin (Promega) at a 1:100 (enzyme:substrate) ratio. Digestion was quenched by addition of trifluoroacetic acid to a final concentration of 0.1% and precipitates were removed by centrifugation at 4,000 rpm for 30 min. Peptides were desalted on SepPak C18 columns (Waters) according to manufacturer's instructions.

Phosphopeptides were enriched by SCX-IMAC (27). Briefly, lyophilized peptides were resuspended in 500 μl SCX buffer A (5 mM KH₂PO₄, pH 2.65, 30% acetonitrile) and injected onto a SCX column (Polysulfoethyl aspartamide, 9.4 mm×200 mm, 5 μM particle size, 200 Å pore size, PolyLC). Gradient was developed over 35 min ranging from 0% to 21% buffer B (5 mM KH₂PO₄, pH 2.65, 30% acetonitrile, 350 mM KCl) at a flow rate of 2 ml/min. Twelve fractions were collected and lyophilized. Peptides were then desalted using SepPak C18 columns and were subjected to IMAC (Sigma) for phosphopeptide enrichment. The eluate was further desalted using STAGE tips (28) and lyophilized.

### Mass Spectrometry Analysis and Data Processing

The rapamycin screen samples were analyzed by LC-MS/MS on an LTQ-Orbitrap mass spectrometer (Thermo, San Jose, Calif.) using the top ten method. The Ku-0063794 screen samples were analyzed on an LTQ-Velos mass spectrometer (Thermo Fischer Scientific, San Jose, Calif.) using the top twenty method. MS/MS spectra were searched against a composite database of the mouse IPI protein database (Version 3.60) and its reversed complement using the Sequest algorithm. Search parameters allowed for a static modification of 57.02146 Da for Cys and a dynamic modification of phosphorylation (79.96633 Da) on Ser, Thr and Tyr, oxidation (15.99491 Da) on Met, stable isotope (10.00827 Da) and (8.01420 Da) on Arg and Lys, respectively. Search results were filtered to include <1% matches to the reverse data base by the linear discriminator function (Huttlin et al., manuscript in preparation) using parameters including Xcorr, dCN, missed cleavage, charge state (exclude 1+ peptides), mass accuracy, peptide length and fraction of ions matched to MS/MS spectra. Phosphorylation site localization was assessed by the Ascore algorithm (29) based on the observation of phosphorylation-specific fragment ions and peptide quantification was performed by using the Vista algorithm (30, 31).

We further filter the peptides according to the following criteria for quantitation of the peptide abundance changes: (1) Vista confidence score must be at least 85, (2) signal to noise ratio (S/N)≧3 for both the heavy and light peptides, (3) in the cases where one of the isotopic species has an S/N of ≦3, S/N of the other was required to be ≧5, and (4) in the cases where only the heavy or light version of a peptide was found, we reported the peak S/N ratio, or its inverse, as a proxy for relative abundance measurement. For such peptides, we also required S/N of at ≧5 for the observed species.

### Plasmids

The cDNA for human Grb10 (NCBI gene symbol GRB10; Gene ID: 2887) was obtained from Invitrogen and amplified by PCR. The product was subcloned into (1) the BamH I and EcoR I sites of pKH3, (2) the BamH I and EcoR I sites of pGEX-4T-3 or (3) the Hind III and EcoR I sites of pLPCX. The Grb10 point mutant constructs were generated using the QuickChange site-directed mutagenesis kit (Stratagene). pRK5-Myc-raptor andpRK5-Myc-Rictor were kindly provided by David Sabatini (MIT). Lentiviral plasmids (Δ8.9 and VSVG) were kind gifts from Andrew Kung (Dana Farber Cancer Institute) and David Baltimore (California Institute of Technology).

### Immunoprecipitation

Cells were extracted with lysis buffer A (40 mM HEPES, pH 7.5, 120 mM NaCl, 1 mM EDTA, 10 mM β-glycerophosphate, 50 mM NaF, 2 mM phenylmethylsulfonyl fluoride, 2 mg/ml aprotinin, 2 mg/ml leupeptin, and 1 mg/ml pepstatin, 1 mM DTT) containing 1% Triton X-100, 1% NP-40, or 0.2% CHAPS. After centrifugation, supernatants were collected and pre-cleared for 1 h with protein A- and G-Sepharose beads (GE Healthcare Biosciences). After centrifugation at 3,000 rpm for 5 min, the supernatants were incubated with the antibody at 4° C. for 2 h, and then incubated with protein A- and G-Sepharose for an additional hour. Beads were washed four times with the lysis buffer and eluted in 2× reducing sample buffer.

Mammalian Lentiviral shRNAs

Lentiviral short hairpin RNA (shRNA) expression vectors were a kind gift from William Hahn (Dana Farber Cancer Institute). To generate the lentiviruses, shRNA plasmids were co-transfected into HEK293TD cells along with packaging (Δ8.9) and envelope (VSVG) expression plasmids using lipofectamine 2000 (Invitrogen). Two days after transfection, viral supernatants were harvested and filtered. Recipient cells were infected in the presence of a serum-containing medium supplemented with 8 μg/ml polybrene. Following infection for 36 h, cells were treated with 2.0 μg/ml puromycin (Sigma) and cell lines that stably expressed the shRNAs were selected. Knockdown efficiencies were examined by immunoblot assay using antibodies against the target protein.

### Immunoblot Analysis

For immunoblot analysis, the cells were extracted in lysis buffer (20 mM HEPES (pH 7.5), 1% Triton X-100, 150 mM NaCl, 10 mM EDTA, 1 mM EGTA, 1 mM sodium orthovanadate, 1 mM NaF, 2 mM phenylmethylsulfonyl fluoride, 2 mg/ml aprotinin, 2 mg/ml leupeptin, and 1 mg/ml pepstatin), and extracts were mixed with the 5× reducing buffer (60 mM Tris-HCl, pH 6.8, 25% glycerol, 2% SDS, 14.4 mM 2-mercaptoethanol, 0.1% bromophenol blue). Samples were boiled for 5 min and subject to electrophoresis using the standard SDS-PAGE method. Proteins were then transferred to a nitrocellulose membrane (Whatman). The membranes were blocked with a TBST buffer (25 mM Tris-HCl, pH 7.5, 150 mM NaCl, 0.05% Tween 20) containing 3% nonfat dried milk, and probed overnight with primary antibodies at 4° C. and for 1 h at RT with peroxidase-conjugated secondary antibodies. Blots were developed using enhanced chemiluminescence, exposed on autoradiograph film and developed using standard methods.

### Recombinant Protein Purification

For the purification of GST-tagged proteins, plasmids were transformed into Escherichia coli strain BL21 (DE3), and purified to homogeneity from crude lysates using glutathione-sepharose beads (GE Healthcare) according to the manufacture's protocol. Briefly, protein production was initiated by adding isopropyl-D-thiogalactopyranoside (Sigma) to the cultures. Bacteria were collected by centrifugation, resuspended in PBS and lysed by sonication. After centrifugation at 13,000 rpm for 15 min, the supernatant was incubated with glutathione-sepharose beads for 1 h. The beads were washed with PBS three times and the recombinant protein was eluted with PBS containing 20 mM reduced glutathione. Proteins were dialyzed against PBS and stored at −80° C. until use.

### Quantitative RT-PCR Analysis

Total cellular RNA was purified from cultured cells using the RNeasy mini kit (Qiagen) following the manufacturer's protocol. For quantitative real-time PCR (qRT-PCR), RNA was reverse-transcribed using the High-Capacity cDNA Reverse Transcription Kit (Applied Biosystems) according to the manufacturer's instructions. The resulting cDNA was analyzed by qRT-PCR using the QuantiTect SYBR Green qPCR System (Qiagen). A QuantiTect Primer Assay for mouse Grb10 was used to amplify the target gene, while the β-acting primers (β-actin forward, ACCCAGATCATGTTTGAGACCT (SEQ ID NO: 25); and β-actin reverse, GCAGTAATCTCCTTCTGCATCC (SEQ ID NO: 26)) were used as a normalization control. All reactions were run on an ABI 7900HT Fast Real-Time PCR instrument with a 15 min hot start at 95° C. followed by 40 cycles of a 3-step thermocycling program: denaturation: 15 s at 94° C., annealing: 30 s at 55° C. and extension: 30 s at 70° C. Melting curve analysis was performed at the end of every run to ensure that a single PCR product of the expected melting temperature was produced in a given well. A total of 3 biological replicates×4 technical replicates/biological replicate were performed for each treatment group. Data analysis utilized the comparative Cₜ method (ΔΔCₜ method).

### Analysis of GRB10 and PTEN Expression in Human Samples.

Microarray expression data from six independent data sets corresponding to patient samples from bladder (GSE3167), glioblastoma (GSE4536), breast (GSE5764), myeloma (GSE5900), pancreatic (GSE1542) carcinoma and matching normal tissues were downloaded from Gene Expression Omnibus (www.nebi.nlm.nih.gov/geo/) and for prostate carcinoma from the Broad Institute cancer program datasets (www.broadinstitute.org/cgi-bin/cancer/datasets.cgi). The correlation in the gene expression between GRB10 and PTEN was performed using Pearson's correlation coefficient analysis and the samples were clustered using the Euclidean distance metric and Ward's linkage algorithm.

### REFERENCES

1. X. M. Ma, J. Blenis, Nat Rev Mol Cell Biol 10, 307 (2009).
2. D. R. Alessi, L. R. Pearce, J. M. Garcia-Martinez, Sci Signal 2, pe27 (2009).
3. A. Y. Choo, J. Blenis, Cell Cycle 8, 567 (2009).
4. T. F. Cloughesy et al., PLoS Med 5, e8 (2008).
5. A. Carracedo et al., J Clin Invest 118, 3065 (2008).
6. O. J. Shah, Z. Wang, T. Hunter, Curr Biol 14, 1650 (2004).
7. D. J. Kwiatkowski, B. D. Manning, Hum Mol Genet. 14 Spec No. 2, R251 (2005).
8. J. T. Murray et al., Biochem J 384, 477 (2004).
9. H. H. Zhang, A. I. Lipovsky, C. C. Dibble, M. Sahin, B. D. Manning, Mol Cell 24, 185 (2006).
10. W. Huang da, B. T. Sherman, R. A. Lempicki, Nat Protoc 4, 44 (2009).
11. M. A. Lim, H. Riedel, F. Liu, Front Biosci 9, 387 (2004).
12. A. Y. Choo, S. O. Yoon, S. G. Kim, P. P. Roux, J. Blenis, Proc Natl Acad Sci USA 105, 17414 (2008).
13. P. Langlais et al., Biochemistry 44, 8890 (2005).
14. D. A. Guertin, D. M. Sabatini, Sci Signal 2, pe24 (2009).
15. M. Charalambous et al., Proc Natl Acad Sci USA 100, 8292 (2003).
16. L. Wang et al., Mol Cell Biol 27, 6497 (2007).
17. A. Tzatsos, K. V. Kandror, Mol Cell Biol 26, 63 (2006).
18. A. Y. Choo et al., Mol Cell 38, 487 (2010).
19. L. H. Saal et al., Cancer Res 65, 2554 (2005).
20. R. T. Peterson, P. A. Beal, M. J. Comb, S. L. Schreiber, J Biol Chem 275, 7416 (2000).
21. T. P. Herbert, A. R. Tee, C. G. Proud, J Biol Chem 277, 11591 (2002).
22. E. Y. Chan, S. Kir, S. A. Tooze, J Biol Chem 282, 25464 (2007).
23. N. Hosokawa et al., Mol Biol Cell 20, 1981 (2009).
24. J. T. Murray, D. G. Campbell, M. Peggie, A. Mora, P. Cohen, Biochem J384, 489 (2004).
25. J. M. Dyson et al., J Cell Biol 155, 1065 (2001).
26. M. S. Woo, Y. Ohta, I. Rabinovitz, T. P. Stossel, J. Blenis, Mol Cell Biol 24, 3025 (2004).
27. J. Villen, S. A. Beausoleil, S. A. Gerber, S. P. Gygi, Proc Natl Acad Sci USA 104, 1488 (2007).
28. J. Rappsilber, Y. Ishihama, M. Mann, Anal Chem 75, 663 (2003).
29. S. A. Beausoleil, J. Villen, S. A. Gerber, J. Rush, S. P. Gygi, Nat Biotechnol 24, 1285 (2006).
30. N. Dephoure et al., Proc Natl Acad Sci USA 105, 10762 (2008).
31. C. E. Bakalarski et al., J Proteome Res 7, 4756 (2008).

### INCORPORATION BY REFERENCE

All publications, patents and sequence database entries mentioned herein, including those items listed below and in the databases and tables provided or referred to herein, are hereby incorporated by reference in their entirety for disclosure of the relevant subject matter indicated, as if each individual database entry, publication, or patent was specifically and individually indicated to be incorporated by reference. The specification provides a number of International Protein Index (IPI) accession numbers, starting with IPI followed by a number. International Protein Index database entries identified by IPI accession number in the specification are incorporated by reference for disclosure of the respective protein sequence and accompanying protein information. The IPI database can be accessed at the European Bioinformatics Institute homepage (www.ebi.ac.uk/), for example, at (www.ebi.ac.uk/IPI/IPIhelp.html). In case of conflict, the present application, including any definitions herein, will control.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. For example, it is to be understood that any of the compositions of the invention can be used for vocal cord repair or other soft tissue repair or augmentation. It is also to be understood that any of the compositions made according to the methods for preparing compositions disclosed herein can be used for vocal cord repair or other soft tissue repair or augmentation. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention, can be excluded from any one or more claims. For purposes of brevity, all of the embodiments in which one or more element, feature, purpose, or aspect is excluded are not set forth explicitly herein.

### TABLES

The Tables provided below are referred to in the specification and the claims. Because some of the tables are lengthy, they are provided in this subsection.

TABLE 1

| Gene Symbol | Annotation |
|---|---|
| 1110013L07Rik | Putative uncharacterized protein |
| 1600027N09Rik | RIKEN cDNA 1600027N09 gene |
| 2610110G12Rik | Isoform 1 of UPF0635 protein C6orf134 homolog |
| Aak1 | Isoform 2 of AP2-associated protein kinase 1 |
| Adamts2 | A disintegrin and metalloproteinase with thrombospondin motifs 2 |
| Ahctf1 | AT-hook-containing transcription factor 1 |
| Ahnak | AHNAK nucleoprotein isoform 1 |
| Aim1l | Absent in melanoma 1-like |
| Akt1s1 | Proline-rich AKT1 substrate 1 |
| Alkbh6 | 24 kDa protein |
| Arhgap17 | Isoform 1 of Rho GTPase-activating protein 17 |
| Atg2a | Autophagy-related protein 2 homolog A |
| B230208H17Rik | Putative GTP-binding protein Parf |
| Bat2l | Isoform 1 of Protein BAT2-like |
| Bcas3 | Isoform 1 of Breast carcinoma-amplified sequence 3 homolog |
| Bclaf1 | Isoform 2 of Bcl-2-associated transcription factor 1 |
| Bcr | Breakpoint cluster region protein |
| Bmp2k | Isoform 1 of BMP-2-inducible protein kinase |
| Bod1l | biorientation of chromosomes in cell division 1-like |
| C130092O11Rik | Isoform 1 of Uncharacterized protein KIAA1680 |
| Cabin1 | calcineurin binding protein 1 |
| Carhspl | Calcium-regulated heat stable protein 1 |
| Ccdc6 | coiled-coil domain containing 6 |
| Ccnl1 | Isoform 1 of Cyclin-L1 |
| Cd2ap | CD2-associated protein |
| Cdgap | Cdc42 GTPase-activating protein |
| Chd1 | Chromodomain-helicase-DNA-binding protein 1 |
| Crkrs | Isoform 2 of Cell division cycle 2-related protein kinase 7 |
| Cttn | Src substrate cortactin |
| Cux1 | cut-like homeobox 1 isoform a |
| D6Wsu116e | Isoform 1 of Protein FAM21 |
| D830015G02Rik | Putative uncharacterized protein |
| Dab2 | Isoform p93 of Disabled homolog 2 |
| Ddx21 | Nucleolar RNA helicase 2 |
| Dennd4a | hypothetical protein LOC102442 |
| Dhx15 | Putative pre-mRNA-splicing factor ATP-dependent RNA helicase DHX15 |
| Dnajc2 | DnaJ homolog subfamily C member 2 |
| Dock11 | Dedicator of cytokinesis protein 11 |
| Edc3 | Enhancer of mRNA-decapping protein 3 |
| Edc4 | Isoform 1 of Enhancer of mRNA-decapping protein 4 |
| Eef2k | Elongation factor 2 kinase |
| Ehbp1 | Isoform 2 of EH domain-binding protein 1 |
| Ehmt2 | Isoform 1 of Histone-lysine N-methyltransferase H3 lysine-9 specific 3 |
| Eif3a | Eukaryotic translation initiation factor 3 subunit A |
| Eif4b | Eukaryotic translation initiation factor 4B |
| Eif4ebp1 | Eukaryotic translation initiation factor 4E-binding protein 1 |
| Eif4ebp2 | Eukaryotic translation initiation factor 4E-binding protein 2 |
| Eif4g1 | Isoform 1 of Eukaryotic translation initiation factor 4 gamma 1 |
| Eif5; LOC100047658 | Eukaryotic translation initiation factor 5 |
| Emg1 | Probable ribosome biogenesis protein NEP1 |
| Eps8l2 | Isoform 1 of Epidermal growth factor receptor kinase substrate 8-like protein 2 |
| Epyc | Epiphycan |
| Erc1; LOC100048600 | Isoform 1 of ELKS/RAB6-interacting/CAST family member 1 |
| Ext1 | Exostosin-1 |
| Fbxw9 | F-box and WD-40 domain protein 9 |
| Fkbp15 | Isoform B of FK506-binding protein 15 |
| FLNA | Filamin-A |
| Fmnl3 | Isoform 1 of Formin-like protein 3 |
| Foxk1 | Forkhead box protein K1 |
| Foxk2 | Isoform 1 of Forkhead box protein K2 |
| Fxr1 | Isoform E of Fragile X mental retardation syndrome-related protein 1 |
| Gbf1 | Golgi-specific brefeldin A-resistance factor 1 |
| Gm13099 | Novel protein similar to preferentially expressed antigen in melanoma-like family |
| Gm13697 | Novel protein containing MIF4G and MA3 domains |
| Gm14085 | Novel protein similar to solute carrier family 28 (Sodium-coupled nucleoside |
| | transporter) member 2 |
| Gm6988 | similar to hCG1640785 |
| Gm9757 | Putative uncharacterized protein |
| Gon4l | RIKEN cDNA 5830417110 gene |
| Grb10 | Isoform 3 of Growth factor receptor-bound protein 10 |
| Gsk3b | Glycogen synthase kinase-3 beta |
| Gtf2f1 | General transcription factor IIF subunit 1 |
| Hdgfrp2 | Isoform 3 of Hepatoma-derived growth factor-related protein 2 |
| Herc1 | hect (homologous to the E6-AP (UBE3A) carboxyl terminus) domain and RCC1 |
| | (CHC1)-like domain (RLD) 1 |
| Hisppd1 | 140 kDa protein |
| Hn1l | Hematological and neurological expressed 1-like protein |
| Ibtk | Isoform 2 of Inhibitor of Bruton tyrosine kinase |
| Inf2 | Isoform 1 of Inverted formin-2 |
| Iqsec1 | IQ motif and Sec7 domain 1 isoform b |
| Irs2 | Insulin receptor substrate 2 |
| Iws1 | Isoform 1 of Protein IWS1 homolog |
| Junb | Transcription factor jun-B |
| Kdm3b | Isoform 2 of Lysine-specific demethylase 3B |
| Kdm6a | Isoform 1 of Lysine-specific demethylase 6A |
| Ktn1 | Isoform 1 of Kinectin |
| Larp1 | Isoform 1 of La-related protein 1 |
| Larp4 | Putative uncharacterized protein |
| Larp7 | Isoform 1 of La-related protein 7 |
| Ldhd | 22 kDa protein |
| Lin9 | lsoform 2 of Lin-9 homolog |
| Llgl1 | lethal giant larvae homolog 1 isoform 1 |
| LOC100048123; Akt2 | RAC-beta serine/threonine-protein kinase |
| LOC100048559; Sfrs1 | Isoform 1 of Splicing factor arginine/serine-rich 1 |
| Luc7l2 | Isoform 1 of Putative RNA-binding protein Luc7-like 2 |
| Macf1 | Isoform 3 of Microtubule-actin cross-linking factor 1 |
| Med1 | Isoform 4 of Mediator of RNA polymerase II transcription subunit 1 |
| Megf11 | Isoform 4 of Multiple epidermal growth factor-like domains 11 |
| Mett10d | Isoform 1 of Putative methyltransferase METT10D |
| Mib1 | E3 ubiquitin-protein ligase MIB1 |
| Micall1 | Isoform 1 of MICAL-like protein 1 |
| Mll2 | similar to myeloid/lymphoid or mixed-lineage leukemia 2 |
| Mllt4 | Isoform 3 of Afadin |
| Mogat1 | 2-acylglycerol O-acyltransferase 1 |
| Mtap1b | Microtubule-associated protein 1B |
| Mtor | Isoform 1 of FKBP12-rapamycin complex-associated protein |
| Myef2 | Isoform 2 of Myelin expression factor 2 |
| Myo5a | Myosin-Va |
| Nacc1 | Nucleus accumbens-associated protein 1 |
| Ndrg3 | Protein NDRG3 |
| Nfic | Isoform 1 of Nuclear factor 1 C-type |
| Npm1 | Nucleophosmin |
| Numa1 | Nuclear mitotic apparatus protein 1 |
| Palm | Isoform 1 of Paralemmin |
| Patl1 | Protein PAT1 homolog 1 |
| Pbx2 | Pre-B-cell leukemia transcription factor 2 |
| Pcbp1 | Poly(rC)-binding protein 1 |
| Pcbp2 | Isoform 1 of Poly(rC)-binding protein 2 |
| Pcm1 | Isoform 1 of Pericentriolar material 1 protein |
| Pdcd11 | Protein RRP5 homolog |
| Pds5b | Isoform 1 of Sister chromatid cohesion protein PDS5 homolog B |
| Peg3 | Isoform 1 of Paternally-expressed gene 3 protein |
| Pgrmc2 | Membrane-associated progesterone receptor component 2 |
| Phf3 | PHD finger protein 3 |
| Phldb1 | Isoform 2 of Pleckstrin homology-like domain family B member 1 |
| Phldb2 | lsoform 1 of Pleckstrin homology-like domain family B member 2 |
| Pi4k2a | Phosphatidylinositol 4-kinase type 2-alpha |
| Pkn2 | Isoform 1 of Serine/threonine-protein kinase N2 |
| Pla2g4a | Cytosolic phospholipase A2 |
| Plekhm1 | Pleckstrin homology domain-containing family M member 1 |
| Pom121 | Nuclear envelope pore membrane protein POM 121 |
| Ppfibp2 | Isoform 4 of Liprin-beta-2 |
| Ppp1r12a | MCG122391 isoform CRA_e |
| Prkd2 | Serine/threonine-protein kinase D2 |
| Ptk2 | Isoform 1 of Focal adhesion kinase 1 |
| Qsox2 | Isoform 3 of Sulfhydryl oxidase 2 |
| Rab1 | Ras-related protein Rab-1A |
| Ranbp10 | Ran-binding protein 10 |
| Ranbp9 | RAN binding protein 9 |
| Rb1 | Retinoblastoma-associated protein |
| Rfc1 | Rfc1 protein |
| Rictor | Isoform 1 of rapamycin-insensitive companion of mTOR |
| Rnf19b | IBR domain containing 3 |
| Rps6 | 29 kDa protein |
| Rps6kb1 | Isoform Alpha I of Ribosomal protein S6 kinase beta-1 |
| RsI1d1 | Putative uncharacterized protein |
| rsp6 | 29 kDa protein |
| Sap30 | Histone deacetylase complex subunit SAP30 |
| Sbno1 | Isoform 2 of Protein strawberry notch homolog 1 |
| Scrib | Isoform 1 of Protein LAP4 |
| Serbp1 | Isoform 1 of Plasminogen activator inhibitor 1 RNA-binding protein |
| Setd1a | SET domain containing 1A |
| Sfrs18 | splicing factor arginine/serine-rich 18 |
| Sfrs8 | splicing factor arginine/serine-rich 8 |
| Sgta | Isoform 1 of Small glutamine-rich tetratricopeptide repeat-containing protein alpha |
| Slc4a1ap | solute carrier family 4 (anion exchanger) member 1 adaptor protein |
| Smarca4 | Putative uncharacterized protein |
| Smarcad1 | Isoform 1 of SWI/SNF-related matrix-associated actin-dependent regulator of |
| | chromatin subfamily A containing DEAD/H box 1 |
| Smarcc2 | Isoform 2 of SWI/SNF complex subunit SMARCC2 |
| Snx17 | Sorting nexin-17 |
| Snx30 | Sorting nexin-30 |
| Sorbs3 | Vinexin |
| Spna2 | Isoform 2 of Spectrin alpha chain brain |
| Sqstm1 | Isoform 1 of Sequestosome-1 |
| Srpk2 | serine/arginine-rich protein-specific kinase 2 |
| Srrm1 | Isoform 2 of Serine/arginine repetitive matrix protein 1 |
| Srrm2 | Isoform 3 of Serine/arginine repetitive matrix protein 2 |
| St5 | Isoform 1 of Suppression of tumorigenicity 5 |
| Strn3 | Striatin-3 |
| Syap1 | Synapse-associated protein 1 |
| Tbc1d4 | 140 kDa protein |
| Tcfeb | Transcription factor EB |
| Tinf2 | Putative uncharacterized protein |
| Tjp2 | Tight junction protein ZO-2 |
| Tmem106b | Transmembrane protein 106B isoform CRA_b |
| Tmpo | Isoform Beta of Lamina-associated polypeptide 2 isoforms beta/delta/epsilon/gamma |
| Tnc | Isoform 1 of Tenascin |
| Tns1 | tensin 1 |
| Tox4 | TOX high mobility group box family member 4 |
| Trim28 | Isoform 1 of Transcription intermediary factor 1-beta |
| Trip10 | Isoform 3 of Cdc42-interacting protein 4 |
| Ubxn7 | UBX domain-containing protein 7 |
| Ulk1 | Putative uncharacterized protein |
| Usp24 | Isoform 1 of Ubiquitin carboxyl-terminal hydrolase 24 |
| Usp36 | Ubiquitin specific peptidase 36 |
| Uvrag | UV radiation resistance associated |
| Vwa5b1 | von Willebrand factor A domain-containing protein 5B1 |
| Wdr91 | WD repeat-containing protein 91 |
| Wnk1 | Serine/threonine-protein kinase WNK1 |
| Zc3h4 | Isoform 2 of Zinc finger CCCH domain-containing protein 4 |
| Zc3hc1 | Isoform 1 of Nuclear-interacting partner of ALK |
| Zeb2 | Zinc finger E-box-binding homeobox 2 |
| Zfp106 | Isoform 1 of Zinc finger protein 106 |
| Zfp516 | Zinc finger protein 516 |
| Znrf2 | E3 ubiquitin-protein ligase ZNRF2 |

Table 1. Names and annotations of proteins identified to harbor phosphopeptides whose intensities decrease after rapamycin treatment (rapamycin screen).
Note
that the rapamycin screen included two biological replicates. For the first replicate, the light cells were controls whereas the heavy cells were treated with rapamycin. For the second replicate experiment, the light cells were treated with rapamycin, and the heavy cells were controls. Table 1 includes all unique proteins identified in both replicates. Protein sequences and phosphorylation sites can be identified from the identifiers provided in the tables and database provided herein.

TABLE 2

| Gene Symbol | Annotation |
|---|---|
| Myo18a | Isoform 4 of Myosin-XVIIIa |
| Brd2 | Isoform 2 of Bromodomain-containing protein 2 |
| 6330577E15Rik | Uncharacterized protein C10orf78 homolog |
| Best3 | Bestrophin-3 |
| Larp1 | Isoform 1 of La-related protein 1 |
| Fam62c | Isoform 1 of Extended synaptotagmin-3 |
| Mdn1 | Midasin homolog |
| Rtn4 | Isoform 2 of Reticulon-4 |
| Pcdh24 | Pcdh24 protein |
| Synj1 | similar to mKIAA0910 protein |
| Zfp318 | zinc finger protein 318 isoform 1 |
| Akt1s1 | Proline-rich AKT1 substrate 1 |
| Alpk3 | myocyte induction differentiation originator |
| Zc3h14 | Isoform 2 of Zinc finger CCCH domain-containing |
| | protein 14 |
| Edc4 | Isoform 1 of Enhancer of mRNA-decapping |
| | protein 4 |
| Srrm2 | Isoform 3 of Serine/arginine repetitive matrix |
| | protein 2 |
| Kif20b | Isoform 1 of M-phase phosphoprotein 1 |
| 1110007A13Rik | UPF0557 protein C10orf119 homolog |
| Pi4k2a | Phosphatidylinositol 4-kinase type 2-alpha |
| Top2b | DNA topoisomerase 2-beta |
| BC021381 | Isoform 2 of Uncharacterized protein KIAA1931 |
| Bbx | Isoform 1 of HMG box transcription factor BBX |
| Vcl | Vinculin |
| Eif4ebp1 | Eukaryotic translation initiation factor 4E-binding |
| | protein 1 |
| Rrp15 | RRP15-like protein |
| D10Wsu102e | Uncharacterized protein C12orf45 homolog |
| Braf | Isoform 1 of B-Raf proto-oncogene serine/ |
| | threonine-protein kinase |
| Atg2b | Isoform 1 of Autophagy-related protein 2 |
| | homolog B |
| Npm1 | Nucleophosmin |
| Pcsk5 | proprotein convertase subtilisin/kexin type 5 |
| Mtap1b | Microtubule-associated protein 1B |
| Ercc6l | DNA excision repair protein ERCC-6-like |
| Serinc1 | Serine incorporator 1 |
| Klf3; LOC100046855 | Krueppel-like factor 3 |
| Smarca4 | Putative uncharacterized protein |
| Aak1 | Uncharacterized protein FLJ45252 homolog |
| Eif4ebp2 | Eukaryotic translation initiation factor 4E-binding |
| | protein 2 |
| Ndrg1 | Protein NDRG1 |
| Melk | Maternal embryonic leucine zipper kinase |
| Arhgef17 | Isoform 1 of Rho guanine nucleotide exchange |
| | factor 17 |
| Grit | Isoform 2 of Rho/Cdc42/Rac GTPase-activating |
| | protein RICS |
| Mdc1 | mediator of DNA damage checkpoint 1 |
| Nfkb2 | NF-kB2 splice variant 4 |
| Pcm1 | Isoform 1 of Pericentriolar material 1 protein |
| Mybbp1a | Myb-binding protein 1A |
| Sf3b1 | Splicing factor 3B subunit 1 |
| Atrx | Transcriptional regulator ATRX |
| Ccdc88a | Isoform 2 of Girdin |
| Baz1b | Isoform 1 of Tyrosine-protein kinase BAZ1B |
| Nedd4l | Isoform 3 of E3 ubiquitin-protein ligase NEDD4- |
| | like |
| Orc6l | Origin recognition complex subunit 6 |
| Trp53bp1 | Transformation related protein 53 binding protein 1 |
| Map3k2 | Mitogen-activated protein kinase kinase kinase 2 |
| Hectd2 | Hectd2 protein |
| Usp10 | Ubiquitin carboxyl-terminal hydrolase 10 |
| D830031N03Rik | similar to mKIAA0754 protein |
| Nck1 | non-catalytic region of tyrosine kinase adaptor |
| | protein 1 |
| Exosc9 | Exosome complex exonuclease RRP45 |
| Dap | Death-associated protein 1 |
| Rps6kb1 | Isoform Alpha I of Ribosomal protein S6 kinase |
| | beta-1 |
| Lmna | Isoform C2 of Lamin-A/C |
| Sltm | Isoform 1 of SAFB-like transcription modulator |
| Sh3pxd2a | Isoform 1 of SH3 and PX domain-containing |
| | protein 2A |
| Flnc | Isoform 1 of Filamin-C |
| Oxr1 | Isoform 2 of Oxidation resistance protein 1 |
| Rin2 | Isoform 1 of Ras and Rab interactor 2 |
| Nek9 | Serine/threonine-protein kinase Nek9 |
| Pebp1 | Phosphatidylethanolamine-binding protein 1 |
| Pop1 | Processing of 1 ribonuclease P/MRP family |
| Serhl | Serine hydrolase-like protein |
| Epb4.1l3 | Isoform 1 of Band 4.1-like protein 3 |
| Hnrpll | Isoform 1 of Heterogeneous nuclear |
| | ribonucleoprotein L-like |
| Samhd1 | SAM domain and HD domain-containing protein 1 |
| Zfp828 | Zinc finger protein 828 |
| Larp7 | Isoform 1 of La-related protein 7 |
| Myc | myc proto-oncogene protein |
| Myo9a | Isoform 2 of Myosin-IXa |
| Gsk3b | Glycogen synthase kinase-3 beta |
| Zfp395 | zinc finger protein 395 |
| Bend3 | BEN domain-containing protein 3 |
| Akap12 | Isoform 1 of A-kinase anchor protein 12 |
| Eif4g1 | Isoform 1 of Eukaryotic translation initiation factor |
| | 4 gamma 1 |
| Eif4b | Eukaryotic translation initiation factor 4B |
| Dock7 | Isoform 2 of Dedicator of cytokinesis protein 7 |
| Patl1 | Protein PAT1 homolog 1 |
| Slc7a11 | Cystine/glutamate transporter |
| Myo9b | Isoform 1 of Myosin-IXb |
| Setd2 | SET domain containing 2 |
| Gphn | Gephyrin |
| Erf | ETS domain-containing transcription factor ERF |
| Spnb2 | Isoform 2 of Spectrin beta chain brain 1 |
| Phip | PH-interacting protein |
| Sdpr | Serum deprivation-response protein |
| Tcof1 | Treacle protein |
| Pwp1 | Periodic tryptophan protein 1 homolog |
| Rbl1 | Isoform Long of Retinoblastoma-like protein 1 |
| Eef1b2 | Elongation factor 1-beta |
| Phactr4 | Isoform 1 of Phosphatase and actin regulator 4 |
| C230081A13Rik | Tyrosine-protein kinase-protein kinase SgK269 |
| Ahnak2 | Putative uncharacterized protein |

Table 2. Names and annotations of proteins identified to harbor phosphopeptides whose intensities decrease after Ku-0063794 treatment (Ku-0063794 screen).
Note
that the light cells were treated with rapamycin and serve as controls whereas the heavy cells were treated with a combination of rapamycin and Ku-0063794. Protein sequences and phosphorylation sites can be identified from the identifiers provided in the tables and the database provided herein.

TABLE 3

| | | SEQ | | |
|---|---|---|---|---|
| | | ID | Gene | |
| IPI Reference | Peptide | NO | Name | Class |
| IPI00121418.1 | K.DGEGPDNLEPACPLSLPLQGNHTAADMYLS*PLRS*PK.K | 30 | Rb1 | 1 |
| IPI00221581.1 | R.TGS*ESS*QTGASATSGR.N | 31 | Eif4b | 1 |
| IPI00929786.1 | R.TAS*ISSS*PSEGTPAVGSYGCT*PQS*LPK@.F | 32 | Larp1 | 1 |
| IPI00929786.1 | R.TAS*ISSS*PSEGTPAVGSYGCT*PQS*LPK@.F | 33 | Larp1 | 1 |
| IPI00929786.1 | R.T*AS*ISSSPSEGTPAVGS*YGCTPQSLPK@.F | 34 | Larp1 | 1 |
| IPI00317401.6 | R.AES*PETSAVESTQST*PQK@.G | 35 | Pds5b | 1 |
| IPI00107958.1 | K.TSDIFGS*PVTATAPLAHPNK@PK@.D | 36 | Hn1l | 1 |
| IPI00108454.2 | R.R\hat{ }LS*S*LR\hat{ }.A | 37 | Rps6 | 1 |
| IPI00225062.2 | R.R\hat{ }SS*SELS*PEVVEK@.V | 38 | Srrm2 | 1 |
| IPI00761759.1 | R.S*SSGS*EHST*EGSVSLGDGPLSR\hat{ }.S | 39 | Larp4 | 1 |
| IPI00761759.1 | R.SSS*GS*EHST*EGSVSLGDGPLSR\hat{ }.S | 40 | Larp4 | 1 |
| IPI00225062.2 | R.RSS*SELS*PEVVEK.V | 41 | Srrm2 | 1 |
| IPI00122594.4 | R.TTPLAS*PSLS*PGR\hat{ }.S | 42 | Ahctf1 | 1 |
| IPI00136107.1 | R.THS*TSS*S*IGSGESPFSR\hat{ }.S | 43 | Ndrg3 | 1 |
| IPI00136107.1 | R.THS*TSSS*IGSGESPFSR\hat{ }.S | 44 | Ndrg3 | 1 |
| IPI00136107.1 | R.THS*TSS*SIGSGESPFSR\hat{ }.S | 45 | Ndrg3 | 1 |
| IPI00136107.1 | R.THS*TSSSIGSGESPFSR\hat{ }.S | 46 | Ndrg3 | 1 |
| IPI00136107.1 | R.T*HST*S*SSIGSGESPFSR\hat{ }.S | 47 | Ndrg3 | 1 |
| IPI00136107.1 | R.T*HSTSSS*IGSGESPFSR\hat{ }.S | 48 | Ndrg3 | 1 |
| IPI00136107.1 | R.THS*TSSS*IGSGESPFSR\hat{ }.S | 49 | Ndrg3 | 1 |
| IPI00136107.1 | R.T*HSTSS*SIGSGESPFSR\hat{ }.S | 50 | Ndrg3 | 1 |
| IPI00136107.1 | R.THS*TSSSIGSGESPFSR\hat{ }.S | 51 | Ndrg3 | 1 |
| IPI00551454.3 | R.ER\hat{ }QES*ESEQELVNK@.R | 52 | Pdcd11 | 1 |
| IPI00117229.3 | R.TS*PAGGTWSSVVSGVPR.L | 53 | Atxn2 | 1 |
| IPI00348442.1 | R.SK@FDS*DEEDEDAENLEAVSSGK@.V | 54 | Sfrs18 | 1 |
| IPI00556837.1 | K.DTVIIVS*EPS*EDEESHDLPSVTR.R | 55 | Smarcad1 | 1 |
| IPI00153986.2 | K.GTS*RPGT*PSAEAASTSSTLR.A | 56 | Gtf2f1 | 1 |
| IPI00225062.2 | R.GCS*PPKS*PEKPPQSTSSESCPPS*PQPTK.V | 57 | Srrm2 | 1 |
| IPI00128904.1 | R.VMTIPYQPMPASS*PVICAGGQDR.C | 58 | Pcbp1 | 1 |
| IPI00121251.7 | R.LSTTPS*PT*NSLHEDGVDDFRR.Q | 59 | Tox4 | 1 |
| IPI00320594.5 | K.SQPHSSTSNQETS*DS*EMEMEAEHYPNGVLESVSTR\hat{ }.I | 60 | Ranbp10 | 1 |
| IPI00808277.2 | R.SAPASPNHAGVLS*AHS*SGAQTPESLS*R\hat{ }.E | 61 | Foxk2 | 1 |
| IPI00137166.1 | R.SR\hat{ }DAT*PPVS*PINMEDQER\hat{ }.I | 62 | Junb | 1 |
| IPI00454104.1 | K.TTEAPCS*PGSQQPPS*PDELPANVK@.Q | 63 | Scrib | 1 |
| IPI00225062.2 | R.GCS*PPK@S*PEK@PPQSTSSESCPPS*PQPTK@.V | 64 | Srrm2 | 1 |
| IPI00153986.2 | K.GTS*R\hat{ }PGTPS*AEAASTSSTLR\hat{ }.A | 65 | Gtf2f1 | 1 |
| IPI00753321.2 | K.SVS*ETSEDK@K@DEES*DEEEEEEEEEEPLGATTR\hat{ }.S | 66 | Bod1l | 1 |
| IPI00153986.2 | K.GT*SR\hat{ }PGT*PSAEAASTSSTLR\hat{ }.A | 67 | Gtf2f1 | 1 |
| IPI00153986.2 | K.GTS*R\hat{ }PGT*PSAEAASTSSTLR\hat{ }.A | 68 | Gtf2f1 | 1 |
| IPI00929786.1 | R.TASIS*SSPS*EGTPAVGSYGCT*PQSLPK@.F | 69 | Larp1 | 1 |
| IPI00929786.1 | R.TASISS*SPSEGT*PAVGSYGCT*PQS*LPK@.F | 70 | Larp1 | 1 |
| IPI00130920.1 | R.RSES*PFECK.N | 71 | Mtap1b | 1 |
| IPI00336973.2 | K.GLNLDGTPALSTLGGFS*PASK@PSS*PR\hat{ }.E | 72 | Ccnl1 | 1 |
| IPI00336973.2 | K.GLNLDGTPALSTLGGFSPAS*KPS*SPR.E | 73 | Ccnl1 | 1 |
| IPI00336973.2 | K.GLNLDGT*PALSTLGGFSPAS*K@PS*SPR\hat{ }.E | 74 | Ccnl1 | 1 |
| IPI00336973.2 | K.GLNLDGTPALSTLGGFSPAS*K@PS*SPR\hat{ }.E | 75 | Ccnl1 | 1 |
| IPI00379844.4 | R.TAS*EGDGGAAGGAGTAGGR\hat{ }PMSVAGS*PLS*PGPVR\hat{ }.A | 76 | Irs2 | 1 |
| IPI00336973.2 | K.GLNLDGTPALSTLGGFSPAS*KPSS*PR.E | 77 | Ccnl1 | 1 |
| IPI00336973.2 | K.GLNLDGT*PALSTLGGFSPAS*KPSS*PR.E | 78 | Ccnl1 | 1 |
| IPI00656285.2 | R.SLVS*PIPSPT*GTISVPNSCPAS*PR\hat{ }.G | 79 | Foxk1 | 1 |
| IPI00656285.2 | R.SLVS*PIPS*PTGTISVPNSCPAS*PR.G | 80 | Foxk1 | 1 |
| IPI00454104.1 | K.TTEAPCS*PGSQQPPS*PDELPANVK@.Q | 81 | Scrib | 1 |
| IPI00753321.2 | K.S*VSETSEDK@K@DEES*DEEEEEEEEEEPLGATTR\hat{ }.S | 82 | Bod1l | 1 |
| IPI00753321.2 | K.SVS*ETSEDK@K@DEES*DEEEEEEEEEEPLGATTR\hat{ }.S | 83 | Bod1l | 1 |
| IPI00313307.3 | R.SYQNS*PSS*EDGIR\hat{ }PLPEYSTEK@.H | 84 | Med1 | 1 |
| IPI00225062.2 | R.GCS*PPK@S*PEK@PPQSTSS*ESCPPSPQPTK@.V | 85 | Srrm2 | 1 |
| IPI00318938.6 | R.NS*PVAK@TPPK@DLPAIPGVTS*PTSDEPPMQASQSQL | 86 | Eif4ebp1 | 1 |
| | PSSPEDK@.R | | | |
| IPI00130920.1 | R.SLMS*SPEDLTK@DFEELK@AEEIDVAK@.D | 87 | Mtap1b | 1 |
| IPI00553798.2 | R.SS*EVVLS*GDDEDYQR\hat{ }.I | 88 | Ahnak | 1 |
| IPI00129264.1 | R.ATSR\hat{ }PINLGPSS*PNTEIHWTPYR\hat{ }.A | 89 | Sorbs3 | 1 |
| IPI00129264.1 | R.ATSRPINLGPS*SPNTEIHWTPYR.A | 90 | Sorbs3 | 1 |
| IPI00123410.5 | R.TIS*AQDTLAYATALLNEK@.E | 91 | Usp24 | 1 |
| IPI00226441.2 | R.LHYT*PPLQS*PIT*DGDPLLGQS*PWR\hat{ }.S | 92 | Lin9 | 1 |
| IPI00309059.7 | R.ST*SPIIGS*PPVR\hat{ }.A | 93 | Patl1 | 1 |
| IPI00336713.1 | R.CS*PVPGLSSS*PSGSPLHGK@.L | 94 | Bcas3 | 1 |
| IPI00320905.7 | R.LGEQGPEPGPT*PPQTPT*PPS*TPPLAK.Q | 95 | Arhgap17 | 1 |
| IPI00309059.7 | R.R\hat{ }S*TS*PIIGS*PPVR\hat{ }.A | 96 | Patl1 | 1 |
| IPI00309059.7 | R.RSTS*PIIGS*PPVR.A | 97 | Patl1 | 1 |
| IPI00309059.7 | R.ST*S*PIIGS*PPVR\hat{ }.A | 98 | Patl1 | 1 |
| IPI00317599.3 | K.SQEDEEEISTS*PGVSEFVSDAFDTCSLNQEDLRK.E | 99 | Syap1 | 1 |
| IPI00317599.3 | K.SQEDEEEIST*SPGVSEFVSDAFDTCSLNQEDLRK.E | 100 | Syap1 | 1 |
| IPI00656285.2 | R.S*LVSPIPSPT*GTISVPNS*CPASPR\hat{ }.G | 101 | Foxk1 | 1 |
| IPI00676574.2 | R.RVS*TDLPEGQDVYTAACNSVIHR.C | 102 | Herc1 | 1 |
| IPI00458958.2 | K.DWDK@ES*EGEEPAGGR\hat{ }.A | 103 | Rrp15 | 2 |
| IPI00230719.8 | R.R\hat{ }DSS*DDWEIPDGQITVGQR\hat{ }.I | 104 | Braf | 2 |
| IPI00318938.6 | R.VALGDGVQLPPGDYSTT*PGGTLFSTTPGGT*R.I | 105 | Eif4ebp1 | 2 |
| IPI00874995.2 | R.AS*DDLGEPDVFATAPFR.S | 106 | Aak1 | 2 |
| IPI00318938.6 | R.VALGDGVQLPPGDYSTT*PGGTLFSTTPGGT*R\hat{ }.I | 107 | Eif4ebp1 | 2 |
| IPI00318938.6 | R.VALGDGVQLPPGDYSTTPGGT*LFSTT*PGGTR.I | 108 | Eif4ebp1 | 2 |
| IPI00318938.6 | R.VALGDGVQLPPGDYSTTPGGT*LFSTT*PGGTR\hat{ }.I | 109 | Eif4ebp1 | 2 |
| IPI00323045.3 | R.S*MDVDLNQAHMEDTPK@.K | 110 | Melk | 2 |
| IPI00623284.4 | K.IWDPTPSHT*PAGAAT*PGRGDT*PGHAT*PGHGGATSSA | 111 | Sf3b1 | 2 |
| | R.K | | | |
| IPI00125960.1 | R.TAS*GSS*VTSLEGTR.S | 112 | Ndrg1 | 2 |
| IPI00318938.6 | R.R\hat{ }VALGDGVQLPPGDYSTT*PGGTLFSTT*PGGTR\hat{ }.I | 113 | Eif4ebp1 | 2 |
| IPI00229571.1 | R.ISS*K@S*PGHMVILNQTK@.G | 114 | Sltm | 2 |
| IPI00120095.2 | R.DFTK@PQDGDIIAPLIT*PLK@.W | 115 | Samhd1 | 2 |
| IPI00340860.5 | R.T*AS*EGSEAETPEAPK.Q | 116 | Larp7 | 2 |
| IPI00664670.4 | R.LGS*FGSITR\hat{ }.Q | 117 | Flnc | 2 |
| IPI00654192.2 | R.S*LSFSEPQQPPPTVK@.S | 118 | Zfp395 | 2 |
| IPI00225062.2 | K.IHTTALTGQSPPLAS*GHQGEGDAPSVEPGATNIQQPSS* | 119 | Srrm2 | 2 |
| | PAPSTK.Q | | | |
| IPI00225062.2 | K.IHTTALTGQS*PPLASGHQGEGDAPSVEPGATNIQQPSS* | 120 | Srrm2 | 2 |
| | PAPST*K@.Q | | | |
| IPI00225062.2 | K.IHTTALTGQS*PPLASGHQGEGDAPSVEPGATNIQQPSS* | 121 | Srrm2 | 2 |
| | PAPST*K.Q | | | |
| IPI00339428.9 | R.MSSHTETS*SFLQTLTGR.L | 122 | Dock7 | 2 |
| IPI00225062.2 | R.DGSGT*PSRHSLS*GS*S*PGMKDTPQT*PSR.G | 123 | Srrm2 | 2 |
| IPI00668709.2 | R.DRS*SSAPNVHINTIEPVNIDDLIR.D | 124 | Braf | 2 |
| IPI00135660.5 | R.RGNNS*AVGS*NADLT*IEEDEEEEPVALQQAQQVR.Y | 125 | Sdpr | 2 |
| IPI00135660.5 | R.R\hat{ }GNNSAVGS*NADLT*IEEDEEEEPVALQQAQQVR\hat{ }.Y | 126 | Sdpr | 2 |
| IPI00404545.2 | R.SLS*S*PTVTLSAPLEGAK@.D | 127 | Nedd4l | 2 |
| IPI00121892.9 | R.R\hat{ }PPS*PDPNTK@.V | 128 | Spnb2 | 2 |
| IPI00322707.5 | K.AKQPVIGDQNSDS*DEMLAVLK.E | 129 | Atrx | 2 |
| IPI00929786.1 | R.S*LPTTVPES*PNYR\hat{ }.N | 130 | Larp1 | 3 |
| IPI00125319.1 | R.TTS*FAESCK@PVQQPSAFGSMK@.V | 131 | Gsk3b | 3 |

Table 3. Categorization of mTOR targets identified in the rapamycin and Ku-0063794 screens. Class 1 includes downstream effectors of rapamycin-sensitive mTORC1. Class 2 includes downstream effectors of rapamycin-insensitive mTORC1 or mTORC2. Class 3 includes the proteins downstream of both mTORC1 and mTORC2. Name of exemplary genes and sequences of respective phosphopeptides are shown.
*represents the site of phosphorylation (Ser, Thr and Tyr);
\hat{ }and @ represent heavy Arg and Lys, respectively.

TABLE 4

| | |
|---|---|
| Extracted Term | IPI References |

Rapamycin screen

| | |
|---|---|
| mTOR signaling pathway | IPI00453603, IPI00318938, IPI00399440, IPI00268673, IPI00467843, IPI00221581, IPI00121335 |
| ErbB signaling pathway | IPI00125319, IPI00453603, IPI00318938, IPI00113563, IPI00268673, IPI00121335 |
| Insulin signaling pathway | IPI00125319, IPI00453603, IPI00116923, IPI00318938, IPI00268673, IPI00379844, IPI00121335 |
| Acute myeloid leukemia | IPI00453603, IPI00318938, IPI00268673, IPI00121335 |
| Focal adhesion | IPI00403938, IPI00125319, IPI00131138, IPI00113563, IPI00671847, IPI00121335 |
| Tight junction | IPI00118143, IPI00380354, IPI00323349, IPI00845596, IPI00121335 |
| Pathways in cancer | IPI00125319, IPI00121418, IPI00381495, IPI00113563, IPI00268673, IPI00380817, IPI00121335 |
| Prostate cancer | IPI00125319, IPI00121418, IPI00268673, IPI00121335 |
| RNA degradation | IPI00169888, IPI00309059, IPI00330066 |
| Glioma | IPI00121418, IPI00268673, IPI00121335 |
| Adipocytokine signaling pathway | IPI00268673, IPI00379844, IPI00121335 |
| VEGF signaling pathway | IPI00111169, IPI00113563, IPI00121335 |
| Chronic myeloid leukemia | IPI00121418, IPI00380817, IPI00121335 |

Ku-0063794 Screen

| | |
|---|---|
| Erb6 signaling pathway | IPI00453999, IPI00125319, IPI00453603, IPI00230719, IPI00318938, IPI00668709, IPI00131999 |
| mTOR signaling pathway | IPI00453603, IPI00230719, IPI00318938, IPI00668709, IPI00221581 |
| Acute myeloid leukemia | IPI00453603, IPI00230719, IPI00318938, IPI00668709, IPI00131999 |
| Cell cycle | IPI00125319, IPI00124717, IPI00137864, IPI00131999 |
| Insulin signaling pathway | IPI00125319, IPI00453603, IPI00230719, IPI00318938, IPI00668709 |
| Endometrial cancer | IPI00125319, IPI00230719, IPI00668709, IPI00131999 |
| MAPK signaling pathway | IPI00230719, IPI00117088, IPI00123474, IPI00668709, IPI00664670, IPI00131999 |
| RNA degradation | IPI00309059, IPI00119442, IPI00330066 |
| Focal adhesion | IPI00125319, IPI00230719, IPI00405227, IPI00668709, IPI00664670 |
| Colorectal cancer | IPI00125319, IPI00230719, IPI00668709, IPI00131999 |
| TGF-beta signaling pathway | IPI00453603, IPI00137864, IPI00131999 |

Table 4. Gene ontology analysis of mTOR targets identified in the rapamycin and Ku-0063794 screens. Pathways that were overrepresented among the rapamycin-sensitive and Ku-sensitive mTOR targets are shown. Also shown is the biological process that the rapamycin-sensitive and Ku-sensitive targets overrepresented.

TABLE 5
Summary of the data. Two biological replicates were obtained for the rapamycin
screen (each contained data from two technical replicates) and one SILAC experiment was
performed for the Ku-0063794 screen. (L), light cells. (H), heavy cells.

| | | | | | # of | # of | # of | | Total |
|---|---|---|---|---|---|---|---|---|---|
| | Bio- | # of tech- | | | phospho- | phospho- | unique | Total | phospho- |
| Screen | repli- | replicate | Cell | Drug | peptides | proteins | phospho- | unique | proteins |
| ID | cate | combined | line | treatment | (FDR %) | (FDR %) | sites | sites | (FDR %) |
| Rapa | #1 | 2 | TSC2-/- | (L) DMSO | 14,635 | 1,615 | 4,484 | | |
| | | | | (H) rapa | (0.25%) | (1.0%) | | | |
| | #2 | 2 | TSC2-/- | (L) rapa | 32,500 | 1,866 | 6,832 | 11,271 | 2,778 |
| | | | | (H) DMSO | (0.28%) | (0.97%) | | | (1.7%) |
| Ku | #1 | 1 | wt MEF | (L) rapa | 34,642 | 1,867 | 6,220 | | |
| | | | | (H) rapa+Ku | (0.28%) | (0.97%) | | | |

TABLE 6

| | | | | | |
|---|---|---|---|---|---|
| Gene | | | SEQ | Amino acid | Median |
| Name | Description | Modified Sequence | ID NO | position | (treated/ctrl) |

Rapa Screen (Proteins with downregulated phosphorylation)

| | | | | | |
|---|---|---|---|---|---|
| RPS6 | Ribosomal protein S6 | RLS*S*LRASTSK | 132 | S235, S236 | 30.5 |
| RPS6 | Ribosomal protein S6 | LSSLRAS*TS*KSES*S*QK | 133 | S240, S242, | 4.5 |
| | | | | S246, S247 | |
| Akt1s1 | Proline-rich AKT1 substrate 1 | S*LPVSVPVWAFK | 134 | S184 | 2.2 |
| Gsk3β | Glycogen synthase kinase-3 | TTS*FAESCKPVQQPSAFGSMK | 135 | S9 | 3.3 |
| | beta | | | | |
| Eif4ebp2 | Eukaryotic translation initiation | RNS*PMAQT*PPCHLPNIPGVT | 136 | S65, T70 | 39.1 |
| | factor 4E-binding protein 2 | SPGALIEDSK | | | |
| Eif4ebp2 | Eukaryotic translation initiation | TVAISDAAQLPDYCTT*PGGT | 137 | T37, T46 | 2.3 |
| | factor 4E-binding protein 2 | LFSTT*PGGTRIIYDRK | | | |
| Eif4b | Eukaryotic translation initiation | SRT*GS*ESSQTGASATSGR | 138 | T420, S422 | 4.3 |
| | factor 4B | | | | |
| Eif4b | Eukaryotic translation initiation | TGS*ESS*QTGASATSGR | 139 | S422, S425 | 4.6 |
| | factor 4B | | | | |
| Eif4ebp1 | Eukaryotic translation initiation | NS*PVAKT*PPKDLPAIPGVTSP | 140 | S64, T69 | 2.8 |
| | factor 4E-binding protein 1 | TSDEPPMQASQSQLPSSPEDK | | | |
| Eif4g1 | Isoform 1 of Eukaryotic | SFS*KEVEER | 141 | S1189 | 2.8 |
| | translation initiation factor 4 | | | | |
| | gamma 1 | | | | |
| Rps6kb1 | Isoform Alpha I of Ribosomal | FIGS*PRT*PVS*PVKFSPGDFW | 142 | S441, T444, | 2.8 |
| | protein S6 kinase beta-1 | GR | | S447 | |
| mTOR | Isoform 1 of FKBP12- | AGTTVPES*IHS*FIGDGLVKPE | 143 | S2478, | 3.6 |
| | rapamycin complex- | ALNKK | | S2481 | |
| | associated protein | | | | |
| ULK1 | Serine/threonine-protein | GGGASS*PAPVVFTVGS*PPSG | 144 | S747, S757, | 4.1 |
| | kinase ULK1 | AT*PPQSTR | | T763 | |
| Grb10 | Isoform 3 of Growth factor | MNILSS*QS*PLHPSTLNAVIHR | 145 | S421, S423 | 27.3 |
| | receptor-bound protein 10 | | | | |
| Grb10 | Isoform 3 of Growth factor | T*ASLPAIPNPFPELTGAAPGS* | 146 | T76, S96, | 5.4 |
| | receptor-bound protein 10 | PPSVAPSS*LPPPPSQPPAK | | S104 | |

Ku Screen (Proteins with downregulated phosphorylation)

| | | | | | |
|---|---|---|---|---|---|
| Flnc | Filamin-C | LGS*FGSITR | 147 | S2234 | 2.4 |
| Gsk3β | Glycogen synthase kinase-3 | TTS*FAESCKPVQQPSAFGSMK | 148 | S9 | 2.2 |
| | beta | | | | |
| Ndrg1 | Protein NDRG1 | TAS*GSS*VTSLEGTR | 149 | S330, S333 | 4.1 |
| Braf | B-Raf protein | DRSSS*APNVHINTIEPVNIDDLIR | 150 | S312 | 2.1 |
| Akt1s1 | Proline-rich AKT1 substrate 1 | LNT*SDFQK | 151 | T247 | 31.3 |
| Eif4b | Eukaryotic translation initiation | TGS*ESSQTGASATSGR | 152 | S422 | 2.2 |
| | factor 4B | | | | |
| Eif4ebp1 | Eukaryotic translation initiation | VALGDGVQLPPGDYSTT*PGGT | 153 | T36, T45 | 7.2 |
| | factor 4E-binding protein 1 | LFSTT*PGGTR | | | |
| Eif4ebp2 | Eukaryotic translation initiation | NS*PMAQTPPCHLPNIPGVTSP | 154 | S65 | 6.8 |
| | factor 4E-bindg protein 2 | GALIEDSK | | | |

Table 6. Representative hits identified in the rapamycin and Ku-0063794 screens. Only protein targets with downregulated phosphorylation are shown. Median fold-changes of the identified phosphopeptides are reported. For the rapamycin screen, hits from the second biological replicate are shown. The control group is DMSO vehicle treated sample whereas the experiment group is the one treated with rapamycin. For the Ku-0063794 screen, the control group was treated with rapamycin whereas the experiment group was treated with a combination of rapamycin and Ku-0063794. An asterisk indicates the site of phosphorylation. Phosphorylation of Grb10 in MNILSS*QS*PLHPSTLNAVIHR (SEQ ID NO: 27) corresponds to S421/S423 in isoform 3 and S501/S503 in isoform 1, respectively. The site designation in the text corresponds to the sites in isoform 1 (mouse sequence).

TABLE 7

| | | | | | |
|---|---|---|---|---|---|
| | | | | | Log2 |
| | | SEQ | | | Area |
| | | ID | Gene | | (H/L) |
| Reference | Ascore Seq | NO: | Name | Annotation | Ratio |

Rapa replicate 1 (light cells were control and heavy cells were treated
with rapamycin. L, light; H, heavy)

| | | | | | |
|---|---|---|---|---|---|
| IPI:IPI00136419.1 | LDGNPINLS*R | 155 | Epyc | Epiphycan | −7.993 |
| IPI:IPI00108454.2 | LSS*LRAS*TSKSESSQK | 156 | rpS6 | 29 kDa protein | −7.693 |
| IPI:IPI00108454.2 | RLS*S*LRASTSK | 157 | rpS6 | 29 kDa protein | −6.817 |
| IPI:IPI00225062.2 | TPQAPTPANLVVGPR\hat{ }S*A | 158 | Srrm2 | Isoform 3 of Serine/arginine repetitive | −6.606 |
| | HGT*APVNIAGS*R\hat{ } | | | matrix protein 2 | |
| IPI:IPI00111497.1 | VTEGSYSCLAHS*PLGVVA | 159 | Igdcc4 | Isoform 2 of Immunoglobulin | −6.062 |
| | SQVAVVK | | | superfamily DCC subclass member 4 | |
| IPI:IPI00874813.1 | MPAT*DTANLTAPWHPR | 160 | Ldhd | 22 kDa protein | −5.745 |
| IPI:IPI00126317.1 | NAS*TS*FQELEDKK | 161 | Dnajc2 | DnaJ homolog subfamily C member 2 | −5.522 |
| IPI:IPI00187443.1 | WLKEAEEES*S*GGEEEDE | 162 | Eif5; | Eukaryotic translation initiation | −5.378 |
| | DENIEVVYSK | | LOC100047658 | factor 5 | |
| IPI:IPI00318938.6 | RVALGDGVQLPPGDYSTT* | 163 | Eif4ebp1 | Eukaryotic translation initiation | −5.301 |
| | PGGTLFS*TTPGGTR | | | factor 4E-binding protein 1 | |
| IPI:IPI00225062.2 | S*AHGT*APVNIAGSRTPAG | 164 | Srrm2 | Isoform 3 of Serine/arginine repetitive | −5.098 |
| | LAPTNLS*SSR | | | matrix protein 2 | |
| IPI:IPI00467423.3 | GYS*YDDSMESR | 165 | Gm13697 | Novel protein containing MIF4G and | −5.093 |
| | | | | MA3 domains | |
| IPI:IPI00467423.3 | GYS*YDDSMESR | 166 | Gm13697 | Novel protein containing MIF4G and | −4.733 |
| | | | | MA3 domains | |
| IPI:IPI00130920.1 | SDISPLT*PR | 167 | Mtap1b | Microtubule-associated protein 1B | −4.668 |
| IPI:IPI00133685.1 | AATATRPPGPPPAPQPPS* | 168 | Akt1s1 | Proline-rich AKT1 substrate 1 | −4.667 |
| | PAPS*PPPRPALAR | | | | |
| IPI:IPI00154084.2 | GFGDS*FGR | 169 | Myef2 | Isoform 2 of Myelin expression factor 2 | −4.553 |
| IPI:IPI00338745.4 | QADVADQQTTELPAENGET | 170 | Hmgn1; | Non-histone chromosomal protein | −4.546 |
| | ENQSPAS*EEEKEAKS*D | | LOC100044391 | HMG-14 | |
| IPI:IPI00109318.1 | RNS*PMAQT*PPCHLPNIPG | 171 | Eif4ebp2 | Eukaryotic translation initiation | −4.531 |
| | VTSPGALIEDSK | | | factor 4E-binding protein 2 | |
| IPI:IPI00154084.2 | GFGDS*FGR | 172 | Myef2 | Isoform 2 of Myelin expression factor 2 | −4.429 |
| IPI:IPI00121519.1 | SYSESGLMGEPQPQGPPS* | 173 | Neurod1 | Neurogenic differentiation factor 1 | −4.393 |
| | WTDECLS*S*QDEEHEADKK | | | | |
| IPI:IPI00317794.5 | KEDS*DEDEDEEDEDDS*D | 174 | Ncl | Nucleolin | −4.345 |
| | EDEDDEEEDEFEPPIVK | | | | |
| IPI:IPI00399953.1 | VFTSDISDPVVASTSQAPG | 175 | Wnk1 | Serine/threonine-protein kinase WNK1 | −4.306 |
| | M#NLSHSASSLS*LQQAFS | | | | |
| | ELK | | | | |
| IPI:IPI00263048.1 | VS*SETHQGPGTPESK | 176 | Numa1 | Nuclear mitotic apparatus protein 1 | −4.079 |
| IPI:IPI00225062.2 | TPPSAPS*QSRM#T*SER | 177 | Srrm2 | Isoform 3 of Serine/arginine repetitive | −4.002 |
| | | | | matrix protein 2 | |
| IPI:IPI00263048.1 | VSS*ETHQGPGTPESK | 178 | Numa1 | Nuclear mitotic apparatus protein 1 | −3.674 |
| IPI:IPI00664808.2 | SSSPT*NSLTQPIEMPTLSS | 179 | Kdm3b | Isoform 2 of Lysine-specific | −3.64 |
| | S*PTEER\hat{ }PTVGPGQQDNP | | | demethylase 3B | |
| | LLK@ | | | | |
| IPI:IPI00225062.2 | RKETPS*PR | 180 | Srrm2 | Isoform 3 of Serine/arginine repetitive | −3.569 |
| | | | | matrix protein 2 | |
| IPI:IPI00453603.1 | TPVS*PVKFS*PGDFWGR | 181 | Rps6kb1 | Isoform Alpha I of Ribosomal protein | −3.424 |
| | | | | S6 kinase beta-1 | |
| IPI:IPI00318938.6 | R\hat{ }VALGDGVQLPPGDY*ST | 182 | Eif4ebp1 | Eukaryotic translation initiation | −3.386 |
| | TPGGTLFSTTPGGT*R\hat{ } | | | factor 4E-binding protein 1 | |
| IPI:IPI00330066.5 | DSQDTSAEQS*DHDDEVAS | 183 | Edc4 | Isoform 1 of Enhancer of mRNA- | −3.347 |
| | LASASGGFGSK | | | decapping protein 4 | |
| IPI:IPI00399953.1 | DAMNLS*GR | 184 | Wnk1 | Serine/threonine-protein kinase WNK1 | −3.343 |
| IPI:IPI00127707.1 | LHQLAMQQSHFPMTHGNT | 185 | Pcbp2 | Isoform 1 of Poly(rC)-binding protein 2 | −3.277 |
| | GFSGIESSS*PEVK | | | | |
| IPI:IPI00458068.6 | SNS*LSEQLTVNSNPDTVK | 186 | Fkbp15 | Isoform B of FK506-binding protein 15 | −3.252 |
| IPI:IPI00116442.1 | S*EGLSLER | 187 | Hdgfrp2 | Isoform 3 of Hepatoma-derived growth | −3.127 |
| | | | | factor-related protein 2 | |
| IPI:IPI00116442.1 | S*EGLSLER\hat{ } | 188 | Hdgfrp2 | Isoform 3 of Hepatoma-derived growth | −3.127 |
| | | | | factor-related protein 2 | |
| IPI:IPI00112101.1 | TTQSLQDFPVADS*EEEAE | 189 | Tfip11 | Tuftelin-interacting protein 11 | −3.096 |
| | EEFQKELSQWR | | | | |
| IPI:IPI00750472.3 | TESASLSQS*QVNGFFASH | 190 | Ptpn13 | Tyrosine-protein phosphatase non- | −3.058 |
| | LGDR | | | receptor type 13 | |
| IPI:IPI00318938.6 | VALGDGVQLPPGDYS*TTP | 191 | Eif4ebp1 | Eukaryotic translation initiation | −3.042 |
| | GGT*LFST*TPGGTR | | | factor 4E-binding protein 1 | |
| IPI:IPI00317599.3 | SQEDEEEISTS*PGVSEFVS | 192 | Syap1 | Synapse-associated protein 1 | −3.022 |
| | DAFDTCS*LNQEDLRK | | | | |
| IPI:IPI00116442.1 | S*EGLSLER | 193 | Hdgfrp2 | Isoform 3 of Hepatoma-derived growth | −3.012 |
| | | | | factor-related protein 2 | |
| IPI:IPI00553798.2 | T*PEMIIQKPKIS*M#QDVDL | 194 | Ahnak | AHNAK nucleoprotein isoform 1 | −2.987 |
| | S*LGSCK | | | | |
| IPI:IPI00751009.1 | TSEFPTPLFSGPLEPVACG | 195 | Srpk2 | serine/arginine-rich protein specific | −2.951 |
| | SVISEGSPLTEQEESSPSH | | | kinase 2 | |
| | DR\hat{ }S*R\hat{ } | | | | |
| IPI:IPI00127976.1 | MNILSSQS*PLHPSTLNAVI | 196 | Grb10 | Isoform 3 of Growth factor receptor- | −2.946 |
| | HR | | | bound protein 10 | |
| IPI:IPI00127976.1 | M#NILSS*QSPLHPSTLNAVI | 197 | Grb10 | Isoform 3 of Growth factor receptor- | −2.944 |
| | HR | | | bound protein 10 | |
| IPI:IPI00656285.2 | SSGLQTPECLS*REGS*PIP | 198 | Foxk1 | Forkhead box protein K1 | −2.896 |
| | HDPDLGSK | | | | |
| IPI:IPI00830829.1 | S*GEQITSS*PVS*PK | 199 | Hisppd1 | 140 kDa protein | −2.883 |
| IPI:IPI00380722.1 | LAWVGDVFTTT*PTDPRPL | 200 | Bat2 | Large proline-rich protein BAT2 | −2.824 |
| | TS*PLR | | | | |
| IPI:IPI00153986.2 | GTS*RPGTPSAEAASTSSTLR | 201 | Gtf2f1 | General transcription factor IIF | −2.802 |
| | | | | subunit 1 | |
| IPI:IPI00129276.2 | LES*LNIQR\hat{ } | 202 | Eif3a | Eukaryotic translation initiation | −2.792 |
| | | | | factor 3 subunit A | |
| IPI:IPI00121277.1 | SAS*ESYTQSFQSR | 203 | Pi4k2a | Phosphatidylinositol 4-kinase type 2- | −2.789 |
| | | | | alpha | |
| IPI:IPI00470003.3 | S*PTPKS*PPSR | 204 | Parva | Alpha-parvin | −2.746 |
| IPI:IPI00308971.1 | AEALSSLHGDDQDS*EDEV | 205 | Igf2r | Cation-independent mannose-6- | −2.738 |
| | LTVPEVK | | | phosphate receptor | |
| IPI:IPI00121277.1 | SASESYT*QSFQSR | 206 | Pi4k2a | Phosphatidylinositol 4-kinase type 2- | −2.723 |
| | | | | alpha | |
| IPI:IPI00130920.1 | VLS*PLR\hat{ }S*PPLLGSESPYE | 207 | Mtap1b | Microtubule-associated protein 1B | −2.715 |
| | DFLSADSK@ | | | | |
| IPI:IPI00129276.2 | LES*LNIQR | 208 | Eif3a | Eukaryotic translation initiation | −2.711 |
| | | | | factor 3 subunit A | |
| IPI:IPI00111169.1 | CSVS*LSNVEAR | 209 | Pla2g4a | Cytosolic phospholipase A2 | −2.664 |
| IPI:IPI00458068.6 | HS*S*GNSM#LLPSMSVTM | 210 | Fkbp15 | Isoform B of FK506-binding protein 15 | −2.641 |
| | ETSM#IM#SNIQR | | | | |
| IPI:IPI00751009.1 | TVSASS*T*GDLPK@ | 211 | Srpk2 | serine/arginine-rich protein-specific | −2.607 |
| | | | | kinase 2 | |
| IPI:IPI00222828.2 | S*SPNPFVGSPPK@ | 212 | Dab2 | Isoform p93 of Disabled homolog 2 | −2.599 |
| IPI:IPI00111169.1 | CSVS*LSNVEAR | 213 | Pla2g4a | Cytosolic phospholipase A2 | −2.579 |
| IPI:IPI00120886.3 | SVGDGET*VEFDVVEGEK | 214 | Ybx1 | Nuclease-sensitive element-binding | −2.553 |
| | | | | protein 1 | |
| IPI:IPI00751009.1 | TVS*ASS*TGDLPK@ | 215 | Srpk2 | serine/arginine-rich protein-specific | −2.534 |
| | | | | kinase 2 | |
| IPI:IPI00133349.1 | DR\hat{ }S*PS*PLR\hat{ }GNVVPS*PL | 216 | Carhsp1 | Calcium-regulated heat stable protein 1 | −2.512 |
| | PTR\hat{ } | | | | |
| IPI:IPI00469392.2 | GSGSVDET*LFALPAASEPV | 217 | Rtn4 | Isoform 1 of Reticulon-4 | −2.475 |
| | IPSSAEK | | | | |
| IPI:IPI00127976.1 | T*ASLPAIPNPFPELTGAAP | 218 | Grb10 | Isoform 3 of Growth factor receptor- | −2.437 |
| | GS*PPSVAPSS*LPPPPSQP | | | bound protein 10 | |
| | PAK | | | | |
| IPI:IPI00751009.1 | TSEFPTPLFSGPLEPVACG | 219 | Srpk2 | serine/arginine-rich protein-specific | −2.408 |
| | SVISEGSPLTEQEESSPSH | | | kinase 2 | |
| | DRS*R | | | | |
| IPI:IPI00761677.1 | S*RSPVDSPVPASMFAPEP | 220 | Gtpbp1 | GTP-binding protein 1 | −2.406 |
| | SS*PGAAR | | | | |
| IPI:IPI00468516.3 | TRT*VLS*LFDEDEDKVEDE | 221 | D6Wsu116e | Isoform 1 of Protein FAM21 | −2.398 |
| | SSTCAPQDGR | | | | |
| IPI:IPI00396728.3 | R\hat{ }VSGS*ATPNSEAPR\hat{ } | 222 | Ddx51 | ATP-dependent RNA helicase DDX51 | −2.351 |
| IPI:IPI00660767.2 | KRSDS*S*GGYTLSDVIQS* | 223 | Ibtk | Isoform 2 of Inhibitor of Bruton | −2.347 |
| | PPSAGLLK | | | tyrosine kinase | |
| IPI:IPI00226750.2 | TSST*CS*NES*LNAGGTPV | 224 | Tbc1d4 | 140 kDa protein | −2.275 |
| | TPR | | | | |
| IPI:IPI00113033.1 | RSPASSGSVAPQPS*SPPS | 225 | Ighmbp2 | DNA-binding protein SMUBP-2 | −2.241 |
| | *PAQAEPEPR | | | | |
| IPI:IPI00136107.1 | THS*TSSS*IGSGESPFSR | 226 | Ndrg3 | Protein NDRG3 | −2.202 |
| IPI:IPI00751009.1 | TSEFPTPLFSGPLEPVACG | 227 | Srpk2 | serine/arginine-rich protein-specific | −2.187 |
| | SVISEGSPLTEQEESSPSH | | | kinase 2 | |
| | DRS*R | | | | |
| IPI:IPI00321647.2 | QNPEQS*ADEDAEKNEEDS | 228 | Eif3c | Eukaryotic translation initiation | −2.165 |
| | *EGS*S*DEDEDEDGVGNTT | | | factor 3 subunit C | |
| | FLKK | | | | |
| IPI:IPI00453800.4 | HSSFADTGAAPSALSPESP | 229 | Zfp828 | Zinc finger protein 828 | −2.147 |
| | VLATS*PEPWGPS*LSASPE | | | | |
| | SR\hat{ } | | | | |
| IPI:IPI00136107.1 | THS*TSSS*IGSGESPFSR\hat{ } | 230 | Ndrg3 | Protein NDRG3 | −2.124 |
| IPI:IPI00377615.2 | RSS*RS*FSLDEPPLFIPDNI | 231 | Phf3 | PHD finger protein 3 | −2.124 |
| | ATVK | | | | |
| IPI:IPI00377615.2 | R\hat{ }SSR\hat{ }S*FS*LDEPPLFIPD | 232 | Phf3 | PHD finger protein 3 | −2.124 |
| | NIATVK@ | | | | |
| IPI:IPI00127415.1 | CGSGPVHISGQHLVAVEED | 233 | Npm1 | Nucleophosmin | −2.101 |
| | AES*EDEDEEDVKLLGM#S | | | | |
| | GK | | | | |
| IPI:IPI00331075.4 | SLNFQEDDDT*S*RETFASD | 234 | Phax | Phosphorylated adapter RNA export | −2.101 |
| | TNEALASLDEAQEGPGETK | | | protein | |
| IPI:IPI00136107.1 | THSTSS*S*IGSGESPFSR\hat{ } | 235 | Ndrg3 | Protein NDRG3 | −2.098 |
| IPI:IPI00136107.1 | THST*SSS*IGSGESPFSR | 236 | Ndrg3 | Protein NDRG3 | −2.098 |
| IPI:IPI00669522.4 | SRS*DIDVNAAASAK | 237 | Clasp1 | Isoform 2 of CLIP-associating protein 1 | −2.073 |
| IPI:IPI00117953.2 | S*TDSPVAGS*EDDLVAAAP | 238 | Bicc1; | Isoform 1 of Protein bicaudal C | −2.07 |
| | LLHSPEWSEER\hat{ } | | 4930533K18Rik | homolog 1 | |
| IPI:IPI00137229.1 | GFQFVSSS*LPDICYR | 239 | Cnbp | Isoform 2 of Cellular nucleic acid- | −2.061 |
| | | | | binding protein | |
| IPI:IPI00380309.3 | AMLDQLMGTS*R | 240 | Luc7l2 | Isoform 1 of Putative RNA-binding | −2.046 |
| | | | | protein Luc7-like 2 | |
| IPI:IPI00153986.2 | GTSRPGT*PSAEAASTSSTLR | 241 | Gtf2f1 | General transcription factor IIF | −2.035 |
| | | | | subunit 1 | |
| IPI:IPI00330262.5 | S*QDDAMVDYFFQR | 242 | Pum1 | Isoform 1 of Pumilio homolog 1 | −2.02 |
| IPI:IPI00312128.3 | LDLDLTS*DSQPPVFK | 243 | Trim28 | Isoform 1 of Transcription intermediary | −2.01 |
| | | | | factor 1-beta | |
| IPI:IPI00667973.3 | IRQPS*IDLT*DDDQTSSVPH | 244 | Cep170 | Isoform 1 of Centrosomal protein | −1.979 |
| | SAISDIMSSDQETYSCK | | | of 170 kDa | |
| IPI:IPI00468516.3 | TRT*VLS*LFDEDEDKVEDE | 245 | D6Wsu116e | Isoform 1 of Protein FAM21 | −1.956 |
| | SSTCAPQDGR | | | | |
| IPI:IPI00136207.6 | YTPTSPSY*SPSSPEYTPAS | 246 | Polr2a | DNA-directed RNA polymerase II | −1.95 |
| | PK@ | | | subunit RPB1 | |
| IPI:IPI00225062.2 | SLLPNSSQDELM#EVEKSE | 247 | Srrm2 | Isoform 3 of Serine/arginine repetitive | −1.937 |
| | QPLSQVLPSLS*PEHK | | | matrix protein 2 | |
| IPI:IPI00222828.2 | S*SPNPFVGSPPK@ | 248 | Dab2 | Isoform p93 of Disabled homolog 2 | −1.915 |
| IPI:IPI00309059.7 | RS*TS*PIIGS*PPVR | 249 | Patl1 | Protein PAT1 homolog 1 | −1.901 |
| IPI:IPI00309059.7 | RST*S*PIIGS*PPVR | 250 | Patl1 | Protein PAT1 homolog 1 | −1.896 |
| IPI:IPI00338745.4 | QADVADQQTTELPAENGET | 251 | Hmgn1; | Non-histone chromosomal protein | −1.895 |
| | ENQSPASEEEKEAKS*D | | LOC100044391 | HMG-14 | |
| IPI:IPI00417158.1 | SQTPQLHFLDT*DDEISPTS | 252 | Smg6 | Telomerase-binding protein EST1A | −1.895 |
| | WGDSR\hat{ } | | | | |
| IPI:IPI00761759.1 | SSS*GSEHSTEGSVSLGDG | 253 | Larp4 | Putative uncharacterized protein | −1.89 |
| | PLSR | | | | |
| IPI:IPI00314502.5 | VHGLPT*TSPSGVNMAELA | 254 | Tcfeb | Transcription factor EB | −1.887 |
| | QQVVK | | | | |
| IPI:IPI00117689.1 | ATEEPSGTGS*DELIK@SD | 255 | Ptrf | Polymerase I and transcript release | −1.875 |
| | QVNGVLVLSLLDK@ | | | factor | |
| IPI:IPI00462949.4 | AENIMAS*LER | 256 | Dock11 | Dedicator of cytokinesis protein 11 | −1.851 |
| IPI:IPI00471475.1 | FDQLFDDES*DPFEVLK@ | 257 | Serbp1 | Isoform 1 of Plasminogen activator | −1.845 |
| | | | | inhibitor 1 RNA-binding protein | |
| IPI:IPI00471475.1 | FDQLFDDES*DPFEVLK | 258 | Serbp1 | Isoform 1 of Plasminogen activator | −1.845 |
| | | | | inhibitor 1 RNA-binding protein | |
| IPI:IPI00309059.7 | RS*TS*PIIGS*PPVR | 259 | Patl1 | Protein PAT1 homolog 1 | −1.837 |
| IPI:IPI00331612.3 | K@PAQETEETSS*QES*AE | 260 | Hmga2 | High mobility group protein HMGI-C | −1.834 |
| | ED | | | | |
| IPI:IPI00929786.1 | SLPT*TVPES*PNYR\hat{ } | 261 | Larp1 | Isoform 1 of La-related protein 1 | −1.823 |
| IPI:IPI00112101.1 | TTQSLQDFPVADS*EEEAE | 262 | Tfip11 | Tuftelin-interacting protein 11 | −1.813 |
| | EEFQKELSQWR | | | | |
| IPI:IPI00133685.1 | SSDEENGPPSS*PDLDR | 263 | Akt1s1 | Proline-rich AKT1 substrate 1 | −1.779 |
| IPI:IPI00761443.2 | SLS*ESYELSSDLQDK | 264 | Iqsec1 | IQ motif and Sec7 domain 1 isoform b | −1.779 |
| IPI:IPI00318938.6 | NS*PVAKTPPKDLPAIPGVT | 265 | Eif4ebp1 | Eukaryotic translation initiation | −1.776 |
| | *SPTSDEPPMQASQSQLPS | | | factor 4E-binding protein 1 | |
| | SPEDK | | | | |
| IPI:IPI00318938.6 | NSPVAKT*PPKDLPAIPGVT | 266 | Eif4ebp1 | Eukaryotic translation initiation | −1.776 |
| | SPT*SDEPPMQASQSQLPS | | | factor 4E-binding protein 1 | |
| | SPEDK | | | | |
| IPI:IPI00421179.1 | SFS*KEVEER | 267 | Eif4g1 | Isoform 1 of Eukaryotic translation | −1.77 |
| | | | | initiation factor 4 gamma 1 | |
| IPI:IPI00379844.4 | TYS*LTTPAR | 268 | Irs2 | Insulin receptor substrate 2 | −1.769 |
| IPI:IPI00379844.4 | TYS*LTTPAR | 269 | Irs2 | Insulin receptor substrate 2 | −1.769 |
| IPI:IPI00127989.1 | FSEM#M#DHMGGDEDVDL | 270 | Ptges3; | Prostaglandin E synthase 3 | −1.762 |
| | PEVDGADDDS*QDS*DDEK | | Gm9769; | | |
| | MPDLE | | LOC100048119 | | |
| IPI:IPI00127989.1 | FSEM#M#DHMGGDEDVDL | 271 | Ptges3; | Prostaglandin E synthase 3 | −1.762 |
| | PEVDGADDDS*QDS*DDEK | | Gm9769; | | |
| | @MPDLE | | LOC100048119 | | |
| IPI:IPI00320208.3 | YGPSSVEDTTGSGAADAK | 272 | Eef1b2 | Elongation factor 1-beta | −1.754 |
| | @DDDDIDLFGS*DDEEESE | | | | |
| | EAK@ | | | | |
| IPI:IPI00318938.6 | NS*PVAK@T*PPK@DLPAI | 273 | Eif4ebp1 | Eukaryotic translation initiation | −1.752 |
| | PGVTSPTSDEPPMQASQS | | | factor 4E-binding protein 1 | |
| | QLPSSPEDK@ | | | | |
| IPI:IPI00317794.5 | KEDS*DEDEDEEDEDDS*D | 274 | Ncl | Nucleolin | −1.75 |
| | EDEDDEEEDEFEPPIVK | | | | |
| IPI:IPI00116331.1 | S*RTPSAS*HEEQQE | 275 | Sgta | Isoform 1 of Small glutamine-rich | −1.747 |
| | | | | tetratricopeptide repeat-containing | |
| | | | | protein alpha | |
| IPI:IPI00116331.1 | S*R\hat{ }TPS*ASHEEQQE | 276 | Sgta | Isoform 1 of Small glutamine-rich | −1.747 |
| | | | | tetratricopeptide repeat-containing | |
| | | | | protein alpha | |
| IPI:IPI00553798.2 | FGT*FGGLGS*K | 277 | Ahnak | AHNAK nucleoprotein isoform 1 | −1.745 |
| IPI:IPI00130510.1 | LQDISELLAT*GVGLS*DSEV | 278 | Ppan | Suppressor of SWI4 1 homolog | −1.743 |
| | EPDGEHNTTELPQAVAGR\hat{ } | | | | |
| IPI:IPI00330262.5 | S*QDDAMVDYFFQR | 279 | Pum1 | Isoform 1 of Pumilio homolog 1 | −1.734 |
| IPI:IPI00459468.2 | LSESSALKQPAT*PTAAES* | 280 | Wmip1 | Isoform 1 of ATPase WRNIP1 | −1.728 |
| | S*EGEGEEGDDGGETESR | | | | |
| IPI:IPI00130573.1 | SLLSHEFQDET*DT*EEETL | 281 | Cpd | Carboxypeptidase D | −1.723 |
| | YSSK | | | | |
| IPI:IPI00468516.3 | TSALLFSS*DEEDQWNIADS | 282 | D6Wsu116e | Isoform 1 of Protein FAM21 | −1.718 |
| | HTK | | | | |
| IPI:IPI00468516.3 | TSALLFSS*DEEDQWNIADS | 283 | D6Wsu116e | Isoform 1 of Protein FAM21 | −1.718 |
| | HTK | | | | |
| IPI:IPI00109676.6 | SSGSSSSGLGTVSS*SPAS | 284 | Phf8 | Isoform 1 of PHD finger protein 8 | −1.715 |
| | QR | | | | |
| IPI:IPI00399953.1 | VFTSDISDPVVAST*SQAPG | 285 | Wnk1 | Serine/threonine-protein kinase WNK1 | −1.704 |
| | MNLSHSASSLSLQQAFSELK | | | | |
| IPI:IPI00399953.1 | VFTSDISDPVVASTSQAPG | 286 | Wnk1 | Serine/threonine-protein kinase WNK1 | −1.704 |
| | MNLSHSASS*LSLQQAFSE | | | | |
| | LK@ | | | | |
| IPI:IPI00225062.2 | RSSS*ELS*PEVVEK | 287 | Srrm2 | Isoform 3 of Serine/arginine repetitive | −1.702 |
| | | | | matrix protein 2 | |
| IPI:IPI00116331.1 | S*RTPS*ASHEEQQE | 288 | Sgta | Isoform 1 of Small glutamine-rich | −1.694 |
| | | | | tetratricopeptide repeat-containing | |
| | | | | protein alpha | |
| IPI:IPI00116331.1 | SRT*PS*ASHEEQQE | 289 | Sgta | Isoform 1 of Small glutamine-rich | −1.69 |
| | | | | tetratricopeptide repeat-containing | |
| | | | | protein alpha | |
| IPI:IPI00116331.1 | S*R\hat{ }TPS*ASHEEQQE | 290 | Sgta | Isoform 1 of Small glutamine-rich | −1.69 |
| | | | | tetratricopeptide repeat-containing | |
| | | | | protein alpha | |
| IPI:IPI00405752.3 | VLQESDVS*PSSSTTSLPIS* | 291 | C130092O11Rik | Isoform 1 of Uncharacterized protein | −1.689 |
| | PLTEEPLPFKDITR | | | KIAA1680 | |
| IPI:IPI00380415.2 | ALS*LS*SADS*TDAKR | 292 | Bat2l | Isoform 1 of Protein BAT2-like | −1.687 |
| IPI:IPI00130510.1 | LQDISELLATGVGLS*DS*EV | 293 | Ppan | Suppressor of SWI4 1 homolog | −1.68 |
| | EPDGEHNTTELPQAVAGR | | | | |
| IPI:IPI00229859.1 | AKPAAQS*EEETATS*PAAS | 294 | Eif3b | Eif3b protein | −1.677 |
| | *PTPQSAERS*PSQEPSAP | | | | |
| | GK | | | | |
| IPI:IPI00224200.1 | RLS*S*TGGQTPR | 295 | Iws1 | Isoform 1 of Protein IWS1 homolog | −1.663 |
| IPI:IPI00225062.2 | R\hat{ }SSS*ELS*PEVVEK@ | 296 | Srrm2 | Isoform 3 of Serine/arginine repetitive | −1.663 |
| | | | | matrix protein 2 | |
| IPI:IPI00331612.3 | K@PAQETEETS*SQES*AE | 297 | Hmga2 | High mobility group protein HMGI-C | −1.658 |
| | ED | | | | |
| IPI:IPI00229645.2 | K@GSS*GNAS*EVSVACLT | 298 | Cytsa | Cytospin-A | −1.657 |
| | ER\hat{ } | | | | |
| IPI:IPI00317794.5 | KEDS*DEDEDEEDEDDS*D | 299 | Ncl | Nucleolin | −1.637 |
| | EDEDDEEEDEFEPPIVK | | | | |
| IPI:IPI00663627.1 | GFLDGVYSFEYYPS*TPGK | 300 | Flnb | Filamin-B | −1.621 |
| IPI:IPI00312128.3 | LDLDLTSDS*QPPVFK | 301 | Trim28 | Isoform 1 of Transcription intermediary | −1.616 |
| | | | | factor 1-beta | |
| IPI:IPI00225062.2 | HSGS*TSPY*PK@ | 302 | Srrm2 | Isoform 3 of Serine/arginine repetitive | −1.603 |
| | | | | matrix protein 2 | |
| IPI:IPI00312414.4 | TSTFCGT*PEFLAPEVLTET | 303 | Pkn2 | Isoform 1 of Serine/threonine-protein | −1.603 |
| | SYTR\hat{ } | | | kinase N2 | |
| IPI:IPI00553798.2 | S*NS*FSDER\hat{ }EFSAPST*PT | 304 | Ahnak | AHNAK nucleoprotein isoform 1 | −1.602 |
| | GT*LEFAGGDAK@ | | | | |
| IPI:IPI00126176.3 | VSLLGPVT*T*PEFQLVKTPL | 305 | Racgap1 | Rac GTPase-activating protein 1 | −1.597 |
| | SSSLSQR | | | | |
| IPI:IPI00225062.2 | GSLSRSS*S*PVTELTAR | 306 | Srrm2 | Isoform 3 of Serine/arginine repetitive | −1.593 |
| | | | | matrix protein 2 | |
| IPI:IPI00399953.1 | VFTSDISDPVVASTSQAPG | 307 | Wnk1 | Serine/threonine-protein kinase WNK1 | −1.588 |
| | MNLSHS*ASSLSLQQAFSE | | | | |
| | LK | | | | |
| IPI:IPI00226155.3 | VSALEEDMDDVES*S*EEEE | 308 | Prpf38a | Isoform 1 of Pre-mRNA-splicing factor | −1.585 |
| | EEDEKLER | | | 38A | |
| IPI:IPI00226155.3 | VSALEEDMDDVES*S*EEEE | 309 | Prpf38a | Isoform 1 of Pre-mRNA-splicing factor | −1.585 |
| | EEDEK@LER\hat{ } | | | 38A | |
| IPI:IPI00116331.1 | S*RTPS*ASHEEQQE | 310 | Sgta | Isoform 1 of Small glutamine-rich | −1.58 |
| | | | | tetratricopeptide repeat-containing | |
| | | | | protein alpha | |
| IPI:IPI00116331.1 | S*R\hat{ }TPS*ASHEEQQE | 311 | Sgta | Isoform 1 of Small glutamine-rich | −1.58 |
| | | | | tetratricopeptide repeat-containing | |
| | | | | protein alpha | |
| IPI:IPI00135660.5 | SLEETLHNVDLS*S*DDELP | 312 | Sdpr | Serum deprivation-response protein | −1.577 |
| | R\hat{ }DEEALEDSAEEK@ | | | | |
| IPI:IPI00323349.2 | HSSHDMLSHS*WEGNR | 313 | Tjp2 | Tight junction protein ZO-2 | −1.566 |
| IPI:IPI00169500.3 | TESILDKEDKVPMAGVGGT | 314 | Atxn2l | Isoform 1 of Ataxin-2-like protein | −1.564 |
| | EGPEQLPAPCPSQTGS*PP | | | | |
| | VGLIK | | | | |
| IPI:IPI00118143.1 | K@QTPPASPS*PQPIEDR\hat{ } | 315 | Cttn | Src substrate cortactin | −1.556 |
| | PPS*SPIYEDAAPFK@ | | | | |
| IPI:IPI00127415.1 | TVSLGAGAK@DELHIVEAE | 316 | Npm1 | Nucleophosmin | −1.555 |
| | AM#NYEGS*PIK@VTLATLK@ | | | | |
| IPI:IPI00225062.2 | SLLPNSSQDELMEVEK@S | 317 | Srrm2 | Isoform 3 of Serine/arginine repetitive | −1.55 |
| | EQPLSQVLPSLS*PEHK@ | | | matrix protein 2 | |
| IPI:IPI00225062.2 | SLLPNSSQDELMEVEKSEQ | 318 | Srrm2 | Isoform 3 of Serine/arginine repetitive | −1.55 |
| | PLSQVLPSLS*PEHK | | | matrix protein 2 | |
| IPI:IPI00320267.1 | GDQVLNFS*DAEDLIDDSK | 319 | Mcrs1 | Microspherule protein 1 | −1.55 |
| | @LK@ | | | | |
| IPI:IPI00320267.1 | GDQVLNFS*DAEDLIDDSKLK | 320 | Mcrs1 | Microspherule protein 1 | −1.55 |
| IPI:IPI00553798.2 | SNS*FSDER\hat{ }EFS*APS*TPT | 321 | Ahnak | AHNAK nucleoprotein isoform 1 | −1.548 |
| | *GTLEFAGGDAK@ | | | | |
| IPI:IPI00116331.1 | S*RTPS*ASHEEQQE | 322 | Sgta | Isoform 1 of Small glutamine-rich | −1.543 |
| | | | | tetratricopeptide repeat-containing | |
| | | | | protein alpha | |
| IPI:IPI00222828.2 | SSPNPFVGS*PPK | 323 | Dab2 | Isoform p93 of Disabled homolog 2 | −1.531 |
| IPI:IPI00317794.5 | KEDS*DEDEDEEDEDDS*D | 324 | Ncl | Nucleolin | −1.531 |
| | EDEDDEEEDEFEPPIVK | | | | |
| IPI:IPI00225062.2 | RSS*SELS*PEVVEK | 325 | Srrm2 | Isoform 3 of Serine/arginine repetitive | −1.524 |
| | | | | matrix protein 2 | |
| IPI:IPI00225062.2 | R\hat{ }SS*S*ELSPEVVEK@ | 326 | Srrm2 | Isoform 3 of Serine/arginine repetitive | −1.524 |
| | | | | matrix protein 2 | |
| IPI:IPI00116331.1 | S*RTPS*ASHEEQQE | 327 | Sgta | Isoform 1 of Small glutamine-rich | −1.52 |
| | | | | tetratricopeptide repeat-containing | |
| | | | | protein alpha | |
| IPI:IPI00380415.2 | ALS*LS*S*ADSTDAKR | 328 | Bat2l | Isoform 1 of Protein BAT2-like | −1.519 |
| IPI:IPI00225062.2 | RSSS*ELS*PEVVEK | 329 | Srrm2 | Isoform 3 of Serine/arginine repetitive | −1.518 |
| | | | | matrix protein 2 | |
| IPI:IPI00225062.2 | R\hat{ }SSS*ELS*PEVVEK@ | 330 | Srrm2 | Isoform 3 of Serine/arginine repetitive | −1.518 |
| | | | | matrix protein 2 | |
| IPI:IPI00399469.2 | VS*PPAS*PTLDVELGAGEA | 331 | Sh3rf1 | Isoform 2 of Putative E3 ubiquitin- | −1.504 |
| | PLQGAVGPELPLGGSHGR | | | protein ligase SH3RF1 | |
| IPI:IPI00225062.2 | R\hat{ }SS*SELS*PEVVEK@ | 332 | Srrm2 | Isoform 3 of Serine/arginine repetitive | −1.501 |
| | | | | matrix protein 2 | |
| IPI:IPI00137501.1 | TEMDKS*PFNS*PSPQDS*PR | 333 | Nfic | Isoform 1 of Nuclear factor 1 C-type | −1.498 |
| IPI:IPI00309059.7 | RSTS*PIIGS*PPVR | 334 | Patl1 | Protein PAT1 homolog 1 | −1.498 |
| IPI:IPI00225062.2 | RSS*SELS*PEVVEK | 335 | Srrm2 | Isoform 3 of Serine/arginine repetitive | −1.497 |
| | | | | matrix protein 2 | |
| IPI:IPI00116331.1 | S*R\hat{ }TPS*ASHEEQQE | 336 | Sgta | Isoform 1 of Small glutamine-rich | −1.493 |
| | | | | tetratricopeptide repeat-containing | |
| | | | | protein alpha | |
| IPI:IPI00470095.1 | HGS*GADS*DYENTQSGDP | 337 | Git1 | ARF GTPase-activating protein GIT1 | −1.488 |
| | LLGLEGK@ | | | | |
| IPI:IPI00470095.1 | HGS*GADS*DYENTQSGDP | 338 | Git1 | ARF GTPase-activating protein GIT1 | −1.488 |
| | LLGLEGK | | | | |
| IPI:IPI00225062.2 | IHTTALTGQS*PPLASGHQG | 339 | Srrm2 | Isoform 3 of Serine/arginine repetitive | −1.479 |
| | EGDAPSVEPGATNIQQPSS | | | matrix protein 2 | |
| | *PAPS*TK | | | | |
| IPI:IPI00116442.1 | TRLAS*ESANDDNEDS | 340 | Hdgfrp2 | Isoform 3 of Hepatoma-derived growth | −1.456 |
| | | | | factor-related protein 2 | |
| IPI:IPI00225062.2 | GSLSRSS*S*PVTELTAR | 341 | Srrm2 | Isoform 3 of Serine/arginine repetitive | −1.454 |
| | | | | matrix protein 2 | |
| IPI:IPI00320267.1 | GDQVLNFS*DAEDLIDDSK | 342 | Mcrs1 | Microspherule protein 1 | −1.454 |
| IPI:IPI00225062.2 | RSSS*ELS*PEVVEK | 343 | Srrm2 | Isoform 3 of Serine/arginine repetitive | −1.445 |
| | | | | matrix protein 2 | |
| IPI:IPI00661508.3 | S*IDPDSIQSALLASGLGSK@ | 344 | Sbno1 | Isoform 2 of Protein strawberry notch | −1.442 |
| | | | | homolog 1 | |
| IPI:IPI00661508.3 | S*IDPDSIQSALLASGLGSK | 345 | Sbno1 | Isoform 2 of Protein strawberry notch | −1.442 |
| | | | | homolog 1 | |
| IPI:IPI00317794.5 | K@EDS*DEDEDEEDEDDS* | 346 | Ncl | Nucleolin | −1.441 |
| | DEDEDDEEEDEFEPPIVK@ | | | | |
| IPI:IPI00225267.6 | ATPTESPEKPPPPAVQRDE | 347 | 2310022B05Rik | Uncharacterized protein C1orf198 | −1.438 |
| | DDDALFS*EPALAQISSSNV | | | homolog | |
| | LLK | | | | |
| IPI:IPI00122594.4 | EVS*VSS*VTEEPKLDSSQL | 348 | Ahctf1 | AT-hook-containing transcription | −1.437 |
| | PLQTGLDVPATPR | | | factor 1 | |
| IPI:IPI00129356.1 | SAFTPATATGS*S*PSPVLG | 349 | Itsn1 | lsoform 1 of Intersectin-1 | −1.43 |
| | QGEK@VEGLQAQALYPWR\hat{ } | | | | |
| IPI:IPI00318938.6 | NS*PVAKT*PPKDLPAIPGV | 350 | Eif4ebp1 | Eukaryotic translation initiation | −1.412 |
| | TSPTSDEPPMQASQSQLPS | | | factor 4E-binding protein 1 | |
| | S*PEDK | | | | |
| IPI:IPI00224200.1 | VVCDADDS*DSDVVSDK@ | 351 | Iws1 | Isoform 1 of Protein IWS1 homolog | −1.403 |
| IPI:IPI00321647.2 | QNPEQSADEDAEK@NEED | 352 | Eif3c | Eukaryotic translation initiation | −1.399 |
| | S*EGS*S*DEDEDEDGVGN | | | factor 3 subunit C | |
| | TTFLK@ | | | | |
| IPI:IPI00664808.2 | VSESVADDS*SS*RDSFTQS | 353 | Kdm3b | Isoform 2 of Lysine-specific | −1.393 |
| | LESLTSGLCK | | | demethylase 3B | |
| IPI:IPI00122863.2 | RGQS*PQPDQGP | 354 | Rarg | Isoform 2 of Retinoic acid receptor | −1.387 |
| | | | | gamma | |
| IPI:IPI00453853.2 | TPVASECTQTDGAEEVAGS | 355 | Bap1 | Ubiquitin carboxyl-terminal hydrolase | −1.38 |
| | CPQTTTHS*PPSK@ | | | BAP1 | |
| IPI:IPI00132028.1 | K@GS*DDDGGDS*PVQDID | 356 | Sap30 | Histone deacetylase complex subunit | −1.376 |
| | TPEVDLYQLQVNTLR\hat{ } | | | SAP30 | |
| IPI:IPI00132028.1 | KGS*DDDGGDS*PVQDIDT | 357 | Sap30 | Histone deacetylase complex subunit | −1.376 |
| | PEVDLYQLQVNTLR | | | SAP30 | |
| IPI:IPI00318938.6 | NS*PVAKT*PPKDLPAIPGV | 358 | Eif4ebp1 | Eukaryotic translation initiation | −1.374 |
| | TSPTSDEPPMQASQSQLPS | | | factor 4E-binding protein 1 | |
| | S*PEDK | | | | |
| IPI:IPI00116442.1 | TRLAS*ESANDDNEDS | 359 | Hdgfrp2 | Isoform 3 of Hepatoma-derived growth | −1.358 |
| | | | | factor-related protein 2 | |
| IPI:IPI00153986.2 | GTS*RPGT*PSAEAASTSST | 360 | Gtf2f1 | General transcription factor IIF | −1.348 |
| | LR | | | subunit 1 | |
| IPI:IPI00226149.6 | GSASES*PSASVAEATTTD | 361 | Rsl1d1 | Putative uncharacterized protein | −1.342 |
| | VQVTPTALLQLDR\hat{ } | | | | |
| IPI:IPI00226149.6 | GS*ASESPSASVAEATTTD | 362 | Rsl1d1 | Putative uncharacterized protein | −1.342 |
| | VQVTPTALLQLDR\hat{ } | | | | |
| IPI:IPI00313307.3 | SYQNSPS*S*EDGIRPLPEY | 363 | Med1 | Isoform 4 of Mediator of RNA | −1.337 |
| | STEK | | | polymerase II transcription subunit 1 | |
| IPI:IPI00656285.2 | S*LVS*PIPSPTGTISVPNSC | 364 | Foxk1 | Forkhead box protein K1 | −1.327 |
| | PAS*PR | | | | |
| IPI:IPI00320208.3 | YGPSSVEDTTGSGAADAK | 365 | Eef1b2 | Elongation factor 1-beta | −1.324 |
| | @DDDDIDLFGSDDEEES*E | | | | |
| | EAK@ | | | | |
| IPI:IPI00318938.6 | NS*PVAKT*PPKDLPAIPGV | 366 | Eif4ebp1 | Eukaryotic translation initiation | −1.322 |
| | TSPTSDEPPMQASQSQLPS | | | factor 4E-binding protein 1 | |
| | S*PEDK | | | | |
| IPI:IPI00136572.4 | SSTHASVS*PASEPSR | 367 | Zc3hav1 | Isoform 1 of Zinc finger CCCH-type | −1.318 |
| | | | | antiviral protein 1 | |
| IPI:IPI00116442.1 | TRLAS*ES*ANDDNEDS | 368 | Hdgfrp2 | Isoform 3 of Hepatoma-derived growth | −1.315 |
| | | | | factor-related protein 2 | |
| IPI:IPI00116442.1 | TR\hat{ }LAS*ES*ANDDNEDS | 369 | Hdgfrp2 | Isoform 3 of Hepatoma-derived growth | −1.315 |
| | | | | factor-related protein 2 | |
| IPI:IPI00226149.6 | GS*ASESPSASVAEATTTD | 370 | Rsl1d1 | Putative uncharacterized protein | −1.314 |
| | VQVTPTALLQLDR\hat{ } | | | | |
| IPI:IPI00169500.3 | R\hat{ }SPGGT*SPPNGGLPGPL | 371 | Atxn2l | Isoform 1 of Ataxin-2-like protein | −1.308 |
| | TAT*AAPPGPPAAVSPCLG | | | | |
| | PAAAAGSGLR\hat{ } | | | | |
| IPI:IPI00467362.6 | S*ESLIDASEDS*QLEAAIR | 372 | Ubxn7 | UBX domain-containing protein 7 | −1.307 |
| IPI:IPI00124753.3 | RNTFIGTPY*WMAPEVIACD | 373 | Mink1 | misshapen-like kinase 1 isoform 2 | −1.302 |
| | ENPDATYDYR | | | | |
| IPI:IPI00110247.7 | SLSQS*FENLLDEPAYGLIQ | 374 | Tbc1d15 | TBC1 domain family member 15 | −1.301 |
| | K@ | | | | |
| IPI:IPI00276222.1 | KLDDQS*EDEALELEGPLIM | 375 | Fermt2 | Fermitin family homolog 2 | −1.293 |
| | PGSGSIYSS*PGLYSK | | | | |
| IPI:IPI00460668.2 | EVDYSDS*LTEK@ | 376 | Smarca4 | Putative uncharacterized protein | −1.293 |
| IPI:IPI00380736.1 | STTPT*PSVITR | 377 | Zfp516 | Zinc finger protein 516 | −1.259 |
| IPI:IPI00153986.2 | GTS*R\hat{ }PGT*PSAEAASTSS | 378 | Gtf2f1 | General transcription factor IIF | −1.251 |
| | TLR\hat{ } | | | subunit 1 | |
| IPI:IPI00230486.3 | LSSKLS*AVS*LR | 379 | Snx17 | Sorting nexin-17 | −1.24 |
| IPI:IPI00116331.1 | SR\hat{ }T*PSAS*HEEQQE | 380 | Sgta | Isoform 1 of Small glutamine-rich | −1.239 |
| | | | | tetratricopeptide repeat-containing | |
| | | | | protein alpha | |
| IPI:IPI00318938.6 | NS*PVAKT*PPKDLPAIPGV | 381 | Eif4ebp1 | Eukaryotic translation initiation | −1.238 |
| | TSPTSDEPPMQASQSQLPS | | | factor 4E-binding protein 1 | |
| | SPEDK | | | | |
| IPI:IPI00318938.6 | NS*PVAKT*PPKDLPAIPGV | 382 | Eif4ebp1 | Eukaryotic translation initiation | −1.238 |
| | TSPTSDEPPMQASQSQLPS | | | factor 4E-binding protein 1 | |
| | SPEDK | | | | |
| IPI:IPI00749954.8 | LR\hat{ }TDNAS*DAS*ESS*DAE | 383 | Jmjd1c | jumonji domain containing 1C | −1.235 |
| | SSSK@ | | | | |
| IPI:IPI00225062.2 | SVSSPR\hat{ }T*K@ | 384 | Srrm2 | Isoform 3 of Serine/arginine repetitive | −1.234 |
| | | | | matrix protein 2 | |
| IPI:IPI00136107.1 | THS*TSSSIGSGESPFSRA | 385 | Ndrg3 | Protein NDRG3 | −1.23 |
| IPI:IPI00136107.1 | THST*SSSIGSGESPFSR | 386 | Ndrg3 | Protein NDRG3 | −1.23 |
| IPI:IPI00113377.1 | K@EESEES*EDDMGFGLFD | 387 | Rplp1 | 60S acidic ribosomal protein P1 | −1.229 |
| IPI:IPI00553798.2 | GGVT*GSPEAS*IS*GSK@G | 388 | Ahnak | AHNAK nucleoprotein isoform 1 | −1.228 |
| | DLK@ | | | | |
| IPI:IPI00330240.7 | S*QDAESYQNVVELK@ | 389 | Peg3 | Isoform 1 of Paternally-expressed gene | −1.221 |
| | | | | 3 protein | |
| IPI:IPI00122845.1 | LGS*QEPER\hat{ } | 390 | | | −1.219 |
| IPI:IPI00224200.1 | VVCDADDS*DSDVVSDK@ | 391 | Iws1 | Isoform 1 of Protein IWS1 homolog | −1.219 |
| IPI:IPI00465879.2 | LCS*SSSSDTSPR\hat{ } | 392 | Zc3hc1 | Isoform 1 of Nuclear-interacting | −1.207 |
| | | | | partner of ALK | |
| IPI:IPI00116442.1 | TR\hat{ }LAS*ES*ANDDNEDS | 393 | Hdgfrp2 | Isoform 3 of Hepatoma-derived growth | −1.206 |
| | | | | factor-related protein 2 | |
| IPI:IPI00116442.1 | TRLAS*ES*ANDDNEDS | 394 | Hdgfrp2 | Isoform 3 of Hepatoma-derived growth | −1.206 |
| | | | | factor-related protein 2 | |
| IPI:IPI00322095.4 | IDEPNT*PYHNMIGDDEDAY | 395 | Ppp1r2 | Putative uncharacterized protein | −1.204 |
| | S*DSEGNEVMTPDILAK | | | | |
| IPI:IPI00322095.4 | IDEPNTPY*HNMIGDDEDAY | 396 | Ppp1r2 | Putative uncharacterized protein | −1.204 |
| | S*DSEGNEVMTPDILAK | | | | |
| IPI:IPI00387384.3 | LLCQEETPEAT*EDERDEER | 397 | Mett10d | Isoform 1 of Putative methyltransferase | −1.202 |
| | | | | METT10D | |
| IPI:IPI00330246.2 | WAAHGTS*PEDFSLTLGAR\hat{ } | 398 | Phldb1 | Isoform 2 of Pleckstrin homology-like | −1.202 |
| | | | | domain family B member 1 | |
| IPI:IPI00330246.2 | WAAHGTS*PEDFSLTLGAR | 399 | Phldb1 | Isoform 2 of Pleckstrin homology-like | −1.202 |
| | | | | domain family B member 1 | |
| IPI:IPI00678133.3 | SSHQDATDPEALWGVHQT | 400 | Inf2 | Isoform 1 of Inverted formin-2 | −1.199 |
| | EADS*T*S*EGPEDEAQR\hat{ } | | | | |
| IPI:IPI00465879.2 | LCS*SSSSDTSPR\hat{ } | 401 | Zc3hc1 | Isoform 1 of Nuclear-interacting | −1.189 |
| | | | | partner of ALK | |
| IPI:IPI00313513.6 | S*VDIFGSTPFQPFSVSASK | 402 | Bmp2k | Isoform 1 of BMP-2-inducible protein | −1.18 |
| | | | | kinase | |
| IPI:IPI00465879.2 | LCS*SSSSDTSPR | 403 | Zc3hc1 | Isoform 1 of Nuclear-interacting | −1.172 |
| | | | | partner of ALK | |
| IPI:IPI00465879.2 | LCS*SSSSDTSPR | 404 | Zc3hc1 | Isoform 1 of Nuclear-interacting | −1.171 |
| | | | | partner of ALK | |
| IPI:IPI00553798.2 | EFSAPST*PT*GTLEFAGGD | 405 | Ahnak | AHNAK nucleoprotein isoform 1 | −1.169 |
| | AK@ | | | | |
| IPI:IPI00317794.5 | K@EDS*DEDEDEEDEDDS* | 406 | Ncl | Nucleolin | −1.167 |
| | DEDEDDEEEDEFEPPIVK@ | | | | |
| IPI:IPI00465879.2 | LCS*SSSSDTSPR\hat{ } | 407 | Zc3hc1 | Isoform 1 of Nuclear-interacting | −1.163 |
| | | | | partner of ALK | |
| IPI:IPI00465879.2 | LCS*SSSSDTSPR | 408 | Zc3hc1 | Isoform 1 of Nuclear-interacting | −1.163 |
| | | | | partner of ALK | |
| IPI:IPI00229739.4 | LVVSS*PT*SPKGK | 409 | Plekhm1 | Pleckstrin homology domain-containing | −1.159 |
| | | | | family M member 1 | |
| IPI:IPI00465879.2 | LCS*SSSSDTSPR | 410 | Zc3hc1 | Isoform 1 of Nuclear-interacting | −1.156 |
| | | | | partner of ALK | |
| IPI:IPI00465879.2 | LCS*SSSSDTSPR\hat{ } | 411 | Zc3hc1 | Isoform 1 of Nuclear-interacting | −1.155 |
| | | | | partner of ALK | |
| IPI:IPI00225616.3 | DGAGS*PLR\hat{ }T*SPPSTPSS | 412 | Dennd2a | DENN domain-containing protein 2A | −1.15 |
| | PDDTFFNLGDLQNGR\hat{ } | | | | |
| IPI:IPI00720110.2 | VLTANSNPSS*PSAAK | 413 | 1600027N09Rik | RIKEN cDNA 1600027N09 gene | −1.148 |
| IPI:IPI00465879.2 | LCS*SSSSDTSPR | 414 | Zc3hc1 | Isoform 1 of Nuclear-interacting | −1.143 |
| | | | | partner of ALK | |
| IPI:IPI00461396.6 | STRHS*PSALQDVFVELLS* | 415 | Baz1a | bromodomain adjacent to zinc finger | −1.134 |
| | PHSK | | | domain 1A | |
| IPI:IPI00222828.2 | SSPNPFVGS*PPK@ | 416 | Dab2 | Isoform p93 of Disabled homolog 2 | −1.134 |
| IPI:IPI00222828.2 | SSPNPFVGS*PPK | 417 | Dab2 | Isoform p93 of Disabled homolog 2 | −1.134 |
| IPI:IPI00222828.2 | SSPNPFVGS*PPK | 418 | Dab2 | Isoform p93 of Disabled homolog 2 | −1.134 |
| IPI:IPI00225062.2 | GCS*PPKS*PEKPPQSTSS* | 419 | Srrm2 | Isoform 3 of Serine/arginine repetitive | −1.133 |
| | ESCPPSPQPTK | | | matrix protein 2 | |
| IPI:IPI00553798.2 | GGVTGS*PEAS*IS*GSK@G | 420 | Ahnak | AHNAK nucleoprotein isoform 1 | −1.132 |
| | DLK@ | | | | |
| IPI:IPI00318671.2 | DK@PVYDELFYTLS*PINGK@ | 421 | Ehd4 | EH-domain containing 4-KJR | −1.131 |
| | | | | (Fragment) | |
| IPI:IPI00129388.1 | HDHAVSLLTAASPT*ISLL | 422 | Scrib | Isoform 3 of Protein LAP4 | −1.13 |
| | LER | | | | |
| IPI:IPI00336973.2 | GLNLDGTPALSTLGGFS*PA | 423 | Ccnl1 | Isoform 1 of Cyclin-L1 | −1.124 |
| | SKPSS*PR | | | | |
| IPI:IPI00336973.2 | GLNLDGTPALSTLGGFS*PA | 424 | Ccnl1 | Isoform 1 of Cyclin-L1 | −1.124 |
| | SK@PSS*PR\hat{ } | | | | |
| IPI:IPI00317401.6 | METVSNASSSSNPSS*PGR\hat{ } | 425 | Pds5b | Isoform 1 of Sister chromatid cohesion | −1.12 |
| | | | | protein PDS5 homolog B | |
| IPI:IPI00317401.6 | METVSNASSSSNPSS*PGR | 426 | Pds5b | Isoform 1 of Sister chromatid cohesion | −1.12 |
| | | | | protein PDS5 homolog B | |
| IPI:IPI00471475.1 | HSGSDR\hat{ }S*SFSHYSGLK@ | 427 | Serbp1 | Isoform 1 of Plasminogen activator | −1.119 |
| | | | | inhibitor 1 RNA-binding protein | |
| IPI:IPI00330240.7 | S*QDAESYQNVVELK@ | 428 | Peg3 | Isoform 1 of Paternally-expressed gene | −1.108 |
| | | | | 3 protein | |
| IPI:IPI00153986.2 | GTS*RPGTPSAEAASTSSTLR | 429 | Gtf2f1 | General transcription factor IIF | −1.102 |
| | | | | subunit 1 | |
| IPI:IPI00453656.2 | KVAPLS*SSLDTSLDFSK | 430 | Uvrag | UV radiation resistance associated | −1.098 |
| IPI:IPI00127976.1 | SVS*ENS*LVAMDFSGQIGR | 431 | Grb10 | Isoform 3 of Growth factor receptor- | −1.097 |
| | | | | bound protein 10 | |
| IPI:IPI00135443.2 | KTS*FDQDS*DVDIFPSDFT | 432 | Top2b | DNA topoisomerase 2-beta | −1.092 |
| | SEPPALPR | | | | |
| IPI:IPI00331612.3 | KPAQETEETSS*QES*AEED | 433 | Hmga2 | High mobility group protein HMGI-C | −1.078 |
| IPI:IPI00331612.3 | K@PAQETEETSS*QES*AE | 434 | Hmga2 | High mobility group protein HMGI-C | −1.078 |
| | ED | | | | |
| IPI:IPI00320208.3 | YGPSSVEDTTGSGAADAK | 435 | Eef1b2 | Elongation factor 1-beta | −1.075 |
| | @DDDDIDLFGS*DDEEESE | | | | |
| | EAK@ | | | | |
| IPI:IPI00321884.1 | ESLPLDLS*DDQSNSK | 436 | Nvl | Nuclear valosin-containing protein-like | −1.071 |
| IPI:IPI00127415.1 | CGSGPVHISGQHLVAVEED | 437 | Npm1 | Nucleophosmin | −1.071 |
| | AES*EDEDEEDVK@LLGMS | | | | |
| | GK@ | | | | |
| IPI:IPI00553798.2 | GGVTGS*PEAS*ISGS*K@G | 438 | Ahnak | AHNAK nucleoprotein isoform 1 | −1.067 |
| | DLK@ | | | | |
| IPI:IPI00136107.1 | THST*SS*SIGSGESPFSR | 439 | Ndrg3 | Protein NDRG3 | −1.054 |
| IPI:IPI00130920.1 | VLS*PLR\hat{ }SPPLLGSES*PYE | 440 | Mtap1b | Microtubule-associated protein 1B | −1.054 |
| | DFLSADSK@ | | | | |
| IPI:IPI00349069.4 | GPQQDS*DS*DDGEALGGN | 441 | B230208H17Rik | Putative GTP-binding protein Parf | −1.048 |
| | PMVAGFQDDVDIEDQTHGK | | | | |
| IPI:IPI00349069.4 | GPQQDS*DS*DDGEALGGN | 442 | B230208H17Rik | Putative GTP-binding protein Parf | −1.048 |
| | PMVAGFQDDVDIEDQTHG | | | | |
| | K@ | | | | |
| IPI:IPI00408803.6 | GR\hat{ }ES*DEDT*EDAS*ETDL | 443 | Suds3 | Sin3 histone deacetylase corepressor | −1.047 |
| | AK@ | | | complex component SDS3 | |
| IPI:IPI00109311.3 | SASS*DTS*EELNS*QDSPK | 444 | Slc9a3r1 | Isoform 1 of Na(+)/H(+) exchange | −1.047 |
| | @R\hat{ } | | | regulatory cofactor NHE-RF1 | |
| IPI:IPI00136107.1 | THSTSS*SIGSGESPFSR\hat{ } | 445 | Ndrg3 | Protein NDRG3 | −1.042 |
| IPI:IPI00114560.5 | FRT*ITSSYYR | 446 | Rab1 | Ras-related protein Rab-1A | −1.042 |
| IPI:IPI00123410.5 | TIS*AQDTLAYATALLNEK | 447 | Usp24 | Isoform 1 of Ubiquitin carboxyl- | −1.04 |
| | | | | terminal hydrolase 24 | |
| IPI:IPI00169477.1 | NT*PSQHSHSIQHS*PER\hat{ } | 448 | Bclaf1 | Isoform 2 of Bcl-2-associated | −1.036 |
| | | | | transcription factor 1 | |
| IPI:IPI00317401.6 | AES*PETSAVESTQST*PQK | 449 | Pds5b | Isoform 1 of Sister chromatid cohesion | −1.036 |
| | | | | protein PDS5 homolog B | |
| IPI:IPI00317401.6 | METVSNASSSSNPSS*PGR\hat{ } | 450 | Pds5b | Isoform 1 of Sister chromatid cohesion | −1.035 |
| | | | | protein PDS5 homolog B | |
| IPI:IPI00317401.6 | METVSNASSSSNPSS*PGR | 451 | Pds5b | Isoform 1 of Sister chromatid cohesion | −1.035 |
| | | | | protein PDS5 homolog B | |
| IPI:IPI00229702.1 | KDS*PPLVT*PPQS*PPSSQ | 452 | Smtnl2 | Smoothelin-like protein 2 | −1.03 |
| | PPAMTQAPR | | | | |
| IPI:IPI00107999.1 | SSPS*ILAVQR | 453 | Chd1 | Chromodomain-helicase-DNA-binding | −1.019 |
| | | | | protein 1 | |
| IPI:IPI00107999.1 | SS*PSILAVQR\hat{ } | 454 | Chd1 | Chromodomain-helicase-DNA-binding | −1.019 |
| | | | | protein 1 | |
| IPI:IPI00118120.1 | TSS*IADEGTYTLDSILR | 455 | Myo5a | Myosin-Va | −1.018 |
| IPI:IPI00453848.5 | SAFK@LDS*DS*DEEDAQF | 456 | Atg2a | Protein | −1.014 |
| | FSMASGVPQTPAPEPSR\hat{ }R\hat{ } | | | | |
| IPI:IPI00330773.3 | AS*YSGTSPSHSFISGEPDR\hat{ } | 457 | Phldb2 | Isoform 1 of Pleckstrin homology-like | −1.013 |
| | | | | domain family B member 2 | |
| IPI:IPI00225062.2 | GCS*PPKS*PEKPPQSTSS* | 458 | Srrm2 | Isoform 3 of Serine/arginine repetitive | −1.003 |
| | ESCPPSPQPTK | | | matrix protein 2 | |
| IPI:IPI00229613.3 | SSS*SASQSNHSCTSMPSS | 459 | Macf1 | Isoform 3 of Microtubule-actin cross- | −1.003 |
| | PATPASGTK | | | linking factor 1 | |
| IPI:IPI00136107.1 | THS*TSSS*IGSGESPFSR\hat{ } | 460 | Ndrg3 | Protein NDRG3 | −1.002 |
| IPI:IPI00121135.5 | R.S*KS*PPKS*PEEEGAVS* | 461 | Sfrs2 | Splicing factor | −1 |
| | S. | | | | |
| IPI:IPI00126338.5 | R.SST*PLPTVS*SS*AENTR | 462 | Tmpo | Isoform Alpha of Lamina-associated | −1 |
| | \hat{ }.Q | | | polypeptide 2 | |
| IPI:IPI00623249.4 | R.T*DSVIIADQT*PT* | 463 | Atf7 | Activating transcription factor 7 | −1 |
| | PTR\hat{ }.F | | | | |

Rapa replicate 2 (Note: heavy cells were control and light cells were
treated with rapamycin. L, light; H, heavy.)

| | | | | | |
|---|---|---|---|---|---|
| IPI:IPI00330112.1 | VAPAGSAIS*NASGER\hat{ } | 464 | Mib1 | E3 ubiquitin-protein ligase MIB1 | 9.6909 |
| IPI:IPI00108454.2 | R\hat{ }LS*S*LR\hat{ }AS*TSK@ | 465 | rpS6 | 29 kDa protein | 9.6182 |
| IPI:IPI00108454.2 | R\hat{ }LS*S*LR\hat{ }AS*TSK@ | 466 | rpS6 | 29 kDa protein | 9.6182 |
| IPI:IPI00225062.2 | ELS*HSPPRENSFESSLEFK | 467 | Srrm2 | Isoform 3 of Serine/arginine repetitive | 9.4619 |
| | | | | matrix protein 2 | |
| IPI:IPI00108454.2 | R\hat{ }LS*S*LR\hat{ }AS*TSK@ | 468 | rpS6 | 29 kDa protein | 9.4253 |
| IPI:IPI00346155.4 | NFLS*MDS*LK@ | 469 | Gm13099 | Novel protein similar to preferentially | 9.2638 |
| | | | | expressed antigen in melanoma-like | |
| | | | | family | |
| IPI:IPI00649156.4 | NLAITGGVTLS*VIASPVI | 470 | Rnf19b | IBR domain containing 3 | 9.1171 |
| | AAVSVGIGVPIMLAY*VY* | | | | |
| | GVVPISLCR\hat{ } | | | | |
| IPI:IPI00108454.2 | R\hat{ }LS*S*LR\hat{ }AS*TSK@ | 471 | rpS6 | 29 kDa protein | 9.0561 |
| IPI:IPI00133349.1 | TR\hat{ }TFS*ATVR\hat{ } | 472 | Carhsp1 | Calcium-regulated heat stable protein 1 | 8.7001 |
| IPI:IPI00133349.1 | TR\hat{ }TFS*ATVR\hat{ } | 473 | Carhsp1 | Calcium-regulated heat stable protein 1 | 8.6779 |
| IPI:IPI00133349.1 | TR\hat{ }TFS*ATVR\hat{ } | 474 | Carhsp1 | Calcium-regulated heat stable protein 1 | 8.6519 |
| IPI:IPI00108454.2 | R\hat{ }LS*S*LR\hat{ }AS*TSK@ | 475 | rpS6 | 29 kDa protein | 8.6271 |
| IPI:IPI00351206.5 | GLCSGPGAGEESPAAT*LP | 476 | Pgrmc2 | Membrane-associated progesterone | 8.5943 |
| | R\hat{ }M#K@ | | | receptor component 2 | |
| IPI:IPI00108454.2 | LS*SLR\hat{ }AS*TSK@ | 477 | rpS6 | 29 kDa protein | 7.5465 |
| IPI:IPI00108454.2 | LSS*LR\hat{ }AS*TSK@ | 478 | rpS6 | 29 kDa protein | 7.5465 |
| IPI:IPI00131138.1 | AHVAPCFDAS*K@ | 479 | | | 7.3937 |
| IPI:IPI00125530.1 | WTNDYSMVLTGAAIY*HK@ | 480 | Ext1 | Exostosin-1 | 7.3545 |
| IPI:IPI00108454.2 | LSS*LR\hat{ }AS*TSK@ | 481 | rpS6 | 29 kDa protein | 7.1711 |
| IPI:IPI00108454.2 | LSS*LR\hat{ }AS*TSK@ | 482 | rpS6 | 29 kDa protein | 7.1711 |
| IPI:IPI00108454.2 | R\hat{ }LSS*LR\hat{ }AS*TSK@ | 483 | rpS6 | 29 kDa protein | 7.0714 |
| IPI:IPI00131138.1 | AHVAPCFDAS*K@ | 484 | | | 7.0413 |
| IPI:IPI00378564.4 | LVY*PEVK@ES*K@FK@ | 485 | Gm14085 | Novel protein similar to solute carrier | 6.9768 |
| | | | | family 28 (Sodium-coupled nucleoside | |
| | | | | transporter) member 2 | |
| IPI:IPI00108454.2 | R\hat{ }LSS*LR\hat{ }AS*TSK@ | 486 | rpS6 | 29 kDa protein | 6.9459 |
| IPI:IPI00133685.1 | AATATRPPGPPPAPQPPS* | 487 | Akt1s1 | Proline-rich AKT1 substrate 1 | 6.9065 |
| | PAPS*PPPRPALAR | | | | |
| IPI:IPI00316922.1 | R\hat{ }LGAAM#T*FPR\hat{ }PPSR\hat{ } | 488 | Gm9757 | Putative uncharacterized protein | 6.6604 |
| IPI:IPI00118875.4 | ATAPQTQHVS*PMR\hat{ } | 489 | Eef1d | eukaryotic translation elongation | 6.5442 |
| | | | | factor 1 delta isoform a | |
| IPI:IPI00109318.1 | R\hat{ }NS*PMAQT*PPCHLPNIP | 490 | Eif4ebp2 | Eukaryotic translation initiation | 6.5094 |
| | GVTSPGALIEDSK@ | | | factor 4E-binding protein 2 | |
| IPI:IPI00109318.1 | R\hat{ }NS*PMAQT*PPCHLPNIP | 491 | Eif4ebp2 | Eukaryotic translation initiation | 6.4811 |
| | GVTSPGALIEDSK@ | | | factor 4E-binding protein 2 | |
| IPI:IPI00131138.1 | AHVAPCFDAS*K@ | 492 | | | 6.4769 |
| IPI:IPI00757687.1 | LK@PDS*K@PFSR\hat{ } | 493 | Gon4l | RIKEN cDNA 5830417I10 gene | 6.4593 |
| IPI:IPI00121027.4 | ES*LS*HVLSK@ | 494 | Mogat1 | 2-acylglycerol O-acyltransferase 1 | 6.0819 |
| IPI:IPI00115681.1 | TECGS*TGS*PASSFHFK@ | 495 | Eef2k | Elongation factor 2 kinase | 5.912 |
| IPI:IPI00115681.1 | TECGS*T*GSPASSFHFK@ | 496 | Eef2k | Elongation factor 2 kinase | 5.912 |
| IPI:IPI00128818.2 | LYTEK@AY*K@ | 497 | Dhx15 | Putative pre-mRNA-splicing factor ATP- | 5.9047 |
| | | | | dependent RNA helicase DHX15 | |
| IPI:IPI00118438.4 | SR\hat{ }VS*VS*PGR\hat{ } | 498 | Srrm1 | Isoform 2 of Serine/arginine repetitive | 5.834 |
| | | | | matrix protein 1 | |
| IPI:IPI00323238.5 | R\hat{ }K@T*VSFS*AAEEAPVPE | 499 | Setd1a | SET domain containing 1A | 5.7891 |
| | PSTAAPLQAK@ | | | | |
| IPI:IPI00323238.5 | R\hat{ }K@T*VSFS*AAEEAPVPE | 500 | Setd1a | SET domain containing 1A | 5.6797 |
| | PSTAAPLQAK@ | | | | |
| IPI:IPI00323238.5 | R\hat{ }K@T*VSFS*AAEEAPVPE | 501 | Setd1a | SET domain containing 1A | 5.6797 |
| | PSTAAPLQAK@ | | | | |
| IPI:IPI00405470.1 | SGHSLLT*WDSAMSALSSA | 502 | D830015G02Rik | Putative uncharacterized protein | 5.6705 |
| | S*SSSCCTLR\hat{ } | | | | |
| IPI:IPI00137148.2 | R\hat{ }K@Y*GMLHTQESSSSVF | 503 | Vwa5b1 | von Willebrand factor A domain- | 5.6371 |
| | YPSQDEGLSPGSGNCAK@ | | | containing protein 5B1 | |
| IPI:IPI00660767.2 | SDSS*GGYTLSDVIQS*PPS | 504 | Ibtk | Isoform 2 of Inhibitor of Bruton | 5.5816 |
| | AGLLK@ | | | tyrosine kinase | |
| IPI:IPI00660767.2 | SDSS*GGYTLSDVIQS*PPS | 505 | Ibtk | Isoform 2 of Inhibitor of Bruton | 5.4166 |
| | AGLLK@ | | | tyrosine kinase | |
| IPI:IPI00351206.5 | GLCSGPGAGEESPAAT*LP | 506 | Pgrmc2 | Membrane-associated progesterone | 5.4066 |
| | R\hat{ }M#K@ | | | receptor component 2 | |
| IPI:IPI00117731.1 | TFWS*PELK@K@ | 507 | Erc1; | Isoform 1 of ELKS/RAB6- | 5.3901 |
| | | | LOC100048600 | interacting/CAST family member 1 | |
| IPI:IPI00115681.1 | TECGS*TGSPAS*SFHFK@ | 508 | Eef2k | Elongation factor 2 kinase | 5.3519 |
| IPI:IPI00351206.5 | GLCSGPGAGEES*PAATLP | 509 | Pgrmc2 | Membrane-associated progesterone | 5.2793 |
| | R\hat{ }M#K@ | | | receptor component 2 | |
| IPI:IPI00108454.2 | LSS*LR\hat{ }AS*TSK@SESSQK@ | 510 | rpS6 | 29 kDa protein | 5.274 |
| IPI:IPI00108454.2 | LSS*LR\hat{ }ASTS*K@SESSQK@ | 511 | rpS6 | 29 kDa protein | 5.274 |
| IPI:IPI00108454.2 | R\hat{ }LS*S*LR\hat{ } | 512 | rpS6 | 29 kDa protein | 5.2699 |
| IPI:IPI00108454.2 | RLS*S*LR | 513 | rpS6 | 29 kDa protein | 5.2699 |
| IPI:IPI00108454.2 | LS*SLR\hat{ }ASTS*K@SESSQK@ | 514 | rpS6 | 29 kDa protein | 5.257 |
| IPI:IPI00331280.1 | AALDQT*SGLPGGAAQDPG | 515 | Adamts2 | A disintegrin and metalloproteinase | 5.2222 |
| | GR\hat{ } | | | with thrombospondin motifs 2 | |
| IPI:IPI00108454.2 | RLS*S*LR | 516 | rpS6 | 29 kDa protein | 5.1769 |
| IPI:IPI00108454.2 | R\hat{ }LS*S*LR\hat{ } | 517 | rpS6 | 29 kDa protein | 5.1745 |
| IPI:IPI00127976.1 | MNILS*SQS*PLHPSTLNAVI | 518 | Grb10 | Isoform 3 of Growth factor receptor- | 5.0363 |
| | HR\hat{ } | | | bound protein 10 | |
| IPI:IPI00127976.1 | MNILSS*QS*PLHPSTLNAVI | 519 | Grb10 | Isoform 3 of Growth factor receptor- | 5.0363 |
| | HR\hat{ } | | | bound protein 10 | |
| IPI:IPI00656285.2 | SLVSPIPS*PTGTISVPNSCP | 520 | Foxk1 | Forkhead box protein K1 | 5.0164 |
| | AS*PR\hat{ } | | | | |
| IPI:IPI00461419.2 | IR\hat{ }S*S*LAR\hat{ } | 521 | Gm6988 | similar to hCG1640785 | 4.998 |
| IPI:IPI00108454.2 | R\hat{ }LS*S*LR\hat{ }ASTSK@ | 522 | rpS6 | 29 kDa protein | 4.9653 |
| IPI:IPI00127976.1 | MNILS*SQS*PLHPSTLNAVI | 523 | Grb10 | Isoform 3 of Growth factor receptor- | 4.9297 |
| | HR\hat{ } | | | bound protein 10 | |
| IPI:IPI00403938.2 | LLDPQEFT*LS*GT*QR\hat{ } | 524 | Tnc | Isoform 1 of Tenascin | 4.9074 |
| IPI:IPI00108454.2 | R\hat{ }LS*S*LR\hat{ }ASTSK@ | 525 | rpS6 | 29 kDa protein | 4.8929 |
| IPI:IPI00929786.1 | TASISSSPS*EGTPAVGS*Y | 526 | Larp1 | Isoform 1 of La-related protein 1 | 4.8902 |
| | GCT*PQSLPK@ | | | | |
| IPI:IPI00169986.4 | SLLVLR\hat{ }GT*AYT*R\hat{ } | 527 | Alkbh6 | 24 kDa protein | 4.8761 |
| IPI:IPI00127976.1 | MNILSS*QS*PLHPSTLNAVI | 528 | Grb10 | Isoform 3 of Growth factor receptor- | 4.774 |
| | HR\hat{ } | | | bound protein 10 | |
| IPI:IPI00929786.1 | T*ASISSS*PSEGTPAVGSY | 529 | Larp1 | Isoform 1 of La-related protein 1 | 4.758 |
| | GCT*PQS*LPK@ | | | | |
| IPI:IPI00929786.1 | T*ASISSS*PSEGTPAVGSY* | 530 | Larp1 | Isoform 1 of La-related protein 1 | 4.758 |
| | GCT*PQSLPK@ | | | | |
| IPI:IPI00115681.1 | TECGS*TGS*PASSFHFK@ | 531 | Eef2k | Elongation factor 2 kinase | 4.7395 |
| IPI:IPI00929786.1 | TAS*ISSS*PSEGTPAVGSY | 532 | Larp1 | Isoform 1 of La-related protein 1 | 4.7282 |
| | GCT*PQS*LPK@ | | | | |
| IPI:IPI00127976.1 | M#NILSS*QS*PLHPSTLNAV | 533 | Grb10 | Isoform 3 of Growth factor receptor- | 4.7179 |
| | IHR\hat{ } | | | bound protein 10 | |
| IPI:IPI00340860.5 | TAS*EGS*EAETPEAPK@Q | 534 | Larp7 | Isoform 1 of La-related protein 7 | 4.7122 |
| | PAK@K@ | | | | |
| IPI:IPI00340860.5 | TAS*EGS*EAETPEAPK@Q | 535 | Larp7 | Isoform 1 of La-related protein 7 | 4.7105 |
| | PAK@K@ | | | | |
| IPI:IPI00127976.1 | MNILSSQS*PLHPSTLNAVIH | 536 | Grb10 | Isoform 3 of Growth factor receptor- | 4.6623 |
| | R\hat{ } | | | bound protein 10 | |
| IPI:IPI00127976.1 | MNILSSQS*PLHPSTLNAVIH | 537 | Grb10 | Isoform 3 of Growth factor receptor- | 4.6623 |
| | R\hat{ } | | | bound protein 10 | |
| IPI:IPI00929786.1 | TASIS*SSPSEGT*PAVGSY | 538 | Larp1 | Isoform 1 of La-related protein 1 | 4.5768 |
| | GCTPQS*LPK@ | | | | |
| IPI:IPI00115681.1 | TECGS*TGSPAS*SFHFK@ | 539 | Eef2k | Elongation factor 2 kinase | 4.562 |
| IPI:IPI00553798.2 | VK@T*PEMIIQK@PK@ | 540 | Ahnak | AHNAK nucleoprotein isoform 1 | 4.557 |
| IPI:IPI00929786.1 | T*ASISSS*PSEGTPAVGSY* | 541 | Larp1 | Isoform 1 of La-related protein 1 | 4.4981 |
| | GCT*PQSLPK@ | | | | |
| IPI:IPI00929786.1 | TAS*ISSS*PSEGTPAVGSY | 542 | Larp1 | Isoform 1 of La-related protein 1 | 4.4981 |
| | GCT*PQS*LPK@ | | | | |
| IPI:IPI00320208.3 | YGPSSVEDTTGSGAADAK | 543 | Eef1b2 | Elongation factor 1-beta | 4.4765 |
| | @DDDDIDLFGS*DDEEESE | | | | |
| | EAK@K@ | | | | |
| IPI:IPI00320208.3 | YGPSSVEDTTGSGAADAKD | 544 | Eef1b2 | Elongation factor 1-beta | 4.4765 |
| | DDDIDLFGS*DDEEESEEAKK | | | | |
| IPI:IPI00474916.3 | AVVPFLGVGFSSLDMS*LCV | 545 | Qsox2 | Isoform 3 of Sulfhydryl oxidase 2 | 4.4747 |
| | VLY*VASS*LFLM#IMYFFFR | | | | |
| | \hat{ }VR\hat{ } | | | | |
| IPI:IPI00399953.1 | K@FS*APGQLCVPMTSNLG | 546 | Wnk1 | Serine/threonine-protein kinase WNK1 | 4.3426 |
| | GSTPISAASATSLGHFTK@ | | | | |
| IPI:IPI00654415.2 | MT*S*T*DYS*LSGTCGM#D | 547 | Megf11 | Isoform 4 of Multiple epidermal growth | 4.325 |
| | R\hat{ } | | | factor-like domains 11 | |
| IPI:IPI00660767.2 | SDS*SGGYTLSDVIQS*PPS | 548 | Ibtk | Isoform 2 of Inhibitor of Bruton | 4.2681 |
| | AGLLK@ | | | tyrosine kinase | |
| IPI:IPI00660767.2 | K@R\hat{ }SDSSGGYT*LSDVIQS | 549 | Ibtk | Isoform 2 of Inhibitor of Bruton | 4.2613 |
| | *PPSAGLLK@ | | | tyrosine kinase | |
| IPI:IPI00127976.1 | MNILSSQS*PLHPSTLNAVIH | 550 | Grb10 | Isoform 3 of Growth factor receptor- | 4.2564 |
| | R\hat{ } | | | bound protein 10 | |
| IPI:IPI00127976.1 | MNILSSQSPLHPS*TLNAVIH | 551 | Grb10 | Isoform 3 of Growth factor receptor- | 4.2564 |
| | R\hat{ } | | | bound protein 10 | |
| IPI:IPI00127976.1 | M#NILSSQS*PLHPSTLNAVI | 552 | Grb10 | Isoform 3 of Growth factor receptor- | 4.2332 |
| | HR\hat{ } | | | bound protein 10 | |
| IPI:IPI00313653.2 | QFCQEGQPHVLEALS*PPQ | 553 | Maf1 | Repressor of RNA polymerase III | 4.2077 |
| | TSGLS*PSR\hat{ } | | | transcription MAF1 homolog | |
| IPI:IPI00929786.1 | TAS*ISSS*PSEGTPAVGSY | 554 | Larp1 | Isoform 1 of La-related protein 1 | 4.18 |
| | GCT*PQS*LPK@ | | | | |
| IPI:IPI00127976.1 | M#NILSSQS*PLHPSTLNAVI | 555 | Grb10 | Isoform 3 of Growth factor receptor- | 4.1504 |
| | HR\hat{ } | | | bound protein 10 | |
| IPI:IPI00127976.1 | M#NILSSQS*PLHPSTLNAVI | 556 | Grb10 | Isoform 3 of Growth factor receptor- | 4.1504 |
| | HR\hat{ } | | | bound protein 10 | |
| IPI:IPI00122594.4 | EVS*VSS*VTEEPK@LDSSQ | 557 | Ahctf1 | AT-hook-containing transcription | 4.144 |
| | LPLQTGLDVPAT*PR\hat{ } | | | factor 1 | |
| IPI:IPI00127976.1 | MNILSSQS*PLHPSTLNAVIH | 558 | Grb10 | Isoform 3 of Growth factor receptor- | 4.133 |
| | R\hat{ } | | | bound protein 10 | |
| IPI:IPI00127976.1 | MNILSSQS*PLHPSTLNAVIH | 559 | Grb10 | Isoform 3 of Growth factor receptor- | 4.133 |
| | R\hat{ } | | | bound protein 10 | |
| IPI:IPI00109318.1 | R\hat{ }NS*PMAQT*PPCHLPNIP | 560 | Eif4ebp2 | Eukaryotic translation initiation | 4.0914 |
| | GVTSPGALIEDSK@ | | | factor 4E-binding protein 2 | |
| IPI:IPI00109318.1 | R\hat{ }NS*PMAQT*PPCHLPNIP | 561 | Eif4ebp2 | Eukaryotic translation initiation | 4.0914 |
| | GVTSPGALIEDSK@ | | | factor 4E-binding protein 2 | |
| IPI:IPI00122521.1 | K@DELSDWS*LAGEDDR\hat{ }E | 562 | Fxr1 | Isoform E of Fragile X mental | 4.0773 |
| | TR\hat{ } | | | retardation syndrome-related protein 1 | |
| IPI:IPI00127976.1 | MNILSS*QSPLHPSTLNAV | 563 | Grb10 | Isoform 3 of Growth factor receptor- | 4.0585 |
| | IHR | | | bound protein 10 | |
| IPI:IPI00127976.1 | MNILS*SQS*PLHPSTLNAVI | 564 | Grb10 | Isoform 3 of Growth factor receptor- | 4.0417 |
| | HR\hat{ } | | | bound protein 10 | |
| IPI:IPI00313653.2 | QFCQEGQPHVLEALS*PPQ | 565 | Maf1 | Repressor of RNA polymerase III | 4.0213 |
| | TSGLS*PSR\hat{ } | | | transcription MAF1 homolog | |
| IPI:IPI00127976.1 | MNILSS*QSPLHPSTLNAV | 566 | Grb10 | Isoform 3 of Growth factor receptor- | 3.9734 |
| | IHR | | | bound protein 10 | |
| IPI:IPI00127976.1 | MNILSSQS*PLHPSTLNAVIH | 567 | Grb10 | Isoform 3 of Growth factor receptor- | 3.9734 |
| | R\hat{ } | | | bound protein 10 | |
| IPI:IPI00127976.1 | MNILSSQS*PLHPSTLNAVIH | 568 | Grb10 | Isoform 3 of Growth factor receptor- | 3.9734 |
| | R\hat{ } | | | bound protein 10 | |
| IPI:IPI00127976.1 | MNILSS*QSPLHPSTLNAV | 569 | Grb10 | Isoform 3 of Growth factor receptor- | 3.9734 |
| | IHR | | | bound protein 10 | |
| IPI:IPI00120691.3 | QADSET*K@EIITEEPS*EEE | 570 | Ddx21 | Nucleolar RNA helicase 2 | 3.9589 |
| | ADMPK@PK@ | | | | |
| IPI:IPI00226441.2 | LHYT*PPLQS*PIT*DGDPLL | 571 | Lin9 | Isoform 2 of Lin-9 homolog | 3.8536 |
| | GQS*PWR\hat{ } | | | | |
| IPI:IPI00127976.1 | MNILSSQS*PLHPSTLNAVIH | 572 | Grb10 | Isoform 3 of Growth factor receptor- | 3.8383 |
| | R\hat{ } | | | bound protein 10 | |
| IPI:IPI00127976.1 | MNILSSQS*PLHPSTLNAVIH | 573 | Grb10 | Isoform 3 of Growth factor receptor- | 3.8383 |
| | R\hat{ } | | | bound protein 10 | |
| IPI:IPI00656285.2 | SSGLQTPECLS*REGS*PIP | 574 | Foxk1 | Forkhead box protein K1 | 3.7948 |
| | HDPDLGSK | | | | |
| IPI:IPI00656285.2 | SSGLQTPECLS*R\hat{ }EGS*PIP | 575 | Foxk1 | Forkhead box protein K1 | 3.7948 |
| | HDPDLGSK@ | | | | |
| IPI:IPI00468516.3 | SHILESVPTLPGS*VEAGVS | 576 | D6Wsu116e | Isoform 1 of Protein FAM21 | 3.7932 |
| | FDLPAQADTLHSANK@ | | | | |
| IPI:IPI00751009.1 | TSEFPTPLFSGPLEPVACGS | 577 | Srpk2 | serine/arginine-rich protein-specific | 3.7653 |
| | VISEGSPLTEQEESSPSHDR | | | kinase 2 | |
| | \hat{ }S*R\hat{ } | | | | |
| IPI:IPI00929786.1 | TASISSSPS*EGTPAVGSYG | 578 | Larp1 | Isoform 1 of La-related protein 1 | 3.756 |
| | CT*PQS*LPK@ | | | | |
| IPI:IPI00929786.1 | TASISSSPS*EGTPAVGSY* | 579 | Larp1 | Isoform 1 of La-related protein 1 | 3.756 |
| | GCT*PQSLPK@ | | | | |
| IPI:IPI00656285.2 | SSGLQTPECLS*R\hat{ }EGS*PIP | 580 | Foxk1 | Forkhead box protein K1 | 3.7214 |
| | HDPDLGSK@ | | | | |
| IPI:IPI00400017.2 | TQS*GNFNTDAPGMAEFR\hat{ } | 581 | Ppfibp2 | Isoform 4 of Liprin-beta-2 | 3.6603 |
| IPI:IPI00660767.2 | SDSS*GGYTLSDVIQS*PPS | 582 | Ibtk | Isoform 2 of Inhibitor of Bruton | 3.6519 |
| | AGLLK@ | | | tyrosine kinase | |
| IPI:IPI00116442.1 | S*EGLSLER\hat{ } | 583 | Hdgfrp2 | Isoform 3 of Hepatoma-derived growth | 3.6501 |
| | | | | factor-related protein 2 | |
| IPI:IPI00379844.4 | TAS*EGDGGAAGGAGTAG | 584 | Irs2 | Insulin receptor substrate 2 | 3.6492 |
| | GR\hat{ }PMSVAGS*PLS*PGPVR\hat{ } | | | | |
| IPI:IPI00929786.1 | T*ASISSS*PSEGTPAVGS*Y | 585 | Larp1 | Isoform 1 of La-related protein 1 | 3.6217 |
| | GCTPQSLPK@ | | | | |
| IPI:IPI00929786.1 | T*ASISSS*PSEGTPAVGS*Y | 586 | Larp1 | Isoform 1 of La-related protein 1 | 3.6217 |
| | GCTPQSLPK@ | | | | |
| IPI:IPI00116442.1 | R\hat{ }S*EGLSLER\hat{ } | 587 | Hdgfrp2 | Isoform 3 of Hepatoma-derived growth | 3.6142 |
| | | | | factor-related protein 2 | |
| IPI:IPI00656285.2 | SLVS*PIPSPTGT*ISVPNSC | 588 | Foxk1 | Forkhead box protein K1 | 3.6065 |
| | PAS*PR\hat{ } | | | | |
| IPI:IPI00656285.2 | SLVS*PIPS*PTGTISVPNSC | 589 | Foxk1 | Forkhead box protein K1 | 3.6065 |
| | PAS*PR\hat{ } | | | | |
| IPI:IPI00656285.2 | SLVSPIPSPT*GTISVPNSCP | 590 | Foxk1 | Forkhead box protein K1 | 3.605 |
| | AS*PR\hat{ } | | | | |
| IPI:IPI00654417.1 | HSS*PPLTVK | 591 | Tinf2 | Putative uncharacterized protein | 3.5562 |
| IPI:IPI00830829.1 | R\hat{ }R\hat{ }SGEQIT*SS*PVS*PK@ | 592 | Hisppd1 | 140 kDa protein | 3.5492 |
| IPI:IPI00118875.4 | GATPAEDDEDKDIDLFGS*D | 593 | Eef1d | eukaryotic translation elongation | 3.5469 |
| | EEEEDKEAAR | | | factor 1 delta isoform a | |
| IPI:IPI00118875.4 | GATPAEDDEDKDIDLFGS*D | 594 | Eef1d | eukaryotic translation elongation | 3.5469 |
| | EEEEDKEAAR | | | factor 1 delta isoform a | |
| IPI:IPI00169888.3 | SQDVAIS*PQQQQCSK@S* | 595 | Edc3 | Enhancer of mRNA-decapping protein 3 | 3.5277 |
| | YVDR\hat{ } | | | | |
| IPI:IPI00134029.8 | VS*SS*VS*VGPSM#SGET*L | 596 | Aim1l | Absent in melanoma 1-like | 3.5092 |
| | PR\hat{ } | | | | |
| IPI:IPI00654417.1 | HSS*PPLTVK | 597 | Tinf2 | Putative uncharacterized protein | 3.4997 |
| IPI:IPI00676574.2 | RVS*TDLPEGQDVYTAACN | 598 | Herc1 | hect (homologous to the E6-AP | 3.4866 |
| | SVIHR | | | (UBE3A) carboxyl terminus) domain | |
| | | | | and RCC1 (CHC1)-like domain (RLD) 1 | |
| IPI:IPI00762234.2 | SGDPR\hat{ }S*CEEES*DPEPDP | 599 | Fbxw9 | F-box and WD-40 domainprotein 9 | 3.4479 |
| | DPDTQAEAYVAR\hat{ } | | | | |
| IPI:IPI00660767.2 | VDTAASSSWLAGS*CS*PVS | 600 | Ibtk | Isoform 2 of Inhibitor of Bruton | 3.398 |
| | PPVVDLR\hat{ } | | | tyrosine kinase | |
| IPI:IPI00169888.3 | SQDVAIS*PQQQQCS*K@S | 601 | Edc3 | Enhancer of mRNA-decapping protein 3 | 3.3665 |
| | YVDR\hat{ } | | | | |
| IPI:IPI00169888.3 | SQDVAISPQQQQCS*K@SY | 602 | Edc3 | Enhancer of mRNA-decapping protein 3 | 3.3665 |
| | *VDR\hat{ } | | | | |
| IPI:IPI00122521.1 | K@DELSDWS*LAGEDDR\hat{ }E | 603 | Fxr1 | Isoform E of Fragile X mental | 3.3379 |
| | TR\hat{ } | | | retardation syndrome-related protein 1 | |
| IPI:IPI00761443.2 | RSS*AGS*LESNVEGSIISS* | 604 | Iqsec1 | IQ motif and Sec7 domain 1 isoform b | 3.3179 |
| | PHMR | | | | |
| IPI:IPI00761443.2 | R\hat{ }S*SAGS*LESNVEGSIISS* | 605 | Iqsec1 | IQ motif and Sec7 domain 1 isoform b | 3.3179 |
| | PHMR\hat{ } | | | | |
| IPI:IPI00828461.1 | GPPDFS*S*DEER\hat{ }EPTPVL | 606 | Tmpo | thymopoietin isoform delta | 3.3038 |
| | GSGASVGR\hat{ } | | | | |
| IPI:IPI00828461.1 | GPPDFS*S*DEER\hat{ }EPTPVL | 607 | Tmpo | thymopoietin isoform delta | 3.3038 |
| | GSGASVGR\hat{ } | | | | |
| IPI:IPI00126317.1 | NAS*TS*FQELEDK@K@EL | 608 | Dnajc2 | DnaJ homolog subfamily C member 2 | 3.2768 |
| | SEESEDEELQLEEFPMLK@ | | | | |
| IPI:IPI00227934.3 | TSASCS*PAPES*PMSSSES | 609 | Cux1 | cut-like homeobox 1 isoform a | 3.1887 |
| | VK@ | | | | |
| IPI:IPI00116442.1 | S*EGLSLER\hat{ } | 610 | Hdgfrp2 | Isoform 3 of Hepatoma-derived growth | 3.1751 |
| | | | | factor-related protein 2 | |
| IPI:IPI00468516.3 | SHILESVPTLPGS*VEAGVS | 611 | D6Wsu116e | Isoform 1 of Protein FAM21 | 3.1635 |
| | FDLPAQADTLHSANK@ | | | | |
| IPI:IPI00468516.3 | SHILESVPTLPGS*VEAGVS | 612 | D6Wsu116e | Isoform 1 of Protein FAM21 | 3.1635 |
| | FDLPAQADTLHSANK@ | | | | |
| IPI:IPI00762234.2 | SGDPR\hat{ }S*CEEES*DPEPDP | 613 | Fbxw9 | F-box and WD-40 domain protein 9 | 3.1508 |
| | DPDTQAEAYVAR\hat{ } | | | | |
| IPI:IPI00320208.3 | YGPSSVEDTTGSGAADAKD | 614 | Eef1b2 | Elongation factor 1-beta | 3.1436 |
| | DDDIDLFGS*DDEEESEEAKK | | | | |
| IPI:IPI00320594.5 | SQPHSSTSNQETS*DS*EM | 615 | Ranbp10 | Ran-binding protein 10 | 3.1377 |
| | EMEAEHYPNGVLESVSTR\hat{ } | | | | |
| IPI:IPI00808277.2 | S*APASPNHAGVLSAHSSG | 616 | Foxk2 | Isoform 1 of Forkhead box protein K2 | 3.1204 |
| | AQTPES*LSR\hat{ }EGS*PAPLE | | | | |
| | PEPGASQPK@ | | | | |
| IPI:IPI00808277.2 | SAPAS*PNHAGVLSAHSSG | 617 | Foxk2 | Isoform 1 of Forkhead box protein K2 | 3.1105 |
| | AQTPES*LSR\hat{ }EGS*PAPLE | | | | |
| | PEPGASQPK@ | | | | |
| IPI:IPI00753321.2 | S*VSETSEDK@K@DEES*D | 618 | Bod1l | biorientation of chromosomes in cell | 3.106 |
| | EEEEEEEEEEPLGATTR\hat{ } | | | division 1-like | |
| IPI:IPI00420464.3 | SNT*PS*PLNLSSTSSK@ | 619 | Zeb2 | Zinc finger E-box-binding homeobox 2 | 3.0996 |
| IPI:IPI00116442.1 | S*EGLSLER\hat{ } | 620 | Hdgfrp2 | Isoform 3 of Hepatoma-derived growth | 3.0974 |
| | | | | factor-related protein 2 | |
| IPI:IPI00808277.2 | SAPAS*PNHAGVLSAHSSG | 621 | Foxk2 | Isoform 1 of Forkhead box protein K2 | 3.0877 |
| | AQT*PES*LSR\hat{ } | | | | |
| IPI:IPI00830829.1 | R\hat{ }R\hat{ }S*GEQITSS*PVS*PK@ | 622 | Hisppd1 | 140 kDa protein | 3.0712 |
| IPI:IPI00117932.2 | S*PPVQPHTPVTISLGTAPS | 623 | Sin3a | Isoform 1 of Paired amphipathic helix | 3.0565 |
| | LQNNQPVEFNHAINYVNK@ | | | protein Sin3a | |
| IPI:IPI00117932.2 | S*PPVQPHTPVTISLGTAPS | 624 | Sin3a | Isoform 1 of Paired amphipathic helix | 3.0565 |
| | LQNNQPVEFNHAINYVNK@ | | | protein Sin3a | |
| IPI:IPI00320594.5 | SQPHSSTSNQETS*DS*EM | 625 | Ranbp10 | Ran-binding protein 10 | 3.0328 |
| | EMEAEHYPNGVLESVSTR\hat{ } | | | | |
| IPI:IPI00227934.3 | TS*ASCSPAPES*PMSSSES | 626 | Cux1 | cut-like homeobox 1 isoform a | 3.0118 |
| | VK@ | | | | |
| IPI:IPI00380817.6 | GR\hat{ }SS*ESSCGLDGDYEDA | 627 | Bcr | Breakpoint cluster region protein | 3.0087 |
| | ELNPR\hat{ } | | | | |
| IPI:IPI00656285.2 | SLVSPIPS*PTGTISVPNSCP | 628 | Foxk1 | Forkhead box protein K1 | 2.9994 |
| | AS*PR\hat{ } | | | | |
| IPI:IPI00656285.2 | SLVS*PIPS*PTGTISVPNSC | 629 | Foxk1 | Forkhead box protein K1 | 2.9989 |
| | PAS*PR\hat{ } | | | | |
| IPI:IPI00127976.1 | MNILS*SQS*PLHPSTLNAVI | 630 | Grb10 | Isoform 3 of Growth factor receptor- | 2.9784 |
| | HR\hat{ } | | | bound protein 10 | |
| IPI:IPI00551454.3 | ER\hat{ }QES*ESEQELVNK@ | 631 | Pdcd11 | Protein RRP5 homolog | 2.9603 |
| IPI:IPI00226155.3 | VSALEEDMDDVES*S*EEEE | 632 | Prpf38a | Isoform 1 of Pre-mRNA-splicing factor | 2.9547 |
| | EEDEKLER | | | 38A | |
| IPI:IPI00753321.2 | SVSETS*EDK@K@DEES*D | 633 | Bod1l | biorientation of chromosomes in cell | 2.9525 |
| | EEEEEEEEEEPLGATTR\hat{ } | | | division 1-like | |
| IPI:IPI00753321.2 | SVSET*SEDK@K@DEES*D | 634 | Bod1l | biorientation of chromosomes in cell | 2.9525 |
| | EEEEEEEEEEPLGATTR\hat{ } | | | division 1-like | |
| IPI:IPI00108454.2 | LSS*LR\hat{ }ASTS*K@S*ESSQ | 635 | — | 29 kDa protein | 2.9394 |
| | K@ | | | | |
| IPI:IPI00553798.2 | HR\hat{ }SNS*FS*DER\hat{ }EFSAPS* | 636 | Ahnak | AHNAK nucleoprotein isoform 1 | 2.9368 |
| | TPTGTLEFAGGDAK@ | | | | |
| IPI:IPI00551454.3 | ER\hat{ }QES*ESEQELVNK@ | 637 | Pdcd11 | Protein RRP5 homolog | 2.9308 |
| IPI:IPI00468516.3 | SHILESVPTLPGS*VEAGVS | 638 | D6Wsu116e | Isoform 1 of Protein FAM21 | 2.9286 |
| | FDLPAQADTLHSANK@ | | | | |
| IPI:IPI00420464.3 | SNT*PS*PLNLSSTSSK@ | 639 | Zeb2 | Zinc finger E-box-binding homeobox 2 | 2.9204 |
| IPI:IPI00656285.2 | SLVS*PIPS*PTGTISVPNSC | 640 | Foxk1 | Forkhead box protein K1 | 2.8837 |
| | PAS*PR\hat{ } | | | | |
| IPI:IPI00330773.3 | K@MLLASTSSDDFDR\hat{ }AS* | 641 | Phldb2 | Isoform 1 of Pleckstrin homology-like | 2.8699 |
| | Y*SGTSPSHSFISGEPDR\hat{ } | | | domain family B member 2 | |
| IPI:IPI00551454.3 | ER\hat{ }QES*ESEQELVNK@ | 642 | Pdcd11 | Protein RRP5 homolog | 2.8488 |
| IPI:IPI00656285.2 | SLVS*PIPSPT*GTISVPN | 643 | Foxk1 | Forkhead box protein K1 | 2.818 |
| | S*CPASPR\hat{ } | | | | |
| IPI:IPI00660767.2 | VDTAASSSWLAGS*CS*PVS | 644 | Ibtk | Isoform 2 of Inhibitor of Bruton | 2.8109 |
| | PPVVDLR\hat{ } | | | tyrosine kinase | |
| IPI:IPI00656285.2 | SLVS*PIPS*PTGTISVPNSC | 645 | Foxk1 | Forkhead box protein K1 | 2.8019 |
| | PAS*PR\hat{ } | | | | |
| IPI:IPI00656285.2 | SLVS*PIPS*PTGTISVPNSC | 646 | Foxk1 | Forkhead box protein K1 | 2.8019 |
| | PAS*PR\hat{ } | | | | |
| IPI:IPI00656285.2 | SLVSPIPSPT*GT*ISVPNSC | 647 | Foxk1 | Forkhead box protein K1 | 2.8019 |
| | PAS*PR | | | | |
| IPI:IPI00107958.1 | TSDIFGS*PVTATAPLAHPN | 648 | Hn1l | Hematological and neurological | 2.7751 |
| | K@PK@ | | | expressed 1-like protein | |
| IPI:IPI00129298.1 | EIDVLEFGESAPAASKENSA | 649 | Palm | Isoform 1 of Paralemmin | 2.7684 |
| | APS*PGRPQSASPAKEEQK | | | | |
| IPI:IPI00320208.3 | YGPSSVEDTTGSGAADAK | 650 | Eef1b2 | Elongation factor 1-beta | 2.7577 |
| | @DDDDIDLFGS*DDEEESE | | | | |
| | EAK@K@ | | | | |
| IPI:IPI00929786.1 | TASISSS*PSEGT*PAVGSY | 651 | Larp1 | Isoform 1 of La-related protein 1 | 2.7577 |
| | GCT*PQS*LPK@ | | | | |
| IPI:IPI00377615.2 | R\hat{ }SS*R\hat{ }S*FSLDEPPLFIPD | 652 | Phf3 | PHD finger protein 3 | 2.7452 |
| | NIATVK@ | | | | |
| IPI:IPI00113563.3 | YMEDST*YY*K@ASK@ | 653 | Ptk2 | Isoform 1 of Focal adhesion kinase 1 | 2.7394 |
| IPI:IPI00553283.1 | MMPS*PSDSSR\hat{ } | 654 | 1110013L07Rik | Putative uncharacterized protein | 2.7037 |
| IPI:IPI00314502.5 | VHGLPTTSPS*GVNMAELA | 655 | Tcfeb | Transcription factor EB | 2.6958 |
| | QQVVK@ | | | | |
| IPI:IPI00314502.5 | VHGLPTTSPS*GVNMAELA | 656 | Tcfeb | Transcription factor EB | 2.6958 |
| | QQVVK@ | | | | |
| IPI:IPI00122594.4 | EVS*VSS*VTEEPK@LDSSQ | 657 | Ahctf1 | AT-hook-containing transcription | 2.6674 |
| | LPLQTGLDVPAT*PR\hat{ } | | | factor 1 | |
| IPI:IPI00377615.2 | S*S*R\hat{ }SFSLDEPPLFIPDNIA | 658 | Phf3 | PHD finger protein 3 | 2.6537 |
| | TVK@K@ | | | | |
| IPI:IPI00468516.3 | SHILESVPTLPGS*VEAGVS | 659 | D6Wsu116e | Isoform 1 of Protein FAM21 | 2.6436 |
| | FDLPAQADTLHSANK@ | | | | |
| IPI:IPI00122521.1 | SSISSVLK@DPDSNPYSLLD | 660 | Fxr1 | Isoform E of Fragile X mental | 2.618 |
| | NTES*DQTADT*DAS*ESHH | | | retardation syndrome-related protein 1 | |
| | STNR\hat{ } | | | | |
| IPI:IPI00625723.2 | TGTGSPFAGNS*PAR\hat{ }EGE | 661 | Zc3h4 | Isoform 2 of Zinc finger CCCH domain- | 2.5583 |
| | QDAGSLK@DVFK@ | | | containing protein 4 | |
| IPI:IPI00625723.2 | TGTGSPFAGNS*PAR\hat{ }EGE | 662 | Zc3h4 | Isoform 2 of Zinc finger CCCH domain- | 2.5583 |
| | QDAGSLK@DVFK@ | | | containing protein 4 | |
| IPI:IPI00122559.3 | EIQNGTLR\hat{ }ES*DSEHVPR\hat{ } | 663 | Ktn1 | isoform 1 of Kinectin | 2.5547 |
| IPI:IPI00136379.1 | GGGSAAAAAAAAASGGGV | 664 | Pbx2 | Pre-B-cell leukemia transcription | 2.554 |
| | S*PDNSIEHSDYR\hat{ } | | factor 2 | | |
| IPI:IPI00330246.2 | SGPLPSSSGSSSSSSQLSV | 665 | Phldb1 | Isoform 2 of Pleckstrin homology-like | 2.5309 |
| | AT*LGR\hat{ }SPS*PK@ | | | domain family B member 1 | |
| IPI:IPI00330246.2 | SGPLPSSSGSSSSSSQLSV | 666 | Phldb1 | Isoform 2 of Pleckstrin homology-like | 2.5309 |
| | AT*LGR\hat{ }SPS*PK@ | | | domain family B member 1 | |
| IPI:IPI00377615.2 | S*S*R\hat{ }SFSLDEPPLFIPDNIA | 667 | Phf3 | PHD finger protein 3 | 2.5121 |
| | TVK@K@ | | | | |
| IPI:IPI00125505.2 | TTGNSHWTLEAPFSSS*CA | 668 | Cdgap | Cdc42 GTPase-activating protein | 2.4733 |
| | NLETER\hat{ } | | | | |
| IPI:IPI00551454.3 | ER\hat{ }QES*ESEQELVNK@ | 669 | Pdcd11 | Protein RRP5 homolog | 2.4392 |
| IPI:IPI00320594.5 | SQPHSSTSNQETS*DS*EM | 670 | Ranbp10 | Ran-binding protein 10 | 2.4384 |
| | EMEAEHYPNGVLESVSTR\hat{ } | | | | |
| IPI:IPI00126317.1 | NAS*TS*FQELEDK@K@EL | 671 | Dnajc2 | DnaJ homolog subfamily C member 2 | 2.438 |
| | SEESEDEELQLEEFPMLK@ | | | | |
| IPI:IPI00129276.2 | LES*LNIQR\hat{ } | 672 | Eif3a | Eukaryotic translation initiation | 2.429 |
| | | | | factor 3 subunit A | |
| IPI:IPI00553798.2 | SS*EVVLS*GDDEDYQR\hat{ } | 673 | Ahnak | AHNAK nucleoprotein isoform 1 | 2.4187 |
| IPI:IPI00226750.2 | T*S*S*TCSNESLNAGGTPV | 674 | Tbc1d4 | 140 kDa protein | 2.4185 |
| | TPR\hat{ } | | | | |
| IPI:IPI00377615.2 | R\hat{ }SSR\hat{ }S*FS*LDEPPLFIPD | 675 | Phf3 | PHD finger protein 3 | 2.409 |
| | NIATVK@ | | | | |
| IPI:IPI00377615.2 | R\hat{ }SSR\hat{ }S*FS*LDEPPLFIPD | 676 | Phf3 | PHD finger protein 3 | 2.409 |
| | NIATVK@ | | | | |
| IPI:IPI00114227.7 | SIST*CGPLDK@DDPGGQK@ | 677 | Dennd4a | hypothetical protein LOC102442 | 2.3733 |
| IPI:IPI00339693.1 | LGDSELALVCSQR\hat{ }PAS*LS | 678 | Wdr91 | WD repeat-containing protein 91 | 2.3532 |
| | QSPR\hat{ } | | | | |
| IPI:IPI00137166.1 | SR\hat{ }DAT*PPVS*PINMEDQE | 679 | Junb | Transcription factor jun-B | 2.3444 |
| | R\hat{ } | | | | |
| IPI:IPI00751009.1 | TVS*ASS*TGDLPK@ | 680 | Srpk2 | serine/arginine-rich protein-specific | 2.3444 |
| | | | | kinase 2 | |
| IPI:IPI00751009.1 | TVS*ASS*TGDLPK@ | 681 | Srpk2 | serine/arginine-rich protein-specific | 2.3444 |
| | | | | kinase 2 | |
| IPI:IPI00751009.1 | TVS*ASS*TGDLPK | 682 | Srpk2 | serine/arginine-rich protein-specific | 2.3444 |
| | | | | kinase 2 | |
| IPI:IPI00226750.2 | TSST*CS*NESLNAGGTPVT | 683 | Tbc1d4 | 140 kDa protein | 2.3429 |
| | PR\hat{ } | | | | |
| IPI:IPI00221581.1 | SR\hat{ }T*GS*ESSQTGASATSG | 684 | Eif4b | Eukaryotic translation initiation | 2.3268 |
| | R\hat{ } | | | factor 4B | |
| IPI:IPI00122521.1 | SSISSVLK@DPDSNPYSLLD | 685 | Fxr1 | Isoform E of Fragile X mental | 2.3266 |
| | NTES*DQT*ADT*DASESHH | | | retardation syndrome-related protein 1 | |
| | STNR\hat{ } | | | | |
| IPI:IPI00116442.1 | K@R\hat{ }S*EGLS*LER\hat{ } | 686 | Hdgfrp2 | Isoform 3 of Hepatoma-derived growth | 2.3042 |
| | | | | factor-related protein 2 | |
| IPI:IPI00828461.1 | GPPDFS*S*DEER\hat{ }EPT*PVL | 687 | Tmpo | thymopoietin isoform delta | 2.3011 |
| | GS*GASVGR\hat{ } | | | | |
| IPI:IPI00463493.1 | S*ATDADMVNSGWLVVGK | 688 | Gbf1 | Golgi-specific brefeldin A-resistance | 2.294 |
| | | | | factor 1 | |
| IPI:IPI00129276.2 | LES*LNIQR\hat{ } | 689 | Eif3a | Eukaryotic translation initiation | 2.2772 |
| | | | | factor 3 subunit A | |
| IPI:IPI00751009.1 | TVS*ASS*TGDLPK@ | 690 | Srpk2 | serine/arginine-rich protein-specific | 2.2686 |
| | | | | kinase 2 | |
| IPI:IPI00137166.1 | SR\hat{ }DAT*PPVS*PINMEDQE | 691 | Junb | Transcription factor jun-B | 2.2666 |
| | R\hat{ } | | | | |
| IPI:IPI00221581.1 | SR\hat{ }TGS*ESS*QTGASATSG | 692 | Eif4b | Eukaryotic translation initiation | 2.2646 |
| | R\hat{ } | | | factor 4B | |
| IPI:IPI00221581.1 | SR\hat{ }TGS*ESS*QTGASATSG | 693 | Eif4b | Eukaryotic translation initiation | 2.2646 |
| | R\hat{ } | | | factor 4B | |
| IPI:IPI00221581.1 | SRT*GS*ESSQTGASATSGR | 694 | Eif4b | Eukaryotic translation initiation | 2.2646 |
| | | | | factor 4B | |
| IPI:IPI00268688.3 | TSS*S*ETEEK@K@TEK@P | 695 | Cd2ap | CD2-associated protein | 2.2567 |
| | LILQPLGSR\hat{ } | | | | |
| IPI:IPI00268688.3 | TSS*S*ETEEKKTEKPLILQP | 696 | Cd2ap | CD2-associated protein | 2.2567 |
| | LGSR | | | | |
| IPI:IPI00226750.2 | TSST*CS*NES*LNAGGTPV | 697 | Tbc1d4 | 140 kDa protein | 2.2548 |
| | TPR\hat{ } | | | | |
| IPI:IPI00221581.1 | SRT*GS*ESSQTGASATSGR | 698 | Eif4b | Eukaryotic translation initiation | 2.2395 |
| | | | | factor 4B | |
| IPI:IPI00221581.1 | SR\hat{ }TGS*ESS*QTGASATSG | 699 | Eif4b | Eukaryotic translation initiation | 2.2395 |
| | R\hat{ } | | | factor 4B | |
| IPI:IPI00122594.4 | EVS*VSS*VTEEPK@LDSSQ | 700 | Ahctf1 | AT-hook-containing transcription | 2.2291 |
| | LPLQTGLDVPATPR\hat{ } | | | factor 1 | |
| IPI:IPI00122594.4 | EVSVS*S*VTEEPKLDSSQL | 701 | Ahctf1 | AT-hook-containing transcription | 2.2291 |
| | PLQTGLDVPATPR | | | factor 1 | |
| IPI:IPI00676574.2 | RVST*DLPEGQDVYTAACN | 702 | Herc1 | hect (homologous to the E6-AP | 2.2125 |
| | SVIHR | | | (UBE3A) carboxyl terminus) domain | |
| | | | | and RCC1 (CHC1)-like domain (RLD) 1 | |
| IPI:IPI00268688.3 | TSS*S*ETEEK@K@TEK@P | 703 | Cd2ap | CD2-associated protein | 2.1992 |
| | LILQPLGSR\hat{ } | | | | |
| IPI:IPI00320905.7 | LGEQGPEPGPT*PPQTPT*P | 704 | Arhgap17 | Isoform 1 of Rho GTPase-activating | 2.1798 |
| | PS*TPPLAK | | | protein 17 | |
| IPI:IPI00108454.2 | LSSLRAS*TS*KSES*S*QK | 705 | — | 29 kDa protein | 2.1655 |
| IPI:IPI00929786.1 | TASIS*S*SPSEGTPAVGSY | 706 | Larp1 | Isoform 1 of La-related protein 1 | 2.1557 |
| | GCT*PQSLPK@ | | | | |
| IPI:IPI00381495.5 | TVSSPIPYTPSPSSSR\hat{ }PIS* | 707 | Ccdc6 | coiled-coil domain containing 6 | 2.1555 |
| | PGLSY*ASHTVGFTPPTSLT | | | | |
| | R\hat{ } | | | | |
| IPI:IPI00221581.1 | TGS*ESS*QTGASATSGR\hat{ } | 708 | Eif4b | Eukaryotic translation initiation | 2.1437 |
| | | | | factor 4B | |
| IPI:IPI00221581.1 | TGS*ESS*QTGASATSGR\hat{ } | 709 | Eif4b | Eukaryotic translation initiation | 2.1437 |
| | | | | factor 4B | |
| IPI:IPI00137166.1 | SR\hat{ }DAT*PPVS*PINMEDQE | 710 | Junb | Transcription factor jun-B | 2.1419 |
| | R\hat{ } | | | | |
| IPI:IPI00929786.1 | S*LPTTVPES*PNYR\hat{ } | 711 | Larp1 | Isoform 1 of La-related protein 1 | 2.1397 |
| IPI:IPI00929786.1 | S*LPTTVPESPNY*R | 712 | Larp1 | Isoform 1 of La-related protein 1 | 2.1397 |
| IPI:IPI00406045.3 | SESSAGICVPLSTS*PQVSE | 713 | Pik3r4 | Phosphoinositide 3-kinase regulatory | 2.1394 |
| | AAHIPSK@K@PVIPVVSST | | | subunit 4 | |
| | VLPSTYQIR\hat{ } | | | | |
| IPI:IPI00122594.4 | ER\hat{ }EVS*VS*SVTEEPK@LD | 714 | Ahctf1 | AT-hook-containing transcription | 2.1312 |
| | SSQLPLQTGLDVPATPR\hat{ } | | | factor 1 | |
| IPI:IPI00929786.1 | S*LPTTVPES*PNYR\hat{ } | 715 | Larp1 | Isoform 1 of La-related protein 1 | 2.1207 |
| IPI:IPI00929786.1 | S*LPTTVPES*PNYR | 716 | Larp1 | Isoform 1 of La-related protein 1 | 2.1207 |
| IPI:IPI00929786.1 | SLPT*TVPES*PNYR\hat{ } | 717 | Larp1 | Isoform 1 of La-related protein 1 | 2.1207 |
| IPI:IPI00929786.1 | S*LPTTVPES*PNYR | 718 | Larp1 | Isoform 1 of La-related protein 1 | 2.1207 |
| IPI:IPI00625723.2 | TGTGSPFAGNS*PAR\hat{ }EGE | 719 | Zc3h4 | Isoform 2 of Zinc finger CCCH domain- | 2.1078 |
| | QDAGSLK@DVFK@ | | | containing protein 4 | |
| IPI:IPI00221581.1 | T*GSESS*QTGASATSGR\hat{ } | 720 | Eif4b | Eukaryotic translation initiation | 2.103 |
| | | | | factor 4B | |
| IPI:IPI00221581.1 | T*GSESS*QTGASATSGR\hat{ } | 721 | Eif4b | Eukaryotic translation initiation | 2.103 |
| | | | | factor 4B | |
| IPI:IPI00221581.1 | TGS*ESS*QTGASATSGR | 722 | Eif4b | Eukaryotic translation initiation | 2.103 |
| | | | | factor 4B | |
| IPI:IPI00625723.2 | TGTGSPFAGNS*PAR\hat{ }EGE | 723 | Zc3h4 | Isoform 2 of Zinc finger CCCH domain- | 2.0988 |
| | QDAGSLK@DVFK@ | | | containing protein 4 | |
| IPI:IPI00379844.4 | GEQGSLAQSQPQPGDK@N | 724 | Irs2 | Insulin receptor substrate 2 | 2.0959 |
| | S*WSR\hat{ } | | | | |
| IPI:IPI00136107.1 | T*HST*SSS*IGSGESPFSR\hat{ } | 725 | Ndrg3 | Protein NDRG3 | 2.0759 |
| IPI:IPI00467843.2 | GGGAS*SPAPVVFTVGS*PP | 726 | Ulk1 | Putative uncharacterized protein | 2.0752 |
| | S*GATPPQSTR\hat{ } | | | | |
| IPI:IPI00127415.1 | LK@CGSGPVHISGQHLVAV | 727 | Npm1 | Nucleophosmin | 2.0591 |
| | EEDAES*EDEDEEDVK@LL | | | | |
| | GM#SGK@ | | | | |
| IPI:IPI00320905.7 | LGEQGPEPGPT*PPQTPT*P | 728 | Arhgap17 | Isoform 1 of Rho GTPase-activating | 2.0572 |
| | PS*TPPLAK | | | protein 17 | |
| IPI:IPI00622946.2 | QFIAAQNLGPASGLPTPTSS | 729 | Palld | Isoform 4 of Palladin | 2.0517 |
| | *PSSSSLPSPLSPT*PR\hat{ }PFG | | | | |
| | R\hat{ } | | | | |
| IPI:IPI00467843.2 | GGGASS*PAPVVFTVGS*PP | 730 | Ulk1 | Putative uncharacterized protein | 2.0359 |
| | S*GATPPOSTR\hat{ } | | | | |
| IPI:IPI00929786.1 | TASISSSPS*EGTPAVGSYG | 731 | Larp1 | Isoform 1 of La-related protein 1 | 2.0315 |
| | CT*PQSLPK@ | | | | |
| IPI:IPI00309059.7 | ST*S*PIIGS*PPVR\hat{ } | 732 | Patl1 | Protein PAT1 homolog 1 | 2.0311 |
| IPI:IPI00929786.1 | TASIS*SSPS*EGTPAVGSY | 733 | Larp1 | Isoform 1 of La-related protein 1 | 2.0277 |
| | GCT*PQS*LPK@ | | | | |
| IPI:IPI00107958.1 | TSDIFGS*PVTATAPLAHPN | 734 | Hn1l | Hematological and neurological | 2.0059 |
| | KPK | | | expressed 1-like protein | |
| IPI:IPI00336713.1 | CS*PVPGLSSS*PSGSPLHG | 735 | Bcas3 | Isoform 1 of Breast carcinoma- | 1.9848 |
| | K@ | | | amplified sequence 3 homolog | |
| IPI:IPI00136107.1 | THST*SSS*IGSGESPFSR\hat{ } | 736 | Ndrg3 | Protein NDRG3 | 1.9707 |
| IPI:IPI00136107.1 | THSTSS*S*IGSGESPFSR\hat{ } | 737 | Ndrg3 | Protein NDRG3 | 1.9707 |
| IPI:IPI00830210.1 | LSESPAS*LPSCLPVETALIN | 738 | Atg2a | Autophagy-related protein 2 | 1.9651 |
| | QR\hat{ } | | | homolog A | |
| IPI:IPI00225062.2 | GCSPPK@S*PEK@PPQST | 739 | Srrm2 | Isoform 3 of Serine/arginine repetitive | 1.9354 |
| | SS*ESCPPS*PQPTK@ | | | matrix protein 2 | |
| IPI:IPI00122594.4 | ER\hat{ }EVS*VSS*VTEEPK@LD | 740 | Ahctf1 | AT-hook-containing transcription | 1.9159 |
| | SSQLPLQTGLDVPATPR\hat{ } | | | factor 1 | |
| IPI:IPI00336973.2 | GLNLDGTPALSTLGGFS*PA | 741 | Ccnl1 | Isoform 1 of Cyclin-L1 | 1.8986 |
| | SKPSS*PR | | | | |
| IPI:IPI00336973.2 | GLNLDGTPALSTLGGFS*PA | 742 | Ccnl1 | Isoform 1 of Cyclin-L1 | 1.896 |
| | SKPSS*PR | | | | |
| IPI:IPI00336973.2 | GLNLDGTPALSTLGGFS*PA | 743 | Ccnl1 | Isoform 1 of Cyclin-L1 | 1.896 |
| | SK@PS*SPR\hat{ } | | | | |
| IPI:IPI00336973.2 | GLNLDGTPALSTLGGFS*PA | 744 | Ccnl1 | Isoform 1 of Cyclin-L1 | 1.896 |
| | S*KPSSPR | | | | |
| IPI:IPI00336973.2 | GLNLDGTPALSTLGGFSPA | 745 | Ccnl1 | Isoform 1 of Cyclin-L1 | 1.896 |
| | S*K@PS*SPR\hat{ } | | | | |
| IPI:IPI00137166.1 | SR\hat{ }DAT*PPVS*PINMEDQE | 746 | Junb | Transcription factor jun-B | 1.8916 |
| | R\hat{ } | | | | |
| IPI:IPI00336973.2 | GLNLDGTPALSTLGGFS*PA | 747 | Ccnl1 | Isoform 1 of Cyclin-L1 | 1.8893 |
| | S*KPSSPR | | | | |
| IPI:IPI00467930.2 | LVIGS*LPAHLSPHLFGGFK@ | 748 | Znrf2 | E3 ubiquitin-protein ligase ZNRF2 | 1.8798 |
| IPI:IPI00225062.2 | R\hat{ }SSS*ELS*PEVVEK@ | 749 | Srrm2 | Isoform 3 of Serine/arginine repetitive | 1.8718 |
| | | | | matrix protein 2 | |
| IPI:IPI00309059.7 | ST*S*PIIGS*PPVR\hat{ } | 750 | Patl1 | Protein PAT1 homolog 1 | 1.8673 |
| IPI:IPI00751009.1 | TSEFPTPLFSGPLEPVACGS | 751 | Srpk2 | serine/arginine-rich protein specific | 1.859 |
| | VISEGSPLT*EQEESSPSHD | | | kinase 2 | |
| | R\hat{ }SR\hat{ } | | | | |
| IPI:IPI00808277.2 | SAPASPNHAGVLSAHSSGA | 752 | Foxk2 | Isoform 1 of Forkhead box protein K2 | 1.8546 |
| | QT*PES*LSR\hat{ }EGS*PAPLEP | | | | |
| | EPGASQPK@ | | | | |
| IPI:IPI00136107.1 | THS*TSSS*IGSGESPFSR\hat{ } | 753 | Ndrg3 | Protein NDRG3 | 1.8482 |
| IPI:IPI00761759.1 | SSSGSEHST*EGSVSLGDG | 754 | Larp4 | Putative uncharacterized protein | 1.8482 |
| | PLSR\hat{ } | | | | |
| IPI:IPI00122594.4 | EVS*VSS*VTEEPK@LDSSQ | 755 | Ahctf1 | AT-hook-containing transcription | 1.8472 |
| | LPLQTGLDVPATPR\hat{ } | | | factor 1 | |
| IPI:IPI00122594.4 | EVS*VSS*VTEEPK@LDSSQ | 756 | Ahctf1 | AT-hook-containing transcription | 1.8472 |
| | LPLQTGLDVPATPR\hat{ } | | | factor 1 | |
| IPI:IPI00268673.5 | AGTTVPES*IHS*FIGDGLVK | 757 | Mtor | Isoform 1 of FKBP12-rapamycin | 1.8442 |
| | @PEALNK@K@ | | | complex-associated protein | |
| IPI:IPI00314502.5 | VHGLPTTS*PSGVNMAELA | 758 | Tcfeb | Transcription factor EB | 1.8426 |
| | QQVVK@ | | | | |
| IPI:IPI00125319.1 | TTS*FAESCK@PVQQPSAF | 759 | Gsk3b | Glycogen synthase kinase-3 beta | 1.8417 |
| | GSMK@ | | | | |
| IPI:IPI00125319.1 | TTS*FAESCK@PVQQPSAF | 760 | Gsk3b | Glycogen synthase kinase-3 beta | 1.8417 |
| | GSMK@ | | | | |
| IPI:IPI00116285.2 | SQDSYPVS*PR\hat{ }PFSSPSMS | 761 | Ranbp9 | RAN binding protein 9 | 1.8399 |
| | *PSHGMSIHSLAPGK@ | | | | |
| IPI:IPI00323510.5 | TDNSVAS*SPSSAISTATPS* | 762 | Kdm6a | Isoform 1 of Lysine-specific | 1.8307 |
| | PK@ | | | demethylase 6A | |
| IPI:IPI00229859.1 | GHPSAGAEEEGGS*DGS*A | 763 | Eif3b | Eif3b protein | 1.826 |
| | AEAEPR | | | | |
| PI:IPI00136107.1 | THS*TSS*SIGSGESPFSR\hat{ } | 764 | Ndrg3 | Protein NDRG3 | 1.8106 |
| IPI:IPI00136107.1 | T*HSTSSSIGS*GESPFSR\hat{ } | 765 | Ndrg3 | Protein NDRG3 | 1.8106 |
| IPI:IPI00225062.2 | GCS*PPK@S*PEK@PPQST | 766 | Srrm2 | Isoform 3 of Serine/arginine repetitive | 1.8106 |
| | SSESCPPS*PQPTK@ | | | matrix protein 2 | |
| IPI:IPI00405752.3 | VLASS*LS*PYR\hat{ }EGR\hat{ } | 767 | C130092O11Rik | Isoform 1 of Uncharacterized protein | 1.8087 |
| | | | | KIAA1680 | |
| IPI:IPI00136107.1 | THSTSS*S*IGSGESPFSR\hat{ } | 768 | Ndrg3 | Protein NDRG3 | 1.8041 |
| IPI:IPI00130920.1 | RSES*PFEGK | 769 | Mtap1b | Microtubule-associated protein 1B | 1.7981 |
| IPI:IPI00136107.1 | THS*TSS*S*IGSGESPFSR\hat{ } | 770 | Ndrg3 | Protein NDRG3 | 1.7967 |
| IPI:IPI00336713.1 | CS*PVPGLSSS*PSGSPLHG | 771 | Bcas3 | Isoform 1 of Breast carcinoma- | 1.7932 |
| | K@ | | | amplified sequence 3 homolog | |
| IPI:IPI00323349.2 | GYPPPIAAK@PAFGR\hat{ }PILK | 772 | Tjp2 | Tight junction protein ZO-2 | 1.7869 |
| | @PST*PVPMPESEEVGES* | | | | |
| | TEEQEDAPR\hat{ } | | | | |
| IPI:IPI00225062.2 | GCS*PPK@S*PEK@PPQST | 773 | Srrm2 | Isoform 3 of Serine/arginine repetitive | 1.7868 |
| | SSESCPPS*PQPTK@ | | | matrix protein 2 | |
| IPI:IPI00225062.2 | GCS*PPKS*PEKPPQSTSSE | 774 | Srrm2 | Isoform 3 of Serine/arginine repetitive | 1.7868 |
| | SCPPS*PQPTK | | | matrix protein 2 | |
| IPI:IPI00808277.2 | SAPASPNHAGVLSAHSSGA | 775 | Foxk2 | Isoform 1 of Forkhead box protein K2 | 1.7848 |
| | QT*PES*LSR\hat{ }EGS*PAPLEP | | | | |
| | EPGASQPK@ | | | | |
| IPI:IPI00136107.1 | THS*TSS*S*IGSGESPFSR\hat{ } | 776 | Ndrg3 | Protein NDRG3 | 1.7706 |
| IPI:IPI00107958.1 | TSDIFGS*PVTATAPLAHPN | 777 | Hn1l | Hematological and neurological | 1.7561 |
| | K@PK@ | | | expressed 1-like protein | |
| IPI:IPI00136107.1 | THS*TSSS*IGSGESPFSR\hat{ } | 778 | Ndrg3 | Protein NDRG3 | 1.7555 |
| IPI:IPI00136107.1 | THS*TSSS*IGSGESPFSR\hat{ } | 779 | Ndrg3 | Protein NDRG3 | 1.7525 |
| IPI:IPI00130920.1 | RSES*PFEGK | 780 | Mtap1b | Microtubule-associated protein 1B | 1.7508 |
| IPI:IPI00130920.1 | RSES*PFEGK | 781 | Mtap1b | Microtubule-associated protein 1B | 1.7508 |
| IPI:IPI00136107.1 | THS*TSS*SIGSGESPFSR\hat{ } | 782 | Ndrg3 | Protein NDRG3 | 1.7499 |
| IPI:IPI00136107.1 | THS*TSS*SIGSGESPFSR | 783 | Ndrg3 | Protein NDRG3 | 1.7499 |
| IPI:IPI00318938.6 | VALGDGVQLPPGDYSTT*P | 784 | Eif4ebp1 | Eukaryotic translation initiation | 1.7456 |
| | GGTLFS*TTPGGTRIIYDR | | | factor 4E-binding protein 1 | |
| IPI:IPI00468516.3 | R\hat{ }LAAQES*SEAEDVTVDR\hat{ } | 785 | D6Wsu116e | Isoform 1 of Protein FAM21 | 1.7446 |
| | GPVAQLSSS*PVLPNGHQP | | | | |
| | LLQPR\hat{ } | | | | |
| IPI:IPI00137501.1 | TEMDKS*PFNSPS*PQDS*PR | 786 | Nfic | Isoform 1 of Nuclear factor 1 C-type | 1.7419 |
| IPI:IPI00225062.2 | GCS*PPK@S*PEK@PPQS* | 787 | Srrm2 | Isoform 3 of Serine/arginine repetitive | 1.7403 |
| | TSSESCPPSPQPTK@ | | | matrix protein 2 | |
| IPI:IPI00225062.2 | GCS*PPK@S*PEK@PPQST | 788 | Srrm2 | Isoform 3 of Serine/arginine repetitive | 1.7403 |
| | SSESCPPS*PQPTK@ | | | matrix protein 2 | |
| IPI:IPI00225062.2 | GCS*PPKS*PEKPPQSTSS* | 789 | Srrm2 | Isoform 3 of Serine/arginine repetitive | 1.7403 |
| | ESCPPSPQPTK | | | matrix protein 2 | |
| IPI:IPI00336973.2 | GLNLDGTPALSTLGGFS*PA | 790 | Ccnl1 | Isoform 1 of Cyclin-L1 | 1.7368 |
| | SK@PS*SPR\hat{ } | | | | |
| IPI:IPI00107958.1 | TSDIFGS*PVTATAPLAHPN | 791 | Hn1l | Hematological and neurological | 1.7353 |
| | K@PK@ | | | expressed 1-like protein | |
| IPI:IPI00227209.3 | R\hat{ }LSS*T*SLASGHSVR\hat{ } | 792 | Prkd2 | Serine/threonine-protein kinase D2 | 1.7328 |
| IPI:IPI00225062.2 | RSS*SELS*PEVVEK | 793 | Srrm2 | Isoform 3 of Serine/arginine repetitive | 1.7324 |
| | | | | matrix protein 2 | |
| IPI:IPI00225062.2 | R\hat{ }SS*SELS*PEVVEK@ | 794 | Srrm2 | Isoform 3 of Serine/arginine repetitive | 1.7324 |
| | | | | matrix protein 2 | |
| IPI:IPI00225062.2 | RSS*SELS*PEVVEK | 795 | Srrm2 | Isoform 3 of Serine/arginine repetitive | 1.7324 |
| | | | | matrix protein 2 | |
| IPI:IPI00225062.2 | R\hat{ }SSS*ELS*PEVVEK@ | 796 | Srrm2 | Isoform 3 of Serine/arginine repetitive | 1.7324 |
| | | | | matrix protein 2 | |
| IPI:IPI00929779.1 | S*EGLSQEATPSQDLIQHSC | 797 | Usp36 | Ubiquitin specific peptidase 36 | 1.7192 |
| | SPVDHSEPEAR\hat{ } | | | | |
| IPI:IPI00467930.2 | LVIGS*LPAHLS*PHLFGGFK | 798 | Znrf2 | E3 ubiquitin-protein ligase ZNRF2 | 1.7137 |
| IPI:IPI00467930.2 | LVIGS*LPAHLS*PHLFG | 799 | Znrf2 | E3 ubiquitin-protein ligase ZNRF2 | 1.7137 |
| | GFK@ | | | | |
| IPI:IPI00459443.5 | DLQSEFGVATDSHHSSFGS | 800 | Tnks1bp1 | 182 kDa tankyrase-1-binding protein | 1.7011 |
| | SSWSQDTSQNYSLGGR\hat{ }S* | | | | |
| | PVGDTGLGK@ | | | | |
| IPI:IPI00785400.1 | LTSHTPGLDDEKEAS*ENET | 801 | Syne2 | Syne2 protein | 1.7003 |
| | *DIEDPREIQADSWR | | | | |
| IPI:IPI00136107.1 | THST*SSSIGSGESPFSR\hat{ } | 802 | Ndrg3 | Protein NDRG3 | 1.6838 |
| IPI:IPI00136107.1 | THST*SSSIGSGESPFSR\hat{ } | 803 | Ndrg3 | Protein NDRG3 | 1.6838 |
| IPI:IPI00336713.1 | QGGRCS*PVPGLSSS*PSG | 804 | Bcas3 | Isoform 1 of Breast carcinoma- | 1.6818 |
| | SPLHGK | | | amplified sequence 3 homolog | |
| IPI:IPI00336713.1 | QGGR\hat{ }CS*PVPGLSS*SPSG | 805 | Bcas3 | Isoform 1 of Breast carcinoma- | 1.6818 |
| | SPLHGK@ | | | amplified sequence 3 homolog | |
| IPI:IPI00380354.1 | LQES*PKLSQANGTR | 806 | Llgl1 | lethal giant larvae homolog 1 isoform 1 | 1.6806 |
| IPI:IPI00128904.1 | VMTIPYQPMPASS*PVICAG | 807 | Pcbp1 | Poly(rC)-binding protein 1 | 1.6782 |
| | GQDR | | | | |
| IPI:IPI00380107.4 | LVIPS*ATTKSPPEITVT* | 808 | Cabin1 | calcineurin binding protein 1 | 1.6782 |
| | PPT*PTLLSPK | | | | |
| IPI:IPI00153375.1 | SACFS*PVSLS*PR\hat{ }PCS*PF | 809 | Pdlim2 | PDZ and LIM domain protein 2 | 1.6659 |
| | STPPPTSPVALSK@ | | | | |
| IPI:IPI00467930.2 | LVIGS*LPAHLSPHLFGGFK@ | 810 | Znrf2 | E3 ubiquitin-protein ligase ZNRF2 | 1.6655 |
| IPI:IPI00467930.2 | LVIGS*LPAHLSPHLFGGFK@ | 811 | Znrf2 | E3 ubiquitin-protein ligase ZNRF2 | 1.6655 |
| IPI:IPI00221581.1 | S*R\hat{ }TGS*ESS*QTGASATS | 812 | Eif4b | Eukaryotic translation initiation | 1.6642 |
| | GR\hat{ } | | | factor 4B | |
| IPI:IPI00318938.6 | NS*PVAK@T*PPK@DLPAIP | 813 | Eif4ebp1 | Eukaryotic translation initiation | 1.6633 |
| | GVTSPTSDEPPMQASQSQL | | | factor 4E-binding protein 1 | |
| | PSSPEDK@ | | | | |
| IPI:IPI00120529.2 | NK@PLS*PIK@LTPTSVLDY | 814 | Rfc1 | Rfc1 protein | 1.6568 |
| | FGTESVQR\hat{ } | | | | |
| IPI:IPI00225062.2 | RSS*SELS*PEVVEK | 815 | Srrm2 | Isoform 3 of Serine/arginine repetitive | 1.6324 |
| | | | | matrix protein 2 | |
| IPI:IPI00225062.2 | R\hat{ }SS*SELS*PEVVEK@ | 816 | Srrm2 | Isoform 3 of Serine/arginine repetitive | 1.6324 |
| | | | | matrix protein 2 | |
| IPI:IPI00225062.2 | R\hat{ }SS*SELS*PEVVEK@ | 817 | Srrm2 | Isoform 3 of Serine/arginine repetitive | 1.6324 |
| | | | | matrix protein 2 | |
| IPI:IPI00225062.2 | RSS*SELS*PEVVEK | 818 | Srrm2 | Isoform 3 of Serine/arginine repetitive | 1.6324 |
| | | | | matrix protein 2 | |
| IPI:IPI00225062.2 | R\hat{ }SS*SELS*PEVVEK@ | 819 | Srrm2 | Isoform 3 of Serine/arginine repetitive | 1.6324 |
| | | | | matrix protein 2 | |
| IPI:IPI00225062.2 | R\hat{ }SS*SELS*PEVVEK@ | 820 | Srrm2 | Isoform 3 of Serine/arginine repetitive | 1.6324 |
| | | | | matrix protein 2 | |
| IPI:IPI00761759.1 | SSSGSEHSTEGS*VSLGDG | 821 | Larp4 | Putative uncharacterized protein | 1.6294 |
| | PLSR\hat{ } | | | | |
| IPI:IPI00309059.7 | R\hat{ }S*TS*PIIGS*PPVR\hat{ } | 822 | Patl1 | Protein PAT1 homolog 1 | 1.6275 |
| IPI:IPI00309059.7 | R\hat{ }S*TS*PIIGS*PPVR\hat{ } | 823 | Patl1 | Protein PAT1 homolog 1 | 1.6245 |
| IPI:IPI00317401.6 | AES*PETSAVESTQS*TPQK | 824 | Pds5b | Isoform 1 of Sister chromatid cohesion | 1.623 |
| | @GR\hat{ } | | | protein PDS5 homolog B | |
| IPI:IPI00317401.6 | AES*PETSAVESTQST*PQK | 825 | Pds5b | Isoform 1 of Sister chromatid cohesion | 1.623 |
| | @GR\hat{ } | | | protein PDS5 homolog B | |
| IPI:IPI00115620.1 | AS*LYNAVTTEDVEK | 826 | Psat1 | Phosphoserine aminotransferase | 1.6216 |
| IPI:IPI00126124.2 | T*FSECS*YPETEEEAEALP | 827 | St5 | Isoform 1 of Suppression of | 1.6159 |
| | GR\hat{ } | | | tumorigenicity 5 | |
| IPI:IPI00421179.1 | SFS*K@EVEER\hat{ } | 828 | Eif4g1 | Isoform 1 of Eukaryotic translation | 1.6081 |
| | | | | initiation factor 4 gamma 1 | |
| IPI:IPI00225062.2 | GCSPPK@S*PEK@PPQST* | 829 | Srrm2 | Isoform 3 of Serine/arginine repetitive | 1.608 |
| | SSESCPPS*PQPTK@ | | | matrix protein 2 | |
| IPI:IPI00227934.3 | AAPSS*EGDS*CDGVEATDA | 830 | Cux1 | cut-like homeobox 1 isoform a | 1.6068 |
| | EEPGGNIVATK@ | | | | |
| IPI:IPI00314240.5 | K@LEK@EEEEGIS*QES*S* | 831 | Hmga1 | Isoform HMG-I of High mobility group | 1.6058 |
| | EEEQ | | | protein HMG-I/HMG-Y | |
| IPI:IPI00111754.6 | LDT*FCGSPPYAAPELFQGK | 832 | Mark2 | Isoform 2 of Serine/threonine-protein | 1.5994 |
| | | | | kinase MARK2 | |
| IPI:IPI00125319.1 | TTS*FAESCK@PVQQPSAF | 833 | Gsk3b | Glycogen synthase kinase-3 beta | 1.5966 |
| | GSMK@ | | | | |
| IPI:IPI00320905.7 | LGEQGPEPGPT*PPQT*PTP | 834 | Arhgap17 | Isoform 1 of Rho GTPase-activating | 1.5946 |
| | PS*TPPLAK | | | protein 17 | |
| IPI:IPI00380737.1 | TSSVS*S*LASACTGGIPSSS | 835 | Pom121 | Nuclear envelope pore membrane | 1.5883 |
| | R\hat{ } | | | protein POM 121 | |
| IPI:IPI00122594.4 | TTPLAS*PSLS*PGR\hat{ } | 836 | Ahctf1 | AT-hook-containing transcription | 1.5845 |
| | | | | factor 1 | |
| IPI:IPI00125319.1 | TTS*FAESCK@PVQQPSAF | 837 | Gsk3b | Glycogen synthase kinase-3 beta | 1.5821 |
| | GSMK@ | | | | |
| IPI:IPI00381019.1 | KRS*PS*PSPTPEAK | 838 | Smarcc2 | Isoform 2 of SWI/SNF complex subunit | 1.5795 |
| | | | | SMARCC2 | |
| IPI:IPI00111754.6 | LDT*FCGSPPYAAPELFQGK@ | 839 | Mark2 | Isoform 2 of Serine/threonine-protein | 1.578 |
| | | | | kinase MARK2 | |
| IPI:IPI00111754.6 | LDT*FCGSPPYAAPELFQGK@ | 840 | Mark2 | Isoform 2 of Serine/threonine-protein | 1.578 |
| | | | | kinase MARK2 | |
| IPI:IPI00136107.1 | THS*TSSSIGSGESPFSR\hat{ } | 841 | Ndrg3 | Protein NDRG3 | 1.576 |
| IPI:IPI00136107.1 | THS*TSSSIGSGESPFSR\hat{ } | 842 | Ndrg3 | Protein NDRG3 | 1.576 |
| IPI:IPI00128904.1 | VMTIPYQPMPASS*PVICAG | 843 | Pcbp1 | Poly(rC)-binding protein 1 | 1.5752 |
| | GQDR | | | | |
| IPI:IPI00225062.2 | S*SS*PVTELTAR\hat{ } | 844 | Srrm2 | Isoform 3 of Serine/arginine repetitive | 1.5701 |
| | | | | matrix protein 2 | |
| IPI:IPI00421179.1 | SFS*KEVEER | 845 | Eif4g1 | Isoform 1 of Eukaryotic translation | 1.5642 |
| | | | | initiation factor 4 gamma 1 | |
| IPI:IPI00421179.1 | SFS*K@EVEER\hat{ } | 846 | Eif4g1 | Isoform 1 of Eukaryotic translation | 1.5642 |
| | | | | initiation factor 4 gamma 1 | |
| IPI:IPI00421179.1 | SFS*KEVEER | 847 | Eif4g1 | Isoform 1 of Eukaryotic translation | 1.5642 |
| | | | | initiation factor 4 gamma 1 | |
| IPI:IPI00421179.1 | SFS*K@EVEER\hat{ } | 848 | Eif4g1 | Isoform 1 of Eukaryotic translation | 1.5642 |
| | | | | initiation factor 4 gamma 1 | |
| IPI:IPI00115492.1 | HSLSSESQAPEDIAPPGS*S | 849 | Eps8l2 | Isoform 1 of Epidermal growth factor | 1.564 |
| | PHANR\hat{ } | | | receptor kinase substrate 8-like | |
| | | | | protein 2 | |
| IPI:IPI00761759.1 | S*SS*GS*EHSTEGSVSLGD | 850 | Larp4 | Putative uncharacterized protein | 1.5621 |
| | GPLSR\hat{ } | | | | |
| IPI:IPI00136107.1 | THSTS*SSIGSGESPFSR\hat{ } | 851 | Ndrg3 | Protein NDRG3 | 1.5584 |
| IPI:IPI00111754.6 | LDT*FCGSPPYAAPELFQGK | 852 | Mark2 | Isoform 2 of Serine/threonine-protein | 1.5403 |
| | | | | kinase MARK2 | |
| IPI:IPI00336973.2 | GLNLDGT*PALSTLGGFS*P | 853 | Ccnl1 | Isoform 1 of Cyclin-L1 | 1.5363 |
| | ASK@PS*SPR\hat{ } | | | | |
| IPI:IPI00229739.4 | LVVSS*PTS*PK@GK@ | 854 | Plekhm1 | Pleckstrin homology domain-containing | 1.5286 |
| | | | | family M member 1 | |
| IPI:IPI00126124.2 | TFSECS*Y*PETEEEAEALP | 855 | St5 | Isoform 1 of Suppression of | 1.526 |
| | GR\hat{ } | | | tumorigenicity 5 | |
| IPI:IPI00122594.4 | TTPLAS*PSLS*PGR\hat{ } | 856 | Ahctf1 | AT-hook-containing transcription | 1.5177 |
| | | | | factor 1 | |
| IPI:IPI00229739.4 | LWSS*PTS*PKGK | 857 | Plekhm1 | Pleckstrin homology domain-containing | 1.515 |
| | | | | family M member 1 | |
| IPI:IPI00421179.1 | SFS*K@EVEER\hat{ } | 858 | Eif4g1 | Isoform 1 of Eukaryotic translation | 1.5113 |
| | | | | initiation factor 4 gamma 1 | |
| IPI:IPI00421179.1 | SFS*KEVEER | 859 | Eif4g1 | Isoform 1 of Eukaryotic translation | 1.5113 |
| | | | | initiation factor 4 gamma 1 | |
| IPI:IPI00349069.4 | LFGT*SPAAEVTPSPPEPAP | 860 | B230208H17Rik | Putative GTP-binding protein Parf | 1.5073 |
| | ALEAPAR\hat{ } | | | | |
| IPI:IPI00605037.1 | KET*ES*EAEDDNLDDLER | 861 | Srrm1 | Isoform 1 of Serine/arginine repetitive | 1.5038 |
| | | | | matrix protein 1 | |
| IPI:IPI00381244.8 | KEELGASS*PGYGPPNLGC | 862 | Mll2 | similar to myeloid/lymphoid or mixed- | 1.4993 |
| | VDS*PSAGPHLGGLELK | | | lineage leukemia 2 | |
| IPI:IPI00421179.1 | SFS*K@EVEER\hat{ } | 863 | Eif4g1 | Isoform 1 of Eukaryotic translation | 1.4913 |
| | | | | initiation factor 4 gamma 1 | |
| IPI:IPI00421179.1 | SFS*KEVEER | 864 | Eif4g1 | Isoform 1 of Eukaryotic translation | 1.4913 |
| | | | | initiation factor 4 gamma 1 | |
| IPI:IPI00421179.1 | SFS*KEVEER | 865 | Eif4g1 | Isoform 1 of Eukaryotic translation | 1.4913 |
| | | | | initiation factor 4 gamma 1 | |
| IPI:IPI00421179.1 | SFS*K@EVEER\hat{ } | 866 | Eif4g1 | Isoform 1 of Eukaryotic translation | 1.4913 |
| | | | | initiation factor 4 gamma 1 | |
| IPI:IPI00136572.4 | YEENPAWPGTSTHNGPNG | 867 | Zc3hav1 | Isoform 1 of Zinc finger CCCH-type | 1.4864 |
| | FSQIMDET*PNVSK@SSPT | | | antiviral protein 1 | |
| | GFGIK@ | | | | |
| IPI:IPI00318938.6 | NS*PVAK@T*PPK@DLPAIP | 868 | Eif4ebp1 | Eukaryotic translation initiation | 1.4819 |
| | GVTSPTSDEPPMQASQSQL | | | factor 4E-binding protein 1 | |
| | PSSPEDK@ | | | | |
| IPI:IPI00318938.6 | NS*PVAK@T*PPK@DLPAIP | 869 | Eif4ebp1 | Eukaryotic translation initiation | 1.4819 |
| | GVTSPTSDEPPMQASQSQL | | | factor 4E-binding protein 1 | |
| | PSSPEDK@ | | | | |
| IPI:IPI00318938.6 | NSPVAKT*PPKDLPAIPGVT* | 870 | Eif4ebp1 | Eukaryotic translation initiation | 1.4819 |
| | SPTSDEPPMQASQSQLPSS | | | factor 4E-binding protein 1 | |
| | PEDK | | | | |
| IPI:IPI00318938.6 | NS*PVAK@T*PPK@DLPAIP | 871 | Eif4ebp1 | Eukaryotic translation initiation | 1.4819 |
| | GVTSPTSDEPPMQASQSQL | | | factor 4E-binding protein 1 | |
| | PSSPEDK@ | | | | |
| IPI:IPI00318938.6 | NS*PVAKT*PPKDLPAIPGVT | 872 | Eif4ebp1 | Eukaryotic translation initiation | 1.4819 |
| | SPTSDEPPMQASQSQLPSS | | | factor 4E-binding protein 1 | |
| | PEDK | | | | |
| IPI:IPI00228948.3 | S*FGDK@DLILPNGGTPAG | 873 | Snx30 | Sorting nexin-30 | 1.4774 |
| | TASPASSSSLLNR\hat{ } | | | | |
| IPI:IPI00330246.2 | WAAHGTS*PEDFSLTLGAR\hat{ } | 874 | Phldb1 | Isoform 2 of Pleckstrin homology-like | 1.474 |
| | | | | domain family B member 1 | |
| IPI:IPI00229739.4 | LVVSS*PTS*PK@GK@ | 875 | Plekhm1 | Pleckstrin homology domain-containing | 1.4728 |
| | | | | family M member 1 | |
| IPI:IPI00229739.4 | LVVSS*PT*SPKGK | 876 | Plekhm1 | Pleckstrin homology domain-containing | 1.4728 |
| | | | | family M member 1 | |
| IPI:IPI00453603.1 | FIGS*PR\hat{ }T*PVS*PVK@FSP | 877 | Rps6kb1 | Isoform Alpha I of Ribosomal protein | 1.4727 |
| | GDFWGR\hat{ } | | | S6 kinase beta-1 | |
| IPI:IPI00136107.1 | THS*TSSSIGSGESPFSR\hat{ } | 878 | Ndrg3 | Protein NDRG3 | 1.4724 |
| IPI:IPI00136107.1 | THS*TSSSIGSGESPFSR\hat{ } | 879 | Ndrg3 | Protein NDRG3 | 1.4724 |
| IPI:IPI00317401.6 | AES*PETSAVESTQST*PQK | 880 | Pds5b | Isoform 1 of Sister chromatid cohesion | 1.4711 |
| | @GR\hat{ } | | | protein PDS5 homolog B | |
| IPI:IPI00317401.6 | AES*PETSAVESTQST*PQK | 881 | Pds5b | Isoform 1 of Sister chromatid cohesion | 1.4711 |
| | @GR\hat{ } | | | protein PDS5 homolog B | |
| IPI:IPI00453603.1 | FIGS*PRT*PVS*PVKFSPGD | 882 | Rps6kb1 | Isoform Alpha I of Ribosomal protein | 1.4689 |
| | FWGR | | | S6 kinase beta-1 | |
| IPI:IPI00453603.1 | FIGS*PR\hat{ }T*PVS*PVK@FSP | 883 | Rps6kb1 | Isoform Alpha I of Ribosomal protein | 1.4689 |
| | GDFWGR\hat{ } | | | S6 kinase beta-1 | |
| IPI:IPI00453603.1 | FIGS*PR\hat{ }T*PVS*PVK@FSP | 884 | Rps6kb1 | Isoform Alpha I of Ribosomal protein | 1.4689 |
| | GDFWGR\hat{ } | | | S6 kinase beta-1 | |
| IPI:IPI00380737.1 | TS*SVSS*LASACTGGIPSSS | 885 | Pom121 | Nuclear envelope pore membrane | 1.4684 |
| | R\hat{ } | | | protein POM 121 | |
| IPI:IPI00380737.1 | TS*SVSS*LASACTGGIPSSS | 886 | Pom121 | Nuclear envelope pore membrane | 1.4684 |
| | R\hat{ } | | | protein POM 121 | |
| IPI:IPI00318938.6 | NS*PVAKT*PPKDLPAIPGVT | 887 | Eif4ebp1 | Eukaryotic translation initiation | 1.4676 |
| | SPTSDEPPMQASQSQLPSS | | | factor 4E-binding protein 1 | |
| | PEDK | | | | |
| IPI:IPI00317401.6 | AES*PETSAVESTQST*PQK | 888 | Pds5b | Isoform 1 of Sister chromatid cohesion | 1.4613 |
| | @GR\hat{ } | | | protein PDS5 homolog B | |
| IPI:IPI00317401.6 | AES*PETSAVESTQST*PQK | 889 | Pds5b | Isoform 1 of Sister chromatid cohesion | 1.4526 |
| | @GR\hat{ } | | | protein PDS5 homolog B | |
| IPI:IPI00317401.6 | AES*PETSAVESTQST*PQK | 890 | Pds5b | Isoform 1 of Sister chromatid cohesion | 1.4526 |
| | @GR\hat{ } | | | protein PDS5 homolog B | |
| IPI:IPI00136107.1 | S*RT*HSTSSSIGSGESPFSR | 891 | Ndrg3 | Protein NDRG3 | 1.451 |
| IPI:IPI00118438.4 | KPPAPPSPVQS*QSPS*T*N | 892 | Srrm1 | Isoform 2 of Serine/arginine repetitive | 1.45 |
| | WS*PAVPAKK | | | matrix protein 1 | |
| IPI:IPI00123410.5 | TIS*AQDTLAYATALLNEK@ | 893 | Usp24 | Isoform 1 of Ubiquitin carboxyl- | 1.4486 |
| | | | | terminal hydrolase 24 | |
| IPI:IPI00411026.1 | SLHPWYGITPTSS*PK@ | 894 | Micall1 | Isoform 1 of MICAL-like protein 1 | 1.4479 |
| IPI:IPI00761759.1 | SSS*GSEHS*T*EGSVSLGD | 895 | Larp4 | Putative uncharacterized protein | 1.4347 |
| | GPLSR\hat{ } | | | | |
| IPI:IPI00313307.3 | SYQNSPS*S*EDGIR\hat{ }PLPEY | 896 | Med1 | Isoform 4 of Mediator of RNA | 1.4214 |
| | STEK@ | | | polymerase II transcription subunit 1 | |
| IPI:IPI00313307.3 | SYQNS*PSS*EDGIR\hat{ }PLPEY | 897 | Med1 | Isoform 4 of Mediator of RNA | 1.4158 |
| | STEK@ | | | polymerase II transcription subunit 1 | |
| IPI:IPI00129264.1 | ATSR\hat{ }PINLGPSS*PNTEIHW | 898 | Sorbs3 | Vinexin | 1.4122 |
| | TPYR\hat{ } | | | | |
| IPI:IPI00330773.3 | ASYSGTS*PSHS*FISGEPD | 899 | Phldb2 | Isoform 1 of Pleckstrin homology-like | 1.403 |
| | R\hat{ } | | | domain family B member 2 | |
| IPI:IPI00116442.1 | TR\hat{ }LAS*ES*ANDDNEDS | 900 | Hdgfrp2 | Isoform 3 of Hepatoma-derived growth | 1.3985 |
| | | | | factor-related protein 2 | |
| IPI:IPI00309059.7 | STS*PIIGS*PPVR\hat{ } | 901 | Patl1 | Protein PAT1 homolog 1 | 1.3955 |
| IPI:IPI00309059.7 | STS*PIIGS*PPVR\hat{ } | 902 | Patl1 | Protein PAT1 homolog 1 | 1.3937 |
| IPI:IPI00309059.7 | STS*PIIGS*PPVR\hat{ } | 903 | Patl1 | Protein PAT1 homolog 1 | 1.3937 |
| IPI:IPI00309059.7 | STS*PIIGS*PPVR | 904 | Patl1 | Protein PAT1 homolog 1 | 1.3937 |
| IPI:IPI00318938.6 | NS*PVAK@TPPK@DLPAIP | 905 | Eif4ebp1 | Eukaryotic translation initiation | 1.3906 |
| | GVTSPTS*DEPPMQASQSQ | | | factor 4E-binding protein 1 | |
| | LPSSPEDK@ | | | | |
| IPI:IPI00318938.6 | NS*PVAKT*PPKDLPAIPGVT | 906 | Eif4ebp1 | Eukaryotic translation initiation | 1.3906 |
| | SPTSDEPPMQASQSQLPSS | | | factor 4E-binding protein 1 | |
| | PEDK | | | | |
| IPI:IPI00108388.1 | R\hat{ }PYTGNPQYTYNNWS*PP | 907 | Usp9x | Probable ubiquitin carboxyl-terminal | 1.3905 |
| | VQSNETSNGYFLER\hat{ } | | | hydrolase FAF-X | |
| IPI:IPI00108388.1 | RPYTGNPQYTYNNWS*PPV | 908 | Usp9x | Probable ubiquitin carboxyl-terminal | 1.3905 |
| | QSNETSNGYFLER | | | hydrolase FAF-X | |
| IPI:IPI00309059.7 | STS*PIIGS*PPVR\hat{ } | 909 | Patl1 | Protein PAT1 homolog 1 | 1.3897 |
| IPI:IPI00309059.7 | STS*PIIGS*PPVR\hat{ } | 910 | Patl1 | Protein PAT1 homolog 1 | 1.3897 |
| IPI:IPI00556837.1 | DTVIIVS*EPS*EDEESHDLP | 911 | Smarcad1 | Isoform 1 of SWI/SNF-related matrix- | 1.3868 |
| | SVTR | | | associated actin-dependent regulator of | |
| | | | | chromatin subfamily A containing | |
| | | | | DEAD/H box 1 | |
| IPI:IPI00318938.6 | NS*PVAKT*PPKDLPAIPGVT | 912 | Eif4ebp1 | Eukaryotic translation initiation | 1.3764 |
| | SPTSDEPPMQASQSQLPSS | | | factor 4E-binding protein 1 | |
| | PEDK | | | | |
| IPI:IPI00467843.2 | GGGASSPAPVVFTVGS*PP | 913 | Ulk1 | Putative uncharacterized protein | 1.3745 |
| | SGAT*PPQSTR\hat{ } | | | | |
| IPI:IPI00467843.2 | GGGASSPAPVVFTVGS*PP | 914 | Ulk1 | Putative uncharacterized protein | 1.3745 |
| | SGATPPQST*R\hat{ } | | | | |
| IPI:IPI00467843.2 | GGGASSPAPVVFTVGS*PP | 915 | Ulk1 | Putative uncharacterized protein | 1.3702 |
| | SGAT*PPQSTR\hat{ } | | | | |
| IPI:IPI00330773.3 | ASYSGTS*PSHSFISGEPDR\hat{ } | 916 | Phldb2 | Isoform 1 of Pleckstrin homology-like | 1.3697 |
| | | | | domain family B member 2 | |
| IPI:IPI00330773.3 | AS*YSGTSPSHSFISGEPDR | 917 | Phldb2 | Isoform 1 of Pleckstrin homology-like | 1.3697 |
| | | | | domain family B member 2 | |
| IPI:IPI00330773.3 | ASYSGTS*PSHSFISGEPDR\hat{ } | 918 | Phldb2 | Isoform 1 of Pleckstrin homology-like | 1.3697 |
| | | | | domain family B member 2 | |
| IPI:IPI00117932.2 | SPPVQPHTPVTIS*LGTAPS | 919 | Sin3a | Isoform 1 of Paired amphipathic helix | 1.3566 |
| | LQNNQPVEFNHAINYVNK@ | | | protein Sin3a | |
| IPI:IPI00309059.7 | STS*PIIGS*PPVR\hat{ } | 920 | Patl1 | Protein PAT1 homolog 1 | 1.3334 |
| IPI:IPI00309059.7 | STS*PIIGS*PPVR\hat{ } | 921 | Patl1 | Protein PAT1 homolog 1 | 1.3334 |
| IPI:IPI00224153.6 | VHHS*VQTFQEDSLPVAHS* | 922 | Ptpn21 | Tyrosine-protein phosphatase non- | 1.3332 |
| | LQEVSEPLTAAR\hat{ } | | | receptor type 21 | |
| IPI:IPI00224153.6 | VHHSVQT*FQEDSLPVAHS* | 923 | Ptpn21 | Tyrosine-protein phosphatase non- | 1.3332 |
| | LQEVSEPLTAAR | | | receptor type 21 | |
| IPI:IPI00136572.4 | FHHNS*LEVLSTVS*PLGSG | 924 | Zc3hav1 | Isoform 1 of Zinc finger CCCH-type | 1.3312 |
| | PPS*PDVTGCK@DPLEDVS | | | antiviral protein 1 | |
| | ADVTQK@ | | | | |
| IPI:IPI00309059.7 | STS*PIIGS*PPVR | 925 | Patl1 | Protein PAT1 homolog 1 | 1.3311 |
| IPI:IPI00309059.7 | STS*PIIGS*PPVR\hat{ } | 926 | Patl1 | Protein PAT1 homolog 1 | 1.3311 |
| IPI:IPI00309059.7 | STS*PIIGS*PPVR\hat{ } | 927 | Patl1 | Protein PAT1 homolog 1 | 1.3311 |
| IPI:IPI00309059.7 | STS*PIIGS*PPVR | 928 | Patl1 | Protein PAT1 homolog 1 | 1.3311 |
| IPI:IPI00309059.7 | R\hat{ }STS*PIIGS*PPVR\hat{ } | 929 | Patl1 | Protein PAT1 homolog 1 | 1.3267 |
| IPI:IPI00229739.4 | LVVSSPT*S*PK@ | 930 | Plekhm1 | Pleckstrin homology domain-containing | 1.3252 |
| | | | | family M member 1 | |
| IPI:IPI00321774.2 | HSSIS*PVR\hat{ }LPLNSSLGAEL | 931 | Crkrs | Isoform 2 of Cell division cycle 2- | 1.3231 |
| | SR\hat{ } | | | related protein kinase 7 | |
| IPI:IPI00129264.1 | ATSR\hat{ }PINLGPS*SPNTEIHW | 932 | Sorbs3 | Vinexin | 1.3226 |
| | TPYR\hat{ } | | | | |
| IPI:IPI00380354.1 | LQES*PKLSQANGTR | 933 | Llgl1 | lethal giant larvae homolog 1 isoform 1 | 1.3225 |
| IPI:IPI00320905.7 | LGEQGPEPGPT*PPQT*PT* | 934 | Arhgap17 | Isoform 1 of Rho GTPase-activating | 1.3166 |
| | PPST*PPLAK | | | protein 17 | |
| IPI:IPI00381495.5 | TVSS*PIPYTPSPS*SSR\hat{ }PIS | 935 | Ccdc6 | coiled-coil domain containing 6 | 1.3165 |
| | *PGLSYASHTVGFT*PPTSL | | | | |
| | TR\hat{ } | | | | |
| IPI:IPI00330773.3 | ASYSGTS*PSHS*FISGEPD | 936 | Phldb2 | Isoform 1 of Pleckstrin homology-like | 1.3149 |
| | R\hat{ } | | | domain family B member 2 | |
| IPI:IPI00660767.2 | VDTAASSSWLAGS*CS*PVS | 937 | Ibtk | Isoform 2 of Inhibitor of Bruton | 1.3143 |
| | PPVVDLR\hat{ } | | | tyrosine kinase | |
| IPI:IPI00309059.7 | RSTS*PIIGS*PPVR | 938 | Patl1 | Protein PAT1 homolog 1 | 1.3126 |
| IPI:IPI00309059.7 | R\hat{ }STS*PIIGS*PPVR\hat{ } | 939 | Patl1 | Protein PAT1 homolog 1 | 1.3126 |
| IPI:IPI00309059.7 | R\hat{ }STS*PIIGS*PPVR\hat{ } | 940 | Patl1 | Protein PAT1 homolog 1 | 1.3126 |
| IPI:IPI00309059.7 | RSTS*PIIGS*PPVR | 941 | Patl1 | Protein PAT1 homolog 1 | 1.3123 |
| IPI:IPI00309059.7 | RSTS*PIIGS*PPVR | 942 | Patl1 | Protein PAT1 homolog 1 | 1.3122 |
| IPI:IPI00309059.7 | R\hat{ }STS*PIIGS*PPVR\hat{ } | 943 | Patl1 | Protein PAT1 homolog 1 | 1.3122 |
| IPI:IPI00309059.7 | R\hat{ }STS*PIIGS*PPVR\hat{ } | 944 | Patl1 | Protein PAT1 homolog 1 | 1.3122 |
| IPI:IPI00112593.2 | S*T*S*S*PK@ | 945 | Spert | Isoform 2 of Spermatid-associated | 1.3085 |
| | | | | protein | |
| IPI:IPI00129264.1 | ATSR\hat{ }PINLGPSS*PNTEIHW | 946 | Sorbs3 | Vinexin | 1.3056 |
| | TPYR\hat{ } | | | | |
| IPI:IPI00129264.1 | ATSRPINLGPSS*PNTEIHW | 947 | Sorbs3 | Vinexin | 1.3056 |
| | TPYR | | | | |
| IPI:IPI00116442.1 | TR\hat{ }LAS*ES*ANDDNEDS | 948 | Hdgfrp2 | Isoform 3 of Hepatoma-derived growth | 1.3036 |
| | | | | factor-related protein 2 | |
| IPI:IPI00313307.3 | SYQNS*PSS*EDGIRPLPEY | 949 | Med1 | Isoform 4 of Mediator of RNA | 1.298 |
| | STEK | | | polymerase II transcription subunit 1 | |
| IPI:IPI00313307.3 | SYQNS*PSS*EDGIR\hat{ }PLPEY | 950 | Med1 | Isoform 4 of Mediator of RNA | 1.298 |
| | STEK@ | | | polymerase II transcription subunit 1 | |
| IPI:IPI00313307.3 | SYQNS*PSS*EDGIR\hat{ }PLPEY | 951 | Med1 | Isoform 4 of Mediator of RNA | 1.298 |
| | STEK@ | | | polymerase II transcription subunit 1 | |
| IPI:IPI00330773.3 | ASYSGTS*PSHSFISGEPDR\hat{ } | 952 | Phldb2 | Isoform 1 of Pleckstrin homology-like | 1.2944 |
| | | | | domain family B member 2 | |
| IPI:IPI00330773.3 | ASYSGTS*PSHSFISGEPDR | 953 | Phldb2 | Isoform 1 of Pleckstrin homology-like | 1.2944 |
| | | | | domain family B member 2 | |
| IPI:IPI00330773.3 | ASYSGTS*PSHSFISGEPDR\hat{ } | 954 | Phldb2 | Isoform 1 of Pleckstrin homology-like | 1.2944 |
| | | | | domain family B member 2 | |
| IPI:IPI00153986.2 | GTS*R\hat{ }PGTPS*AEAASTSS | 955 | Gtf2f1 | General transcription factor IIF | 1.2876 |
| | TLR\hat{ } | | | subunit 1 | |
| IPI:IPI00153986.2 | GTSRPGT*PS*AEAASTSST | 956 | Gtf2f1 | General transcription factor IIF | 1.2876 |
| | LR | | | subunit 1 | |
| IPI:IPI00318938.6 | VALGDGVQLPPGDYSTT*P | 957 | Eif4ebp1 | Eukaryotic translation initiation | 1.2862 |
| | GGTLFS*TTPGGTR\hat{ }IIYDR\hat{ } | | | factor 4E-binding protein 1 | |
| | K@ | | | | |
| IPI:IPI00221581.1 | SR\hat{ }TGS*ESSQTGASATSG | 958 | Eif4b | Eukaryotic translation initiation | 1.2845 |
| | R\hat{ } | | | factor 4B | |
| IPI:IPI00454104.1 | TTEAPCSPGS*QQPPS*PDE | 959 | Scrib | Isoform 1 of Protein LAP4 | 1.2842 |
| | LPANVK@ | | | | |
| IPI:IPI00454104.1 | TTEAPCS*PGSQQPPS*PDE | 960 | Scrib | Isoform 1 of Protein LAP4 | 1.2842 |
| | LPANVK | | | | |
| IPI:IPI00454104.1 | TTEAPCS*PGSQQPPS*PDE | 961 | Scrib | Isoform 1 of Protein LAP4 | 1.2842 |
| | LPANVK | | | | |
| IPI:IPI00320905.7 | LGEQGPEPGPTPPQT*PT*P | 962 | Arhgap17 | Isoform 1 of Rho GTPase-activating | 1.2828 |
| | PST*PPLAK@ | | | protein 17 | |
| IPI:IPI00381495.5 | TVSS*PIPYTPS*PSSSR\hat{ }PIS | 963 | Ccdc6 | coiled-coil domain containing 6 | 1.2808 |
| | *PGLSYASHTVGFTPPTS*L | | | | |
| | TR\hat{ } | | | | |
| IPI:IPI00381495.5 | TVSS*PIPYTPS*PSS*SR | 964 | Ccdc6 | coiled-coil domain containing 6 | 1.2808 |
| | PISPGLSYASHTVGFT* | | | | |
| | PPTSLTR | | | | |
| IPI:IPI00381495.5 | TVSS*PIPYTPS*PSSSRPIS | 965 | Ccdc6 | coiled-coil domain containing 6 | 1.2808 |
| | PGLS*YASHTVGFT*PPTSL | | | | |
| | TR | | | | |
| IPI:IPI00309059.7 | RSTS*PIIGS*PPVR | 966 | Patl1 | Protein PAT1 homolog 1 | 1.2745 |
| IPI:IPI00336973.2 | GLNLDGT*PALSTLGGFS*P | 967 | Ccnl1 | Isoform 1 of Cyclin-L1 | 1.273 |
| | ASK@PS*SPR\hat{ } | | | | |
| IPI:IPI00336973.2 | GLNLDGT*PALSTLGGFS*P | 968 | Ccnl1 | Isoform 1 of Cyclin-L1 | 1.273 |
| | ASKPSS*PR | | | | |
| IPI:IPI00309059.7 | R\hat{ }STS*PIIGS*PPVR\hat{ } | 969 | Patl1 | Protein PAT1 homolog 1 | 1.2725 |
| IPI:IPI00309059.7 | R\hat{ }STS*PIIGS*PPVR\hat{ } | 970 | Patl1 | Protein PAT1 homolog 1 | 1.2725 |
| IPI:IPI00309059.7 | RSTS*PIIGS*PPVR | 971 | Patl1 | Protein PAT1 homolog 1 | 1.2725 |
| IPI:IPI00309059.7 | R\hat{ }STS*PIIGS*PPVR\hat{ } | 972 | Patl1 | Protein PAT1 homolog 1 | 1.2723 |
| IPI:IPI00309059.7 | RSTS*PIIGS*PPVR | 973 | Patl1 | Protein PAT1 homolog 1 | 1.2723 |
| IPI:IPI00309059.7 | R\hat{ }STS*PIIGS*PPVR\hat{ } | 974 | Patl1 | Protein PAT1 homolog 1 | 1.2723 |
| IPI:IPI00309059.7 | RSTS*PIIGS*PPVR | 975 | Patl1 | Protein PAT1 homolog 1 | 1.2723 |
| IPI:IPI00129264.1 | ATSR\hat{ }PINLGPS*SPNTEIHW | 976 | Sorbs3 | Vinexin | 1.2719 |
| | TPYR\hat{ } | | | | |
| IPI:IPI00318938.6 | NS*PVAK@T*PPK@DLPAIP | 977 | Eif4ebp1 | Eukaryotic translation initiation | 1.2719 |
| | GVTSPTSDEPPM#QASQSQ | | | factor 4E-binding protein 1 | |
| | LPSSPEDK@ | | | | |
| IPI:IPI00115492.1 | HSLSSESQAPEDIAPPGS*S | 978 | Eps8l2 | Isoform 1 of Epidermal growth factor | 1.2678 |
| | PHANR\hat{ } | | | receptor kinase substrate 8-like | |
| | | | | protein 2 | |
| IPI:IPI00381495.5 | TVSS*PIPYTPS*PSSSR\hat{ }PIS | 979 | Ccdc6 | coiled-coil domain containing 6 | 1.2638 |
| | *PGLSYASHTVGFT*PPTSL | | | | |
| | TR\hat{ } | | | | |
| IPI:IPI00381495.5 | TVSS*PIPYTPS*PSSSRP | 980 | Ccdc6 | coiled-coil domain containing 6 | 1.2638 |
| | IS*PGLSYASHTVGFT* | | | | |
| | PPTSLTR | | | | |
| IPI:IPI00381495.5 | TVSS*PIPYTPS*PSSSRP | 981 | Ccdc6 | coiled-coil domain containing 6 | 1.2638 |
| | IS*PGLSYASHTVGFT* | | | | |
| | PPTSLTR | | | | |
| IPI:IPI00381495.5 | TVSS*PIPYTPSPSS*SR\hat{ }PIS | 982 | Ccdc6 | coiled-coil domain containing 6 | 1.2638 |
| | PGLSYAS*HTVGFTPPTS*L | | | | |
| | TR\hat{ } | | | | |
| IPI:IPI00133917.1 | YAQGFLPEK@PPQQDHTT* | 983 | Phc2 | Isoform 1 of Polyhomeotic-like | 1.2624 |
| | TTDSEMEEPYLQESK@EE | | | protein 2 | |
| | GTPLK@ | | | | |
| IPI:IPI00227209.3 | R\hat{ }LS*S*TSLASGHSVR\hat{ } | 984 | Prkd2 | Serine/threonine-protein kinase D2 | 1.2605 |
| IPI:IPI00318938.6 | VALGDGVQLPPGDYSTT*P | 985 | Eif4ebp1 | Eukaryotic translation initiation | 1.2599 |
| | GGTLFST*TPGGTR\hat{ } | | | factor 4E-binding protein 1 | |
| IPI:IPI00230368.1 | SSS*LGNS*PDR\hat{ }GPLR\hat{ }PF | 986 | 2610110G12Rik | Isoform 1 of UPF0635 protein C6orf134 | 1.2571 |
| | VPEQELLR\hat{ } | | | homolog | |
| IPI:IPI00845596.1 | LFS*QGQDVSDK@VK@ | 987 | Mllt4 | Isoform 3 of Afadin | 1.2551 |
| IPI:IPI00378026.4 | VSSPSCDS*QGLHPEEAPS* | 988 | Nacc1 | Nucleus accumbens-associated protein 1 | 1.253 |
| | SEPQSPVAQTLGWPACSTP | | | | |
| | LPLVSR\hat{ } | | | | |
| IPI:IPI00458460.1 | SLS*ESS*VVMDR\hat{ } | 989 | Zfp106 | Isoform 1 of Zinc finger protein 106 | 1.2522 |
| IPI:IPI00399440.2 | HLEDAGST*PS*IGENDLK@ | 990 | Rictor | Isoform 1 of Rapamycin-insensitive | 1.2463 |
| | FPK@ | | | companion of mTOR | |
| IPI:IPI00109318.1 | TVAISDAAQLPQDYCTT*PG | 991 | Eif4ebp2 | Eukaryotic translation initiation | 1.2423 |
| | GTLFSTT*PGGTR\hat{ }IIYDR\hat{ }K@ | | | factor 4E-binding protein 2 | |
| IPI:IPI00109318.1 | TVAISDAAQLPQDYCTT*PG | 992 | Eif4ebp2 | Eukaryotic translation initiation | 1.2423 |
| | GTLFST*TPGGTRIIYDRK | | | factor 4E-binding protein 2 | |
| IPI:IPI00379682.1 | R\hat{ }ANTLS*HFPVECPAPPEP | 993 | Tbc1d1 | Isoform 1 of TBC1 domain family | 1.2355 |
| | AQS*SPGVSQR\hat{ } | | | member 1 | |
| IPI:IPI00454104.1 | TTEAPCS*PGSQQPPS*PDE | 994 | Scrib | Isoform 1 of Protein LAP4 | 1.2352 |
| | LPANVK | | | | |
| IPI:IPI00454104.1 | TTEAPCS*PGSQQPPS*PDE | 995 | Scrib | Isoform 1 of Protein LAP4 | 1.2352 |
| | LPANVK@ | | | | |
| IPI:IPI00454104.1 | TTEAPCS*PGSQQPPS*PDE | 996 | Scrib | Isoform 1 of Protein LAP4 | 1.2352 |
| | LPANVK@ | | | | |
| IPI:IPI00109318.1 | TVAISDAAQLPQDYCT*TPG | 997 | Eif4ebp2 | Eukaryotic translation initiation | 1.2336 |
| | GTLFSTT*PGGTR\hat{ }IIYDR\hat{ }K@ | | | factor 4E-binding protein 2 | |
| IPI:IPI00378438.6 | S*QSVPGAWPGASPLSSQP | 998 | Tns1 | tensin 1 | 1.2303 |
| | LLGSS*R\hat{ }QSHPLTQSR\hat{ } | | | | |
| IPI:IPI00118143.1 | KQT*PPAS*PS*PQPIEDRPP | 999 | Cttn | Src substrate cortactin | 1.2254 |
| | SS*PIYEDAAPFKAEPSYR | | | | |
| IPI:IPI00330773.3 | ASYSGTSPS*HSFISGEPDR\hat{ } | 1000 | Phldb2 | Isoform 1 of Pleckstrin homology-like | 1.2251 |
| | | | | domain family B member 2 | |
| IPI:IPI00330773.3 | ASYSGTS*PSHSFISGEPDR\hat{ } | 1001 | Phldb2 | Isoform 1 of Pleckstrin homology-like | 1.2251 |
| | | | | domain family B member 2 | |
| IPI:IPI00127071.3 | GATEEEQQDS*GS*EPR\hat{ }G | 1002 | Ddx41 | Probable ATP-dependent RNA helicase | 1.223 |
| | DEDDIPLGPQSNVSLLDQH | | | DDX41 | |
| | QHLK@ | | | | |
| IPI:IPI00153986.2 | GTSR\hat{ }PGT*PS*AEAASTSS | 1003 | Gtf2f1 | General transcription factor IIF | 1.2226 |
| | TLR\hat{ } | | | subunit 1 | |
| IPI:IPI00153986.2 | GTS*R\hat{ }PGT*PSAEAASTSS | 1004 | Gtf2f1 | General transcription factor IIF | 1.2226 |
| | TLR\hat{ } | | | subunit 1 | |
| IPI:IPI00153986.2 | GTS*RPGT*PSAEAASTSST | 1005 | Gtf2f1 | General transcription factor IIF | 1.2226 |
| | LR | | | subunit 1 | |
| IPI:IPI00117277.2 | R\hat{ }GS*GDTSISMDTEASIR\hat{ } | 1006 | Lrrfip1 | Isoform 2 of Leucine-rich repeat | 1.2213 |
| | | | | flightless-interacting protein 1 | |
| IPI:IPI00661508.3 | LYSLLGIDLTAPSNNS*SPR\hat{ } | 1007 | Sbno1 | Isoform 2 of Protein strawberry notch | 1.2147 |
| | DS*PCK@ENK@ | | | homolog 1 | |
| IPI:IPI00318938.6 | NS*PVAKT*PPKDLPAIPGVT | 1008 | Eif4ebp1 | Eukaryotic translation initiation | 1.2095 |
| | *SPTSDEPPMQASQSQLPS | | | factor 4E-binding protein 1 | |
| | S*PEDK | | | | |
| IPI:IPI00116442.1 | TRLAS*ES*ANDDNEDS | 1009 | Hdgfrp2 | Isoform 3 of Hepatoma derived growth | 1.1963 |
| | | | | factor-related protein 2 | |
| IPI:IPI00116442.1 | TRLAS*ES*ANDDNEDS | 1010 | Hdgfrp2 | Isoform 3 of Hepatoma-derived growth | 1.1963 |
| | | | | factor-related protein 2 | |
| IPI:IPI00929761.1 | R\hat{ }CPS*QSSSR\hat{ }PATGISQP | 1011 | Larp1 | Isoform 2 of La-related protein 1 | 1.1959 |
| | PTTPTGQATR\hat{ } | | | | |
| IPI:IPI00320905.7 | LGEQGPEPGPT*PPQT*PT* | 1012 | Arhgap17 | Isoform 1 of Rho GTPase-activating | 1.1918 |
| | PPS*TPPLAK@ | | | protein 17 | |
| IPI:IPI00420509.2 | S*IEDLQPPNALSAPFTNS | 1013 | Fmnl3 | Isoform 1 of Formin-like protein 3 | 1.1892 |
| | LAR | | | | |
| IPI:IPI00153986.2 | GTS*RPGT*PSAEAASTSST | 1014 | Gtf2f1 | General transcription factor IIF | 1.1888 |
| | | | | subunit 1 | |
| IPI:IPI00153986.2 | GT*SR\hat{ }PGT*PSAEAASTSS | 1015 | Gtf2f1 | General transcription factor IIF | 1.1888 |
| | TLR\hat{ } | | | subunit 1 | |
| IPI:IPI00153986.2 | GTS*RPGT*PSAEAASTSST | 1016 | Gtf2f1 | General transcription factor IIF | 1.1888 |
| | LR | | | subunit 1 | |
| IPI:IPI00153986.2 | GTS*R\hat{ }PGTPS*AEAASTSS | 1017 | Gtf2f1 | General transcription factor IIF | 1.1888 |
| | TLR\hat{ } | | | subunit 1 | |
| IPI:IPI00320905.7 | LGEQGPEPGPT*PPQT*PT* | 1018 | Arhgap17 | Isoform 1 of Rho GTPase-activating | 1.1882 |
| | PPST*PPLAK | | | protein 17 | |
| IPI:IPI00330773.3 | ASYSGTS*PSHSFISGEPDR | 1019 | Phldb2 | Isoform 1 of Pleckstrin homology-like | 1.1879 |
| | | | | domain family B member 2 | |
| IPI:IPI00330773.3 | ASYSGTSPS*HSFISGEPDR\hat{ } | 1020 | Phldb2 | Isoform 1 of Pleckstrin homology-like | 1.1879 |
| | | | | domain family B member 2 | |
| IPI:IPI00330773.3 | ASYSGTS*PSHSFISGEPDR\hat{ } | 1021 | Phldb2 | Isoform 1 of Pleckstrin homology-like | 1.1879 |
| | | | | domain family B member 2 | |
| IPI:IPI00330773.3 | SDELLGDLTRT*PPSSSAAF | 1022 | Phldb2 | Isoform 1 of Pleckstrin homology-like | 1.185 |
| | LK | | | domain family B member 2 | |
| IPI:IPI00330773.3 | SDELLGDLTR\hat{ }T*PPSSSAA | 1023 | Phldb2 | Isoform 1 of Pleckstrin homology-like | 1.185 |
| | FLK@ | | | domain family B member 2 | |
| IPI:IPI00340860.5 | TAS*EGS*EAETPEAPK@Q | 1024 | Larp7 | Isoform 1 of La-related protein 7 | 1.1845 |
| | PAK@K@ | | | | |
| IPI:IPI00340860.5 | TAS*EGS*EAETPEAPK@Q | 1025 | Larp7 | Isoform 1 of La-related protein 7 | 1.1845 |
| | PAK@K@ | | | | |
| IPI:IPI00129264.1 | ATSRPINLGPSS*PNTEIHW | 1026 | Sorbs3 | Vinexin | 1.175 |
| | TPYR | | | | |
| IPI:IPI00129264.1 | ATSR\hat{ }PINLGPSS*PNTEIHW | 1027 | Sorbs3 | Vinexin | 1.175 |
| | TPYR\hat{ } | | | | |
| IPI:IPI00129264.1 | ATSRPINLGPS*SPNTEIHW | 1028 | Sorbs3 | Vinexin | 1.175 |
| | TPYR | | | | |
| IPI:IPI00381495.5 | T*VSSPIPYTPS*PSSS*R\hat{ }PI | 1029 | Ccdc6 | coiled-coil domain containing 6 | 1.1749 |
| | SPGLSYASHTVGFT*PPTSL | | | | |
| | TR\hat{ } | | | | |
| IPI:IPI00153986.2 | GTS*R\hat{ }PGT*PSAEAASTSS | 1030 | Gtf2f1 | General transcription factor IIF | 1.1748 |
| | TLR\hat{ } | | | subunit 1 | |
| IPI:IPI00153986.2 | GTS*RPGT*PSAEAASTSST | 1031 | Gtf2f1 | General transcription factor IIF | 1.1748 |
| | LR | | | subunit 1 | |
| IPI:IPI00153986.2 | GTS*R\hat{ }PGT*PSAEAASTSS | 1032 | Gtf2f1 | General transcription factor IIF | 1.1748 |
| | TLR\hat{ } | | | subunit 1 | |
| IPI:IPI00153986.2 | GTS*RPGT*PSAEAASTSST | 1033 | Gtf2f1 | General transcription factor IIF | 1.1748 |
| | LR | | | subunit 1 | |
| IPI:IPI00136572.4 | YEENPAWPGTSTHNGPNG | 1034 | Zc3hav1 | Isoform 1 of Zinc finger CCCH-type | 1.1738 |
| | FSQIMDETPNVSK@SS*PT | | | antiviral protein 1 | |
| | GFGIK@ | | | | |
| IPI:IPI00225062.2 | GSLS*R\hat{ }SS*S*PVTELTAR\hat{ } | 1035 | Srrm2 | Isoform 3 of Serine/arginine repetitive | 1.1701 |
| | | | | matrix protein 2 | |
| IPI:IPI00133685.1 | S*LPVSVPVWAFK@ | 1036 | Akt1s1 | Proline-rich AKT1 substrate 1 | 1.1669 |
| IPI:IPI00133685.1 | S*LPVSVPVWAFK@ | 1037 | Akt1s1 | Proline-rich AKT1 substrate 1 | 1.1669 |
| IPI:IPI00133685.1 | S*LPVSVPVWAFK@ | 1038 | Akt1s1 | Proline-rich AKT1 substrate 1 | 1.1669 |
| IPI:IPI00225062.2 | GSLS*RSS*S*PVTELTAR | 1039 | Srrm2 | Isoform 3 of Serine/arginine repetitive | 1.1669 |
| | | | | matrix protein 2 | |
| IPI:IPI00225062.2 | GSLS*R\hat{ }SS*S*PVTELTAR\hat{ } | 1040 | Srrm2 | Isoform 3 of Serine/arginine repetitive | 1.1669 |
| | | | | matrix protein 2 | |
| IPI:IPI00929786.1 | S*LPTTVPESPNYR\hat{ } | 1041 | Larp1 | Isoform 1 of La-related protein 1 | 1.1655 |
| IPI:IPI00929786.1 | S*LPTTVPESPNYR | 1042 | Larp1 | Isoform 1 of La-related protein 1 | 1.1655 |
| IPI:IPI00116442.1 | TRLAS*ES*ANDDNEDS | 1043 | Hdgfrp2 | Isoform 3 of Hepatoma-derived growth | 1.1626 |
| | | | | factor-related protein 2 | |
| IPI:IPI00116442.1 | TR\hat{ }LAS*ES*ANDDNEDS | 1044 | Hdgfrp2 | Isoform 3 of Hepatoma-derived growth | 1.1626 |
| | | | | factor-related protein 2 | |
| IPI:IPI00116442.1 | TR\hat{ }LAS*ES*ANDDNEDS | 1045 | Hdgfrp2 | Isoform 3 of Hepatoma-derived growth | 1.1626 |
| | | | | factor-related protein 2 | |
| IPI:IPI00116442.1 | TRLAS*ES*ANDDNEDS | 1046 | Hdgfrp2 | Isoform 3 of Hepatoma-derived growth | 1.1626 |
| | | | | factor-related protein 2 | |
| IPI:IPI00317599.3 | SQEDEEEIST*SPGVSEFVS | 1047 | Syap1 | Synapse-associated protein 1 | 1.1567 |
| | DAFDTCSLNQEDLRK | | | | |
| IPI:IPI00317599.3 | SQEDEEEISTS*PGVSEFVS | 1048 | Syap1 | Synapse-associated protein 1 | 1.1567 |
| | DAFDTCSLNQEDLR\hat{ }K@ | | | | |
| IPI:IPI00317599.3 | SQEDEEEISTS*PGVSEFVS | 1049 | Syap1 | Synapse-associated protein 1 | 1.1567 |
| | DAFDTCSLNQEDLR\hat{ }K@ | | | | |
| IPI:IPI00317599.3 | SQEDEEEISTS*PGVSEFVS | 1050 | Syap1 | Synapse-associated protein 1 | 1.1567 |
| | DAFDTCSLNQEDLRK | | | | |
| IPI:IPI00126124.2 | T*FSECS*YPETEEEAEALP | 1051 | St5 | Isoform 1 of Suppression of | 1.1565 |
| | GR\hat{ } | | | tumorigenicity 5 | |
| IPI:IPI00312600.2 | LSSDENSNPDLS*GDENDD | 1052 | Eed | Isoform 1 of Polycomb protein EED | 1.1553 |
| | AVSIESGTNTER\hat{ }PDTPTNT | | | | |
| | PNAPGR\hat{ }K@ | | | | |
| IPI:IPI00312600.2 | LSSDENSNPDLS*GDENDD | 1053 | Eed | Isoform 1 of Polycomb protein EED | 1.1553 |
| | AVSIESGTNTERPDTPTNTP | | | | |
| | NAPGRK | | | | |
| IPI:IPI00229990.7 | RPPGDVLETFNFLENADDS* | 1054 | Strn3 | Striatin-3 | 1.1544 |
| | DEEENDMIEGIPEGKDK | | | | |
| IPI:IPI00229990.7 | R\hat{ }PPGDVLETFNFLENADDS | 1055 | Strn3 | Striatin-3 | 1.1544 |
| | *DEEENDMIEGIPEGK@DK@ | | | | |
| IPI:IPI00121418.1 | DGEGPDNLEPACPLSLPLQ | 1056 | Rb1 | Retinoblastoma-associated protein | 1.152 |
| | GNHTAADMYLS*PLRS*PK | | | | |
| IPI:IPI00121418.1 | DGEGPDNLEPACPLSLPLQ | 1057 | Rb1 | Retinoblastoma-associated protein | 1.152 |
| | GNHTAADMYLS*PLR\hat{ }S*PK@ | | | | |
| IPI:IPI00121418.1 | DGEGPDNLEPACPLSLPLQ | 1058 | Rb1 | Retinoblastoma-associated protein | 1.152 |
| | GNHTAADMYLS*PLR\hat{ }S*PK@ | | | | |
| IPI:IPI00136386.2 | GDT*S*ET*ASAT*PAY*R\hat{ } | 1059 | Tssk4 | Isoform 2 of Testis-specific | 1.15 |
| | | | | serine/threonine-protein kinase 4 | |
| IPI:IPI00515576.5 | TGVNENTVVSAGKDLSTS*P | 1060 | Ehbp1 | Isoform 2 of EH domain-binding | 1.1492 |
| | KPS*PIPS*PVLGQKPNASQ | | | protein 1 | |
| | SLLAWCR | | | | |
| IPI:IPI00420143.1 | STSSVDSDILSSSHS*S*DTL | 1061 | Usp47 | Isoform 2 of Ubiquitin carboxyl- | 1.1474 |
| | CNADSAQIPLANGLDSHSIT | | | terminal hydrolase 47 | |
| | SSR\hat{ } | | | | |
| IPI:IPI00109318.1 | TVAISDAAQLPQDYCTT*PG | 1062 | Eif4ebp2 | Eukaryotic translation initiation | 1.147 |
| | GTLFSTT*PGGTRIIYDRK | | | factor 4E-binding protein 2 | |
| IPI:IPI00109318.1 | TVAISDAAQLPQDYCT*TPG | 1063 | Eif4ebp2 | Eukaryotic translation initiation | 1.147 |
| | GTLFSTT*PGGTR\hat{ }IIYDR\hat{ }K@ | | | factor 4E-binding protein 2 | |
| IPI:IPI00330773.3 | SDELLGDLTR\hat{ }T*PPSSSAA | 1064 | Phldb2 | Isoform 1 of Pleckstrin homology-like | 1.1466 |
| | FLK@ | | | domain family B member 2 | |
| IPI:IPI00330773.3 | SDELLGDLTRT*PPSSSAAF | 1065 | Phldb2 | Isoform 1 of Pleckstrin homology-like | 1.1466 |
| | LK | | | domain family B member 2 | |
| IPI:IPI00229697.3 | GT*FSDQELDAQS*LDDEDD | 1066 | — | 68 kDa protein | 1.1465 |
| | SLQHAVHPALNRFS*PS*PR | | | | |
| IPI:IPI00133685.1 | S*LPVSVPVWAFK@ | 1067 | Akt1s1 | Proline-rich AKT1 substrate 1 | 1.1417 |
| IPI:IPI00133685.1 | S*LPVSVPVWAFK | 1068 | Akt1s1 | Proline-rich AKT1 substrate 1 | 1.1417 |
| IPI:IPI00133685.1 | S*LPVSVPVWAFK@ | 1069 | Akt1s1 | Proline-rich AKT1 substrate 1 | 1.1417 |
| IPI:IPI00133685.1 | S*LPVSVPVWAFK | 1070 | Akt1s1 | Proline-rich AKT1 substrate 1 | 1.1417 |
| IPI:IPI00130920.1 | SLMSS*PEDLTK@DFEELK | 1071 | Mtap1b | Microtubule-associated protein 1B | 1.1401 |
| | @AEEIDVAK@ | | | | |
| IPI:IPI00130920.1 | SLMSSPEDLT*KDFEELKAE | 1072 | Mtap1b | Microtubule-associated protein 1B | 1.1401 |
| | EIDVAK | | | | |
| IPI:IPI00828355.1 | GTLVHTT*SDSDS*EDGDQE | 1073 | Fmn1 | Isoform 5 of Formin-1 | 1.1401 |
| | AEEESSLDTQK@PTTVVLC | | | | |
| | EPSQEPK@ | | | | |
| IPI:IPI00381495.5 | T*VSSPIPYTPSPS*SSR\hat{ }PIS | 1074 | Ccdc6 | coiled-coil domain containing 6 | 1.1376 |
| | PGLSYASHT*VGFT*PPTSL | | | | |
| | TR\hat{ } | | | | |
| IPI:IPI00929761.1 | CPS*QSSSRPATGISQPPTT | 1075 | Larp1 | Isoform 2 of La-related protein 1 | 1.1372 |
| | PTGQATR | | | | |
| IPI:IPI00379682.1 | R\hat{ }ANTLS*HFPVECPAPPEP | 1076 | Tbc1d1 | Isoform 1 of TBC1 domain family | 1.1351 |
| | AQSS*PGVSQR\hat{ } | | | member 1 | |
| IPI:IPI00379682.1 | RANT*LSHFPVECPAPPEPA | 1077 | Tbc1d1 | Isoform 1 of TBC1 domain family | 1.1351 |
| | QSS*PGVSQR | | | member 1 | |
| IPI:IPI00381495.5 | T*VSSPIPYTPSPSS*SR\hat{ }PIS | 1078 | Ccdc6 | coiled-coil domain containing 6 | 1.1342 |
| | PGLS*YASHTVGFT*PPTSL | | | | |
| | TR\hat{ } | | | | |
| IPI:IPI00381495.5 | T*VSSPIPYTPS*PSSSRPIS | 1079 | Ccdc6 | coiled-coil domain containing 6 | 1.1342 |
| | PGLSYASHT*VGFT*PPTSL | | | | |
| | TR | | | | |
| IPI:IPI00225062.2 | M#SCFS*R\hat{ }PSMS*PTPLDR\hat{ } | 1080 | Srrm2 | Isoform 3 of Serine/arginine repetitive | 1.1322 |
| | | | | matrix protein 2 | |
| IPI:IPI00133374.5 | EVDPSTGELQS*LQMPES*E | 1081 | Sqstm1 | Isoform 1 of Sequestosome-1 | 1.1315 |
| | GPS*SLDPSQEGPTGLK@ | | | | |
| IPI:IPI00133374.5 | EVDPSTGELQS*LQMPES*E | 1082 | Sqstm1 | Isoform 1 of Sequestosome-1 | 1.1315 |
| | GPS*SLDPSQEGPTGLK@ | | | | |
| IPI:IPI00133374.5 | EVDPSTGELQS*LQMPES*E | 1083 | Sqstm1 | Isoform 1 of Sequestosome-1 | 1.1315 |
| | GPS*SLDPSQEGPTGLK@ | | | | |
| IPI:IPI00133374.5 | EVDPSTGELQS*LQMPES*E | 1084 | Sqstm1 | Isoform 1 of Sequestosome-1 | 1.1315 |
| | GPS*SLDPSQEGPTGLK | | | | |
| IPI:IPI00465879.2 | LCS*SSS*SDTSPR\hat{ } | 1085 | Zc3hc1 | Isoform 1 of Nuclear-interacting | 1.1277 |
| | | | | partner of ALK | |
| IPI:IPI00468802.5 | YEMACAIADAFNLPSSHLR\hat{ } | 1086 | Mat2b | Isoform 1 of Methionine | 1.1262 |
| | PITDS*PVIGAQR\hat{ }PK@ | | | adenosyltransferase 2 subunit beta | |
| IPI:IPI00121251.7 | LSTTPS*PTNS*LHEDGVDD | 1087 | Tox4 | TOX high mobility group box family | 1.1209 |
| | FR\hat{ }R\hat{ } | | | member 4 | |
| IPI:IPI00121251.7 | LSTTPS*PTNS*LHEDGVDD | 1088 | Tox4 | TOX high mobility group box family | 1.1209 |
| | FR\hat{ }R\hat{ } | | | member 4 | |
| IPI:IPI00121251.7 | LSTTPS*PTNS*LHEDGVDD | 1089 | Tox4 | TOX high mobility group box family | 1.1209 |
| | FRR | | | member 4 | |
| IPI:IPI00127554.1 | R\hat{ }FS*VQEQDWETT*PPK@ | 1090 | Emg1 | Probable ribosome biogenesis protein | 1.1179 |
| | K@ | | | NEP1 | |
| IPI:IPI00127554.1 | RFS*VQEQDWET*TPPKK | 1091 | Emg1 | Probable ribosome biogenesis protein | 1.1179 |
| | | | | NEP1 | |
| IPI:IPI00459443.5 | GEGVSQVGPGTPPAPES*P | 1092 | Tnks1bp1 | 182 kDa tankyrase-1-binding protein | 1.1175 |
| | RKPISGVQGNDPGISLPQR | | | | |
| IPI:IPI00225062.2 | MELGT*PLR\hat{ }HSGSTS*PYP | 1093 | Srrm2 | Isoform 3 of Serine/arginine repetitive | 1.1138 |
| | K@ | | | matrix protein 2 | |
| IPI:IPI00225062.2 | MELGT*PLRHSGST*SPYPK | 1094 | Srrm2 | Isoform 3 of Serine/arginine repetitive | 1.1138 |
| | | | | matrix protein 2 | |
| IPI:IPI00225062.2 | MELGT*PLR\hat{ }HSGSTS*PYP | 1095 | Srrm2 | Isoform 3 of Serine/arginine repetitive | 1.1138 |
| | K@ | | | matrix protein 2 | |
| IPI:IPI00127764.2 | EIS*QSR\hat{ }NPS*VSEHLPDEK@ | 1096 | Pcm1 | Isoform 1 of Pericentriolar material 1 | 1.1126 |
| | | | | protein | |
| IPI:IPI00318938.6 | NS*PVAK@T*PPK@DLPAIP | 1097 | Eif4ebp1 | Eukaryotic translation initiation | 1.1074 |
| | GVTSPTSDEPPMQASQSQL | | | factor 4E-binding protein 1 | |
| | PSS*PEDK@ | | | | |
| IPI:IPI00116923.2 | SGFAR\hat{ }PGDLEFEDFS*QVI | 1098 | Trip10 | Isoform 3 of Cdc42-interacting | 1.1069 |
| | NR\hat{ }VPSDSSLGT*PDGR\hat{ }PE | | | protein 4 | |
| | LR\hat{ } | | | | |
| IPI:IPI00121335.1 | CGS*PSDSSTSEMMEVAVN | 1099 | LOC100048123; | RAC-beta serine/threonine-protein | 1.1028 |
| | K@ | | Akt2 | kinase | |
| IPI:IPI00356608.4 | VGS*LT*PPS*SPK@ | 1100 | Aak1 | Isoform 2 of AP2-associated protein | 1.1012 |
| | | | | kinase 1 | |
| IPI:IPI00828461.1 | GPPDFS*SDEER\hat{ }EPT*PVL | 1101 | Tmpo | thymopoietin isoform delta | 1.1001 |
| | GSGASVGR\hat{ } | | | | |
| IPI:IPI00381495.5 | TVSSPIPYTPS*PS*SSR\hat{ }PIS | 1102 | Ccdc6 | coiled-coil domain containing 6 | 1.0993 |
| | PGLSYASHTVGFTPPTSLTR\hat{ } | | | | |
| IPI:IPI00381495.5 | TVSSPIPYTPS*PSSSRPISP | 1103 | Ccdc6 | coiled-coil domain containing 6 | 1.0993 |
| | GLSY*ASHTVGFTPPTSLTR | | | | |
| IPI:IPI00127554.1 | R\hat{ }FS*VQEQDWETTPPK@K@ | 1104 | Emg1 | Probable ribosome biogenesis protein | 1.0981 |
| | | | | NEP1 | |
| IPI:IPI00127554.1 | R\hat{ }FS*VQEQDWETTPPK@K@ | 1105 | Emg1 | Probable ribosome biogenesis protein | 1.0981 |
| | | | | NEP1 | |
| IPI:IPI00121251.7 | LSTT*PSPTNS*LHEDGVDD | 1106 | Tox4 | TOX high mobility group box family | 1.0844 |
| | FRR | | | member 4 | |
| IPI:IPI00121251.7 | LSTTPS*PTNS*LHEDGVDD | 1107 | Tox4 | TOX high mobility group box family | 1.0844 |
| | FR\hat{ }R\hat{ } | | | member 4 | |
| IPI:IPI00121251.7 | LSTTPS*PTNS*LHEDGVDD | 1108 | Tox4 | TOX high mobility group box family | 1.0844 |
| | FR\hat{ }R\hat{ } | | | member 4 | |
| IPI:IPI00318938.6 | NS*PVAK@T*PPK@DLPAIP | 1109 | Eif4ebp1 | Eukaryotic translation initiation | 1.0842 |
| | GVTSPTSDEPPMQASQSQL | | | factor 4E-binding protein 1 | |
| | PSS*PEDK@ | | | | |
| IPI:IPI00318938.6 | NS*PVAKT*PPKDLPAIPGVT | 1110 | Eif4ebp1 | Eukaryotic translation initiation | 1.0842 |
| | S*PTSDEPPMQASQSQLPS | | | factor 4E-binding protein 1 | |
| | SPEDK | | | | |
| IPI:IPI00169771.1 | S*SASGTHSES*PEK@LQC | 1111 | BC031781 | UPF0667 protein C1orf55 homolog | 1.0823 |
| | PVTEPGQGILENTGTEPGE | | | | |
| | TSDK@ECNER\hat{ } | | | | |
| IPI:IPI00318938.6 | NS*PVAK@T*PPK@DLPAIP | 1112 | Eif4ebp1 | Eukaryotic translation initiation | 1.0778 |
| | GVTSPTSDEPPMQASQSQL | | | factor 4E-binding protein 1 | |
| | PSS*PEDK@ | | | | |
| IPI:IPI00127764.2 | EISQS*RNPS*VSEHLPDEK | 1113 | Pcm1 | Isoform 1 of Pericentriolar material 1 | 1.074 |
| | | | | protein | |
| IPI:IPI00112597.1 | SQSS*EGVSSLSSS*PSNSL | 1114 | | | 1.0712 |
| | ETQSQSLSR\hat{ } | | | | |
| IPI:IPI00112597.1 | SQS*SEGVSSLSSS*PSNSL | 1115 | | | 1.0712 |
| | ETQSQSLSR\hat{ } | | | | |
| IPI:IPI00225062.2 | S*RSSSPDS*KMELGTPLR | 1116 | Srrm2 | Isoform 3 of Serine/arginine repetitive | 1.0696 |
| | | | | matrix protein 2 | |
| IPI:IPI00330773.3 | ASYSGTS*PSHS*FISGEPD | 1117 | Phldb2 | Isoform 1 of Pleckstrin homology-like | 1.0688 |
| | R\hat{ } | | | domain family B member 2 | |
| IPI:IPI00330246.2 | WAAHGTS*PEDFSLTLGAR\hat{ } | 1118 | Phldb1 | Isoform 2 of Pleckstrin homology-like | 1.068 |
| | | | | domain family B member 1 | |
| IPI:IPI00122594.4 | NGVSLFNSPK@T*EQPSPV | 1119 | Ahctf1 | AT-hook-containing transcription | 1.0661 |
| | VHSFPHPELPEAFVGTPISN | | | factor 1 | |
| | TSQR\hat{ } | | | | |
| IPI:IPI00122594.4 | NGVSLFNS*PKTEQPSPVV | 1120 | Ahctf1 | AT-hook-containing transcription | 1.0661 |
| | HSFPHPELPEAFVGTPISNT | | | factor 1 | |
| | SQR | | | | |
| IPI:IPI00122594.4 | NGVSLFNSPK@TEQPSPVV | 1121 | Ahctf1 | AT-hook-containing transcription | 1.0661 |
| | HS*FPHPELPEAFVGTPISN | | | factor 1 | |
| | TSQR\hat{ } | | | | |
| IPI:IPI00121418.1 | DGEGPDNLEPACPLSLPLQ | 1122 | Rb1 | Retinoblastoma-associated protein | 1.0618 |
| | GNHTAADMYLS*PLR\hat{ }S*PK@ | | | | |
| IPI:IPI00121418.1 | DGEGPDNLEPACPLSLPLQ | 1123 | Rb1 | Retinoblastoma-associated protein | 1.0618 |
| | GNHTAADMYLS*PLRS*PK | | | | |
| IPI:IPI00320905.7 | LGEQGPEPGPT*PPQT*PT* | 1124 | Arhgap17 | Isoform 1 of Rho GTPase-activating | 1.0618 |
| | PPST*PPLAK | | | protein 17 | |
| IPI:IPI00403116.9 | NGS*PVPVPDISQEPDGPAL | 1125 | Ncaph2 | Isoform 3 of Condensin-2 complex | 1.0598 |
| | S*GGEEDAEDGAEPLEVAL | | | subunit H2 | |
| | EPAEPR\hat{ } | | | | |
| IPI:IPI00403116.9 | NGS*PVPVPDISQEPDGPAL | 1126 | Ncaph2 | Isoform 3 of Condensin-2 complex | 1.0598 |
| | S*GGEEDAEDGAEPLEVAL | | | subunit H2 | |
| | EPAEPR | | | | |
| IPI:IPI00403116.9 | NGS*PVPVPDIS*QEPDGPA | 1127 | Ncaph2 | Isoform 3 of Condensin-2 complex | 1.0598 |
| | LSGGEEDAEDGAEPLEVAL | | | subunit H2 | |
| | EPAEPR\hat{ } | | | | |
| IPI:IPI00230237.1 | TAVTATNVSAHGSQANS*P | 1128 | Pbx1 | Isoform PBX1b of Pre-B-cell leukemia | 1.054 |
| | STPNSAGGY*PSPCYQPDR | | | transcription factor 1 | |
| | \hat{ }R\hat{ } | | | | |
| IPI:IPI00421139.8 | SS*SHLDPAATPHSTLQGSS | 1129 | Dlg5 | Putative uncharacterized protein | 1.0498 |
| | AGTPEHPSVIDPLMEQDEG | | | | |
| | PGTPPAK@ | | | | |
| IPI:IPI00121251.7 | LSTTPS*PTNS*LHEDGVDD | 1130 | Tox4 | TOX high mobility group box family | 1.0342 |
| | FRR | | | member 4 | |
| IPI:IPI00121251.7 | LSTTPS*PTNS*LHEDGVDD | 1131 | Tox4 | TOX high mobility group box family | 1.0342 |
| | FR\hat{ }R\hat{ } | | | member 4 | |
| IPI:IPI00121431.4 | S*INYSELDQFPSELEK | 1132 | Hells | Isoform 1 of Lymphocyte specific | 1.0332 |
| | | | | helicase | |
| IPI:IPI00121431.4 | S*INYSELDQFPSELEK@ | 1133 | Hells | Isoform 1 of Lymphocyte-specific | 1.0332 |
| | | | | helicase | |
| IPI:IPI00121431.4 | S*INYSELDQFPSELEK@ | 1134 | Hells | Isoform 1 of Lymphocyte-specific | 1.0332 |
| | | | | helicase | |
| IPI:IPI00454039.3 | VGS*EHSLLDPPGK | 1135 | Erbb2ip | Isoform 1 of Protein LAP2 | 1.0307 |
| IPI:IPI00109318.1 | TVAISDAAQLPQDYCTT*PG | 1136 | Eif4ebp2 | Eukaryotic translation initiation | 1.0271 |
| | GTLFSTT*PGGTR\hat{ }IIYDR\hat{ } | | | factor 4E-binding protein 2 | |
| IPI:IPI00109318.1 | TVAISDAAQLPQDYCTT*PG | 1137 | Eif4ebp2 | Eukaryotic translation initiation | 1.0271 |
| | GTLFSTT*PGGTRIIYDR | | | factor 4E-binding protein 2 | |
| IPI:IPI00226228.5 | ANNAGASIYPTGPADPCPP | 1138 | Azi1 | 5-azacytidine-induced protein 1 | 1.0268 |
| | ASES*S*PEQWQSPEDKPQ | | | | |
| | DIHSQGEAR | | | | |
| IPI:IPI00317401.6 | AES*PETSAVESTQST*PQK@ | 1139 | Pds5b | Isoform 1 of Sister chromatid cohesion | 1.0246 |
| | | | | protein PDS5 homolog B | |
| IPI:IPI00317401.6 | AES*PETSAVESTQST*PQK@ | 1140 | Pds5b | Isoform 1 of Sister chromatid cohesion | 1.0246 |
| | | | | protein PDS5 homolog B | |
| IPI:IPI00317401.6 | AES*PETSAVESTQST*PQK | 1141 | Pds5b | Isoform 1 of Sister chromatid cohesion | 1.0246 |
| | | | | protein PDS5 homolog B | |
| IPI:IPI00113389.5 | HNLFEDNM#ALPSESVSS*L | 1142 | Fam129a | Protein Niban | 1.0232 |
| | TDLK | | | | |
| IPI:IPI00318938.6 | NS*PVAK@T*PPK@DLPAIP | 1143 | Eif4ebp1 | Eukaryotic translation initiation | 1.0227 |
| | GVTSPTSDEPPM#QASQSQ | | | factor 4E-binding protein 1 | |
| | LPSSPEDK@ | | | | |
| IPI:IPI00463074.6 | SRS*S*PSTDHYSQEVPVEP | 1144 | Shroom2 | shroom family member 2 | 1.0176 |
| | NR | | | | |
| IPI:IPI00463074.6 | SRS*S*PSTDHYSQEVPVEP | 1145 | Shroom2 | shroom family member 2 | 1.0176 |
| | NR | | | | |
| IPI:IPI00463074.6 | SR\hat{ }S*S*PSTDHYSQEVPVE | 1146 | Shroom2 | shroom family member 2 | 1.0176 |
| | PNR\hat{ } | | | | |
| IPI:IPI00553798.2 | S*NS*FSDERAEFSAPSTPT | 1147 | Ahnak | AHNAK nucleoprotein isoform 1 | 1.0143 |
| | GTLEFAGGDAK@ | | | | |
| IPI:IPI00553798.2 | S*NS*FSDEREFSAPSTPTG | 1148 | Ahnak | AHNAK nucleoprotein isoform 1 | 1.0143 |
| | TLEFAGGDAK | | | | |
| IPI:IPI00553798.2 | S*NS*FSDEREFSAPSTPTG | 1149 | Ahnak | AHNAK nucleoprotein isoform 1 | 1.0143 |
| | TLEFAGGDAK | | | | |
| IPI:IPI00318938.6 | NS*PVAKT*PPKDLPAIPGVT | 1150 | Eif4ebp1 | Eukaryotic translation initiation | 1.0129 |
| | SPT*SDEPPMQASQSQLPS | | | factor 4E-binding protein 1 | |
| | S*PEDK | | | | |
| IPI:IPI00127131.6 | GPS*PSPVGS*PASVAQSR\hat{ } | 1151 | Arid1a | AT rich interactive domain 1A | 1.0107 |
| IPI:IPI00113389.5 | HNLFEDNMALPSES*VSSLT | 1152 | Fam129a | Protein Niban | 1.0048 |
| | DLK@TAMGS*NQAS*PAR\hat{ } | | | | |
| | R\hat{ } | | | | |
| IPI:IPI00113389.5 | HNLFEDNMALPSESVSSLT* | 1153 | Fam129a | Protein Niban | 1.0048 |
| | DLK@TAMGS*NQAS*PAR\hat{ } | | | | |
| | R\hat{ } | | | | |
| IPI:IPI00454109.2 | FPPST*PSEVLS*PTEDPRS* | 1154 | Erf | ETS domain-containing transcription | 1.0039 |
| | PPACSSSSSSLFSAVVAR | | | factor ERF | |
| IPI:IPI00120095.2 | CDGS*PR\hat{ }T*PPS*TPPATAN | 1155 | Samhd1 | SAM domain and HD domain- | 1.0027 |
| | LSADDDFQNTDLR\hat{ } | | | containing protein 1 | |
| IPI:IPI00117229.3 | TS*PAGGTWSSVVSGVPR\hat{ } | 1156 | Atxn2 | ataxin 2 | 1.0015 |
| IPI:IPI00117229.3 | TS*PAGGTWSSVVSGVPR | 1157 | Atxn2 | ataxin 2 | 1.0015 |
| IPI:IPI00320594.5 | SQDSYPGS*PSLS*PR | 1158 | Ranbp10 | Ran-binding protein 10 | 1.0009 |
| IPI:IPI00320594.5 | SQDSYPGS*PSLS*PR\hat{ } | 1159 | Ranbp10 | Ran-binding protein 10 | 1.0009 |
| IPI:IPI00320594.5 | SQDSYPGS*PSLS*PR\hat{ } | 1160 | Ranbp10 | Ran-binding protein 10 | 1.0009 |
| IPI:IPI00320594.5 | SQDS*YPGS*PSLSPR | 1161 | Ranbp10 | Ran-binding protein 10 | 1.0009 |
| IPI:IPI00320594.5 | SQDSYPGS*PSLS*PR\hat{ } | 1162 | Ranbp10 | Ran-binding protein 10 | 1.0009 |
| IPI:IPI00121251.7 | LSTTPS*PTNS*LHEDGVDD | 1163 | Tox4 | TOX high mobility group box family | 1.0005 |
| | FR\hat{ }R\hat{ } | | | member 4 | |
| IPI:IPI00121251.7 | LSTTPS*PTNS*LHEDGVDD | 1164 | Tox4 | TOX high mobility group box family | 1.0005 |
| | FRR | | | member 4 | |

Table 7. Phosphopeptides whose intensities decrease after rapamycin treatment (Rapamycin screen).

TABLE 8

| | | | | | |
|---|---|---|---|---|---|
| | | | | | Log2 |
| | | SEQ | | | Area |
| | | ID | Gene | | (H/L) |
| Reference | Ascore Seq | NO | Name | Annotation | Ratio |

Ku (light cells: control (rapamycin treated); heavy cells: treated with
rapamycin + Ku-0063794. L, light; H, heavy)

| | | | | | |
|---|---|---|---|---|---|
| IPI:IPI00648918.3 | DQEEELDEQAGS*IQM#LEQ | 1165 | Myo18a | Isoform 4 of Myosin-XVIIIa | −7.171 |
| | LK | | | | |
| IPI:IPI00318938.6 | VALGDGVQLPPGDYSTT*P | 1166 | Eif4ebp1 | Eukaryotic translation | −7.159 |
| | GGTLFSTT*PGGTR | | | initiation factor 4E-binding protein 1 | |
| IPI:IPI00318938.6 | VALGDGVQLPPGDYS*TT*P | 1167 | Eif4ebp1 | Eukaryotic translation initiation | −7.127 |
| | GGTLFSTTPGGTR | | | factor 4E-binding protein 1 | |
| IPI:IPI00622700.2 | TAPPVLPTGYDS*EEEEESR | 1168 | Brd2 | Isoform 2 of Bromodomain-containing | −6.823 |
| | \hat{ }PMSYDEK@ | | | protein 2 | |
| IPI:IPI00458153.2 | ENPPSPPT*SPAAPQPR\hat{ } | 1169 | 6330577E15Rik | Uncharacterized protein C10orf78 | −6.819 |
| | | | | homolog | |
| IPI:IPI00454090.1 | DSVDLQS*LM#TEM#NR | 1170 | Best3 | Bestrophin-3 | −6.498 |
| IPI:IPI00274140.4 | LSPFFTLDLS*PTDDKSSKP | 1171 | Grit | Isoform 2 of Rho/Cdc42/Rac GTPase- | −6.465 |
| | SSFTEK | | | activating protein RICS | |
| IPI:IPI00929786.1 | SLPT*TVPES*PNYR | 1172 | Larp1 | Isoform 1 of La-related protein 1 | −6.159 |
| IPI:IPI00224969.2 | VAPS*MSSLNS*LAS*S*CFD | 1173 | Fam62c | Isoform 1 of Extended synaptotagmin-3 | −6.023 |
| | LTDVSLNTEAGDSR | | | | |
| IPI:IPI00929786.1 | S*LPTTVPES*PNYR | 1174 | Larp1 | Isoform 1 of La-related protein 1 | −5.98 |
| IPI:IPI00458039.3 | IFTEANLVS*VGS*KK | 1175 | Mdn1 | Midasin homolog | −5.907 |
| IPI:IPI00318938.6 | NS*PVAKT*PPKDLPAIPGVT | 1176 | Eif4ebp1 | Eukaryotic translation initiation | −5.874 |
| | SPTSDEPPMQASQSQLPS* | | | factor 4E-binding protein 1 | |
| | SPEDK | | | | |
| IPI:IPI00130920.1 | TLEVVSPSQSVTGS*AGHTP | 1177 | Mtap1b | Microtubule-associated protein 1B | −5.86 |
| | YYQS*PTDEK | | | | |
| IPI:IPI00318938.6 | VALGDGVQLPPGDYSTT*P | 1178 | Eif4ebp1 | Eukaryotic translation initiation | −5.685 |
| | GGTLFSTT*PGGTR | | | factor 4E-binding protein 1 | |
| IPI:IPI00270767.3 | VVK@EDGVMS*PEK@ | 1179 | Rtn4 | Isoform 2 of Reticulon-4 | −5.681 |
| IPI:IPI00121430.2 | DLKPETDYVVNVY*S*VVED | 1180 | Col12a1 | Isoform 1 of Collagen alpha-1(XII) | −5.655 |
| | EYSEPLKGT*EK | | | chain | |
| IPI:IPI00169506.7 | QAVY*Y*LTLQAT*DGGNQS | 1181 | Pcdh24 | Pcdh24 protein | −5.499 |
| | TT*TALEITLLDINDNPPVVR | | | | |
| IPI:IPI00133685.1 | LNT*SDFQK | 1182 | Akt1s1 | Proline-rich AKT1 substrate 1 | −5.419 |
| IPI:IPI00876080.1 | LPDLRS*R | 1183 | Gpr157 | Putative uncharacterized protein | −5.334 |
| | | | | (Fragment) | |
| IPI:IPI00850983.1 | TPGPPS*SQGSPVDTQPAA | 1184 | Synj1 | similar to mKIAA0910 protein | −5.321 |
| | QK | | | | |
| IPI:IPI00318938.6 | NS*PVAKT*PPKDLPAIPGVT | 1185 | Eif4ebp1 | Eukaryotic translation initiation | −4.991 |
| | SPTSDEPPMQASQSQLPSS | | | factor 4E-binding protein 1 | |
| | *PEDK | | | | |
| IPI:IPI00282808.7 | T*PGVPKQKPCSPLS*EPDA | 1186 | Zfp318 | zinc finger protein 318 isoform 1 | −4.923 |
| | FLK | | | | |
| IPI:IPI00133374.5 | EVDPSTGELQSLQMPESEG | 1187 | Sqstm1 | Isoform 1 of Sequestosome-1 | −4.888 |
| | PS*SLDPSQEGPTGLK@ | | | | |
| IPI:IPI00133374.5 | EVDPSTGELQSLQMPESEG | 1188 | Sqstm1 | Isoform 1 of Sequestosome-1 | −4.882 |
| | PS*SLDPSQEGPTGLK@ | | | | |
| IPI:IPI00133685.1 | LNT*SDFQK | 1189 | Akt1s1 | Proline-rich AKT1 substrate 1 | −4.871 |
| IPI:IPI00623114.5 | DMPAAGS*LGSSS*R | 1190 | Fat1 | FAT tumor suppressor homolog 1 | −4.85 |
| IPI:IPI00318938.6 | VALGDGVQLPPGDYSTT*P | 1191 | Eif4ebp1 | Eukaryotic translation initiation | −4.78 |
| | GGT*LFSTTPGGTR | | | factor 4E-binding protein 1 | |
| IPI:IPI00125442.4 | VPMVSS*QPTS*SM#VPPPI | 1192 | Alpk3 | myocyte induction differentiation | −4.621 |
| | KPLNR | | | originator | |
| IPI:IPI00380641.3 | KLPVVSS*VVK | 1193 | Zc3h14 | Isoform 2 of Zinc finger CCCH domain- | −4.368 |
| | | | | containing protein 14 | |
| IPI:IPI00338745.4 | QADVADQQTTELPAENGET | 1194 | Hmgn1; | Non-histone chromosomal protein | −4.324 |
| | ENQSPAS*EEEKEAK | | LOC100044391 | HMG-14 | |
| IPI:IPI00338745.4 | QADVADQQTTELPAENGET | 1195 | Hmgn1; | Non-histone chromosomal protein | −4.324 |
| | ENQS*PASEEEKEAK | | LOC100044391 | HMG-14 | |
| IPI:IPI00271998.4 | GPNS*PPPGMPLR | 1196 | 2310057M21Rik | Uncharacterized protein C10orf88 | −4.316 |
| | | | | homolog | |
| IPI:IPI00338976.3 | FTISAIS*K | 1197 | Fcrls | Fc receptor-like protein 2 scavenger | −4.254 |
| | | | | isoform | |
| IPI:IPI00352984.4 | IVHFS*VNACLT*PICSLHHV | 1198 | Xdh | Xanthine dehydrogenase/oxidase | −4.21 |
| | AVTT*VEGIGNTKK | | | | |
| IPI:IPI00318938.6 | VALGDGVQLPPGDYS*TTP | 1199 | Eif4ebp1 | Eukaryotic translation initiation | −4.196 |
| | GGTLFSTT*PGGTR | | | factor 4E-binding protein 1 | |
| IPI:IPI00330246.2 | K@GSFS*GR\hat{ }LS*PAYSLGS | 1200 | Phldb1 | Isoform 2 of Pleckstrin homology-like | −4.16 |
| | LTGASPR\hat{ } | | | domain family B member 1 | |
| IPI:IPI00330066.5 | T*RSPDVISSASTALSQDIPE | 1201 | Edc4 | Isoform 1 of Enhancer of mRNA- | −4.045 |
| | IASEALSR | | | decapping protein 4 | |
| IPI:IPI00225062.2 | RKETPS*PR | 1202 | Srrm2 | Isoform 3 of Serine/arginine repetitive | −4.005 |
| | | | | matrix protein 2 | |
| IPI:IPI00405665.6 | K@IIETM#SS*PK@ | 1203 | Kif20b | Isoform 1 of M-phase phosphoprotein 1 | −3.928 |
| IPI:IPI00311187.4 | S*AASGS*SGES*MDSVSVS | 1204 | Rgl1 | Ral guanine nucleotide dissociation | −3.911 |
| | *S*CESNHS*EAEEGPVTPM | | | stimulator-like 1 | |
| | DTPDEPQKK | | | | |
| IPI:IPI00153724.1 | VHS*PPASLVPR\hat{ } | 1205 | 1110007A13Rik | UPF0557 protein C10orf119 homolog | −3.905 |
| IPI:IPI00121277.1 | VAAAAGSGPS*PPCS*PGH | 1206 | Pi4k2a | Phosphatidylinositol 4-kinase type 2- | −3.904 |
| | DR | | | alpha | |
| IPI:IPI00331556.5 | MQVDQEEPHTEEQQQQPQ | 1207 | Hspa4 | Heat shock 70 kDa protein 4 | −3.897 |
| | TPAENKAESEEM#ET*SQA | | | | |
| | GSK | | | | |
| IPI:IPI00135443.2 | KTSFDQDS*DVDIFPSDFTS | 1208 | Top2b | DNA topoisomerase 2-beta | −3.873 |
| | EPPALPR | | | | |
| IPI:IPI00469938.3 | VGSGAEPGEGS*PGSR\hat{ }PG | 1209 | BC021381 | Isoform 2 of Uncharacterized protein | −3.843 |
| | PIQADS*PK@ | | | KIAA1931 | |
| IPI:IPI00331361.2 | SPT*KAEPATPAEAAQSDR | 1210 | Mybbp1a | Myb-binding protein 1A | −3.83 |
| IPI:IPI00625898.3 | RQS*S*ESDIESVMYT | 1211 | Bbx | Isoform 1 of HMG box transcription | −3.809 |
| | IEAVAK | | | factor BBX | |
| IPI:IPI00405227.3 | S*LGEIAALTSK | 1212 | Vcl | Vinculin | −3.804 |
| IPI:IPI00109318.1 | RNS*PMAQTPPCHLPNIPGV | 1213 | Eif4ebp2 | Eukaryotic translation initiation | −3.791 |
| | TSPGALIEDSK | | | factor 4E-binding protein 2 | |
| IPI:IPI00318938.6 | VALGDGVQLPPGDYSTT*P | 1214 | Eif4ebp1 | Eukaryotic translation initiation | −3.751 |
| | GGTLFSTT*PGGTR | | | factor 4E-binding protein 1 | |
| IPI:IPI00318938.6 | RVALGDGVQLPPGDY*STT | 1215 | Eif4ebp1 | Eukaryotic translation initiation | −3.726 |
| | PGGTLFSTT*PGGTR | | | factor 4E-binding protein 1 | |
| IPI:IPI00318938.6 | RVALGDGVQLPPGDYSTT* | 1216 | Eif4ebp1 | Eukaryotic translation initiation | −3.726 |
| | PGGTLFSTT*PGGTR | | | factor 4E-binding protein 1 | |
| IPI:IPI00318938.6 | VALGDGVQLPPGDYSTTPG | 1217 | Eif4ebp1 | Eukaryotic translation initiation | −3.705 |
| | GTLFS*TT*PGGTR | | | factor 4E-binding protein 1 | |
| IPI:IPI00929814.1 | KEANEVLS*DS*AGEDHPAE | 1218 | Morc2a | microrchidia 2A isoform 2 | −3.673 |
| | LR | | | | |
| IPI:IPI00229645.2 | S*NDDLLAGMAGGVNVTNG | 1219 | Cytsa | Cytospin-A | −3.632 |
| | IK | | | | |
| IPI:IPI00553798.2 | AGAIS*ASGPELEGAGHSK | 1220 | Ahnak | AHNAK nucleoprotein isoform 1 | −3.608 |
| IPI:IPI00756424.3 | TAR\hat{ }PNSEAPLS*GS*EDAD | 1221 | Eif5b | Isoform 1 of Eukaryotic translation | −3.543 |
| | DSNK@ | | | initiation factor 5B | |
| IPI:IPI00121760.5 | LKTEEGEIVY*SAEESENR | 1222 | Hnrpll | Isoform 1 of Heterogeneous nuclear | −3.51 |
| | | | | ribonucleoprotein L-like | |
| IPI:IPI00458958.2 | DWDKES*EGEEPAGGR | 1223 | Rrp15 | RRP15-like protein | −3.46 |
| IPI:IPI00458958.2 | DWDK@ES*EGEEPAGGR\hat{ } | 1224 | Rrp15 | RRP15-like protein | −3.46 |
| IPI:IPI00318938.6 | RVALGDGVQLPPGDYSTT* | 1225 | Eif4ebp1 | Eukaryotic translation initiation | −3.429 |
| | PGGTLFSTT*PGGTR | | | factor 4E-binding protein 1 | |
| IPI:IPI00318938.6 | RVALGDGVQLPPGDYSTTP | 1226 | Eif4ebp1 | Eukaryotic translation initiation | −3.429 |
| | GGT*LFSTT*PGGTR | | | factor 4E-binding protein 1 | |
| IPI:IPI00318938.6 | RVALGDGVQLPPGDYSTT* | 1227 | Eif4ebp1 | Eukaryotic translation initiation | −3.429 |
| | PGGTLFSTT*PGGTR | | | factor 4E-binding protein 1 | |
| IPI:IPI00109695.3 | IEILDS*PASK | 1228 | D10Wsu102e | Uncharacterized protein C12orf45 | −3.403 |
| | | | | homolog | |
| IPI:IPI00230719.8 | RDS*SDDWEIPDGQITVGQR | 1229 | Braf | Isoform 1 of B-Raf proto-oncogene | −3.372 |
| | | | | serine/threonine-protein kinase | |
| IPI:IPI00377925.2 | SS*PVCSTAPVETEPK | 1230 | Atg2b | Isoform 1 of Autophagy-related protein | −3.353 |
| | | | | 2 homolog B | |
| IPI:IPI00221723.1 | MGQEFVES*K | 1231 | Wasf2 | Wiskott-Aldrich syndrome protein family | −3.314 |
| | | | | member 2 | |
| IPI:IPI00165794.1 | LWTHTSDQQVSAISNPSPC | 1232 | Tmem48 | Nucleoporin NDC1 | −3.255 |
| | AS*VTAEGK | | | | |
| IPI:IPI00848747.1 | DDS*TCLGGILPAGLEPGS* | 1233 | 4930568B11Rik | hypothetical protein LOC75873 | −3.253 |
| | S*T*R | | | | |
| IPI:IPI00318938.6 | VALGDGVQLPPGDYSTT*P | 1234 | Eif4ebp1 | Eukaryotic translation initiation | −3.239 |
| | GGTLFSTT*PGGTR | | | factor 4E-binding protein 1 | |
| IPI:IPI00127415.1 | CGSGPVHISGQHLVAVEED | 1235 | Npm1 | Nucleophosmin | −3.232 |
| | AES*EDEDEEDVK@ | | | | |
| IPI:IPI00318938.6 | VALGDGVQLPPGDYST*TP | 1236 | Eif4ebp1 | Eukaryotic translation initiation | −3.209 |
| | GGTLFST*TPGGTR | | | factor 4E-binding protein 1 | |
| IPI:IPI00318938.6 | VALGDGVQLPPGDYS*TTP | 1237 | Eif4ebp1 | Eukaryotic translation initiation | −3.209 |
| | GGTLFSTT*PGGTR | | | factor 4E-binding protein 1 | |
| IPI:IPI00318938.6 | VALGDGVQLPPGDYSTT*P | 1238 | Eif4ebp1 | Eukaryotic translation initiation | −3.175 |
| | GGTLFSTT*PGGTR | | | factor 4E-binding protein 1 | |
| IPI:IPI00318938.6 | VALGDGVQLPPGDYSTT*P | 1239 | Eif4ebp1 | Eukaryotic translation initiation | −3.175 |
| | GGTLFS*TTPGGTR | | | factor 4E-binding protein 1 | |
| IPI:IPI00120133.1 | EDGDILAS*S*FSVDTQVAYI | 1240 | Nat15 | N-acetyltransferase 15 | −3.163 |
| | LSLGVVK | | | | |
| IPI:IPI00154109.2 | SNS*APLIHGLSDSS*PVFQA | 1241 | Fam122a | Protein FAM122A | −3.124 |
| | EAPSAR | | | | |
| IPI:IPI00138199.5 | DGY*Y*GES*T*S*GR\hat{ } | 1242 | Pcsk5 | proprotein convertase subtilisin/kexin | −3.103 |
| | | | | type 5 | |
| IPI:IPI00318938.6 | RVALGDGVQLPPGDYSTT* | 1243 | Eif4ebp1 | Eukaryotic translation initiation | −3.092 |
| | PGGTLFSTT*PGGTR | | | factor 4E-binding protein 1 | |
| IPI:IPI00130920.1 | TELS*PSFINPNPLEWFAGE | 1244 | Mtap1b | Microtubule-associated protein 1B | −3.089 |
| | EPTEESEKPLTQSGGAPPP | | | | |
| | SGGK | | | | |
| IPI:IPI00131725.3 | HQIVCINNDLS*EEES*DDES | 1245 | Npm3 | Nucleoplasmin-3 | −3.087 |
| | *EEDEIK@LCGILPAK@ | | | | |
| IPI:IPI00330066.5 | T*RSPDVISSASTALSQDIPE | 1246 | Edc4 | Isoform 1 of Enhancer of mRNA- | −3.084 |
| | IASEALSR | | | decapping protein 4 | |
| IPI:IPI00756765.3 | ALVIQESESPPS*PPPS*PDR | 1247 | Ehmt2 | Euchromatic histone lysine N- | −3.071 |
| | \hat{ }R\hat{ } | | | methyltransferase 2 | |
| IPI:IPI00221889.2 | ILS*DDEDEDEEDAFK | 1248 | Ercc6l | DNA excision repair protein ERCC-6- | −3.063 |
| | | | | like | |
| IPI:IPI00130920.1 | TELS*PSFINPNPLEWFAGE | 1249 | Mtap1b | Microtubule-associated protein 1B | −3.052 |
| | EPTEESEKPLTQSGGAPPP | | | | |
| | SGGK | | | | |
| IPI:IPI00318938.6 | VALGDGVQLPPGDYSTT*P | 1250 | Eif4ebp1 | Eukaryotic translation initiation | −3.033 |
| | GGTLFSTTPGGT*R | | | factor 4E-binding protein 1 | |
| IPI:IPI00318938.6 | RVALGDGVQLPPGDYSTT* | 1251 | Eif4ebp1 | Eukaryotic translation initiation | −3.031 |
| | PGGTLFSTT*PGGTR | | | factor 4E-binding protein 1 | |
| IPI:IPI00318938.6 | RVALGDGVQLPPGDYSTT* | 1252 | Eif4ebp1 | Eukaryotic translation initiation | −3.031 |
| | PGGTLFSTT*PGGTR | | | factor 4E-binding protein 1 | |
| IPI:IPI00318938.6 | RVALGDGVQLPPGDYSTT* | 1253 | Eif4ebp1 | Eukaryotic translation initiation | −3.031 |
| | PGGTLFSTT*PGGTR | | | factor 4E-binding protein 1 | |
| IPI:IPI00137465.1 | S*DGSLDDGDGIHR | 1254 | Serinc1 | Serine incorporator 1 | −3.027 |
| IPI:IPI00130920.1 | QGFPDRES*PVSDLTSTGLY | 1255 | Mtap1b | Microtubule-associated protein 1B | −3 |
| | QDK | | | | |
| IPI:IPI00318938.6 | VALGDGVQLPPGDYSTT*P | 1256 | Eif4ebp1 | Eukaryotic translation initiation | −2.995 |
| | GGTLFSTTPGGT*R | | | factor 4E-binding protein 1 | |
| IPI:IPI00109318.1 | RNSPMAQT*PPCHLPNIPGV | 1257 | Eif4ebp2 | Eukaryotic translation initiation | −2.988 |
| | TSPGALIEDSK | | | factor 4E-binding protein 2 | |
| IPI:IPI00122757.1 | TDYYPEEMS*PPLMNPVSP | 1258 | Klf3; | Krueppel-like factor 3 | −2.986 |
| | PQALLQENHPS*VIVQPGK@ | | LOC100046855 | | |
| IPI:IPI00318938.6 | VALGDGVQLPPGDYSTT*P | 1259 | Eif4ebp1 | Eukaryotic translation initiation | −2.972 |
| | GGTLFSTT*PGGTR | | | factor 4E-binding protein 1 | |
| IPI:IPI00126006.6 | TQT*PPLGQTPQLGLK | 1260 | Eif4g2 | Isoform 1 of Eukaryotic translation | −2.956 |
| | | | | initiation factor 4 gamma 2 | |
| IPI:IPI00460668.2 | KAENAEGQTPAIGPDGEPL | 1261 | Smarca4 | Putative uncharacterized protein | −2.915 |
| | DETSQM#S*DLPVK | | | | |
| IPI:IPI00318938.6 | NS*PVAKT*PPKDLPAIPGVT | 1262 | Eif4ebp1 | Eukaryotic translation initiation | −2.908 |
| | SPTSDEPPMQASQSQLPS* | | | factor 4E-binding protein 1 | |
| | SPEDK | | | | |
| IPI:IPI00127764.2 | EGENVKPVNSGTWVAS*NS | 1263 | Pcm1 | Isoform 1 of Pericentriolar material 1 | −2.9 |
| | *ELTPSESLVTT*DDETFEK | | | protein | |
| IPI:IPI00874995.2 | AS*DDLGEPDVFATAPFR | 1264 | Aak1 | Uncharacterized protein FLJ45252 | −2.899 |
| | | | | homolog | |
| IPI:IPI00318938.6 | NS*PVAKT*PPKDLPAIPGVT | 1265 | Eif4ebp1 | Eukaryotic translation initiation | −2.888 |
| | SPTSDEPPMQASQSQLPSS | | | factor 4E-binding protein 1 | |
| | PEDK | | | | |
| IPI:IPI00318938.6 | VALGDGVQLPPGDYSTTPG | 1266 | Eif4ebp1 | Eukaryotic translation initiation | −2.884 |
| | GT*LFSTT*PGGTR | | | factor 4E-binding protein 1 | |
| IPI:IPI00128564.1 | R\hat{ }AAAASAAEAGIAT*PGTE | 1267 | Psmd4 | Isoform Rpn10A of 26S proteasome | −2.861 |
| | DSDDALLK@ | | | non-ATPase regulatory subunit 4 | |
| IPI:IPI00318938.6 | VALGDGVQLPPGDYSTTPG | 1268 | Eif4ebp1 | Eukaryotic translation initiation | −2.846 |
| | GT*LFSTT*PGGTR | | | factor 4E-binding protein 1 | |
| IPI:IPI00318938.6 | VALGDGVQLPPGDYSTT*P | 1269 | Eif4ebp1 | Eukaryotic translation initiation | −2.84 |
| | GGTLFSTT*PGGTR | | | factor 4E-binding protein 1 | |
| IPI:IPI00406741.5 | AAVGVTGNDITTPPNKEPPP | 1270 | Mtap4 | Isoform 4 of Microtubule-associated | −2.832 |
| | S*PEKK | | | protein 4 | |
| IPI:IPI00318938.6 | VALGDGVQLPPGDYSTT*P | 1271 | Eif4ebp1 | Eukaryotic translation initiation | −2.799 |
| | GGTLFSTT*PGGTR | | | factor 4E-binding protein 1 | |
| IPI:IPI00318938.6 | VALGDGVQLPPGDYSTT*P | 1272 | Eif4ebp1 | Eukaryotic translation initiation | −2.799 |
| | GGTLFST*TPGGTR | | | factor 4E-binding protein 1 | |
| IPI:IPI00318938.6 | VALGDGVQLPPGDYSTT*P | 1273 | Eif4ebp1 | Eukaryotic translation initiation | −2.794 |
| | GGTLFSTT*PGGTR | | | factor 4E-binding protein 1 | |
| IPI:IPI00318938.6 | VALGDGVQLPPGDYSTTPG | 1274 | Eif4ebp1 | Eukaryotic translation initiation | −2.776 |
| | GT*LFSTT*PGGTR | | | factor 4E-binding protein 1 | |
| IPI:IPI00109318.1 | NS*PMAQTPPCHLPNIPGVT | 1275 | Eif4ebp2 | Eukaryotic translation initiation | −2.763 |
| | SPGALIEDSK | | | factor 4E-binding protein 2 | |
| IPI:IPI00318938.6 | VALGDGVQLPPGDYSTT*P | 1276 | Eif4ebp1 | Eukaryotic translation initiation | −2.762 |
| | GGTLFSTT*PGGTR | | | factor 4E-binding protein 1 | |
| IPI:IPI00318938.6 | VALGDGVQLPPGDYSTT*P | 1277 | Eif4ebp1 | Eukaryotic translation initiation | −2.756 |
| | GGTLFSTT*PGGTR | | | factor 4E-binding protein 1 | |
| IPI:IPI00331177.2 | VSS*TPETPLTK@ | 1278 | Pola2 | DNA polymerase alpha subunit B | −2.747 |
| IPI:IPI00109318.1 | NSPMAQT*PPCHLPNIPGVT | 1279 | Eif4ebp2 | Eukaryotic translation initiation | −2.714 |
| | SPGALIEDSK | | | factor 4E-binding protein 2 | |
| IPI:IPI00318938.6 | VALGDGVQLPPGDYSTTPG | 1280 | Eif4ebp1 | Eukaryotic translation initiation | −2.672 |
| | GT*LFSTT*PGGTR | | | factor 4E-binding protein 1 | |
| IPI:IPI00318938.6 | VALGDGVQLPPGDYSTT*P | 1281 | Eif4ebp1 | Eukaryotic translation initiation | −2.672 |
| | GGTLFSTTPGGT*R | | | factor 4E-binding protein 1 | |
| IPI:IPI00318938.6 | VALGDGVQLPPGDYSTT*P | 1282 | Eif4ebp1 | Eukaryotic translation initiation | −2.672 |
| | GGTLFSTT*PGGTR | | | factor 4E-binding protein 1 | |
| IPI:IPI00322707.5 | IK@PVTENLVLPSHTGFCQ | 1283 | Atrx | Transcriptional regulator ATRX | −2.663 |
| | S*S*GDEALSK@ | | | | |
| IPI:IPI00322707.5 | IK@PVTENLVLPSHTGFCQ | 1284 | Atrx | Transcriptional regulator ATRX | −2.663 |
| | S*S*GDEALSK@ | | | | |
| IPI:IPI00918973.1 | IGFPETAEEELEEIASENS*D | 1285 | Strap | Serine-threonine kinase receptor- | −2.612 |
| | SIYSSTPEVK | | | associated protein | |
| IPI:IPI00318938.6 | VALGDGVQLPPGDYS*TTP | 1286 | Eif4ebp1 | Eukaryotic translation initiation | −2.565 |
| | GGTLFSTT*PGGTR | | | factor 4E-binding protein 1 | |
| IPI:IPI00125960.1 | TAS*GSS*VTS*LEGTR | 1287 | Ndrg1 | Protein NDRG1 | −2.557 |
| IPI:IPI00125960.1 | TAS*GSS*VTS*LEGTR | 1288 | Ndrg1 | Protein NDRG1 | −2.557 |
| IPI:IPI00125960.1 | TAS*GSSVTS*LEGTR | 1289 | Ndrg1 | Protein NDRG1 | −2.546 |
| IPI:IPI00323045.3 | S*MDVDLNQAHMEDTPK@ | 1290 | Melk | Maternal embryonic leucine zipper | −2.53 |
| | | | | kinase | |
| IPI:IPI00465761.5 | SQLVHGCSQDSDELNPGGL | 1291 | Arhgef17 | Isoform 1 of Rho guanine nucleotide | −2.526 |
| | GSAGGVGS*PEPPTS*PR\hat{ } | | | exchange factor 17 | |
| IPI:IPI00465761.5 | SQLVHGCSQDSDELNPGGL | 1292 | Arhgef17 | Isoform 1 of Rho guanine nucleotide | −2.513 |
| | GSAGGVGS*PEPPTS*PR\hat{ } | | | exchange factor 17 | |
| IPI:IPI00274140.4 | LSPFFTLDLSPT*DDK@SSK | 1293 | Grit | Isoform 2 of Rho/Cdc42/Rac GTPase- | −2.507 |
| | @PSSFTEK@ | | | activating protein RICS | |
| IPI:IPI00318938.6 | RVALGDGVQLPPGDYSTT* | 1294 | Eif4ebp1 | Eukaryotic translation initiation | −2.506 |
| | PGGTLFSTFPGGTR | | | factor 4E-binding protein 1 | |
| IPI:IPI00314240.5 | K@QPPVS*PGTALVGSQK@ | 1295 | Hmga1 | Isoform HMG-I of High mobility group | −2.493 |
| | | | | protein HMG-I/HMG-Y | |
| IPI:IPI00318938.6 | VALGDGVQLPPGDYSTT*P | 1296 | Eif4ebp1 | Eukaryotic translation initiation | −2.484 |
| | GGTLFSTT*PGGTR | | | factor 4E-binding protein 1 | |
| IPI:IPI00135379.3 | VSPMLPSSSLES*PKDK | 1297 | Myo9b | Isoform 1 of Myosin-IXb | −2.452 |
| IPI:IPI00753701.3 | SQSGS*PAAPVEQVVIHTDT | 1298 | Mdc1 | mediator of DNA damage checkpoint 1 | −2.44 |
| | SGDPTLPQR\hat{ } | | | | |
| IPI:IPI00221889.2 | R\hat{ }ILS*DDEDEDEEDAFK@ | 1299 | Ercc6l | DNA excision repair protein ERCC-6- | −2.438 |
| | | | | like | |
| IPI:IPI00318938.6 | RVALGDGVQLPPGDYSTT* | 1300 | Eif4ebp1 | Eukaryotic translation initiation | −2.437 |
| | PGGTLFSTT*PGGTR | | | factor 4E-binding protein 1 | |
| IPI:IPI00318938.6 | RVALGDGVQLPPGDYS*TT | 1301 | Eif4ebp1 | Eukaryotic translation initiation | −2.437 |
| | PGGTLFSTT*PGGTR | | | factor 4E-binding protein 1 | |
| IPI:IPI00454138.4 | K@QQHVISTEEGDM#M#ET | 1302 | Sap130 | Isoform 1 of Histone deacetylase | −2.434 |
| | *NST*DDEK@ | | | complex subunit SAP130 | |
| IPI:IPI00454138.4 | K@QQHVISTEEGDM#M#ET | 1303 | Sap130 | Isoform 1 of Histone deacetylase | −2.434 |
| | *NS*TDDEK@ | | | complex subunit SAP130 | |
| IPI:IPI00753701.3 | SQSGS*PAAPVEQVVIHTDT | 1304 | Mdc1 | mediator of DNA damage checkpoint 1 | −2.433 |
| | SGDPTLPQR\hat{ } | | | | |
| IPI:IPI00654388.2 | AENQR\hat{ }PAEDSALS*PGPLA | 1305 | Lrrfip1 | Isoform 1 of Leucine-rich repeat | −2.425 |
| | GAK@ | | | flightless-interacting protein 1 | |
| IPI:IPI00654388.2 | AENQRPAEDSALS*PGPLA | 1306 | Lrrfip1 | Isoform 1 of Leucine-rich repeat | −2.425 |
| | GAK | | | flightless-interacting protein 1 | |
| IPI:IPI00330121.4 | GLPYADHNYGAPPPPT*PP | 1307 | Setd5 | Isoform 2 of SET domain-containing | −2.414 |
| | AS*PPVQTIIPR\hat{ } | | | protein 5 | |
| IPI:IPI00323256.3 | HGS*SSYPPVIYSPLMPK | 1308 | Zfpm2 | Isoform 1 of Zinc finger protein ZFPM2 | −2.405 |
| IPI:IPI00323256.3 | HGSSS*YPPVIYSPLMPK | 1309 | Zfpm2 | Isoform 1 of Zinc finger protein ZFPM2 | −2.405 |
| IPI:IPI00323256.3 | HGSS*SYPPVIYSPLMPK@ | 1310 | Zfpm2 | Isoform 1 of Zinc finger protein ZFPM2 | −2.395 |
| IPI:IPI00123474.2 | AGADIHAENEEPLCPLPSPS | 1311 | Nfkb2 | NF-kB2 splice variant 4 | −2.389 |
| | TSGSDS*DSEGPER\hat{ } | | | | |
| IPI:IPI00117689.1 | EGDELGEGERPEDDTAAIE | 1312 | Ptrf | Polymerase I and transcript release | −2.385 |
| | LS*S*DEAVEVEEVIEESR | | | factor | |
| IPI:IPI00117689.1 | EGDELGEGERPEDDTAAIE | 1313 | Ptrf | Polymerase I and transcript release | −2.385 |
| | LS*S*DEAVEVEEVIEESR | | | factor | |
| IPI:IPI00127764.2 | RSS*LVDEAPEDEEFEQK | 1314 | Pcm1 | Isoform 1 of Pericentriolar material 1 | −2.38 |
| | | | | protein | |
| IPI:IPI00330121.4 | GLPYADHNYGAPPPPT*PP | 1315 | Setd5 | Isoform 2 of SET domain-containing | −2.359 |
| | AS*PPVQTIIPR | | | protein 5 | |
| IPI:IPI00121277.1 | VAAAAGSGPS*PPCS*PGH | 1316 | Pi4k2a | Phosphatidylinositol 4-kinase type 2- | −2.354 |
| | DR | | | alpha | |
| IPI:IPI00331361.2 | SPT*KAEPATPAEAAQSDR | 1317 | Mybbp1a | Myb-binding protein 1A | −2.35 |
| IPI:IPI00318938.6 | RVALGDGVQLPPGDYSTT* | 1318 | Eif4ebp1 | Eukaryotic translation initiation | −2.341 |
| | PGGTLFSTT*PGGTR | | | factor 4E-binding protein 1 | |
| IPI:IPI00330121.4 | GLPYADHNYGAPPPPT*PP | 1319 | Setd5 | Isoform 2 of SET domain-containing | −2.334 |
| | AS*PPVQTIIPIR\hat{ } | | | protein 5 | |
| IPI:IPI00623284.4 | IWDPTPS*HTPAGAAT*PGR | 1320 | Sf3b1 | Splicing factor 3B subunit 1 | −2.329 |
| | GDT*PGHAT*PGHGGATSS | | | | |
| | AR | | | | |
| IPI:IPI00153715.5 | TEDEEFLIQHLLQAPS*PPR | 1321 | Prr12 | proline rich 12 | −2.321 |
| IPI:IPI00116268.3 | SGS*CSS*PQPKPNY*PPLS* | 1322 | N4bp1 | N4bp1 protein | −2.307 |
| | PPLPLPQLLPSVT*EAR | | | | |
| IPI:IPI00322707.5 | AK@QPVIGDQNS*DSDEM# | 1323 | Atrx | Transcriptional regulator ATRX | −2.262 |
| | LAVLK@ | | | | |
| IPI:IPI00322707.5 | AKQPVIGDQNS*DSDEM#LA | 1324 | Atrx | Transcriptional regulator ATRX | −2.262 |
| | VLK | | | | |
| IPI:IPI00461244.3 | DTTS*FEDISPQGISDDSST | 1325 | Ccdc88a | Isoform 2 of Girdin | −2.242 |
| | GSR | | | | |
| IPI:IPI00317794.5 | KEDS*DEDEDEEDEDDS*D | 1326 | Ncl | Nucleolin | −2.198 |
| | EDEDDEEEDEFEPPIVK | | | | |
| IPI:IPI00317794.5 | K@EDS*DEDEDEEDEDDS* | 1327 | Ncl | Nucleolin | −2.198 |
| | DEDEDDEEEDEFEPPIVK@ | | | | |
| IPI:IPI00400381.9 | AHS*LLFENSDSFSEDTGTL | 1328 | Phactr4 | Isoform 1 of Phosphatase and actin | −2.193 |
| | GR\hat{ } | | | regulator 4 | |
| IPI:IPI00320208.3 | YGPSSVEDTTGSGAADAKD | 1329 | Eef1b2 | Elongation factor 1-beta | −2.192 |
| | DDDIDLFGS*DDEEESEEAK | | | | |
| IPI:IPI00923656.1 | R\hat{ }DS*SSLSSPLNPK@ | 1330 | Baz1b | Isoform 1 of Tyrosine-protein kinase | −2.17 |
| | | | | BAZ1B | |
| IPI:IPI00318938.6 | VALGDGVQLPPGDYSTTPG | 1331 | Eif4ebp1 | Eukaryotic translation initiation | −2.17 |
| | GT*LFST*TPGGTR | | | factor 4E-binding protein 1 | |
| IPI:IPI00108389.5 | ETVVSGPLGVEDISPSMS*P | 1332 | Trp53bp1 | Transformation related protein 53 | −2.161 |
| | DDK@ | | | binding protein 1 | |
| IPI:IPI00415558.1 | AASPAKPS*SLDLVPNLPR | 1333 | Synpo | Isoform 3 of Synaptopodin | −2.151 |
| IPI:IPI00404545.2 | SCSVT*DTVAEQAHLPPPSV | 1334 | Nedd4I | Isoform 3 of E3 ubiquitin-protein | −2.113 |
| | AYVHTTPGLPSGWEER | | | ligase NEDD4-like | |
| IPI:IPI00416203.1 | AVPFVPM#SYQLS*QS*Y*Y | 1335 | Gtf3c1 | Isoform 2 of General transcription | −2.105 |
| | S*LGK | | | factor 3C polypeptide 1 | |
| IPI:IPI00355042.3 | IPMPPSS*PQPR | 1336 | Ppp1r13l | RelA-associated inhibitor | −2.104 |
| IPI:IPI00320208.3 | YGPSSVEDTTGSGAADAKDK | 1337 | Eef1b2 | Elongation factor 1-beta | −2.096 |
| | DDDIDLFGS*DDEEESEEAKK | | | | |
| IPI:IPI00623382.2 | LMKPPS*PEHQS*PDTQQE | 1338 | Morc2a | MORC family CW-type zinc finger | −2.069 |
| | GGEEEEAMVAR | | | protein 2A | |
| IPI:IPI00125960.1 | TAS*GSS*VTSLEGTR | 1339 | Ndrg1 | Protein NDRG1 | −2.055 |
| IPI:IPI00125960.1 | TAS*GSS*VTSLEGTR | 1340 | Ndrg1 | Protein NDRG1 | −2.055 |
| IPI:IPI00125960.1 | TAS*GSS*VTSLEGTR\hat{ } | 1341 | Ndrg1 | Protein NDRG1 | −2.055 |
| IPI:IPI00330121.4 | GLPYADHNYGAPPPPT*PP | 1342 | Setd5 | Isoform 2 of SET domain-containing | −2.045 |
| | AS*PPVQTIIPR\hat{ } | | | protein 5 | |
| IPI:IPI00318938.6 | RVALGDGVQLPPGDYSTT* | 1343 | Eif4ebp1 | Eukaryotic translation initiation | −2.035 |
| | PGGTLFSTT*PGGTR | | | factor 4E-binding protein 1 | |
| IPI:IPI00226701.3 | ILGDHLLLDPAHELPPY*TPP | 1344 | Trerf1 | Isoform 4 of Transcriptional-regulating | −2.03 |
| | PMLS*PVR | | | factor 1 | |
| IPI:IPI00348488.5 | SCS*M#ELHGEGNQEPGSP | 1345 | Dennd4c | DENN/MADD domain containing 4C | −2.012 |
| | AVFAHPLER\hat{ } | | | | |
| IPI:IPI00318938.6 | RVALGDGVQLPPGDYSTT* | 1346 | Eif4ebp1 | Eukaryotic translation initiation | −2.01 |
| | PGGTLFSTT*PGGTR | | | factor 4E-binding protein 1 | |
| IPI:IPI00317794.5 | K@EDS*DEDEDEEDEDDS* | 1347 | Ncl | Nucleolin | −1.999 |
| | DEDEDDEEEDEFEPPIVK@ | | | | |
| IPI:IPI00124717.1 | K@PEFS*PTLK@ | 1348 | Orc6l | Origin recognition complex subunit 6 | −1.969 |
| IPI:IPI00380722.1 | SET*PPVPPPPPYLANYPGF | 1349 | Bat2 | Large proline-rich protein BAT2 | −1.961 |
| | PENGTPGPPISR | | | | |
| IPI:IPI00553798.2 | SNSFS*DEREFSAPSTPTGT | 1350 | Ahnak | AHNAK nucleoprotein isoform 1 | −1.95 |
| | LEFAGGDAK | | | | |
| IPI:IPI00108389.5 | LVS*PETEASEESLQFSLEK | 1351 | Trp53bp1 | Transformation related protein 53 | −1.94 |
| | @PTTAER\hat{ } | | | binding protein 1 | |
| IPI:IPI00124272.1 | LTTTGQVTS*PVK@ | 1352 | Lig3 | Isoform Alpha of DNA ligase 3 | −1.932 |
| IPI:IPI00406741.5 | AAVGVTGNDITTPPNKEPPP | 1353 | Mtap4 | Isoform 4 of Microtubule-associated | −1.932 |
| | S*PEKK | | | protein 4 | |
| IPI:IPI00321774.2 | HLLTDLPLPPELPGGDPSPP | 1354 | Crkrs | Isoform 2 of Cell division cycle 2- | −1.921 |
| | DS*PEPK@ | | | related protein kinase 7 | |
| IPI:IPI00321774.2 | HLLTDLPLPPELPGGDPSPP | 1355 | Crkrs | Isoform 2 of Cell division cycle 2- | −1.921 |
| | DS*PEPK@ | | | related protein kinase 7 | |
| IPI:IPI00124914.1 | S*LHKPMY*FFLINL | 1356 | Olfr521 | Olfactory receptor Olfr521 | −1.913 |
| | S*ALDILFTT*TTVPK | | | | |
| IPI:IPI00123881.1 | VETPDVNLDQEEEIQM#ET | 1357 | Mcm6 | DNA replication licensing factor MCM6 | −1.909 |
| | DEGQGGVNGHADS*PAPV | | | | |
| | NR\hat{ } | | | | |
| IPI:IPI00123881.1 | VETPDVNLDQEEEIQM#ET | 1358 | Mcm6 | DNA replication licensing factor MCM6 | −1.909 |
| | DEGQGGVNGHADS*PAPV | | | | |
| | NR | | | | |
| IPI:IPI00117932.2 | S*PPVQPHTPVTISLGTAPS | 1359 | Sin3a | Isoform 1 of Paired amphipathic helix | −1.896 |
| | LQNNQPVEFNHAINYVNK | | | protein Sin3a | |
| IPI:IPI00229571.1 | ISS*K@S*PGHM#VILNQTK@ | 1360 | Sltm | Isoform 1 of SAFB-like transcription | −1.88 |
| | | | | modulator | |
| IPI:IPI00124753.3 | SLLLADSNGYTNLPDVVQP | 1361 | Mink1 | misshapen-like kinase 1 isoform 2 | −1.868 |
| | SHSPTENS*K | | | | |
| IPI:IPI00117088.2 | LS*VVGPPNR\hat{ } | 1362 | Map3k2 | Mitogen-activated protein kinase kinase | −1.86 |
| | | | | kinase 2 | |
| IPI:IPI00127602.1 | VVVADDDSEAPER\hat{ }PVNGA | 1363 | Slc30a4 | Zinc transporter 4 | −1.822 |
| | HPALQADDDS*LLDQDLPLT | | | | |
| | NSQLSLK@ | | | | |
| IPI:IPI00127602.1 | VVVADDDSEAPER\hat{ }PVNGA | 1364 | Slc30a4 | Zinc transporter 4 | −1.822 |
| | HPALQADDDS*LLDQDLPLT | | | | |
| | NSQLSLK@ | | | | |
| IPI:IPI00228942.4 | DLS*PGAPPAVAAAAPEER\hat{ } | 1365 | Hectd2 | Hectd2 protein | −1.792 |
| IPI:IPI00225062.2 | EMPGSNIES*S*PEVEERPA | 1366 | Srrm2 | Isoform 3 of Serine/arginine repetitive | −1.785 |
| | VLSALDQSQSQPSK | | | matrix protein 2 | |
| IPI:IPI00127602.1 | VVVADDDSEAPERPVNGAH | 1367 | Slc30a4 | Zinc transporter 4 | −1.782 |
| | PALQADDDS*LLDQDLPLTN | | | | |
| | SQLSLK | | | | |
| IPI:IPI00127602.1 | VVVADDDSEAPERPVNGAH | 1368 | Slc30a4 | Zinc transporter 4 | −1.782 |
| | PALQADDDS*LLDQDLPLTN | | | | |
| | SQLSLK | | | | |
| IPI:IPI00551082.3 | EK@PPFLPEEPSS*S*SEED | 1369 | Rbbp6 | Isoform 2 of Retinoblastoma-binding | −1.768 |
| | DPIPDELLCLICK@ | | | protein 6 | |
| IPI:IPI00318938.6 | RVALGDGVQLPPGDYSTT* | 1370 | Eif4ebp1 | Eukaryotic translation initiation | −1.759 |
| | PGGTLFSTT*PGGTR | | | factor 4E-binding protein 1 | |
| IPI:IPI00318938.6 | RVALGDGVQLPPGDYSTT* | 1371 | Eif4ebp1 | Eukaryotic translation initiation | −1.759 |
| | PGGTLFSTT*PGGTR | | | factor 4E-binding protein 1 | |
| IPI:IPI00135660.5 | FQHPNTDMLQEK@PSSPS | 1372 | Sdpr | Serum deprivation-response protein | −1.749 |
| | PMPSST*PSPSLNLGSTEEA | | | | |
| | IR\hat{ } | | | | |
| IPI:IPI00131725.3 | HQIVCINNDLS*EEES*DDES | 1373 | Npm3 | Nucleoplasmin-3 | −1.749 |
| | *EEDEIKLCGILPAK | | | | |
| IPI:IPI00320208.3 | YGPSSVEDTTGSGAADAKD | 1374 | Eef1b2 | Elongation factor 1-beta | −1.741 |
| | DDDIDLFGS*DDEEESEEAKK | | | | |
| IPI:IPI00321774.2 | HLLTDLPLPPELPGGDPS*P | 1375 | Crkrs | Isoform 2 of Cell division cycle 2- | −1.734 |
| | PDSPEPK@ | | | related protein kinase 7 | |
| IPI:IPI00420601.5 | T*CDSPQNPVDFISGPVPDS | 1376 | Usp10 | Ubiquitin carboxyl-terminal hydrolase | −1.734 |
| | PFPR\hat{ } | | | 10 | |
| IPI:IPI00127415.1 | CGSGPVHISGQHLVAVEED | 1377 | Npm1 | Nucleophosmin | −1.718 |
| | AES*EDEDEEDVK@ | | | | |
| IPI:IPI00464324.2 | AS*CEENLPVFIASELAK@ | 1378 | D830031N03Rik | similar to mKIAA0754 protein | −1.718 |
| IPI:IPI00318048.5 | SSPKEEVAS*EPEEAAS*PT | 1379 | Nop56 | Nucleolar protein 56 | −1.708 |
| | T*PK | | | | |
| IPI:IPI00115257.1 | QVDTEEAGMVTAAT*ASNV | 1380 | Psip1 | Isoform 1 of PC4 and SFRS1- | −1.694 |
| | KASPK | | | interacting protein | |
| IPI:IPI00317794.5 | KEDS*DEDEDEEDEDDS*D | 1381 | Ncl | Nucleolin | −1.692 |
| | EDEDDEEEDEFEPPIVK | | | | |
| IPI:IPI00125745.1 | HAELSGS*PLK@ | 1382 | Mta3 | Isoform 1 of Metastasis associated | −1.69 |
| | | | | protein MTA3 | |
| IPI:IPI00461272.6 | RGS*PGGVEMNVELPQQE | 1383 | 2310047M10Rik | Putative uncharacterized protein | −1.686 |
| | GDDDDDEDEEAAAGR | | | | |
| IPI:IPI00453999.2 | K@PS*VPDTASPADDSFVD | 1384 | Nck1 | non-catalytic region of tyrosine kinase | −1.682 |
| | PGER\hat{ } | | | adaptor protein 1 | |
| IPI:IPI00119442.1 | M#DTGEVSDIGSQGAPIVLS | 1385 | Exosc9 | Exosome complex exonuclease RRP45 | −1.674 |
| | *DS*EEEEMIILEPEK@NPK@ | | | | |
| IPI:IPI00654388.2 | AENQRPAEDSALS*PGPLA | 1386 | Lrrfip1 | Isoform 1 of Leucine-rich repeat | −1.673 |
| | GAK | | | flightless-interacting protein 1 | |
| IPI:IPI00128975.3 | DK@DDQEWEST*SPPK@P | 1387 | Dap | Death-associated protein 1 | −1.669 |
| | TVFISGVIAR\hat{ } | | | | |
| IPI:IPI00153375.1 | SACFSPVSLS*PR\hat{ }PCSPFS | 1388 | Pdlim2 | PDZ and LIM domain protein 2 | −1.659 |
| | T*PPPTS*PVALSK@ | | | | |
| IPI:IPI00515576.5 | LDGELAITKPNVSS*PSK | 1389 | Ehbp1 | Isoform 2 of EH domain-binding | −1.659 |
| | | | | protein 1 | |
| IPI:IPI00169521.4 | ALAEEDS*EDELPS*DVDFN | 1390 | Esf1 | ESF1 homolog | −1.656 |
| | DPYFAEEVKK | | | | |
| IPI:IPI00405756.8 | SISGTS*TSEK@PNSMDTA | 1391 | Rasal2 | RAS protein activator like 2 | −1.654 |
| | NTSPFK@ | | | | |
| IPI:IPI00108508.5 | ET*PPTPT*S*SPAEFSPK | 1392 | 4921517D21Rik | RIKEN cDNA 4921517D21 gene | −1.652 |
| IPI:IPI00652649.1 | IRPPPELETS*LTERPSS*PS | 1393 | Als2cr4 | Amyotrophic lateral sclerosis 2 | −1.649 |
| | LLR | | | chromosomal region candidate gene 4 | |
| | | | | protein homolog | |
| IPI:IPI00127415.1 | LKCGSGPVHISGQHLVAVE | 1394 | Npm1 | Nucleophosmin | −1.638 |
| | EDAES*EDEDEEDVK | | | | |
| IPI:IPI00453603.1 | TPVS*PVKFS*PGDFWGR | 1395 | Rps6kb1 | Isoform Alpha I of Ribosomal protein | −1.635 |
| | | | | S6 kinase beta-1 | |
| IPI:IPI00313307.3 | LSSS*DSIGPDVTDILSDIAE | 1396 | Med1 | Isoform 4 of Mediator of RNA | −1.624 |
| | EASK@LPSTSDDCPPIGT*P | | | polymerase II transcription subunit 1 | |
| | VR\hat{ } | | | | |
| IPI:IPI00379682.1 | ANTLSHFPVECPAPPEPAQ | 1397 | Tbc1d1 | Isoform 1 of TBC1 domain family | −1.623 |
| | SS*PGVSQR\hat{ } | | | member 1 | |
| IPI:IPI00379682.1 | ANTLSHFPVECPAPPEPAQ | 1398 | Tbc1d1 | Isoform 1 of TBC1 domain family | −1.623 |
| | S*SPGVSQR\hat{ } | | | member 1 | |
| IPI:IPI00318938.6 | VALGDGVQLPPGDYSTT*P | 1399 | Eif4ebp1 | Eukaryotic translation initiation | −1.621 |
| | GGTLFSTT*PGGTR | | | factor 4E-binding protein 1 | |
| IPI:IPI00351206.5 | LLK@PGEEPSEY*T*DEEDT | 1400 | Pgrmc2 | Membrane-associated progesterone | −1.62 |
| | K@ | | | receptor component 2 | |
| IPI:IPI00225062.2 | NSGPVSEVNTGFS*PEVK@ | 1401 | Srrm2 | Isoform 3 of Serine/arginine repetitive | −1.616 |
| | | | | matrix protein 2 | |
| IPI:IPI00112339.9 | R\hat{ }LS*ENNCSLDDWEIGAGH | 1402 | Lima1 | LIM domain and actin binding 1 | −1.615 |
| | LSSSAFNSEK@ | | | isoform a | |
| IPI:IPI00112339.9 | R\hat{ }LS*ENNCSLDDWEIGAGH | 1403 | Lima1 | LIM domain and actin binding 1 | −1.615 |
| | LSSSAFNSEK@ | | | isoform a | |
| IPI:IPI00515576.5 | LDGELAITKPNVSS*PSK | 1404 | Ehbp1 | Isoform 2 of EH domain-binding | −1.612 |
| | | | | protein 1 | |
| IPI:IPI00229472.5 | VEEQVEDS*DDEEDDDS*H | 1405 | Rbm28 | RNA-binding protein 28 | −1.609 |
| | DDEEER | | | | |
| IPI:IPI00229472.5 | VEEQVEDS*DDEEDDDS*H | 1406 | Rbm28 | RNA-binding protein 28 | −1.609 |
| | DDEEER\hat{ } | | | | |
| IPI:IPI00318938.6 | NS*PVAKT*PPKDLPAIPGVT | 1407 | Eif4ebp1 | Eukaryotic translation initiation | −1.6 |
| | SPTSDEPPM#QASQSQLPS | | | factor 4E-binding protein 1 | |
| | SPEDK | | | | |
| IPI:IPI00127415.1 | LKCGSGPVHISGQHLVAVE | 1408 | Npm1 | Nucleophosmin | −1.599 |
| | EDAES*EDEDEEDVK | | | | |
| IPI:IPI00453837.2 | KLPPPPPQAPPEEENES*EP | 1409 | Kdm1 | Lysine-specific histone demethylase 1 | −1.587 |
| | EEPSGVEGAAFQSR | | | | |
| IPI:IPI00319933.4 | DGTAPPPQSPSSPGSGQD | 1410 | Pacsin3 | Protein kinase C and casein kinase II | −1.565 |
| | EDWS*DEESPR | | | substrate protein 3 | |
| IPI:IPI00125662.2 | S*PQVPAAQQMLNFPEK | 1411 | Smarcc1 | Isoform 1 of SWI/SNF complex subunit | −1.554 |
| | | | | SMARCC1 | |
| IPI:IPI00230435.1 | SLTM#VEDNEDDDEDGEEL | 1412 | Lmna | Isoform C2 of Lamin-A/C | −1.546 |
| | LHHHR\hat{ }VS*GSR\hat{ } | | | | |
| IPI:IPI00228748.1 | LDR\hat{ }AS*PDLWPEQLPGVAE | 1413 | Lin52; | Protein lin-52 homolog | −1.545 |
| | FAASFK@ | | Gm7020 | | |
| IPI:IPI00622364.4 | LPPPFPGMDPESEGAAGAS | 1414 | Snx1 | Sorting nexin-1 | −1.535 |
| | EPEAGDSDTEGEDIFTGAA | | | | |
| | AAT*K@PQS*PK@ | | | | |
| IPI:IPI00117689.1 | GEATDLLRGS*S*PDVHTLL | 1415 | Ptrf | Polymerase I and transcript release | −1.532 |
| | EITEESDAVLVDK | | | factor | |
| IPI:IPI00117689.1 | GEATDLLR\hat{ }GS*S*PDVHTLL | 1416 | Ptrf | Polymerase I and transcript release | −1.532 |
| | EITEESDAVLVDK@ | | | factor | |
| IPI:IPI00606555.2 | KPEPEAGQAEPLS*PRPPP | 1417 | Rreb1 | Isoform 3 of Ras-responsive element- | −1.526 |
| | CPTLSVTVEPK | | | binding protein 1 | |
| IPI:IPI00606555.2 | KPEPEAGQAEPLS*PRPPP | 1418 | Rreb1 | Isoform 3 of Ras-responsive element- | −1.526 |
| | CPTLSVTVEPK | | | binding protein 1 | |
| IPI:IPI00380394.5 | IEEPVSM#EMDNHLSDKDE | 1419 | Ints3 | Integrator complex subunit 3 | −1.525 |
| | SCYDNAEAAFS*DDEEDLN | | | | |
| | SK | | | | |
| IPI:IPI00229571.1 | IS*SK@S*PGHM#VILNQTK@ | 1420 | Sltm | Isoform 1 of SAFB-like transcription | −1.524 |
| | | | | modulator | |
| IPI:IPI00551082.3 | EKPPFLPEEPS*S*SSEEDD | 1421 | Rbbp6 | Isoform 2 of Retinoblastoma-binding | −1.522 |
| | PIPDELLCLICK | | | protein 6 | |
| IPI:IPI00468896.7 | KGELMENDQDAMEYS*S*E | 1422 | Ddx46 | Isoform 1 of Probable ATP-dependent | −1.517 |
| | EEEVDLQTALTGYQTK | | | RNA helicase DDX46 | |
| IPI:IPI00280250.5 | VGES*SEDVALEEETIYENE | 1423 | Sh3pxd2a | Isoform 1 of SH3 and PX domain- | −1.513 |
| | GFR\hat{ }PYTEDTLSAR\hat{ } | | | containing protein 2A | |
| IPI:IPI00280250.5 | VGES*SEDVALEEETIYENE | 1424 | Sh3pxd2a | Isoform 1 of SH3 and PX domain- | −1.513 |
| | GFRPYTEDTLSAR | | | containing protein 2A | |
| IPI:IPI00664670.4 | LGS*FGSITR | 1425 | Flnc | Isoform 1 of Filamin-C | −1.512 |
| IPI:IPI00117929.1 | STEESLSEDAFTESELS*PIR | 1426 | Oxr1 | Isoform 2 of Oxidation resistance | −1.51 |
| | \hat{ }EELLSSEPR\hat{ } | | | protein 1 | |
| IPI:IPI00117929.1 | STEESLSEDAFTESELS*PIR | 1427 | Oxr1 | Isoform 2 of Oxidation resistance | −1.51 |
| | \hat{ }EELLSSEPR\hat{ } | | | protein 1 | |
| IPI:IPI00664670.4 | LGS*FGSITR\hat{ } | 1428 | Flnc | Isoform 1 of Filamin-C | −1.509 |
| IPI:IPI00130920.1 | AS*LS*PMDEPVPDSESPVE | 1429 | Mtap1b | Microtubule-associated protein 1B | −1.509 |
| | K@ | | | | |
| IPI:IPI00113362.2 | NSNSYGIPEPAHAY*AQPQT | 1430 | Crkl | Crk-like protein | −1.508 |
| | TTPLPTVASTPGAAINPLPS | | | | |
| | TQNGPVFAK | | | | |
| IPI:IPI00331334.3 | VSSAPIPCPS*PSPAPSAVP | 1431 | Bag3 | BAG family molecular chaperone | −1.508 |
| | S*PPK | | | regulator 3 | |
| IPI:IPI00331209.1 | NSS*FVLPK | 1432 | Rin2 | Isoform 1 of Ras and Rab interactor 2 | −1.504 |
| IPI:IPI00225062.2 | GHTQTWPDTSSPEVMQT*Q | 1433 | Srrm2 | Isoform 3 of Serine/arginine repetitive | −1.504 |
| | VES*PLLQSK | | | matrix protein 2 | |
| IPI:IPI00664670.4 | LGS*FGSITR\hat{ } | 1434 | Flnc | Isoform 1 of Filamin-C | −1.502 |
| IPI:IPI00664670.4 | LGS*FGSITR | 1435 | Flnc | Isoform 1 of Filamin-C | −1.502 |
| IPI:IPI00664670.4 | LGS*FGSITR | 1436 | Flnc | Isoform 1 of Filamin-C | −1.502 |
| IPI:IPI00664670.4 | LGS*FGSITR | 1437 | Flnc | lsoform 1 of Filamin-C | −1.497 |
| IPI:IPI00126006.6 | TQT*PPLGQTPQLGLK@ | 1438 | Eif4g2 | Isoform 1 of Eukaryotic translation | −1.492 |
| | | | | initiation factor 4 gamma 2 | |
| IPI:IPI00113798.2 | ATWGDGGDNS*PSNVVSK | 1439 | Snap23 | Synaptosomal-associated protein | −1.49 |
| IPI:IPI00664670.4 | LGS*FGSITR\hat{ } | 1440 | Flnc | Isoform 1 of Filamin-C | −1.483 |
| IPI:IPI00169771.1 | EDGIDAVEVAADR\hat{ }PGS*PR\hat{ } | 1441 | BC031781 | UPF0667 protein C1orf55 homolog | −1.482 |
| IPI:IPI00169806.3 | GMEGLIS*PTEAVGNSCGA | 1442 | Nek9 | Serine/threonine-protein kinase Nek9 | −1.478 |
| | SSSCPGWLR\hat{ } | | | | |
| IPI:IPI00117932.2 | S*PPVQPHTPVTISLGTAPS | 1443 | Sin3a | Isoform 1 of Paired amphipathic helix | −1.477 |
| | LQNNQPVEFNHAINYVNK@ | | | protein Sin3a | |
| IPI:IPI00225062.2 | NSGPVSEVNTGFS*PEVK@ | 1444 | Srrm2 | Isoform 3 of Serine/arginine repetitive | −1.475 |
| | | | | matrix protein 2 | |
| IPI:IPI00128062.1 | RGQEELEVPVDPLPS*SPA | 1445 | Snx15 | Sorting nexin-15 | −1.472 |
| | QEALDLLFSCDSTEEASSSL | | | | |
| | AR | | | | |
| IPI:IPI00224570.3 | GVNFAEEPMRS*DS*ENGE | 1446 | Prkar2b | cAMP-dependent protein kinase type II- | −1.468 |
| | EEEAAEAGAFNAPVINR | | | beta regulatory subunit | |
| IPI:IPI00380722.1 | SET*PPVPPPPPYLANYPGF | 1447 | Bat2 | Large proline-rich protein BAT2 | −1.463 |
| | PENGTPGPPISR\hat{ } | | | | |
| IPI:IPI00330695.5 | GLLAQDLQAESSPPAS*PLL | 1448 | Fam129b | Niban-like protein 1 | −1.46 |
| | NGAPVQESSQPVAVPEAS* | | | | |
| | PPASPLR\hat{ } | | | | |
| IPI:IPI00750546.1 | GGGQSS*PQEEPTWK@ | 1449 | Cwc22 | Novel protein | −1.452 |
| IPI:IPI00129356.1 | SGS*GMSVISSSSVDQR | 1450 | Itsn1 | Isoform 1 of Intersectin-1 | −1.451 |
| IPI:IPI00753701.3 | SPT*LGEDS*DTEVDEDHKP | 1451 | Mdc1 | mediator of DNA damage checkpoint 1 | −1.447 |
| | GFADS*ETDVEEER | | | | |
| IPI:IPI00137730.7 | VLTPTQVMNR\hat{ }PSS*ISWDG | 1452 | Pebp1 | Phosphatidylethanolamine-binding | −1.431 |
| | LDPGK@ | | | protein 1 | |
| IPI:IPI00123802.5 | NIQQDNSEAGTQPQVQTDG | 1453 | Hsph1 | Isoform HSP105-alpha of Heat shock | −1.428 |
| | QQTSQSPPS*PELTSEESK | | | protein 105 kDa | |
| | @TPDADK@ | | | | |
| IPI:IPI00421179.1 | EATLPPVS*PPK@ | 1454 | Eif4g1 | Isoform 1 of Eukaryotic translation | −1.421 |
| | | | | initiation factor 4 gamma 1 | |
| IPI:IPI00421179.1 | EATLPPVS*PPK | 1455 | Eif4g1 | Isoform 1 of Eukaryotic translation | −1.421 |
| | | | | initiation factor 4 gamma 1 | |
| IPI:IPI00318006.5 | R\hat{ }IS*WPENSFDFVSK@ | 1456 | Serhl | Serine hydrolase-like protein | −1.411 |
| IPI:IPI00318006.5 | R\hat{ }IS*WPENSFDFVSK@ | 1457 | Serhl | Serine hydrolase-like protein | −1.411 |
| IPI:IPI00125501.1 | KAEEAT*PVTALR | 1458 | Epb4.1l3 | Isoform 1 of Band 4.1-like protein 3 | −1.408 |
| IPI:IPI00405665.6 | K@IIETM#SS*PK@ | 1459 | Kif20b | Isoform 1 of M-phase phosphoprotein 1 | −1.399 |
| IPI:IPI00405665.6 | KIIETM#SS*PK | 1460 | Kif20b | Isoform 1 of M-phase phosphoprotein 1 | −1.399 |
| IPI:IPI00119442.1 | M#DTGEVSDIGSQGAPIVLS | 1461 | Exosc9 | Exosome complex exonuclease RRP45 | −1.396 |
| | *DS*EEEEMIILEPEK@NPK@ | | | | |
| IPI:IPI00121760.5 | LKTEEGEIVYS*AEESENR | 1462 | Hnrpll | Isoform 1 of Heterogeneous nuclear | −1.387 |
| | | | | ribonucleoprotein L-like | |
| IPI:IPI00320208.3 | YGPSSVEDTTGSGAADAKD | 1463 | Eef1b2 | Elongation factor 1-beta | −1.381 |
| | DDDIDLFGS*DDEEESEEAK | | | | |
| IPI:IPI00664670.4 | LGS*FGSITR\hat{ } | 1464 | Flnc | Isoform 1 of Filamin-C | −1.373 |
| IPI:IPI00664670.4 | LGS*FGSITR | 1465 | Flnc | Isoform 1 of Filamin-C | −1.373 |
| IPI:IPI00125348.2 | AEEQLPPLLSPPS*PSTPHSR | 1466 | Mprip | Isoform 1 of Myosin phosphatase Rho- | −1.371 |
| | | | | interacting protein | |
| IPI:IPI00120095.2 | DFTKPQDGDIIAPLIT*PLK | 1467 | Samhd1 | SAM domain and HD domain- | −1.371 |
| | | | | containing protein 1 | |
| IPI:IPI00756765.3 | ALVIQESESPPS*PPPS* | 1468 | Ehmt2 | Euchromatic histone lysine N- | −1.368 |
| | PDRR | | | methyltransferase 2 | |
| IPI:IPI00135708.1 | RKPVLPALTINPTIAEGPS*P | 1469 | Map2k2 | Dual specificity mitogen-activated | −1.365 |
| | TSEGASEANLVDLQK | | | protein kinase kinase 2 | |
| IPI:IPI00317794.5 | KEDS*DEDEDEEDEDDS*D | 1470 | Ncl | Nucleolin | −1.364 |
| | EDEDDEEEDEFEPPIVK | | | | |
| IPI:IPI00330695.5 | GLLAQDLQAESSPPASPLL | 1471 | Fam129b | Niban-like protein 1 | −1.36 |
| | NGAPVQES*S*QPVAVPEAS | | | | |
| | *PPASPLR\hat{ } | | | | |
| IPI:IPI00330695.5 | GLLAQDLQAES*S*PPASPL | 1472 | Fam129b | Niban-like protein 1 | −1.36 |
| | LNGAPVQESSQPVAVPEAS | | | | |
| | *PPASPLR | | | | |
| IPI:IPI00453800.4 | K@PSSS*PDLWK@VS*PDQ | 1473 | Zfp828 | Zinc finger protein 828 | −1.358 |
| | R\hat{ } | | | | |
| IPI:IPI00135443.2 | AS*PITNDGEDEFVPSDGLD | 1474 | Top2b | DNA topoisomerase 2-beta | −1.357 |
| | KDEYAFSSGK | | | | |
| IPI:IPI00227267.2 | EVENEQTPVS*EPEEEKGS | 1475 | Epb4.1l2 | Putative uncharacterized protein | −1.357 |
| | QPGPPVER | | | | |
| IPI:IPI00649691.3 | LASEDAALVDDDEES*DTPA | 1476 | Cc2d1b | Coiled-coil and C2 domain-containing | −1.353 |
| | QAPLAK@ | | | protein 1B | |
| IPI:IPI00340860.5 | TAS*EGSEAETPEAPK@ | 1477 | Larp7 | Isoform 1 of La-related protein 7 | −1.347 |
| IPI:IPI00320208.3 | YGPSSVEDTTGSGAADAKD | 1478 | Eef1b2 | Elongation factor 1-beta | −1.336 |
| | DDDIDLFGS*DDEEESEEAK | | | | |
| IPI:IPI00320208.3 | YGPSSVEDTTGSGAADAKD | 1479 | Eef1b2 | Elongation factor 1-beta | −1.336 |
| | DDDIDLFGS*DDEEESEEAK | | | | |
| IPI:IPI00750546.1 | GGGQSS*PQEEPTWK | 1480 | Cwc22 | Novel protein | −1.327 |
| IPI:IPI00127415.1 | CGSGPVHISGQHLVAVEED | 1481 | Npm1 | Nucleophosmin | −1.324 |
| | AES*EDEDEEDVK | | | | |
| IPI:IPI00123871.2 | VHEPPR\hat{ }EDTVPPK@PVPP | 1482 | Ncor2 | nuclear receptor co-repressor 2 | −1.31 |
| | VPPPTQHLQPEGDVSQQS | | | | |
| | GGS*PR\hat{ } | | | | |
| IPI:IPI00458583.3 | AKS*PQPPVEEEDEHFDDT | 1483 | Hnrnpu | Putative uncharacterized protein | −1.308 |
| | VVCLDTYNCDLHFK | | | | |
| IPI:IPI00124959.1 | GPIQTLGHTDESASDKGPT | 1484 | Mki67 | Ki-67 protein | −1.306 |
| | QMPCNSLQPEQVDSFQSS* | | | | |
| | PR | | | | |
| IPI:IPI00664670.4 | LGS*FGSITR | 1485 | Flnc | Isoform 1 of Filamin-C | −1.29 |
| IPI:IPI00664670.4 | LGS*FGSITR\hat{ } | 1486 | Flnc | Isoform 1 of Filamin-C | −1.289 |
| IPI:IPI00664670.4 | LGS*FGSITR\hat{ } | 1487 | Flnc | Isoform 1 of Filamin-C | −1.289 |
| IPI:IPI00664670.4 | LGS*FGSITR | 1488 | Flnc | Isoform 1 of Filamin-C | −1.289 |
| IPI:IPI00664670.4 | LGS*FGSITR | 1489 | Flnc | Isoform 1 of Filamin-C | −1.289 |
| IPI:IPI00270767.3 | M#K@ESETFSDSS*PIEIIDE | 1490 | Rtn4 | Isoform 2 of Reticulon-4 | −1.286 |
| | FPTFVSAK@ | | | | |
| IPI:IPI00318048.5 | SSPKEEVAS*EPEEAASPT* | 1491 | Nop56 | Nucleolar protein 56 | −1.284 |
| | TPK | | | | |
| IPI:IPI00276222.1 | LSIMTSENHLNNSDKEVDEV | 1492 | Fermt2 | Fermitin family homolog 2 | −1.279 |
| | DAALS*DLEITLEGGK | | | | |
| IPI:IPI00475055.3 | RCPS*T*DPEEAVEDAEGPS | 1493 | Zfhx3 | AT motif binding factor 1 | −1.274 |
| | EASADPEELAK | | | | |
| IPI:IPI00664670.4 | LGS*FGSITR\hat{ } | 1494 | Flnc | Isoform 1 of Filamin-C | −1.273 |
| IPI:IPI00664670.4 | LGS*FGSITR | 1495 | Flnc | Isoform 1 of Filamin-C | −1.273 |
| IPI:IPI00664670.4 | LGS*FGSITR\hat{ } | 1496 | Flnc | Isoform 1 of Filamin-C | −1.273 |
| IPI:IPI00664670.4 | LGS*FGSITR | 1497 | Flnc | Isoform 1 of Filamin-C | −1.273 |
| IPI:IPI00664670.4 | LGS*FGSITR\hat{ } | 1498 | Flnc | Isoform 1 of Filamin-C | −1.273 |
| IPI:IPI00664670.4 | LGS*FGSITR | 1499 | Flnc | Isoform 1 of Filamin-C | −1.273 |
| IPI:IPI00664670.4 | LGS*FGSITR\hat{ } | 1500 | Flnc | Isoform 1 of Filamin-C | −1.272 |
| IPI:IPI00127415.1 | CGSGPVHISGQHLVAVEED | 1501 | Npm1 | Nucleophosmin | −1.271 |
| | AES*EDEDEEDVK@ | | | | |
| IPI:IPI00121277.1 | VAAAAGSGPS*PPCS*PGH | 1502 | Pi4k2a | Phosphatidylinositol 4-kinase type 2- | −1.266 |
| | DR | | | alpha | |
| IPI:IPI00121277.1 | VAAAAGSGPS*PPCS*PGH | 1503 | Pi4k2a | Phosphatidylinositol 4-kinase type 2- | −1.266 |
| | DR\hat{ } | | | alpha | |
| IPI:IPI00317794.5 | K@EDS*DEDEDEEDEDDS* | 1504 | Ncl | Nucleolin | −1.255 |
| | DEDEDDEEEDEFEPPIVK@ | | | | |
| IPI:IPI00270767.3 | VVKEDGVM#S*PEK | 1505 | Rtn4 | Isoform 2 of Reticulon-4 | −1.252 |
| IPI:IPI00318938.6 | NS*PVAKT*PPKDLPAIPGVT | 1506 | Eif4ebp1 | Eukaryotic translation initiation | −1.238 |
| | SPTSDEPPM#QASQSQLPS | | | factor 4E-binding protein 1 | |
| | SPEDK | | | | |
| IPI:IPI00114352.1 | VNHEPEPASGAS*PGATIPK | 1507 | Bin1 | Isoform 1 of Myc box-dependent- | −1.237 |
| | @S*PSQLR\hat{ } | | | interacting protein 1 | |
| IPI:IPI00114352.1 | VNHEPEPASGAS*PGATIPK | 1508 | Bin1 | Isoform 1 of Myc box-dependent- | −1.237 |
| | S*PSQLR | | | interacting protein 1 | |
| IPI:IPI00110435.2 | TPS*PEPVDK@DFYSEFGD | 1509 | Nisch | Isoform 1 of Nischarin | −1.229 |
| | K@ | | | | |
| IPI:IPI00127415.1 | CGSGPVHISGQHLVAVEED | 1510 | Npm1 | Nucleophosmin | −1.226 |
| | AES*EDEDEEDVK@ | | | | |
| IPI:IPI00127415.1 | CGSGPVHISGQHLVAVEED | 1511 | Npm1 | Nucleophosmin | −1.226 |
| | AES*EDEDEEDVK@ | | | | |
| IPI:IPI00132386.1 | ANS*TSDSMFTETPSPVLK@ | 1512 | Cdc42ep3 | Cdc42 effector protein 3 | −1.225 |
| IPI:IPI00131999.2 | FELLPTPPLSPS*RR | 1513 | Myc | myc proto-oncogene protein | −1.219 |
| IPI:IPI00282957.4 | EPAAPAS*PAPS*PVPSPT*P | 1514 | Mtap7d1 | Isoform 1 of MAP7 domain-containing | −1.217 |
| | AQPQK | | | protein 1 | |
| IPI:IPI00318938.6 | NS*PVAKT*PPKDLPAIPGVT | 1515 | Eif4ebp1 | Eukaryotic translation initiation | −1.212 |
| | SPTSDEPPMQASQSQLPSS | | | factor 4E-binding protein 1 | |
| | PEDK | | | | |
| IPI:IPI00226220.2 | TFS*LDAAPADHSLGPSR\hat{ } | 1516 | Rtkn | Isoform 1 of Rhotekin | −1.207 |
| IPI:IPI00318938.6 | NS*PVAKT*PPKDLPAIPGVT | 1517 | Eif4ebp1 | Eukaryotic translation initiation | −1.203 |
| | SPTSDEPPM#QASQSQLPS | | | factor 4E-binding protein 1 | |
| | SPEDK | | | | |
| IPI:IPI00664670.4 | LGS*FGS*ITR\hat{ } | 1518 | Flnc | Isoform 1 of Filamin-C | −1.194 |
| IPI:IPI00473912.4 | NSTHSNLHTS*LGNS*VWG | 1519 | Gigyf2 | Isoform 1 of PERQ amino acid-rich with | −1.193 |
| | SINT*GPS*NQWASELVSSI | | | GYF domain-containing protein 2 | |
| | WSNADTK | | | | |
| IPI:IPI00227380.1 | TRS*EPLPPSATASPLLAPL | 1520 | Hdac7 | Isoform 4 of Histone deacetylase 7 | −1.178 |
| | QPR | | | | |
| IPI:IPI00119442.1 | M#DTGEVSDIGSQGAPIVLS | 1521 | Exosc9 | Exosome complex exonuclease RRP45 | −1.177 |
| | *DS*EEEEMIILEPEK@NPK@ | | | | |
| IPI:IPI00119442.1 | M#DTGEVSDIGSQGAPIVLS | 1522 | Exosc9 | Exosome complex exonuclease RRP45 | −1.177 |
| | *DS*EEEEMIILEPEKNPK | | | | |
| IPI:IPI00119442.1 | MDTGEVSDIGSQGAPIVLS* | 1523 | Exosc9 | Exosome complex exonuclease RRP45 | −1.177 |
| | DS*EEEEM#IILEPEKNPK | | | | |
| IPI:IPI00221882.1 | DHNS*EDEDEDKYADDIDM | 1524 | Slu7 | Pre-mRNA-splicing factor SLU7 | −1.167 |
| | #PGQNFDSK | | | | |
| IPI:IPI00675346.2 | R\hat{ }QSGT*DLQEDVIVR\hat{ } | 1525 | Myo9a | Isoform 2 of Myosin-IXa | −1.166 |
| IPI:IPI00125319.1 | TTS*FAESCKPVQQPSAFG | 1526 | Gsk3b | Glycogen synthase kinase-3 beta | −1.165 |
| | SMK | | | | |
| IPI:IPI00654192.2 | SLS*FSEPQQPPPTVK | 1527 | Zfp395 | zinc finger protein 395 | −1.165 |
| IPI:IPI00128975.3 | DKDDQEWEST*SPPKPTVFI | 1528 | Dap | Death-associated protein 1 | −1.165 |
| | SGVIAR | | | | |
| IPI:IPI00330897.1 | S*PVVITIDS*DS* | 1529 | Topors | E3 ubiquitin-protein ligase Topors | −1.161 |
| | DGESEVK@ | | | | |
| IPI:IPI00330133.2 | INDELEGLGLEGGS*EGEAPR | 1530 | Bend3 | BEN domain-containing protein 3 | −1.159 |
| IPI:IPI00330133.2 | INDELEGLGLEGGS*EGEAPR | 1531 | Bend3 | BEN domain-containing protein 3 | −1.159 |
| IPI:IPI00127415.1 | CGSGPVHISGQHLVAVEED | 1532 | Npm1 | Nucleophosmin | −1.157 |
| | AES*EDEDEEDVK@ | | | | |
| IPI:IPI00127415.1 | CGSGPVHISGQHLVAVEED | 1533 | Npm1 | Nucleophosmin | −1.157 |
| | AES*EDEDEEDVK | | | | |
| IPI:IPI00314749.2 | ISENYS*DKSDVENADESSS | 1534 | Slc4a4 | Isoform 1 of Electrogenic sodium | −1.156 |
| | SILKPLISPAAER | | | bicarbonate cotransporter 1 | |
| IPI:IPI00123709.1 | GPSEAPQEAEAEEGATS*D | 1535 | Akap12 | Isoform 1 of A-kinase anchor protein 12 | −1.154 |
| | GEK | | | | |
| IPI:IPI00321774.2 | HLLTDLPLPPELPGGDPS*P | 1536 | Crkrs | Isoform 2 of Cell division cycle 2- | −1.15 |
| | PDS*PEPK | | | related protein kinase 7 | |
| IPI:IPI00420601.5 | SDLIEDEELEDTGKGS*EDE | 1537 | Usp10 | Ubiquitin carboxyl-terminal hydrolase | −1.149 |
| | WEQVGPK | | | 10 | |
| IPI:IPI00421179.1 | EATLPPVS*PPK@ | 1538 | Eif4g1 | Isoform 1 of Eukaryotic translation | −1.146 |
| | | | | initiation factor 4 gamma 1 | |
| IPI:IPI00269136.2 | KGLVAASGS*DS*EDEDSM | 1539 | Tgif1 | Isoform 1 of Homeobox protein TGIF1 | −1.143 |
| | DSPLDLSSSAASGK | | | | |
| IPI:IPI00221581.1 | TGS*ESSQTGASATSGR | 1540 | Eif4b | Eukaryotic translation initiation | −1.142 |
| | | | | factor 4B | |
| IPI:IPI00756765.3 | ALVIQESESPPS*PPPS*PDR | 1541 | Ehmt2 | Euchromatic histone lysine N- | −1.141 |
| | \hat{ }R\hat{ } | | | methyltransferase 2 | |
| IPI:IPI00137668.1 | M#LPHAPGVQM#QAIPEDA | 1542 | Hdac2 | Histone deacetylase 2 | −1.123 |
| | VHEDS*GDEDGEDPDKR | | | | |
| IPI:IPI00225062.2 | IHTTALTGQS*PPLASGHQG | 1543 | Srrm2 | Isoform 3 of Serine/arginine repetitive | −1.123 |
| | EGDAPSVEPGATNIQQPSS* | | | matrix protein 2 | |
| | PAPSTK@ | | | | |
| IPI:IPI00225062.2 | IHTTALTGQS*PPLASGHQG | 1544 | Srrm2 | Isoform 3 of Serine/arginine repetitive | −1.123 |
| | EGDAPSVEPGATNIQQPSS* | | | matrix protein 2 | |
| | PAPSTK | | | | |
| IPI:IPI00225062.2 | IHTTALTGQSPPLASGHQG | 1545 | Srrm2 | Isoform 3 of Serine/arginine repetitive | −1.123 |
| | EGDAPS*VEPGATNIQQPSS | | | matrix protein 2 | |
| | *PAPSTK | | | | |
| IPI:IPI00339428.9 | MSSHT*ETSSFLQTLTGR\hat{ } | 1546 | Dock7 | Isoform 2 of Dedicator of cytokinesis | −1.12 |
| | | | | protein 7 | |
| IPI:IPI00339428.9 | MS*SHTETSSFLQTLTGR\hat{ } | 1547 | Dock7 | Isoform 2 of Dedicator of cytokinesis | −1.12 |
| | | | | protein 7 | |
| IPI:IPI00339428.9 | MS*SHTETSSFLQTLTGR | 1548 | Dock7 | Isoform 2 of Dedicator of cytokinesis | −1.12 |
| | | | | protein 7 | |
| IPI:IPI00127415.1 | CGSGPVHISGQHLVAVEED | 1549 | Npm1 | Nucleophosmin | −1.119 |
| | AES*EDEDEEDVK@ | | | | |
| IPI:IPI00127415.1 | CGSGPVHISGQHLVAVEED | 1550 | Npm1 | Nucleophosmin | −1.119 |
| | AES*EDEDEEDVK@ | | | | |
| IPI:IPI00331361.2 | S*PSLLQSGVK | 1551 | Mybbp1a | Myb-binding protein 1A | −1.116 |
| IPI:IPI00309059.7 | ST*S*PIIGSPPVR\hat{ } | 1552 | Patl1 | Protein PAT1 homolog 1 | −1.115 |
| IPI:IPI00339428.9 | MSSHTETS*SFLQTLTGR\hat{ } | 1553 | Dock7 | Isoform 2 of Dedicator of cytokinesis | −1.114 |
| | | | | protein 7 | |
| IPI:IPI00123747.1 | LPS*M#GDQEPPGQEK | 1554 | Slc7a11 | Cystine/glutamate transporter | −1.114 |
| IPI:IPI00322095.4 | IDEPNTPYHNMIGDDEDAYS | 1555 | Ppp1r2 | Putative uncharacterized protein | −1.107 |
| | DS*EGNEVM#TPDILAK | | | | |
| IPI:IPI00135379.3 | LSEGEPGPVAAGEQLSEHP | 1556 | Myo9b | Isoform 1 of Myosin-IXb | −1.106 |
| | VEDPESLGVEAETWMNKS* | | | | |
| | PDGMS*PK | | | | |
| IPI:IPI00125319.1 | TTS*FAESCKPVQQPSAFG | 1557 | Gsk3b | Glycogen synthase kinase-3 beta | −1.105 |
| | SMK | | | | |
| IPI:IPI00125319.1 | TTS*FAESCK@PVQQPSAF | 1558 | Gsk3b | Glycogen synthase kinase-3 beta | −1.105 |
| | GSMK@ | | | | |
| IPI:IPI00125319.1 | TTS*FAESCKPVQQPSAFG | 1559 | Gsk3b | Glycogen synthase kinase-3 beta | −1.103 |
| | SMK | | | | |
| IPI:IPI00320208.3 | YGPSSVEDTTGSGAADAKD | 1560 | Eef1b2 | Elongation factor 1-beta | −1.102 |
| | DDDIDLFGS*DDEEESEEAKK | | | | |
| IPI:IPI00331361.2 | SPAPSNPTLS*PS*TPAK@T | 1561 | Mybbp1a | Myb-binding protein 1A | −1.101 |
| | PK@ | | | | |
| IPI:IPI00331361.2 | SPAPSNPTLS*PST*PAKTPK | 1562 | Mybbp1a | Myb-binding protein 1A | −1.101 |
| IPI:IPI00668709.2 | SSS*APNVHINTI | 1563 | Braf | B-Raf protein | −1.097 |
| | EPVNIDDLIR | | | | |
| IPI:IPI00462466.6 | K@LDES*PVLK@PEFIGHD | 1564 | Setd2 | SET domain containing 2 | −1.096 |
| | GR\hat{ } | | | | |
| IPI:IPI00462466.6 | KLDES*PVLKPEFIGHDGR | 1565 | Setd2 | SET domain containing 2 | −1.096 |
| IPI:IPI00108389.5 | LLDGPTGS*S*EEEEEFLEIP | 1566 | Trp53bp1 | Transformation related protein 53 | −1.094 |
| | PFNK | | | binding protein 1 | |
| IPI:IPI00668709.2 | SSS*APNVHINTIEP | 1567 | Braf | B-Raf protein | −1.088 |
| | VNIDDLIR | | | | |
| IPI:IPI00225062.2 | DGSGT*PSRHSLS*GSS*PG | 1568 | Srrm2 | Isoform 3 of Serine/arginine repetitive | −1.081 |
| | M#KDTPQT*PSR | | | matrix protein 2 | |
| IPI:IPI00135971.1 | VQIPVSHPDPEPVS*DNEDD | 1569 | Tjp1 | Tight junction protein ZO-1 | −1.08 |
| | S*YDEEVHDPR | | | | |
| IPI:IPI00127415.1 | CGSGPVHISGQHLVAVEED | 1570 | Npm1 | Nucleophosmin | −1.077 |
| | AES*EDEDEEDVK | | | | |
| IPI:IPI00111169.1 | HIVSNDSSDS*DDEAQGPK@ | 1571 | Pla2g4a | Cytosolic phospholipase A2 | −1.072 |
| IPI:IPI00225670.1 | EVHDELEDLPS*PPPPLSPP | 1572 | Gphn | Gephyrin | −1.07 |
| | PT*TSPHK | | | | |
| IPI:IPI00225670.1 | EVHDELEDLPS*PPPPLS*P | 1573 | Gphn | Gephyrin | −1.07 |
| | PPTTSPHK@ | | | | |
| IPI:IPI00123709.1 | AEDS*GAEQLASEIEPSR | 1574 | Akap12 | Isoform 1 of A-kinase anchor protein 12 | −1.067 |
| IPI:IPI00123709.1 | AEDS*GAEQLASEIEPSR | 1575 | Akap12 | Isoform 1 of A-kinase anchor protein 12 | −1.067 |
| IPI:IPI00454109.2 | S*PPACSSSSSSLFSAVVAR\hat{ } | 1576 | Erf | ETS domain-containing transcription | −1.066 |
| | | | | factor ERF | |
| IPI:IPI00313517.1 | GPPS*EDEGMDIHFEEGVLS | 1577 | | | −1.066 |
| | PSAADMR\hat{ }PEPPNSLDLNG | | | | |
| | SHPR\hat{ } | | | | |
| IPI:IPI00313517.1 | GPPS*EDEGMDIHFEEGVLS | 1578 | | | −1.066 |
| | PSAADMRPEPPNSLDLNGS | | | | |
| | HPR | | | | |
| IPI:IPI00311375.6 | S*GITSLLFGEDDLEALK | 1579 | Sgsm3 | small G protein signaling modulator 3 | −1.061 |
| IPI:IPI00311490.4 | VLS*DS*EEEEKDADVPGTS | 1580 | Phip | PH-interacting protein | −1.059 |
| | TR | | | | |
| IPI:IPI00553798.2 | GHYEVT*GSDDEAGKLQGS | 1581 | Ahnak | AHNAK nucleoprotein isoform 1 | −1.057 |
| | GVSLASK | | | | |
| IPI:IPI00553798.2 | GHYEVTGS*DDEAGK@LQ | 1582 | Ahnak | AHNAK nucleoprotein isoform 1 | −1.057 |
| | GSGVSLASK@ | | | | |
| IPI:IPI00336281.1 | RDSICS*SVSMESSLAEPQD | 1583 | Golga3 | Isoform 1 of Golgin subfamily A | −1.056 |
| | ELLQILK | | | member 3 | |
| IPI:IPI00229859.1 | ALENGEADEPS*FS*DPEDF | 1584 | Eif3b | Eif3b protein | −1.053 |
| | VDDVSEEELLGDVLK | | | | |
| IPI:IPI00668709.2 | DRSSS*APNVHINTIEPVNID | 1585 | Braf | B-Raf protein | −1.048 |
| | DLIR | | | | |
| IPI:IPI00668709.2 | DR\hat{ }SSS*APNVHINTIEPVNI | 1586 | Braf | B-Raf protein | −1.048 |
| | DDLIR\hat{ } | | | | |
| IPI:IPI00125319.1 | T*TSFAESCKPVQQPSAFG | 1587 | Gsk3b | Glycogen synthase kinase-3 beta | −1.047 |
| | SMK | | | | |
| IPI:IPI00135660.5 | RGNNSAVGS*NADLT*IEED | 1588 | Sdpr | Serum deprivation-response protein | −1.046 |
| | EEEEPVALQQAQQVR | | | | |
| IPI:IPI00339428.9 | MSS*HTETSSFLQTLTGR | 1589 | Dock7 | Isoform 2 of Dedicator of cytokinesis | −1.042 |
| | | | | protein 7 | |
| IPI:IPI00127415.1 | CGSGPVHISGQHLVAVEED | 1590 | Npm1 | Nucleophosmin | −1.039 |
| | AES*EDEDEEDVK | | | | |
| IPI:IPI00115660.1 | GPILAT*PGK@ | 1591 | Tcof1 | Treacle protein | −1.038 |
| IPI:IPI00115660.1 | GPILAT*PGK | 1592 | Tcof1 | Treacle protein | −1.038 |
| IPI:IPI00115660.1 | GPILAT*PGK | 1593 | Tcof1 | Treacle protein | −1.038 |
| IPI:IPI00115660.1 | GPILAT*PGK@ | 1594 | Tcof1 | Treacle protein | −1.038 |
| IPI:IPI00230704.4 | KES*APQVLLPEEEK | 1595 | Arhgef7 | Isoform C of Rho guanine nucleotide | −1.036 |
| | | | | exchange factor 7 | |
| IPI:IPI00320422.3 | LQEEGGS*EEEEAGNPSED | 1596 | Pwp1 | Periodic tryptophan protein 1 homolog | −1.035 |
| | GM#QSGPTQAPPR | | | | |
| IPI:IPI00270767.3 | VVKEDGVM#S*PEK | 1597 | Rtn4 | Isoform 2 of Reticulon-4 | −1.035 |
| IPI:IPI00331306.7 | ARQPSQADTGEEDSDEDY* | 1598 | Sh3bp2 | SH3-domain binding protein 2 isoform c | −1.034 |
| | EKVPLPNSVFVNTTESCEV | | | | |
| | ER | | | | |
| IPI:IPI00137864.1 | YDLSNQDHIMDAPPLS*PFP | 1599 | Rbl1 | Isoform Long of Retinoblastoma-like | −1.03 |
| | HIK@ | | | protein 1 | |
| IPI:IPI00137864.1 | YDLSNQDHIMDAPPLS*PFP | 1600 | Rbl1 | Isoform Long of Retinoblastoma-like | −1.03 |
| | HIK | | | protein 1 | |
| IPI:IPI00320208.3 | YGPSSVEDTTGSGAADAK | 1601 | Eef1b2 | Elongation factor 1-beta | −1.028 |
| | @DDDDIDLFGS*DDEEESE | | | | |
| | EAK@K@ | | | | |
| IPI:IPI00404545.2 | SLS*S*PTVTLSAPLEGAK | 1602 | Nedd4l | Isoform 3 of E3 ubiquitin-protein | −1.027 |
| | | | | ligase NEDD4-like | |
| IPI:IPI00127764.2 | VIEDEDGAAAAATVSNSEET | 1603 | Pcm1 | Isoform 1 of Pericentriolar material 1 | −1.024 |
| | PIIENHNS*PQPISDVSAVPC | | | protein | |
| | PR\hat{ } | | | | |
| IPI:IPI00131884.1 | TT*PAPS*PGS*ANESFFAPS | 1604 | Mid1ip1 | Mid1-interacting protein 1 | −1.024 |
| | R\hat{ } | | | | |
| IPI:IPI00131884.1 | TT*PAPS*PGS*ANESFFAPS | 1605 | Mid1ip1 | Mid1-interacting protein 1 | −1.024 |
| | R\hat{ } | | | | |
| IPI:IPI00131884.1 | TT*PAPS*PGS*ANESF | 1606 | Mid1ip1 | Mid1-interacting protein 1 | −1.024 |
| | FAPSR | | | | |
| IPI:IPI00400381.9 | LGST*GS*QPNSEAEPGPEH | 1607 | Phactr4 | Isoform 1 of Phosphatase and actin | −1.02 |
| | APK | | | regulator 4 | |
| IPI:IPI00127415.1 | CGSGPVHISGQHLVAVEED | 1608 | Npm1 | Nucleophosmin | −1.018 |
| | AES*EDEDEEDVK | | | | |
| IPI:IPI00756765.3 | ALVIQESESPPS*PPPS*PDR | 1609 | Ehmt2 | Euchromatic histone lysine N- | −1.017 |
| | \hat{ }R\hat{ } | | | methyltransferase 2 | |
| IPI:IPI00136917.5 | TDQEGLNASQPT*PPPLPK@ | 1610 | C230081A13Rik | Tyrosine-protein kinase-protein kinase | −1.016 |
| IPI:IPI00127415.1 | CGSGPVHISGQHLVAVEED | 1611 | Npm1 | Nucleophosmin | −1.015 |
| | AES*EDEDEEDVK | | | | |
| IPI:IPI00453673.4 | RAS*DGGANIQLHAQQLLK | 1612 | BC033915 | serine/threonine-protein kinase QSK | −1.015 |
| IPI:IPI00453837.2 | KLPPPPPQAPPEEENES*EP | 1613 | Kdm1 | Lysine-specific histone demethylase 1 | −1.014 |
| | EEPSGVEGAAFQSR | | | | |
| IPI:IPI00227900.1 | FKGPGDTSNFDDYEEEEIR | 1614 | Prkaca | Isoform 2 of cAMP-dependent protein | −1.01 |
| | VS*INEK | | | kinase catalytic subunit alpha | |
| IPI:IPI00125662.2 | VDPTYGLESSCIAGTGPDE | 1615 | Smarcc1 | Isoform 1 of SWI/SNF complex subunit | −1.007 |
| | PEK@LEGS*EEEK@M#ETD | | | SMARCC1 | |
| | PDGQQPEK@ | | | | |
| IPI:IPI00677756.1 | KLS*FSMPR | 1616 | Ahnak2 | Putative uncharacterized protein | −1.006 |
| IPI:IPI00395213.1 | LDFLPEMMVDHCS*LNSSP | 1617 | Cugbp1 | Isoform 4 of CUG-BP- and ETR-3-like | −1.002 |
| | VSK@K@ | | | factor 1 | |

Table 8. Gene names and sequences of the phosphopeptides whose intensities decrease after Ku-0063794 treatment. Note that the light cells were treated with rapamycin and serve as controls whereas the heavy cells were treated with a combination of rapamycin and Ku-0063794.

Table 9(a)

| | | | SEQ ID | Gene | | |
|---|---|---|---|---|---|---|
| ID | Reference | Ascore Seq | NO | Name | Annotation | Class |
| 1 | IPI:IPI00121418.1 | DGEGPDNLEPACPLSLPLQ | 1618 | Rb1 | Retinoblastoma-associated | 1 |
| | | GNHTAADMYLS*PLRS*PK | | | protein | |
| 2 | IPI:IPI00221581.1 | TGS*ESS*QTGASATSGR | 1619 | Eif4b | Eukaryotic translation initiation | 1 |
| | | | | | factor 4B | |
| 3 | IPI:IPI00929786.1 | T*ASISSS*PSEGTPAVGSY | 1620 | Larp1 | Isoform 1 of La-related protein 1 | 1 |
| | | GCT*PQS*LPK@ | | | | |
| 4 | IPI:IPI00929786.1 | T*ASISSS*PSEGTPAVGSY* | 1621 | Larp1 | Isoform 1 of La-related protein 1 | 1 |
| | | GCT*PQSLPK@ | | | | |
| 5 | IPI:IPI00929786.1 | T*ASISSS*PSEGTPAVGS*Y | 1622 | Larp1 | Isoform 1 of La-related protein 1 | 1 |
| | | GCTPQSLPK@ | | | | |
| 6 | IPI:IPI00317401.6 | AES*PETSAVESTQST*PQK@ | 1623 | Pds5b | Isoform 1 of Sister chromatid | 1 |
| | | | | | cohesion protein PDS5 homolog B | |
| 7 | IPI:IPI00107958.1 | TSDIFGS*PVTATAPLAHPN | 1624 | Hn1l | Hematological and neurological | 1 |
| | | K@PK@ | | | expressed 1-like protein | |
| 8 | IPI:IPI00108454.2 | R\hat{ }LS*S*LR\hat{ } | 1625 | Rps6 | 29 kDa protein | 1 |
| 9 | IPI:IPI00225062.2 | R\hat{ }SSS*ELS*PEVVEK@ | 1626 | Srrm2 | Isoform 3 of Serine/arginine | 1 |
| | | | | | repetitive matrix protein 2 | |
| 10 | IPI:IPI00761759.1 | S*SS*GS*EHSTEGSVSLGD | 1627 | Larp4 | Putative uncharacterized protein | 1 |
| | | GPLSR\hat{ } | | | | |
| 11 | IPI:IPI00761759.1 | SSS*GSEHS*T*EGSVSLGD | 1628 | Larp4 | Putative uncharacterized protein | 1 |
| | | GPLSR\hat{ } | | | | |
| 12 | IPI:IPI00225062.2 | RSS*SELS*PEVVEK | 1629 | Srrm2 | Isoform 3 of Serine/arginine | 1 |
| | | | | | repetitive matrix protein 2 | |
| 13 | IPI:IPI00122594.4 | TTPLAS*PSLS*PGR\hat{ } | 1630 | Ahctf1 | AT-hook-containing transcription | 1 |
| | | | | | factor 1 | |
| 14 | IPI:IPI00136107.1 | THS*TSS*S*IGSGESPFSR\hat{ } | 1631 | Ndrg3 | Protein NDRG3 | 1 |
| 15 | IPI:IPI00136107.1 | THS*TSSS*IGSGESPFSR\hat{ } | 1632 | Ndrg3 | Protein NDRG3 | 1 |
| 16 | IPI:IPI00136107.1 | THS*TSS*SIGSGESPFSR\hat{ } | 1633 | Ndrg3 | Protein NDRG3 | 1 |
| 17 | IPI:IPI00136107.1 | THS*TSSSIGSGESPFSR\hat{ } | 1634 | Ndrg3 | Protein NDRG3 | 1 |
| 18 | IPI:IPI00136107.1 | T*HST*SSS*IGSGESPFSR\hat{ } | 1635 | Ndrg3 | Protein NDRG3 | 1 |
| 19 | IPI:IPI00136107.1 | THST*SSS*IGSGESPFSR\hat{ } | 1636 | Ndrg3 | Protein NDRG3 | 1 |
| 20 | IPI:IPI00136107.1 | THSTSS*S*IGSGESPFSR\hat{ } | 1637 | Ndrg3 | Protein NDRG3 | 1 |
| 21 | IPI:IPI00136107.1 | T*HSTSSSIGS*GESPFSR\hat{ } | 1638 | Ndrg3 | Protein NDRG3 | 1 |
| 22 | IPI:IPI00136107.1 | THST*SSSIGSGESPFSR\hat{ } | 1639 | Ndrg3 | Protein NDRG3 | 1 |
| 23 | IPI:IPI00551454.3 | ER\hat{ }QES*ESEQELVNK@ | 1640 | Pdcd11 | Protein RRP5 homolog | 1 |
| 24 | IPI:IPI00117229.3 | TS*PAGGTWSSVVSGVPR | 1641 | Atxn2 | ataxin 2 | 1 |
| 25 | IPI:IPI00348442.1 | SK@FDS*DEEDEDAENLEA | 1642 | Sfrs18 | splicing factor arginine/serine-rich | 1 |
| | | VSSGK@ | | | 18 | |
| 26 | IPI:IPI00556837.1 | DTVIIVS*EPS*EDEESHDLP | 1643 | Smarcad1 | Isoform 1 of SWI/SNF-related | 1 |
| | | SVTR | | | matrix-associated actin- | |
| | | | | | dependent regulator of chromatin | |
| | | | | | subfamily A containing DEAD/H | |
| | | | | | box 1 | |
| 27 | IPI:IPI00153986.2 | GTS*RPGT*PSAEAASTSST | 1644 | Gtf2f1 | General transcription factor IIF | 1 |
| | | LR | | | subunit 1 | |
| 28 | IPI:IPI00225062.2 | GCS*PPKS*PEKPPQSTSS* | 1645 | Srrm2 | Isoform 3 of Serine/arginine | 1 |
| | | ESCPPSPQPTK | | | repetitive matrix protein 2 | |
| 29 | IPI:IPI00128904.1 | VMTIPYQPMPASS*PVICAG | 1646 | Pcbp1 | Poly(rC)-binding protein 1 | 1 |
| | | GQDR | | | | |
| 30 | IPI:IPI00121251.7 | LSTTPS*PTNS*LHEDGVDD | 1647 | Tox4 | TOX high mobility group box | 1 |
| | | FRR | | | family member 4 | |
| 31 | IPI:IPI00320594.5 | SQPHSSTSNQETS*DS*EM | 1648 | Ranbp10 | Ran-binding protein 10 | 1 |
| | | EMEAEHYPNGVLESVSTR\hat{ } | | | | |
| 32 | IPI:IPI00808277.2 | SAPAS*PNHAGVLSAHSSG | 1649 | Foxk2 | Isoform 1 of Forkhead box | 1 |
| | | AQT*PES*LSR\hat{ } | | | protein K2 | |
| 33 | IPI:IPI00137166.1 | SR\hat{ }DAT*PPVS*PINMEDQE | 1650 | Junb | Transcription factor jun-B | 1 |
| | | R\hat{ } | | | | |
| 34 | IPI:IPI00454104.1 | TTEAPCSPGS*QQPPS*PD | 1651 | Scrib | Isoform 1 of Protein LAP4 | 1 |
| | | ELPANVK@ | | | | |
| 35 | IPI:IPI00225062.2 | GCS*PPK@S*PEK@PPQST | 1652 | Srrm2 | Isoform 3 of Serine/arginine | 1 |
| | | SSESCPPS*PQPTK@ | | | repetitive matrix protein 2 | |
| 36 | IPI:IPI00153986.2 | GTSR\hat{ }PGT*PS*AEAASTSS | 1653 | Gtf2f1 | General transcription factor IIF | 1 |
| | | TLR\hat{ } | | | subunit 1 | |
| 37 | IPI:IPI00753321.2 | SVSET*SEDK@K@DEES*D | 1654 | Bod1l | biorientation of chromosomes in | 1 |
| | | EEEEEEEEEEPLGATTR\hat{ } | | | cell division 1-like | |
| 38 | IPI:IPI00153986.2 | GT*SR\hat{ }PGT*PSAEAASTSS | 1655 | Gtf2f1 | General transcription factor IIF | 1 |
| | | TLR\hat{ } | | | subunit 1 | |
| 39 | IPI:IPI00153986.2 | GTS*R\hat{ }PGTPS*AEAASTSS | 1656 | Gtf2f1 | General transcription factor IIF | 1 |
| | | TLR\hat{ } | | | subunit 1 | |
| 40 | IPI:IPI00929786.1 | TASIS*S*SPSEGTPAVGSY | 1657 | Larp1 | Isoform 1 of La-related protein 1 | 1 |
| | | GCT*PQSLPK@ | | | | |
| 41 | IPI:IPI00929786.1 | TAS*ISSS*PSEGTPAVGSY | 1658 | Larp1 | Isoform 1 of La-related protein 1 | 1 |
| | | GCT*PQS*LPK@ | | | | |
| 42 | IPI:IPI00130920.1 | RSES*PFEGK | 1659 | Mtap1b | Microtubule-associated protein | 1 |
| | | | | | 1B | |
| 43 | IPI:IPI00336973.2 | GLNLDGTPALSTLGGFS*PA | 1660 | Ccnl1 | Isoform 1 of Cyclin-L1 | 1 |
| | | SK@PS*SPR\hat{ } | | | | |
| 44 | IPI:IPI00336973.2 | GLNLDGTPALSTLGGFS*PA | 1661 | Ccnl1 | Isoform 1 of Cyclin-L1 | 1 |
| | | S*KPSSPR | | | | |
| 45 | IPI:IPI00336973.2 | GLNLDGT*PALSTLGGFS*P | 1662 | Ccnl1 | Isoform 1 of Cyclin-L1 | 1 |
| | | ASK@PS*SPR\hat{ } | | | | |
| 46 | IPI:IPI00336973.2 | GLNLDGTPALSTLGGFSPA | 1663 | Ccnl1 | Isoform 1 of Cyclin-L1 | 1 |
| | | S*K@PS*SPR\hat{ } | | | | |
| 47 | IPI:IPI00379844.4 | TAS*EGDGGAAGGAGTAG | 1664 | Irs2 | Insulin receptor substrate 2 | 1 |
| | | GR\hat{ }PMSVAGS*PLS*PGPV | | | | |
| | | R\hat{ } | | | | |
| 48 | IPI:IPI00336973.2 | GLNLDGTPALSTLGGFS*PA | 1665 | Ccnl1 | Isoform 1 of Cyclin-L1 | 1 |
| | | SKPSS*PR | | | | |
| 49 | IPI:IPI00336973.2 | GLNLDGT*PALSTLGGFS*P | 1666 | Ccnl1 | Isoform 1 of Cyclin-L1 | 1 |
| | | ASKPSS*PR | | | | |
| 50 | IPI:IPI00656285.2 | SLVS*PIPS*PTGTISVPNSC | 1667 | Foxk1 | Forkhead box protein K1 | 1 |
| | | PAS*PR\hat{ } | | | | |
| 51 | IPI:IPI00656285.2 | SLVSPIPSPT*GT*ISVPNSC | 1668 | Foxk1 | Forkhead box protein K1 | 1 |
| | | PAS*PR | | | | |
| 52 | IPI:IPI00454104.1 | TTEAPCS*PGSQQPPS*PD | 1669 | Scrib | Isoform 1 of Protein LAP4 | 1 |
| | | ELPANVK@ | | | | |
| 53 | IPI:IPI00753321.2 | S*VSETSEDK@K@DEES*D | 1670 | Bod1l | biorientation of chromosomes in | 1 |
| | | EEEEEEEEEEPLGATTR\hat{ } | | | cell division 1-like | |
| 54 | IPI:IPI00753321.2 | SVSETS*EDK@K@DEES*D | 1671 | Bod1l | biorientation of chromosomes in | 1 |
| | | EEEEEEEEEEPLGATTR\hat{ } | | | cell division 1-like | |
| 55 | IPI:IPI00313307.3 | SYQNS*PSS*EDGIR\hat{ }PLPE | 1672 | Med1 | Isoform 4 of Mediator of RNA | 1 |
| | | YSTEK@ | | | polymerase II transcription | |
| | | | | | subunit 1 | |
| 56 | IPI:IPI00225062.2 | GCS*PPK@S*PEK@PPQS* | 1673 | Srrm2 | Isoform 3 of Serine/arginine | 1 |
| | | TSSESCPPSPQPTK@ | | | repetitive matrix protein 2 | |
| 57 | IPI:IPI00318938.6 | NS*PVAK@TPPK@DLPAIP | 1674 | Eif4ebp1 | Eukaryotic translation initiation | 1 |
| | | GVTSPTS*DEPPMQASQSQ | | | factor 4E-binding protein 1 | |
| | | LPSSPEDK@ | | | | |
| 58 | IPI:IPI00130920.1 | SLMSS*PEDLTK@DFEELK | 1675 | MtaP1b | Microtubule-associated protein | 1 |
| | | @AEEIDVAK@ | | | 1B | |
| 59 | IPI:IPI00553798.2 | SS*EVVLS*GDDEDYQR\hat{ } | 1676 | Ahnak | AHNAK nucleoprotein isoform 1 | 1 |
| 60 | IPI:IPI00129264.1 | ATSR\hat{ }PINLGPSS*PNTEIH | 1677 | Sorbs3 | Vinexin | 1 |
| | | WTPYR\hat{ } | | | | |
| 61 | IPI:IPI00129264.1 | ATSRPINLGPS*SPNTEIHW | 1678 | Sorbs3 | Vinexin | 1 |
| | | TPYR | | | | |
| 62 | IPI:IPI00123410.5 | TIS*AQDTLAYATALLNEK@ | 1679 | Usp24 | Isoform 1 of Ubiquitin carboxyl- | 1 |
| | | | | | terminal hydrolase 24 | |
| 63 | IPI:IPI00226441.2 | LHYT*PPLQS*PIT*DGDPLL | 1680 | Lin9 | Isoform 2 of Lin-9 homolog | 1 |
| | | GQS*PWR\hat{ } | | | | |
| 64 | IPI:IPI00309059.7 | STS*PIIGS*PPVR\hat{ } | 1681 | Patl1 | Protein PAT1 homolog 1 | 1 |
| 65 | IPI:IPI00336713.1 | CS*PVPGLSSS*PSGSPLHG | 1682 | Bcas3 | Isoform 1 of Breast carcinoma- | 1 |
| | | K@ | | | amplified sequence 3 homolog | |
| 66 | IPI:IPI00320905.7 | LGEQGPEPGPT*PPQTPT* | 1683 | Arhgap17 | Isoform 1 of Rho GTPase- | 1 |
| | | PPS*TPPLAK | | | activating protein 17 | |
| 67 | IPI:IPI00309059.7 | R\hat{ }S*TS*PIIGS*PPVR\hat{ } | 1684 | Patl1 | Protein PAT1 homolog 1 | 1 |
| 68 | IPI:IPI00309059.7 | RSTS*PIIGS*PPVR | 1685 | Patl1 | Protein PAT1 homolog 1 | 1 |
| 69 | IPI:IPI00309059.7 | ST*S*PIIGS*PPVR\hat{ } | 1686 | Patl1 | Protein PAT1 homolog 1 | 1 |
| 70 | IPI:IPI00317599.3 | SQEDEEEIST*SPGVSEFVS | 1687 | Syap1 | Synapse-associated protein 1 | 1 |
| | | DAFDTCSLNQEDLRK | | | | |
| 71 | IPI:IPI00317599.3 | SQEDEEEISTS*PGVSEFVS | 1688 | Syap1 | Synapse-associated protein 1 | 1 |
| | | DAFDTCSLNQEDLRK | | | | |
| 72 | IPI:IPI00656285.2 | SLVS*PIPSPT*GTISVPNS* | 1689 | Foxk1 | Forkhead box protein K1 | 1 |
| | | CPASPR\hat{ } | | | | |
| 73 | IPI:IPI00676574.2 | RVS*TDLPEGQDVYTAACN | 1690 | Herc1 | hect (homologous to the E6-AP | 1 |
| | | SVIHR | | | (UBE3A) carboxyl terminus) | |
| | | | | | domain and RCC1 (CHC1)-like | |
| | | | | | domain (RLD) 1 | |
| 74 | IPI:IPI00458958.2 | DWDK@ES*EGEEPAGGR\hat{ } | 1691 | Rrp15 | RRP15-like protein | 2 |
| 75 | IPI:IPI00230719.8 | R\hat{ }DS*SDDWEIPDGQITVGQ | 1692 | Braf | Isoform 1 of B-Raf proto- | 2 |
| | | R\hat{ } | | | oncogene serine/threonine- | |
| | | | | | protein kinase | |
| 76 | IPI:IPI00318938.6 | VALGDGVQLPPGDYSTT*P | 1693 | Eif4ebp1 | Eukaryotic translation initiation | 2 |
| | | GGTLFSTTPGGT*R | | | factor 4E-binding protein 1 | |
| 77 | IPI:IPI00874995.2 | AS*DDLGEPDVFATAPFR | 1694 | Aak1 | Uncharacterized protein | 2 |
| | | | | | FLJ45252 homolog | |
| 78 | IPI:IPI00318938.6 | VALGDGVQLPPGDYSTT*P | 1695 | Eif4ebp1 | Eukaryotic translation initiation | 2 |
| | | GGTLFSTT*PGGTR\hat{ } | | | factor 4E-binding protein 1 | |
| 79 | IPI:IPI00318938.6 | VALGDGVQLPPGDYSTTPG | 1696 | Eif4ebp1 | Eukaryotic translation initiation | 2 |
| | | GT*LFSTT*PGGTR | | | factor 4E-binding protein 1 | |
| 80 | IPI:IPI00318938.6 | VALGDGVQLPPGDYS*TTP | 1697 | Eif4ebp1 | Eukaryotic translation initiation | 2 |
| | | GGTLFSTT*PGGTR\hat{ } | | | factor 4E-binding protein 1 | |
| 81 | IPI:IPI00323045.3 | S*MDVDLNQAHMEDTPK@ | 1698 | Melk | Maternal embryonic leucine | 2 |
| | | | | | zipper kinase | |
| 82 | IPI:IPI00623284.4 | IWDPTPS*HTPAGAAT*PGR | 1699 | Sf3b1 | Splicing factor 3B subunit 1 | 2 |
| | | GDT*PGHAT*PGHGGATSS | | | | |
| | | AR | | | | |
| 83 | IPI:IPI00125960.1 | TAS*GSS*VTSLEGTR | 1700 | Ndrg1 | Protein NDRG1 | 2 |
| 84 | IPI:IPI00318938.6 | R\hat{ }VALGDGVQLPPGDYSTT | 1701 | Eif4ebp1 | Eukaryotic translation initiation | 2 |
| | | *PGGTLFSTT*PGGTR\hat{ } | | | factor 4E-binding protein 1 | |
| 85 | IPI:IPI00229571.1 | IS*SK@S*PGHMVILNQTK@ | 1702 | Sltm | Isoform 1 of SAFB-like | 2 |
| | | | | | transcription modulator | |
| 86 | IPI:IPI00120095.2 | DFTK@PQDGDIIAPLIT*PLK@ | 1703 | Samhd1 | SAM domain and HD domain- | 2 |
| | | | | | containing protein 1 | |
| 87 | IPI:IPI00340860.5 | T*AS*EGSEAETPEAPK | 1704 | Larp7 | Isoform 1 of La-related protein 7 | 2 |
| 88 | IPI:IPI00664670.4 | LGS*FGSITR\hat{ } | 1705 | Flnc | Isoform 1 of Filamin-C | 2 |
| 89 | IPI:IPI00654192.2 | SLS*FSEPQQPPPTVK@ | 1706 | Zfp395 | zinc finger protein 395 | 2 |
| 90 | IPI:IPI00225062.2 | IHTTALTGQS*PPLASGHQG | 1707 | Srrm2 | Isoform 3 of Serine/arginine | 2 |
| | | EGDAPSVEPGATNIQQPSS | | | repetitive matrix protein 2 | |
| | | *PAPSTK | | | | |
| 91 | IPI:IPI00225062.2 | IHTTALTGQS*PPLASGHQG | 1708 | Srrm2 | Isoform 3 of Serine/arginine | 2 |
| | | EGDAPSVEPGATNIQQPSS | | | repetitive matrix protein 2 | |
| | | *PAPS*TK@ | | | | |
| 92 | IPI:IPI00225062.2 | IHTTALTGQS*PPLASGHQG | 1709 | Srrm2 | Isoform 3 of Serine/arginine | 2 |
| | | EGDAPSVEPGATNIQQPSS | | | repetitive matrix protein 2 | |
| | | *PAPST*K | | | | |
| 93 | IPI:IPI00339428.9 | MSSHT*ETSSFLQTLTGR | 1710 | Dock7 | Isoform 2 of Dedicator of | 2 |
| | | | | | cytokinesis protein 7 | |
| 94 | IPI:IPI00225062.2 | DGSGT*PSRHSLS*GS*S*P | 1711 | Srrm2 | Isoform 3 of Serine/arginine | 2 |
| | | GMKDTPQT*PSR | | | repetitive matrix protein 2 | |
| 95 | IPI:IPI00668709.2 | DRSSS*APNVHINTIEPVNID | 1712 | Braf | B-Raf protein | 2 |
| | | DLIR | | | | |
| 96 | IPI:IPI00135660.5 | RGNNS*AVGS*NADLT*IEE | 1713 | Sdpr | Serum deprivation-response | 2 |
| | | DEEEEPVALQQAQQVR | | | protein | |
| 97 | IPI:IPI00135660.5 | R\hat{ }GNNSAVGS*NADLT*IEE | 1714 | Sdpr | Serum deprivation-response | 2 |
| | | DEEEEPVALQQAQQVR\hat{ } | | | protein | |
| 98 | IPI:IPI00404545.2 | SLS*S*PTVTLSAPLEGAK@ | 1715 | Nedd4l | Isoform 3 of E3 ubiquitin-protein | 2 |
| | | | | | ligase NEDD4-like | |
| 99 | IPI:IPI00121892.9 | R\hat{ }PPS*PDPNTK@ | 1716 | Spnb2 | Isoform 2 of Spectrin beta chain | 2 |
| | | | | | brain 1 | |
| 100 | IPI:IPI00322707.5 | AKQPVIGDQNS*DSDEMLA | 1717 | Atrx | Transcriptional regulator ATRX | 2 |
| | | VLK | | | | |
| 101 | IPI:IPI00929786.1 | SLPT*TVPES*PNYR\hat{ } | 1718 | Larp1 | Isoform 1 of La-related protein 1 | 3 |
| 102 | IPI:IPI00125319.1 | TTS*FAESCK@PVQQPSAF | 1719 | Gsk3b | Glycogen synthase kinase-3 beta | 3 |
| | | GSMK@ | | | | |

Table 9(b)

| | rapa median area | | | |
|---|---|---|---|---|
| ID | ratio | rapa median SN ratio | KU median area ratio | KU median SN ratio |
| 1 | −1.15E+00 | −1.14E+00 | −0.8183 | −0.9318 |
| 2 | −2.1437 | −2.4238 | −0.78695 | −0.9492 |
| 3 | −4.758 | −4.9643 | −0.6283 | −0.5475 |
| 4 | −4.62805 | −4.47925 | −0.6283 | −0.5475 |
| 5 | −3.6217 | −3.885 | −0.6283 | −0.5475 |
| 6 | −1.0246 | −1.0724 | −0.5666 | 0.1321 |
| 7 | −1.881 | −1.90875 | −0.5354 | −0.5361 |
| 8 | −5.2234 | −5.13485 | −0.4254 | −0.6826 |
| 9 | −1.8021 | −2.01515 | −0.4151 | −0.4336 |
| 10 | −1.5621 | −1.0464 | −0.3725 | −0.7405 |
| 11 | −1.4347 | −1.3061 | −0.3725 | −0.7405 |
| 12 | −1.6324 | −1.5779 | −0.3224 | −0.0637 |
| 13 | −1.5511 | −1.71335 | −0.2831 | −0.0584 |
| 14 | −1.78365 | −2.00075 | −0.2767 | 0.0092 |
| 15 | −1.7555 | −2.0749 | −0.2767 | 0.0092 |
| 16 | −1.7499 | −1.7737 | −0.2767 | 0.0092 |
| 17 | −1.5242 | −1.76425 | −0.2767 | 0.0092 |
| 18 | −2.0759 | −2.2586 | −0.2661 | −0.1505 |
| 19 | −1.9707 | −1.7471 | −0.2661 | −0.1505 |
| 20 | −1.8874 | −1.85315 | −0.2661 | −0.1505 |
| 21 | −1.8106 | −1.691 | −0.2661 | −0.1505 |
| 22 | −1.6838 | −1.5207 | −0.2661 | −0.1505 |
| 23 | −2.8898 | −2.7784 | −0.2201 | −0.1513 |
| 24 | −1.0015 | −0.8287 | −0.2135 | −0.3206 |
| 25 | −3.15205 | −2.9693 | −0.213 | 0.1493 |
| 26 | −1.3868 | −0.6688 | −0.1992 | −0.1577 |
| 27 | −1.1818 | −1.15455 | −0.1474 | −0.0308 |
| 28 | −1.7403 | −1.8968 | −0.1417 | 0.6197 |
| 29 | −1.6267 | −1.0033 | −0.139 | −0.2714 |
| 30 | −1.0844 | −1.1514 | −0.106 | −0.2609 |
| 31 | −2.78805 | −1.56635 | −0.1017 | −0.8833 |
| 32 | −3.0877 | −2.2749 | −0.099 | 0.6127 |
| 33 | −2.20425 | −2.1077 | −0.085 | 0.023 |
| 34 | −1.2842 | −0.6546 | −0.0822 | 0.1059 |
| 35 | −1.7868 | −2.1396 | −0.0816 | 0.1618 |
| 36 | −1.2551 | −1.22675 | −0.0656 | −0.106 |
| 37 | −2.9525 | −3.6913 | −0.0637 | 0.7614 |
| 38 | −1.1888 | −1.2446 | −0.014 | −0.0308 |
| 39 | −1.2382 | −1.25285 | −0.0136 | 0.0107 |
| 40 | −2.1557 | −2.1257 | −0.01 | −0.0601 |
| 41 | −4.4981 | −3.9942 | 0.0259 | −0.0762 |
| 42 | −1.7508 | −0.9897 | 0.0386 | 0.3631 |
| 43 | −1.896 | −2.1685 | 0.0563 | 0.2174 |
| 44 | −1.89265 | −2.1434 | 0.0563 | 0.1073 |
| 45 | −1.40465 | −1.02655 | 0.0563 | 0.2174 |
| 46 | −1.896 | −2.1685 | 0.0814 | 0.17005 |
| 47 | −3.6492 | −3.6492 | 0.0966 | 0.0223 |
| 48 | −1.8973 | −2.1434 | 0.1063 | 0.2306 |
| 49 | −1.273 | −0.7556 | 0.1063 | 0.2306 |
| 50 | −2.8428 | −2.9973 | 0.1159 | 0.0362 |
| 51 | −2.8019 | −2.9973 | 0.1159 | 0.0362 |
| 52 | −1.2352 | −0.0592 | 0.1368 | 0.1632 |
| 53 | −3.106 | −3.2482 | 0.1555 | 0.3337 |
| 54 | −2.9525 | −3.6913 | 0.1555 | 0.3337 |
| 55 | −1.298 | −1.4299 | 0.2219 | 0.413 |
| 56 | −1.7403 | −1.8968 | 0.22985 | 0.1085 |
| 57 | −1.3906 | −1.2466 | 0.2704 | 1.5615 |
| 58 | −1.1401 | −1.2853 | 0.2835 | 0.1358 |
| 59 | −2.4187 | −0.9488 | 0.307 | 0.366 |
| 60 | −1.3056 | −1.2452 | 0.3118 | 0.4053 |
| 61 | −1.2719 | −1.0234 | 0.3118 | 0.4053 |
| 62 | −1.4486 | −1.5455 | 0.3219 | 0.2377 |
| 63 | −3.8536 | −3.8536 | 0.3485 | 0.0243 |
| 64 | −1.36155 | −1.26665 | 0.3516 | 0.4392 |
| 65 | −1.889 | −1.727 | 0.3696 | 1.0368 |
| 66 | −2.0572 | −2.2717 | 0.3908 | 0.1481 |
| 67 | −1.626 | −2.0445 | 0.51825 | 0.767 |
| 68 | −1.29335 | −1.2722 | 0.51825 | 0.767 |
| 69 | −1.9492 | −2.05475 | 0.5526 | 0.865 |
| 70 | −1.1567 | −1.0106 | 0.5969 | 0.9976 |
| 71 | −1.1567 | −1.0106 | 0.5969 | 0.9976 |
| 72 | −2.818 | −2.9973 | 1.1365 | 1.0801 |
| 73 | −3.4866 | −3.4138 | 1.2714 | 1.4529 |
| 74 | 0.0401 | −0.1138 | −3.4595 | −5.0415 |
| 75 | 0.011 | −0.1502 | −3.3715 | −3.3715 |
| 76 | −0.22145 | −0.31255 | −2.9948 | −2.3042 |
| 77 | 0.2389 | 0.2797 | −2.8989 | −2.0993 |
| 78 | −0.2639 | −0.32955 | −2.8403 | −2.8893 |
| 79 | −0.2514 | −0.2836 | −2.7755 | −2.167 |
| 80 | −0.2469 | −0.3415 | −2.5654 | −2.1344 |
| 81 | −0.58015 | −0.1451 | −2.5295 | −2.6811 |
| 82 | −0.1752 | 0.3008 | −2.3289 | −2.6763 |
| 83 | 0.2073 | 0.2067 | −2.0546 | −2.0769 |
| 84 | −0.3079 | −0.4553 | −1.759 | −1.6004 |
| 85 | 0.13985 | 0.04745 | −1.5237 | −1.538 |
| 86 | −0.1736 | −0.41725 | −1.3706 | −0.9742 |
| 87 | −0.1646 | −0.2464 | −1.3468 | −1.5444 |
| 88 | 0.3745 | 0.3233 | −1.2893 | −1.2512 |
| 89 | −0.5295 | −0.8185 | −1.1648 | −1.0131 |
| 90 | −0.4199 | −0.3301 | −1.1227 | −0.4758 |
| 91 | −0.2531 | −0.1626 | −1.1227 | −0.4758 |
| 92 | −0.1997 | −0.1626 | −1.1227 | −0.4758 |
| 93 | 0.1923 | 0.3451 | −1.1197 | −1.2356 |
| 94 | −0.0084 | 0.0364 | −1.0814 | −1.582 |
| 95 | −0.3065 | −0.4384 | −1.0477 | −1.431 |
| 96 | −0.18955 | −0.4136 | −1.0458 | −1.0184 |
| 97 | −0.15495 | −0.3246 | −1.0458 | −1.0184 |
| 98 | −0.573 | −0.2978 | −1.0265 | −0.7599 |
| 99 | 0.0109 | −0.0378 | −1.0209 | −0.674 |
| 100 | 0.10215 | 0.0699 | −1.011 | −2.1889 |
| 101 | −2.1207 | −2.1939 | −6.1589 | −5.4183 |
| 102 | −1.71915 | −1.62995 | −1.07715 | −2.06845 |

Table 9. Classification of mTOR targets identified in the Rapamycin and Ku-0063794 screens. Class 1 represents downstream effectors of rapamycin-sensitive mTORC1. Class 2 represents downstream effectors of rapamycin-insensitive mTORC1 or mTORC2. Class 3 represents the proteins downstream of both mTORC1 and mTORC2. Name of the genes and the sequences of the phosphopeptides are shown. Table 9 is provided in two parts because the colums span more than a single page. Table 9(a) contains colums 1-6 of Table 9 and Table 9(b) contains columns 7-10, wherein corresponding entries are identified in an ID column in each section. Information provided in (a) for ID 1 relates to the information provided in (b) for ID 1 and vice versa, information provided in (a) for ID 2 relates to the information provided in (b) for ID 2 and vice versa, and so on.

TABLE 10
Gene ontology analysis of the hits identified in the rapamycin and Ku-0063794 screens. The Table contains
four subsections: Biological processes that the rapamycin-sensitive and Ku-0063794-sensitive hits overrepresented
are identified in the “Rapa BP GO” and “Ku BP GO” subsection, respectively. Pathways that the Rapamycin-
senstitive and Ku-0063794-sensitive hits overrepresented are shown under “Rapa KEGG pathways” and “Ku
KEGG pathways,” respectively. Subsections are displayed in three parts, (a), (b), and (c), because the Table
columns span more than one page. Corresponding entries are identified by an ID number. For example, entries
under ID number 1 in Rapa BP GO (a), (b), and (c) refer to the same entry, entries under ID number 2 in Rapa BP
GO (a), (b), and (c) refer to the same entry, entries under ID number 1 in Rapa KEGG pathways (a), (b), and (c)
refer to the same entry, and so forth.
Rapa BP GO pathways (a):

| ID | Term | Count | % | PValue |
|---|---|---|---|---|
| 1 | GO:0010605~negative regulation of macromolecule | 20 | 10.41666667 | 4.18E−07 |
| | metabolic process | | | |
| 2 | GO:0031327~negative regulation of cellular | 17 | 8.854166667 | 4.17E−06 |
| | biosynthetic process | | | |
| 3 | GO:0010558~negative regulation of macromolecule | 16 | 8.333333333 | 1.31E−05 |
| | biosynthetic process | | | |
| 4 | GO:0045934~negative regulation of nucleobase, | 15 | 7.8125 | 0.0000314 |
| | nucleoside, nucleotide and nucleic acid metabolic | | | |
| | process | | | |
| 5 | GO:0032868~response to insulin stimulus | 6 | 3.125 | 0.000264 |
| 6 | GO:0016481~negative regulation of transcription | 13 | 6.770833333 | 0.000271 |
| 7 | GO:0016192~vesicle-mediated transport | 14 | 7.291666667 | 0.000603 |
| 8 | GO:0032869~cellular response to insulin stimulus | 5 | 2.604166667 | 0.000783 |
| 9 | GO:0051253~negative regulation of RNA | 11 | 5.729166667 | 0.000886 |
| | metabolic process | | | |
| 10 | GO:0051248~negative regulation of protein | 6 | 3.125 | 0.00096 |
| | metabolic process | | | |
| 11 | GO:0006898~receptor-mediated endocytosis | 4 | 2.083333333 | 0.001048574 |
| 12 | GO:0006468~protein amino acid phosphorylation | 16 | 8.333333333 | 0.00137311 |
| 13 | GO:0051276~chromosome organization | 12 | 6.25 | 0.001936745 |
| 14 | GO:0016568~chromatin modification | 9 | 4.6875 | 0.002136023 |
| 15 | GO:0043434~response to peptide hormone stimulus | 6 | 3.125 | 0.002310739 |
| 16 | GO:0032870~cellular response to hormone stimulus | 5 | 2.604166667 | 0.002406516 |
| 17 | GO:0008286~insulin receptor signaling pathway | 4 | 2.083333333 | 0.002457872 |
| 18 | GO:0045892~negative regulation of transcription, | 10 | 5.208333333 | 0.003123471 |
| | DNA dependent | | | |
| 19 | GO:0006325~chromatin organization | 10 | 5.208333333 | 0.003622675 |
| 20 | GO:0046777~protein amino acid autophosphory- | 5 | 2.604166667 | 0.003851564 |
| | lation | | | |
| 21 | GO:0010627~regulation of protein kinase cascade | 7 | 3.645833333 | 0.004027038 |
| 22 | GO:0016310~phosphorylation | 16 | 8.333333333 | 0.004128421 |
| 23 | GO:0051656~establishment of organelle localization | 4 | 2.083333333 | 0.004303328 |
| 24 | GO:0007010~cytoskeleton organization | 10 | 5.208333333 | 0.004531801 |
| 25 | GO:0032535~regulation of cellular component size | 7 | 3.645833333 | 0.004843432 |
| 26 | GO:0022402~cell cycle process | 11 | 5.729166667 | 0.00498568 |
| 27 | GO:0045926~negative regulation of growth | 5 | 2.604166667 | 0.005003024 |
| 28 | GO:0032269~negative regulation of cellular protein | 5 | 2.604166667 | 0.005258125 |
| | metabolic process | | | |
| 29 | GO:0065003~macromolecular complex assembly | 10 | 5.208333333 | 0.005717288 |
| 30 | GO:0032268~regulation of cellular protein metabolic | 9 | 4.6875 | 0.006003754 |
| | process | | | |
| 31 | GO:0000122~negative regulation of transcription | 8 | 4.166666667 | 0.007342706 |
| | from RNA polymerase II promoter | | | |
| 32 | GO:0032319~regulation of Rho GTPase activity | 3 | 1.5625 | 0.007895767 |
| 33 | GO:0046578~regulation of Ras protein signal | 7 | 3.645833333 | 0.008452332 |
| | transduction | | | |
| 34 | GO:0043933~macromolecular complex subunit | 10 | 5.208333333 | 0.009572997 |
| | organization | | | |
| 35 | GO:0006793~phosphorus metabolic process | 17 | 8.854166667 | 0.009756342 |
| 36 | GO:0006796~phosphate metabolic process | 17 | 8.854166667 | 0.009756342 |
| 37 | GO:0006897~endocytosis | 7 | 3.645833333 | 0.010081394 |
| 38 | GO:0010324~membrane invagination | 7 | 3.645833333 | 0.010081394 |
| 39 | GO:0006396~RNA processing | 11 | 5.729166667 | 0.01020022 |
| 40 | GO:0007169~transmembrane receptor protein | 7 | 3.645833333 | 0.01110676 |
| | tyrosine kinase signaling pathway | | | |
| 41 | GO:0010608~posttranscriptional regulation of gene | 6 | 3.125 | 0.01469374 |
| | expression | | | |
| 42 | GO:0045792~negative regulation of cell size | 4 | 2.083333333 | 0.014742447 |
| 43 | GO:0001701~in utero embryonic development | 8 | 4.166666667 | 0.015417559 |
| 44 | GO:0051640~organelle localization | 4 | 2.083333333 | 0.015500935 |
| 45 | GO:0051493~regulation of cytoskeleton organization | 5 | 2.604166667 | 0.015757695 |
| 46 | GO:0007049~cell cycle | 13 | 6.770833333 | 0.015795779 |
| 47 | GO:0006417~regulation of translation | 5 | 2.604166667 | 0.016293879 |
| 48 | GO:0017148~negative regulation of translation | 3 | 1.5625 | 0.019054263 |
| 49 | GO:0034622~cellular macromolecular complex | 7 | 3.645833333 | 0.019238583 |
| | assembly | | | |
| 50 | GO:0008361~regulation of cell size | 5 | 2.604166667 | 0.020996944 |
| 51 | GO:0009725~response to hormone stimulus | 6 | 3.125 | 0.022401918 |
| 52 | GO:0051056~regulation of small GTPase mediated | 7 | 3.645833333 | 0.023850905 |
| | signal transduction | | | |
| 53 | GO:0030029~actin filament-based process | 6 | 3.125 | 0.028573614 |
| 54 | GO:0048729~tissue morphogenesis | 7 | 3.645833333 | 0.028642761 |
| 55 | GO:0001932~regulation of protein amino acid | 5 | 2.604166667 | 0.030261806 |
| | phosphorylation | | | |
| 56 | GO:0008104~protein localization | 14 | 7.291666667 | 0.031319732 |
| 57 | GO:0000278~mitotic cell cycle | 7 | 3.645833333 | 0.031803074 |
| 58 | GO:0034621~cellular macromolecular complex | 7 | 3.645833333 | 0.032351058 |
| | subunit organization | | | |
| 59 | GO:0009719~response to endogenous stimulus | 6 | 3.125 | 0.03367924 |
| 60 | GO:0045449~regulation of transcription | 31 | 16.14583333 | 0.03551581 |
| 61 | GO:0007163~establishment or maintenance of cell | 3 | 1.5625 | 0.036247509 |
| | polarity | | | |
| 62 | GO:0080135~regulation of cellular response to stress | 4 | 2.083333333 | 0.036466875 |
| 63 | GO:0006357~regulation of transcription from RNA | 12 | 6.25 | 0.037453702 |
| | polymerase II promoter | | | |
| 64 | GO:0032314~regulation of Rac GTPase activity | 2 | 1.041666667 | 0.038299417 |
| 65 | GO:0040007~growth | 6 | 3.125 | 0.040063168 |
| 66 | GO:0022403~cell cycle phase | 8 | 4.166666667 | 0.040932988 |
| 67 | GO:0031328-positive regulation of cellular | 11 | 5.729166667 | 0.042602185 |
| | biosynthetic process | | | |
| 68 | GO:0032318~regulation of Ras GTPase activity | 4 | 2.083333333 | 0.042864282 |
| 69 | GO:0010604~positive regulation of macromolecule | 12 | 6.25 | 0.044146336 |
| | metabolic process | | | |
| 70 | GO:0022613~ribonucleoprotein complex biogenesis | 5 | 2.604166667 | 0.044519805 |
| 71 | GO:0009891~positive regulation of biosynthetic | 11 | 5.729166667 | 0.044819532 |
| | process | | | |
| 72 | GO:0046822~regulation of nucleocytoplasmic | 3 | 1.5625 | 0.045236118 |
| | transport | | | |
| 73 | GO:0006346~methylation-dependent chromatin | 2 | 1.041666667 | 0.047644564 |
| | silencing | | | |
| 74 | GO:0016044~membrane organization | 7 | 3.645833333 | 0.049524346 |
| 75 | GO:0007167~enzyme linked receptor protein | 7 | 3.645833333 | 0.05025049 |
| | signaling pathway | | | |
| 76 | GO:0035023~regulation of Rho protein signal | 4 | 2.083333333 | 0.051230918 |
| | transduction | | | |
| 77 | GO:0043009~chordate embryonic development | 9 | 4.6875 | 0.052874272 |
| 78 | GO:0006916~anti-apoptosis | 4 | 2.083333333 | 0.054183284 |
| 79 | GO:0009792~embryonic development ending in | 9 | 4.6875 | 0.055294577 |
| | birth or egg hatching | | | |
| 80 | GO:0044087~regulation of cellular component | 4 | 2.083333333 | 0.055689578 |
| | biogenesis | | | |
| 81 | GO:0006350~transcription | 25 | 13.02083333 | 0.055970599 |
| 82 | GO:0043062~extracellular structure organization | 5 | 2.604166667 | 0.057303217 |
| 83 | GO:0043122~regulation of I-kappaB kinase/ | 3 | 1.5625 | 0.057483236 |
| | NF-kappaB cascade | | | |
| 84 | GO:0051726~regulation of cell cycle | 6 | 3.125 | 0.057658627 |
| 85 | GO:0046907~intracellular transport | 9 | 4.6875 | 0.059056688 |
| 86 | GO:0006260~DNA replication | 5 | 2.604166667 | 0.060776557 |
| 87 | GO:0048598~embryonic morphogenesis | 8 | 4.166666667 | 0.060833555 |
| 88 | GO:0043087~regulation of GTPase activity | 4 | 2.083333333 | 0.061912428 |
| 89 | GO:0051129~negative regulation of cellular | 4 | 2.083333333 | 0.061912428 |
| | component organization | | | |
| 90 | GO:0032386~regulation of intracellular transport | 3 | 1.5625 | 0.062669535 |
| 91 | GO:0033043~regulation of organelle organization | 5 | 2.604166667 | 0.063153096 |
| 92 | GO:0042325~regulation of phosphorylation | 7 | 3.645833333 | 0.063602017 |
| 93 | GO:0045786~negative regulation of cell cycle | 3 | 1.5625 | 0.065320236 |
| 94 | GO:0001570~vasculogenesis | 3 | 1.5625 | 0.065320236 |
| 95 | GO:0045947~negative regulation of translational | 2 | 1.041666667 | 0.066065335 |
| | initiation | | | |
| 96 | GO:0006461~protein complex assembly | 6 | 3.125 | 0.070461851 |
| 97 | GO:0070271~protein complex biogenesis | 6 | 3.125 | 0.070461851 |
| 98 | GO:0030833~regulation of actin filament | 3 | 1.5625 | 0.070731878 |
| | polymerization | | | |
| 99 | GO:0010557~positive regulation of macromolecule | 10 | 5.208333333 | 0.073220823 |
| | biosynthetic process | | | |
| 100 | GO:0019220~regulation of phosphate metabolic | 7 | 3.645833333 | 0.073259901 |
| | process | | | |
| 101 | GO:0051174~regulation of phosphorus metabolic | 7 | 3.645833333 | 0.073259901 |
| | process | | | |
| 102 | GO:0048589~developmental growth | 4 | 2.083333333 | 0.073541659 |
| 103 | GO:0030838~positive regulation of actin filament | 2 | 1.041666667 | 0.075142677 |
| | polymerization | | | |
| 104 | GO:0030308~negative regulation of cell growth | 3 | 1.5625 | 0.076284196 |
| 105 | GO:0030036~actin cytoskeleton organization | 5 | 2.604166667 | 0.077082848 |
| 106 | GO:0031399~regulation of protein modification | 5 | 2.604166667 | 0.077082848 |
| | process | | | |
| 107 | GO:0000902~cell morphogenesis | 7 | 3.645833333 | 0.080785789 |
| 108 | GO:0016477~cell migration | 6 | 3.125 | 0.08471051 |
| 109 | GO:0008064~regulation of actin polymerization or | 3 | 1.5625 | 0.084860387 |
| | depolymerization | | | |
| 110 | GO:0030832~regulation of actin filament length | 3 | 1.5625 | 0.08778162 |
| 111 | GO:0002009~morphogenesis of an epithelium | 5 | 2.604166667 | 0.088103636 |
| 112 | GO:0006333~chromatin assembly or disassembly | 4 | 2.083333333 | 0.089801855 |
| 113 | GO:0010638~positive regulation of organelle | 3 | 1.5625 | 0.090732662 |
| | organization | | | |
| 114 | GO:0045941~positive regulation of transcription | 9 | 4.6875 | 0.091555467 |
| 115 | GO:0006887~exocytosis | 4 | 2.083333333 | 0.091694717 |
| 116 | GO:0008283~cell proliferation | 6 | 3.125 | 0.092969193 |
| 117 | GO:0035020~regulation of Rac protein signal | 2 | 1.041666667 | 0.093035503 |
| | transduction | | | |
| 118 | GO:0000226~microtubule cytoskeleton organization | 4 | 2.083333333 | 0.093604179 |
| 119 | GO:0060562~epithelial tube morphogenesis | 4 | 2.083333333 | 0.093604179 |

Rapa BP GO pathways (b):

| ID | Genes |
|---|---|
| 1 | BCLAF1,FOXK1,GM5611,ZEB2,SAP30,EIP4EBP1,SORBS3,EIF4EBP2,GM7289,FAM129A,HELLS, |
| | IBTK,LOC100046628,NACC1,GM9118,TRIM28,RB1,EHMT2,FLNA,FXR1,GM6477,NFIC,CUX1,LOC |
| | 633387,SMARCA4 |
| 2 | PALM,BCLAF1,NACC1,FOXK1,TRIM28,ZEB2,RB1,EHMT2,FXR1,SAP30,EIF4EBP1,SORBS3, |
| | EIF4EBP2,CUX1,NFIC,HELLS,SMARCA4 |
| 3 | BCLAF1,NACC1,FOXK1,TRIM28,ZEB2,RB1,EHMT2,FXR1,SAP30,EIF4EBP2,SORBS3,EIF4EBP2, |
| | CUX1,NFIC,HELLS,SMARCA4 |
| 4 | PALM,LOC100046628,BCLAF1,NACC1,FOXK1,GM9118,TRIM28,GM5611,ZEB2,RB1,EHMT2, |
| | GM6477,SAP30,SORBS3,NPM1,GM7289,CUX1,NFIC,LOC633387,HELLS,SMARCA4 |
| 5 | ZFP106,IRS2,EIF4EBP1,EIF4EBP2,LOC100048123,LOC544757,MTOR,AKT2 |
| 6 | NACC1,BCLAF1,FOXK1,TRIM28,ZEB2,RB1,EHMT2,SAP30,SORBS3,NFIC,CUX1,HELLS,SMARCA4 |
| 7 | MYO5A,GM8786,LOC100048600,SNX17,ARHGAP17,LLGL1,SCRIB,FLNA,DAB2,CTTN,ULK1,RAB1, |
| | FKBP15,CUX1,ERC1,TRIP10 |
| 8 | ZFP106,IRS2,EIF4EBP1,EIF4DBP2,LOC100048123,LOC544757,AKT2 |
| 9 | LOC100046628,FOXK1,GM9118,TRIM28,GM5611,RB1,EHMT2,GM6477,SAP30,SORBS3,NPM1,GM7 |
| | 289,CUX1,NFIC,LOC633387,HELLS,SMARCA4 |
| 10 | IBTK,EIF4EBP1,EIF4EBP2,FAM129A,FLNA,FXR1 |
| 11 | DAB2,CTTN,GM8786,ULK1,SNX17 |
| 12 | SRPK2,GM4521,BCR,TRIM28,LOC544757,PKN2,WNK1,RPS6KB1,PRKD2,PTK2,LOC100048123,ULK |
| | 1,AAK1,GSK3B,EEF2K,BMP2K,CDK12,MTOR,AKT2 |
| 13 | KDM6A,PDS5B,ARID1A,RB1,EHMT2,1600027N09RIK,SMARCC2,LOC675933,CHD1,KDM3B,TINF2, |
| | HELLS,SMARCA4 |
| 14 | KDM6A,SMARCC2,CHD1,KDM3B,RB1,1600027N09RIK,EHMT2,HELLS,SMARCA4 |
| 15 | ZFP106,IRS2,EIF4EBP1,EIF4EBP2,LOC100048123,LOC544757,MTOR,AKT2 |
| 16 | ZFP106,IRS2,EIF4EBP1,EIF4EBP2,LOC100048123,LOC544757,AKT2 |
| 17 | ZFP106,IRS2,EIF4EBP1,EIF4EBP2 |
| 18 | SAP30,SORBS3,FOXK1,TRIM28,RB1,CUX1,EHMT2,NFIC,HELLS,SMARCA4 |
| 19 | KDM6A,SMARCC2,LOC675933,CHD1,ARID1A,KDM3B,RB1,1600027N09RIK,EHMT2,HELLS, |
| | SMARCA4 |
| 20 | PTK2,ULK1,TRIM28,CDK12,MTOR |
| 21 | PDCD11,SORBS3,LOC100048123,SQSTM1,LOC544757,ZEB2,RICTOR,FLNA,AKT2 |
| 22 | SRPK2,GM4521,BCR,TRIM28,LOC544757,PKN2,WNK1,RPS6KB1,PRKD2,LOC100048123,ULK1, |
| | AAK1,GSK3B,EEF2K,BMP2K,CDK12,MTOR,AKT2 |
| 23 | MYO5A,LOC100046628,PTK2,GM9118,NPM1,GM5611,GM7289,SCRIB,LOC633387,GM6477 |
| 24 | INF2,PALM,LOC100046628,FMNL3,GM9118,GM5611,ARHGAP17,RICTOR,INF2Q,FLNA,GM6477, |
| | PTK2,NPM1,MTAP1B,GM7289,LOC633387,RANBP10 |
| 25 | LOC100046628,GM9118,FOXK1,GM5611,RICTOR,GM6477,ULK1,NPM1,GM7289,SPNA2,MTOR, |
| | DNAJC2,LOC633387 |
| 26 | LOC100046628,PDS5B,ZC3HC1,GM9118,AHCTF1,GM5611,RB1,EHMT2,CD2AP,GM6477,MACF1, |
| | GSK3B,NPM1,GM7289,DNAJC2,LOC633387,HELLS |
| 27 | ATXN2,PTK2,FOXK1,ULK1,DNAJC2 |
| 28 | IBTK,EIF4EBP1,EIF4EBP2,FAM129A,FXR1 |
| 29 | SRPK2,LOC100046628,NACC1,GM9118,AHCTF1,GM5611,FLNA,GM6477,EIF3A,PTK2,GTF2F1, |
| | NPM1,GM7289,LOC633387,HELLS,SMARCA4 |
| 30 | IBTK,EIF4G1,EIF4EBP1,EIF4EBP2,ZEB2,MTOR,RICTOR,FAM129A,FXR1 |
| 31 | SAP30,SORBS3,TRIM28,RB1,CUX1,EHMT2,NFIC,SMARCA4 |
| 32 | MTOR,RICTOR,SCRIB |
| 33 | BCR,GBF1,TBC1D4,MTOR,RICTOR,SCRIB,IQSEC1 |
| 34 | SRPK2,LOC100046628,NACC1,GM9118,AHCTF1,GM5611,FLNA,GM6477,EIF3A,PTK2,GTF2F1,NPM1, |
| | GM7289,LOC633387,HELLS,SMARCA4 |
| 35 | SRPK2,GM4521,BCR,TRIM28,LOC544757,PKN2,WNK1,RPS6KB1,PRKD2,PTK2,LOC100048123,UKL1, |
| | AAK1,GSK3B,CDK12,EEF2K,BMP2K,PPP1R12A,MTOR,AKT2 |
| 36 | SRPK2,GM4521,BCR,TRIM28,LOC544757,PKN2,WNK1,RPS6KB1,PTK2,LOC100048123,ULK1, |
| | AAK1,GSK3B,CDK12,EEF2K,BMP2K,PPP1R12A,MTOR,AKT2 |
| 37 | DAB2,CTTN,GM8786,ULK1,SNX17,FKBP15,TRIP10,SCRIB |
| 38 | DAB2,CTTN,GM8786,ULK1,SNX17,FKBP15,TRIP10,SCRIB |
| 39 | LOC100046766,SRPK2,PDCD11,LOC100046744,EMG1,SFRS1,LOC100047322,RSL1D1,SFRS8,PCBP1, |
| | SRRM2,LARP7,LOC100046735,DHX15,SRRM1,LOC100048559 |
| 40 | ZFP106,GRB10,PTK2,IRS2,EIF4EBP1,EIF4EBP2,FLNA |
| 41 | EIF4G1,EIF4EBP1,EIF4EBP2,FAM129A,FLNA,FXR1 |
| 42 | FOXK1,ULK1,MTOR,DNAJC2 |
| 43 | MIB1,DAB2,SRFS1,TCFEB,JUNB,LOC100048559,MLL2,MED1,SMARCA4 |
| 44 | MYO5A,LOC100046628,PTK2,GM9118,NPM1,GM5611,GM7289,SCRIB,LOC633387,GM6477 |
| 45 | LOC100046628,GM9118,MTAP1B,NPM1,GM5611,SPNA2,GM7289,MTOR,RICTOR,LOC633387, |
| | GM6477 |
| 46 | LOC100046628,ZC3HC1,PDS5B,GM9118,LIN9,GM5611,AHCTF1,RB1,EHMT2,CD2AP,GM6477,NUM |
| | A1,MACF1,GSK3B,NPM1,GM7289,DNAJC2,HELLS,LOC633387 |
| 47 | EIF4G1,EIF4EBP1,EIF4EBP2,FAM129A,FXR1 |
| 48 | EIF4EBP1,EIF4EBP2,FXR1 |
| 49 | SRPK2,PTK2,EIF3A,AHCTF1,HELLS,FLNA,SMARCA4 |
| 50 | LOC100046628,FOXK1,GM9118,ULK1,NPM1,GM5611,GM7289,MTOR,DNAJC2,LOC633387,GM6477 |
| 51 | ZFP106,IRS2,EIF4EBP1,EIF4EBP2,LOC100048123,LOC544757,MTOR,AKT2 |
| 52 | BCR,GBF1,TBC1D4,MTOR,RICTOR,SCRIB,IQSEC1 |
| 53 | MYO5A,INF2,FMNL3,ARHGAP17,RICTOR,INF2Q,FLNA |
| 54 | MIB1,MACF1,TNC,ZEB2,SCRIB,MED1,SMARCA4 |
| 55 | IBTK,ZEB2,MTOR,RICTOR,FAM129A |
| 56 | PALM,LOC100046628,LOC100048600,GM9118,SNX17,GM5611,AHCTF1,FLNA,SCRIB,GM6477, |
| | POM121,ERBB2IP,MACF1,ULK1,RAB1,GSK3B,NPM1,GM7289,SNX30,ERC1,LOC633387 |
| 57 | ZC3HC1,PDS5B,AHCTF1,RB1,CD2AP,DNAJC2,HELLS |
| 58 | SRPK2,PTK2,EIF3A,AHCTF1,HELLS,FLNA,SMARCA4 |
| 59 | ZFP106,IRS2,EIF4EBP1,EIF4EBP2,LOC100048123,LOC1000544757,MTOR,AKT2 |
| 60 | BCLAF1,CARHSP1,FOXK1,FOXK2,AHCTF1,ZEB2,1600027N09RIK,SAP30,SORBS3,GM9791,KDM3B, |
| | TCFEB,DNAJC2,HELLS,NACC1,PDCD11,TRIM28,CCNL1,RB1,EHMT2,JUNB,FLNA,RFC1,GTF2F1, |
| | SMARCC2,TMPO,ZFP516,CUX1,NFIC,PBX2,SMARCA4,MED1 |
| 61 | MACF1,SCRIB,LLGL1 |
| 62 | LOC100046628,LOC100048123,GM9118,NPM1,LOC544757,GM5611,ZEB2,GM7289,MTOR,LOC633387, |
| | AKT2,GM6477 |
| 63 | SAP30,SORBS3,FOXK1,TRIM28,RB1,CUX1,TCFEB,EHMT2,NFIC,PBX2,MED1,SMARCA4 |
| 64 | MTOR,RICTOR |
| 65 | MYO5A,LOC100046628,GM9118,ULK1,NPM1,GM5611,GM7289,EHMT2,LOC633387,MED1,GM6477, |
| | SMARCA4 |
| 66 | ZC3HC1,PDS5B,ACHTF1,RB1,EHTMC2,CD2AP,DNAJC2,HELLS |
| 67 | LOC100046628,GM9118,FOXK1,TRIM28,AHCTF1,GM5611,RB1,GM6477,GTF2F1,NPM1,GM7289, |
| | TCFEB,FAM129A,NFIC,PBX2,LOC633387,MED1 |
| 68 | TBC1D4,MTOR,RICTOR,SCRIB |
| 69 | FOXK1,GTF2F1,TRIM28,AHCTF1,MTOR,RB1,RICTOR,TCFEB,FAM129A,NFIC,PBX2,MED1 |
| 70 | SRPK2,LOC100046628,PDCD11,EIF3A,GM9118,EMG1,NPM1,GM5611,LOC633387,GM6477 |
| 71 | LOC100046628,GM9118,FOXK1,TRIM28,AHCTF1,GM5611,RB1,GM6477,GTF2F1,NPM1,GM7289, |
| | TCFEB,FAM129A,NFIC,PBX2,LOC633387,MED1 |
| 72 | GSK3B,PPP1R12A,FLNA |
| 73 | HELLS,SMARCA4 |
| 74 | DAB2,CTTN,GM8786,ULK1,SNX17,FKBP15,TRIP10,SCRIB |
| 75 | ZFP106,GRB10,PTK2,IRS2,EIF4EBP1,EIF4EBP2,FLNA |
| 76 | BCR,MTOR,RICTOR,SCRIB |
| 77 | MIB1,DAB2,ZEB2,SFRS1,TBFEB,JUNB,LOC100048559,MLL2,MED1,SMARCA4 |
| 78 | AKT1S1,ZC3HC1,GSK3B,HELLS |
| 79 | MIB1,DAB2,ZEB2,SFRS1,TCFEB,JUNB,LOC100048559,MLL2,MED1,SMARCA4 |
| 80 | PTK2,SPNA2,MTOR,RICTOR |
| 81 | BLCAF1,FOXK1,FOXK2,MYEF2,AHCTF1,ZEB2,1600027N09RIK,SAP30,KDM3B,TCFEB,HELLS, |
| | NACC1,TRIM28,CCNL1,RB1,JUNB,PHF3,RFC1,GTF2F1,SMARCC2,ZFP516,NFIC,CUX1,PBX2, |
| | MED1 |
| 82 | MYO5A,PTK2,TNC,LOC100048740,ADAMTS2,SMARCA4 |
| 83 | PDCD11,SQSTM1,FLNA |
| 84 | LOC100046628,BCR,FOXK1,GM9118,NPM1,GM5611,GM7289,RB1,SCRIB,JUNB,LOC633387, |
| | GM6477 |
| 85 | MYO5A,LOC100046628,LOC100048600,GM9118,GM5611,FLNA,GM6477,MACF1,ERBB2IP,GSK3B, |
| | RAB1,NPM1,GM7289,CUX1,ERC1,LOC633387 |
| 86 | RFC1,LIN9,NFIC,DNAJC2,MED1 |
| 87 | MIB1,MACF1,ZEB2,EXT1,PBX2,SCRIB,MED1,SMARCA4 |
| 88 | TBC1D4,MTOR,RICTOR,SCRIB |
| 89 | PTK2,ULK1,MTAP1B,SPNA2 |
| 90 | GSK3B,PPP1R12A,FLNA |
| 91 | LOC100046628,GM9118,MTAP1B,NPM1,GM5611,SPNA2,GM7289,MTOR,RICTOR,LOC633387, |
| | GM6477 |
| 92 | IBTK,LOC100046628,GM9118,GM5611,ZEB2,RICTOR,RB1,GM6477,NPM1,GM7289,MTOR, |
| | FAM129A,LOC633387 |
| 93 | FOXK1,RB1,SCRIB |
| 94 | PTK2,JUNB,SMARCA4 |
| 95 | EIF4EBP1,EIF4EBP2 |
| 96 | LOC100046628,PTK2,NACC1,GM9118,GTF2F1,NPM1,AHCTF1,GM5611,GM7289,LOC633387,FLNA, |
| | GM6477 |
| 97 | LOC100046628,PTK2,NACC1,GM9118,GTF2F1,NPM1,AHCTF1,GM5611,GM7289,LOC633387,FLNA, |
| | GM6477 |
| 98 | SPNA2,MTOR,RICTOR |
| 99 | FOXK1,GTF2F1,TRIM28,AHCTF1,RB1,TCFEB,FAM129A,NFIC,PBX2,MED1 |
| 100 | IBTK,LOC100046628,GM9118,GM5611,ZEB2,RICTOR,RB1,GM6477,NPM1,GM7289,MTOR, |
| | FAM129A,LOC633387 |
| 101 | IBTK,LOC100046628,GM9118,GM5611,ZEB2,RICTOR,RB1,GM6477,NPM1,GM7289,MTOR, |
| | FAM129A,LOC633387 |
| 102 | MYO5A,ULK1,MED1,SMARCA4 |
| 103 | MTOR,RICTOR |
| 104 | FOXK1,ULK1,DNAJC2 |
| 105 | INF2,FMNL3,ARHGAP17,RICTOR,INF2Q,FLNA |
| 106 | IBTK,ZEB2,MTOR,RICTOR,FAM129A |
| 107 | DAB2,PTK2,MACF1,ULK1,TRIM28,SCRIB,LLGL1 |
| 108 | PTK2,TNS1,ULK1,ZEB2,CD2AP,SCRIB |
| 109 | SPNA2,MTOR,RICTOR |
| 110 | SPNA2,MTOR,RICTOR |
| 111 | MIB1,TNC,ZEB2,SCRIB,MED1 |
| 112 | SMARCC2,CHD1,HELLS,SMARCA4 |
| 113 | LOC100046628,GM9118,NPM1,GM5611,GM7289,MTOR,RICTOR,LOC633387,GM6477 |
| 114 | FOXK1,GTF2F1,TRIM28,AHCTF1,RB1,TCFEB,NFIC,PBX2,MED1 |
| 115 | MYO5A,ARHGAP17,SCRIB,LLGL1 |
| 116 | IRS2,GSK3B,ZEB2,SCRIB,HELLS,MED1 |
| 117 | MTOR,RICTOR |
| 118 | LOC100046628,PTK2,GM9118,MTAP1B,NPM1,GM5611,GM7289,LOC633387,RANBP10,GM6477 |
| 119 | MIB1,ZEB2,SCRIB,MED1 |

Rapa BP GO pathways (c):

| | List | Pop | Pop | Fold | | | |
|---|---|---|---|---|---|---|---|
| ID | Total | Hits | Total | Enrichment | Bonferroni | Benjamini | FDR |
| 1 | 133 | 506 | 13588 | 4.038158638 | 0.000424 | 4.24E−04 | 6.62E-04 |
| 2 | 133 | 430 | 13588 | 4.039097744 | 0.00422262 | 0.002113544 | 0.006599221 |
| 3 | 133 | 418 | 13588 | 3.910637839 | 0.013202715 | 0.003317148 | 0.020725619 |
| 4 | 133 | 397 | 13588 | 3.860154164 | 0.031339368 | 0.006347957 | 0.049646045 |
| 5 | 133 | 59 | 13588 | 10.38970307 | 0.234820063 | 0.037513168 | 0.4165415 |
| 6 | 133 | 372 | 13588 | 3.57029671 | 0.240156236 | 0.033747706 | 0.427409635 |
| 7 | 133 | 466 | 13588 | 3.069347188 | 0.458108033 | 0.065811007 | 0.950983483 |
| 8 | 133 | 43 | 13588 | 11.87969925 | 0.548457185 | 0.069730046 | 1.232339007 |
| 9 | 133 | 310 | 13588 | 3.625224351 | 0.593238056 | 0.072219958 | 1.393086083 |
| 10 | 133 | 78 | 13588 | 7.858877964 | 0.622688909 | 0.072234052 | 1.508600184 |
| 11 | 133 | 21 | 13588 | 19.46007877 | 0.655224183 | 0.073240783 | 1.647016896 |
| 12 | 133 | 640 | 13588 | 2.554135338 | 0.752083804 | 0.08878612 | 2.151600307 |
| 13 | 133 | 404 | 13588 | 3.034616244 | 0.860222342 | 0.115719883 | 3.022171324 |
| 14 | 133 | 236 | 13588 | 3.896138652 | 0.885866021 | 0.119855821 | 3.328229194 |
| 15 | 133 | 95 | 13588 | 6.452552434 | 0.904450787 | 0.122300298 | 3.595818606 |
| 16 | 133 | 58 | 13588 | 8.807363236 | 0.913322224 | 0.120774231 | 3.742213886 |
| 17 | 133 | 28 | 13588 | 14.59505908 | 0.917735112 | 0.117406498 | 3.820625016 |
| 18 | 133 | 308 | 13588 | 3.317058881 | 0.958217785 | 0.140327713 | 4.831479072 |
| 19 | 133 | 315 | 13588 | 3.243346461 | 0.974869971 | 0.154173087 | 5.583086162 |
| 20 | 133 | 66 | 13588 | 7.739804056 | 0.980097008 | 0.156587702 | 5.925840566 |
| 21 | 133 | 155 | 13588 | 4.613921902 | 0.983355842 | 0.15688676 | 6.187818747 |
| 22 | 133 | 718 | 13588 | 2.276666597 | 0.984989728 | 0.154613045 | 6.33886821 |
| 23 | 133 | 34 | 13588 | 12.01946042 | 0.987440816 | 0.154948588 | 6.59892672 |
| 24 | 133 | 326 | 13588 | 3.133908391 | 0.990050495 | 0.156967149 | 6.937610234 |
| 25 | 133 | 161 | 13588 | 4.441974501 | 0.992759227 | 0.161382833 | 7.397709501 |
| 26 | 133 | 393 | 13588 | 2.859591727 | 0.993737166 | 0.16048863 | 7.607019539 |
| 27 | 133 | 71 | 13588 | 7.194747432 | 0.993846998 | 0.156076846 | 7.632510001 |
| 28 | 133 | 72 | 13588 | 7.094820384 | 0.995256964 | 0.158538831 | 8.006669044 |
| 29 | 133 | 338 | 13588 | 3.022645371 | 0.997031505 | 0.166286638 | 8.676553117 |
| 30 | 133 | 280 | 13588 | 3.283888292 | 0.997784278 | 0.169076444 | 9.092166954 |
| 31 | 133 | 231 | 13588 | 3.53819614 | 0.999435936 | 0.197489303 | 11.01137247 |
| 32 | 133 | 14 | 13588 | 21.89258861 | 0.999679622 | 0.205375826 | 11.79297815 |
| 33 | 133 | 181 | 13588 | 3.95114859 | 0.99981874 | 0.212838341 | 12.57303844 |
| 34 | 133 | 367 | 13588 | 2.783798734 | 0.999942481 | 0.231930955 | 14.12415408 |
| 35 | 133 | 866 | 13588 | 2.005556617 | 0.999952335 | 0.230394008 | 14.37545544 |
| 36 | 133 | 866 | 13588 | 2.005556617 | 0.999952335 | 0.230394008 | 14.37545544 |
| 37 | 133 | 188 | 13588 | 3.804031355 | 0.999965843 | 0.231800837 | 14.81929399 |
| 38 | 133 | 188 | 13588 | 3.804031355 | 0.999965843 | 0.231800837 | 14.81929399 |
| 39 | 133 | 437 | 13588 | 2.571669448 | 0.999969761 | 0.229071539 | 14.98100495 |
| 40 | 133 | 192 | 13588 | 3.724780702 | 0.99998807 | 0.241565162 | 16.20528578 |
| 41 | 133 | 148 | 13588 | 4.141841089 | 0.999999702 | 0.300740537 | 20.88938812 |
| 42 | 133 | 53 | 13588 | 7.710597248 | 0.999999716 | 0.295721164 | 20.95127119 |
| 43 | 133 | 267 | 13588 | 3.061135986 | 0.999999858 | 0.301224134 | 21.80434851 |
| 44 | 133 | 54 | 13588 | 7.56780841 | 0.99999987 | 0.296980281 | 21.90910181 |
| 45 | 133 | 99 | 13588 | 5.15986937 | 0.9999999 | 0.295639893 | 22.230872 |
| 46 | 133 | 611 | 13588 | 2.173732203 | 0.999999904 | 0.290960801 | 22.27849302 |
| 47 | 133 | 100 | 13588 | 5.108270677 | 0.999999943 | 0.293467107 | 22.89881268 |
| 48 | 133 | 22 | 13588 | 13.9316473 | 0.999999997 | 0.328676558 | 26.2532865 |
| 49 | 133 | 217 | 13588 | 3.295658501 | 0.999999997 | 0.325881168 | 26.47233348 |
| 50 | 133 | 108 | 13588 | 4.729880256 | 1 | 0.344482216 | 28.53155823 |
| 51 | 133 | 165 | 13588 | 3.715105947 | 1 | 0.357404888 | 30.13796653 |
| 52 | 133 | 228 | 13588 | 3.136657433 | 1 | 0.370168333 | 31.75922119 |
| 53 | 133 | 176 | 13588 | 3.482911825 | 1 | 0.420100589 | 36.80211781 |
| 54 | 133 | 238 | 13588 | 3.004865104 | 1 | 0.41509568 | 36.87329018 |
| 55 | 133 | 121 | 13588 | 4.221711303 | 1 | 0.427055137 | 38.51844258 |
| 56 | 133 | 753 | 13588 | 1.89948976 | 1 | 0.432566345 | 39.57163653 |
| 57 | 133 | 244 | 13588 | 2.930974978 | 1 | 0.431978548 | 40.04717075 |
| 58 | 133 | 245 | 13588 | 2.919011815 | 1 | 0.432065576 | 40.58206094 |
| 59 | 133 | 184 | 13588 | 3.331480876 | 1 | 0.439852811 | 41.86001448 |
| 60 | 133 | 2227 | 13588 | 1.422149897 | 1 | 0.452125635 | 43.58475519 |
| 61 | 133 | 31 | 13588 | 9.886975503 | 1 | 0.453613528 | 44.25845315 |
| 62 | 133 | 75 | 13588 | 5.448822055 | 1 | 0.450365735 | 44.4589569 |
| 63 | 133 | 616 | 13588 | 1.990235329 | 1 | 0.454144887 | 45.35260328 |
| 64 | 133 | 4 | 13588 | 51.08270677 | 1 | 0.456547995 | 46.10772521 |
| 65 | 133 | 193 | 13588 | 3.176126846 | 1 | 0.466771241 | 47.65120316 |
| 66 | 133 | 328 | 13588 | 2.491839354 | 1 | 0.469084762 | 48.39705082 |
| 67 | 133 | 552 | 13588 | 2.03590498 | 1 | 0.477872288 | 49.80054252 |
| 68 | 133 | 80 | 13588 | 5.108270677 | 1 | 0.475050206 | 50.01764292 |
| 69 | 133 | 633 | 13588 | 1.936785091 | 1 | 0.480392471 | 51.06697676 |
| 70 | 133 | 137 | 13588 | 3.728664728 | 1 | 0.478500703 | 51.36875 |
| 71 | 133 | 557 | 13588 | 2.017629352 | 1 | 0.476086095 | 51.60967499 |
| 72 | 133 | 35 | 13588 | 8.757035446 | 1 | 0.474622436 | 51.94267678 |
| 73 | 133 | 5 | 13588 | 40.86616541 | 1 | 0.488078471 | 53.82618784 |
| 74 | 133 | 272 | 13588 | 2.629256966 | 1 | 0.497115601 | 55.24796589 |
| 75 | 133 | 273 | 13588 | 2.619625988 | 1 | 0.497699806 | 55.7861226 |
| 76 | 133 | 86 | 13588 | 4.751879699 | 1 | 0.50004125 | 56.50311848 |
| 77 | 133 | 421 | 13588 | 2.184058722 | 1 | 0.506829853 | 57.6805401 |
| 78 | 133 | 88 | 13588 | 4.643882433 | 1 | 0.511161168 | 58.59697919 |
| 79 | 133 | 425 | 13588 | 2.163502875 | 1 | 0.514070595 | 59.36036942 |
| 80 | 133 | 89 | 13588 | 4.591703979 | 1 | 0.512284313 | 59.62851936 |
| 81 | 133 | 1772 | 13588 | 1.441385631 | 1 | 0.509804241 | 59.81828305 |
| 82 | 133 | 149 | 13588 | 3.428369582 | 1 | 0.51404321 | 60.70683174 |
| 83 | 133 | 40 | 13588 | 7.662406015 | 1 | 0.510980221 | 60.82544158 |
| 84 | 133 | 214 | 13588 | 2.864450847 | 1 | 0.507942029 | 60.94067925 |
| 85 | 133 | 431 | 13588 | 2.133384505 | 1 | 0.512485719 | 61.84796511 |
| 86 | 133 | 152 | 13588 | 3.360704393 | 1 | 0.518823826 | 62.93700974 |
| 87 | 133 | 359 | 13588 | 2.276666597 | 1 | 0.515146769 | 62.97259844 |
| 88 | 133 | 93 | 13588 | 4.394211335 | 1 | 0.51755278 | 63.64021648 |
| 89 | 133 | 93 | 13588 | 4.394211335 | 1 | 0.51755278 | 63.64021648 |
| 90 | 133 | 42 | 13588 | 7.297529538 | 1 | 0.518038348 | 64.10197022 |
| 91 | 133 | 154 | 13588 | 3.317058881 | 1 | 0.516945411 | 64.39400928 |
| 92 | 133 | 290 | 13588 | 2.466061706 | 1 | 0.515677628 | 64.66313432 |
| 93 | 133 | 43 | 13588 | 7.127819549 | 1 | 0.521573895 | 65.67569163 |
| 94 | 133 | 43 | 13588 | 7.127819549 | 1 | 0.521573895 | 65.67569163 |
| 95 | 133 | 7 | 13588 | 29.19011815 | 1 | 0.521941211 | 66.10627789 |
| 96 | 133 | 227 | 13588 | 2.700407406 | 1 | 0.541900011 | 68.54571328 |
| 97 | 133 | 227 | 13588 | 2.700407406 | 1 | 0.541900011 | 68.54571328 |
| 98 | 133 | 45 | 13588 | 6.811027569 | 1 | 0.539576096 | 68.69004358 |
| 99 | 133 | 530 | 13588 | 1.927649312 | 1 | 0.548723845 | 69.99148633 |
| 100 | 133 | 301 | 13588 | 2.37593985 | 1 | 0.545243603 | 70.01150996 |
| 101 | 133 | 301 | 13588 | 2.37593985 | 1 | 0.545243603 | 70.01150996 |
| 102 | 133 | 100 | 13588 | 4.086616541 | 1 | 0.543036347 | 70.15551118 |
| 103 | 133 | 8 | 13588 | 25.54135338 | 1 | 0.547458295 | 70.9615401 |
| 104 | 133 | 47 | 13588 | 6.521196609 | 1 | 0.549517711 | 71.52373165 |
| 105 | 133 | 165 | 13588 | 3.095921622 | 1 | 0.549873189 | 71.91098296 |
| 106 | 133 | 165 | 13588 | 3.095921622 | 1 | 0.549873189 | 71.91098296 |
| 107 | 133 | 309 | 13588 | 2.314426844 | 1 | 0.563991497 | 73.64287114 |
| 108 | 133 | 240 | 13588 | 2.554135338 | 1 | 0.578472896 | 75.36897392 |
| 109 | 133 | 50 | 13588 | 6.129924812 | 1 | 0.575662811 | 75.43274213 |
| 110 | 133 | 51 | 13588 | 6.009730208 | 1 | 0.58511535 | 76.64517662 |
| 111 | 133 | 173 | 13588 | 2.952757617 | 1 | 0.583088756 | 76.77534105 |
| 112 | 133 | 109 | 13588 | 3.749189487 | 1 | 0.586996658 | 77.45061982 |
| 113 | 133 | 52 | 13588 | 5.894158473 | 1 | 0.587580562 | 77.81289666 |
| 114 | 133 | 475 | 13588 | 1.93576573 | 1 | 0.58770496 | 78.12859078 |
| 115 | 133 | 110 | 13588 | 3.715105947 | 1 | 0.584982961 | 78.1816002 |
| 116 | 133 | 247 | 13588 | 2.481750936 | 1 | 0.587000314 | 78.66120379 |
| 117 | 133 | 10 | 13588 | 20.43308271 | 1 | 0.584028864 | 78.68588514 |
| 118 | 133 | 111 | 13588 | 3.681636524 | 1 | 0.583150293 | 78.89645491 |
| 119 | 133 | 111 | 13588 | 3.681636524 | 1 | 0.583150293 | 78.89645491 |

Rapa KEGG pathways (a):

| ID | Term | Count | % | PValue | minuslog10P |
|---|---|---|---|---|---|
| 1 | mmu04150:mTOR signaling pathway | 7 | 3.51758794 | 1.57E−06 | 5.8 |
| 2 | mmu04012:ErbB signaling pathway | 6 | 3.015075377 | 3.09E−04 | 3.51 |
| 3 | mmu04910:Insulin signaling pathway | 7 | 3.51758794 | 3.40E−04 | 3.47 |
| 4 | mmu05221:Acute myeloid leukemia | 4 | 2.010050251 | 0.007078848 | 2.15 |
| 5 | mmu04510:Focal adhesion | 6 | 3.015075377 | 0.011465215 | 1.94 |
| 6 | mmu04530:Tight junction | 5 | 2.512562814 | 0.013906132 | 1.86 |
| 7 | mmu052GO:Pathways in cancer | 7 | 3.51758794 | 0.02305239 | 1.64 |
| 8 | mmu05215:Prostate cancer | 4 | 2.010050251 | 0.024273508 | 1.61 |
| 9 | mmu03018:RNA degradation | 3 | 1.507537688 | 0.065096102 | 1.19 |
| 10 | mmu05214:Glioma | 3 | 1.507537688 | 0.072876584 | 1.14 |
| 11 | mmu04920:Adipocytokine signaling | 3 | 1.507537688 | 0.078901015 | 1.1 |
| | pathway | | | | |
| 12 | mmu04370:VEGF signaling pathway | 3 | 1.507537688 | 0.097853719 | 1.01 |
| 13 | mmu05220:Chronic myeloid leukemia | 3 | 1.507537688 | 0.097853719 | 1.01 |

Rapa KEGG pathways (b):

| ID | Genes |
|---|---|
| 1 | IPI00453603,IPI00318938,IPI00399440,IPI00268673,IPI00467843,IPI00221581,IPI00121335 |
| 2 | IPI00125319,IPI00453603,IPI00318938,IPI00113563,IPI00268673,IPI00121335 |
| 3 | IPI00125319,IPI00453603,IPI00116923,IPI00318938,IPI00268673,IPI00379844,IPI00121335 |
| 4 | IPI00453603,IPI00318938,IPI00268673,IPI00121335 |
| 5 | IPI00403938,IPI00125319,IPI00131138,IPI00113563,IPI00671847,IPI00121335 |
| 6 | IPI00118143,IPI00380354,IPI00323349,IPI00845596,IPI00121335 |
| 7 | IPI00125319,IPI00121418,IPI00381495,IPI00113563,IPI00268673,IPI00380817,IPI00121335 |
| 8 | IPI00125319,IPI00121418,IP100268673,IPI00121335 |
| 9 | IPI00169888,IPI00309059,IPI00330066 |
| 10 | IPI00121418,IPI00268673,IPI00121335 |
| 11 | IPI00268673,IPI00379844,IPI00121335 |
| 12 | IPI00111169,IPI00113563,IPI00121335 |
| 13 | IPI00121418,IPI00380817,IPI00121335 |

Rapa KEGG pathways (c):

| ID | List Total | Pop Hits | Pop Total | Fold Enrichment | Bonferroni | Benjamini | FDR |
|---|---|---|---|---|---|---|---|
| 1 | 41 | 54 | 5738 | 18.14182475 | 1.10E−04 | 1.10E−04 | 0.001624292 |
| 2 | 41 | 87 | 5738 | 9.651808242 | 0.02142063 | 0.010768293 | 0.319622391 |
| 3 | 41 | 138 | 5738 | 7.098974903 | 0.023507108 | 0.007897915 | 0.35107298 |
| 4 | 41 | 57 | 5738 | 9.821138211 | 0.391818277 | 0.116903139 | 7.088295684 |
| 5 | 41 | 198 | 5738 | 4.240946046 | 0.553895165 | 0.149082636 | 11.24945571 |
| 6 | 41 | 135 | 5738 | 5.1833785 | 0.624786839 | 0.150728903 | 13.49142317 |
| 7 | 41 | 323 | 5738 | 3.032998565 | 0.804570905 | 0.208022678 | 21.44454537 |
| 8 | 41 | 90 | 5738 | 6.220054201 | 0.820953112 | 0.193469404 | 22.45479997 |
| 9 | 41 | 60 | 5738 | 6.997560976 | 0.991011485 | 0.407576887 | 50.1731853 |
| 10 | 41 | 64 | 5738 | 6.560213415 | 0.994992413 | 0.411206711 | 54.30152323 |
| 11 | 41 | 67 | 5738 | 6.266472515 | 0.99682721 | 0.407268104 | 57.28300003 |
| 12 | 41 | 76 | 5738 | 5.524390244 | 0.999259735 | 0.451577052 | 65.55273205 |
| 13 | 41 | 76 | 5738 | 5.524390244 | 0.999259735 | 0.451577052 | 65.55273205 |

Ku BP GO (a):

| ID | Term | Count | % | PValue |
|---|---|---|---|---|
| 1 | GO:0030032~lamellipodium assembly | 3 | 3.092783505 | 3.25E−03 |
| 2 | GO:0006468~protein amino acid phosphorylation | 10 | 10.30927835 | 7.09E−03 |
| 3 | GO:0008286~insulin receptor signaling pathway | 3 | 3.092783505 | 9.83E−03 |
| 4 | GO:0030029~actin filament-based process | 5 | 5.154639175 | 0.014807537 |
| 5 | GO:0006913~nucleocytoplasmic transport | 4 | 4.12371134 | 0.014880914 |
| 6 | GO:0051493~regulation of cytoskeleton | 4 | 4.12371134 | 0.016150894 |
| | organization | | | |
| 7 | GO:0006796~phosphate metabolic process | 11 | 11.34020619 | 0.016942852 |
| 8 | GO:0006259~DNA metabolic process | 7 | 7.216494845 | 0.025415897 |
| 9 | GO:0045947~negative regulation of transla- | 2 | 2.06185567 | 0.037014038 |
| | tional initiation | | | |
| 10 | GO:0007049~cell cycle | 8 | 8.24742268 | 0.04530749 |
| 11 | GO:0033043~regulation of organelle organization | 4 | 4.12371134 | 0.049959502 |
| 12 | GO:0043484~regulation of RNA splicing | 2 | 2.06185567 | 0.052460451 |
| 13 | GO:0010605~negative regulation of macro- | 7 | 7.216494845 | 0.05420299 |
| | molecule metabolic process | | | |
| 14 | GO:0030031~cell projection assembly | 3 | 3.092783505 | 0.054397233 |
| 15 | GO:0022402~cell cycle process | 6 | 6.18556701 | 0.059968997 |
| 16 | GO:0033554~cellular response to stress | 6 | 6.18556701 | 0.065899107 |
| 17 | GO:0045884~regulation of survival gene product | 2 | 2.06185567 | 0.072676117 |
| | expression | | | |
| 18 | GO:0010558~negative regulation of macro- | 6 | 6.18556701 | 0.073909203 |
| | molecule biosynthetic process | | | |
| 19 | GO:0006916~anti-apoptosis | 3 | 3.092783505 | 0.081129603 |
| 20 | GO:0031327~negative regulation of cellular | 6 | 6.18556701 | 0.081184023 |
| | biosynthetic process | | | |
| 21 | GO:0046907~intracellular transport | 6 | 6.18556701 | 0.081807159 |
| 22 | GO:0009890~negative regulation of biosynthetic | 6 | 6.18556701 | 0.083692072 |
| | process | | | |
| 23 | GO:0006396~RNA processing | 6 | 6.18556701 | 0.085600178 |
| 24 | GO:0051129~negative regulation of cellular | 3 | 3.092783505 | 0.089153114 |
| | component organization | | | |
| 25 | GO:0006325~chromatin organization | 5 | 5.154639175 | 0.08921682 |
| 26 | GO:0030030~cell projection organization | 5 | 5.154639175 | 0.092408475 |
| 27 | GO:0043434~response to peptide hormone | 3 | 3.092783505 | 0.092426596 |
| | stimulus | | | |
| 28 | GO:0016569~covalent chromatin modification | 3 | 3.092783505 | 0.094076504 |
| 29 | GO:0043271~negative regulation of ion transport | 2 | 2.06185567 | 0.097348357 |
| 30 | GO:0007010~cytoskeleton organization | 5 | 5.154639175 | 0.098124703 |

Ku BP GO (b):

| ID | Genes |
|---|---|
| 1 | CCDC88A,NCK1,VCL |
| 2 | ALPK3,BAZ1B,BRAF,MAP3K2,SPNB2,GSK3B,NEK9,RPS6KB1,C230081A13RIK,MELK |
| 3 | PHIP,EIF4EBPI,EIF4EBP2 |
| 4 | EPB4.1L3,NCK1,ARHGEF17,MY09B,FLNC |
| 5 | LOCI 00046628,GM9118,SPNB2,GSK3B,NPM1,GM5611,GM7289,MYBBP1A,LOC633387,GM6477 |
| 6 | LOCI 00046628, CCDC88A,GM9118,SPNB2,MTAP1B,NPM1,GM5611,GM7289,LOC633387, |
| | GM6477 |
| 7 | ALPK3,BAZ1B,BRAF,MAP3K2,SPNB2,GSK3B,SYNJ1,NEK9,RPS6KB1,C230081A13R1K,MELK |
| 8 | ATRX,TRP53BP1,CCDC88A,MDC1,ORC6L,TOP2B,MYC |
| 9 | ElF4EBP1,E1F4EBP2 |
| 10 | LOC100046628,GM9118,RBL1,GM5611,ZFP318,GM6477,MDC1,GSK3B,NPM1,KIF20B,NEK9, |
| | GM7289,LOC633387,ERCC6L |
| 11 | LOC100046628,CCDC88A,GM9118,SPNB2,MTAP1B,NPM1,GM5611,GM7289,LOC633387,GM6477 |
| 12 | LOC100046628,GM9118,GPX4,NPM1,GM5611,GM7289,HNRPLL,LOC633387,GM6477 |
| 13 | LOC100046628,GM9118,RBL1,GM5611,GM6477,EIF4EBP1,EIF4EBP2,NPM1,GM7289,MYC,MYBB |
| | P1A, LOC633387,SMARCA4 |
| 14 | CCDC88A,NCK1,VCL |
| 15 | LOC100046628,GM9118,GSK3B,NPM1,KIF20B,NEK9,GM5611,GM7289,ZFP318,LOC633387, |
| | GM6477,ERCC6L |
| 16 | ATRX,TRP53BP1,BAZ1B,MDC1,GPX4,GSK3B,HNRPLL |
| 17 | AKT1S1,MYC |
| 18 | EIF4EBP1,EIF4EBP2,RBL1,MYBBP1A,MYC,SMARCA4 |
| 19 | AKT1S1,GSK3B,MYC |
| 20 | EIF4EBP1,EIF4EBP2,RBL1,MYBBP1A,MYC,SMARCA4 |
| 21 | LOC100046628,GM9118,SPNB2,GSK3B,NPM1,AKAP12,GM5611,MYO9B,GM7289,MYBBP1A, |
| | LOC633387,GM6477 |
| 22 | EIF4EBP1,EIF4EBP2,RBL1,MYBBP1A,MYC,SMARCA4 |
| 23 | LOC100046766,SF3B1,EXOSC9,LOC100046744,GPX4,LARP7,SRRM2,LOC100046735,POP1, |
| | LOC675032,LOC100047322,HNRPLL |
| 24 | RTN4,SPNB2,MTAP1B |
| 25 | BAZ1B,GPX4,RBL1,SETD2,HNRPLL,SMARCA4 |
| 26 | CCDC88A,NCK1,MTAP1B,TOP2B,VCL |
| 27 | PHIP,EIF4EBP1,EIF4EBP2 |
| 28 | BAZ1B,SETD2,SMARCA4 |
| 29 | BEST3,NEDD4L |
| 30 | EPB4.1L3,LOC100046628,GM9118,NCK1,MTAP1B,NPM1,ARHGEF17,GM5611,GM7289,LOC633387, |
| | GM6477 |

Ku BP GO (c):

| ID | List Total | Pop Hits | Pop Total | Fold Enrichment | Bonferroni | Benjamini | FDR |
|---|---|---|---|---|---|---|---|
| 1 | 74 | 16 | 13588 | 34.42905405 | 0.882872164 | 8.83E−01 | 4.76E+3000 |
| 2 | 74 | 640 | 13588 | 2.869087838 | 0.990722194 | 0.903678632 | 10.0912277 |
| 3 | 74 | 28 | 13588 | 19.67374517 | 0.998498942 | 0.885501679 | 13.73744045 |
| 4 | 74 | 176 | 13588 | 5.216523342 | 0.99994544 | 0.859597701 | 19.99753834 |
| 5 | 74 | 96 | 13588 | 7.650900901 | 0.99994805 | 0.806833826 | 20.08660666 |
| 6 | 74 | 99 | 13588 | 7.419055419 | 0.999977769 | 0.737959309 | 21.61357964 |
| 7 | 74 | 866 | 13588 | 2.332376256 | 0.999986913 | 0.713301589 | 22.55196875 |
| 8 | 74 | 421 | 13588 | 3.053091096 | 0.999999956 | 0.785616066 | 31.95670058 |
| 9 | 74 | 7 | 13588 | 52.46332046 | 1 | 0.87357656 | 43.11139373 |
| 10 | 74 | 611 | 13588 | 2.404211085 | 1 | 0.869178376 | 50.01437111 |
| 11 | 74 | 154 | 13588 | 4.769392769 | 1 | 0.878480874 | 53.53582478 |
| 12 | 74 | 10 | 13588 | 36.72432432 | 1 | 0.875783402 | 55.33191067 |
| 13 | 74 | 506 | 13588 | 2.540220062 | 1 | 0.869599238 | 56.5448088 |
| 14 | 74 | 70 | 13588 | 7.869498069 | 1 | 0.855869756 | 56.678091 |
| 15 | 74 | 393 | 13588 | 2.803383536 | 1 | 0.869269287 | 60.34271545 |
| 16 | 74 | 404 | 13588 | 2.727053786 | 1 | 0.881875859 | 63.92395423 |
| 17 | 74 | 14 | 13588 | 26.23166023 | 1 | 0.895305874 | 67.64630011 |
| 18 | 74 | 418 | 13588 | 2.635717057 | 1 | 0.888826293 | 68.28378078 |
| 19 | 74 | 88 | 13588 | 6.25982801 | 1 | 0.901699824 | 71.78744367 |
| 20 | 74 | 430 | 13588 | 2.562162162 | 1 | 0.892310062 | 71.81242243 |
| 21 | 74 | 431 | 13588 | 2.55621747 | 1 | 0.884669916 | 72.09697589 |
| 22 | 74 | 434 | 13588 | 2.538547764 | 1 | 0.881167309 | 72.94148547 |
| 23 | 74 | 437 | 13588 | 2.521120663 | 1 | 0.877904651 | 73.77204657 |
| 24 | 74 | 93 | 13588 | 5.923278117 | 1 | 0.879820716 | 75.2555379 |
| 25 | 74 | 315 | 13588 | 2.914628915 | 1 | 0.871223772 | 75.28140884 |
| 26 | 74 | 319 | 13588 | 2.878081844 | 1 | 0.872300131 | 76.54568607 |
| 27 | 74 | 95 | 13588 | 5.798577525 | 1 | 0.863873178 | 76.55268842 |
| 28 | 74 | 96 | 13588 | 5.738175676 | 1 | 0.860545996 | 77.18216045 |
| 29 | 74 | 19 | 13588 | 19.32859175 | 1 | 0.862220501 | 78.38406819 |
| 30 | 74 | 326 | 13588 | 2.81628254 | 1 | 0.856532331 | 78.66045069 |

Ku KEGG pathways (a):

| ID | Term | Count | % | PValue | minuslogP |
|---|---|---|---|---|---|
| 1 | mmu04012:ErbB signaling pathway | 6 | 6.060606061 | 2.42E−05 | 4.62 |
| 2 | mmu04150:mTOR signaling pathway | 4 | 4.04040404 | 1.39E−03 | 2.86 |
| 3 | mmu05221:Acute myeloid leukemia | 4 | 4.04040404 | 1.62E−03 | 2.79 |
| 4 | mmu04110:Cell cycle | 4 | 4.04040404 | 0.015586032 | 1.81 |
| 5 | mmu04910:Insulin signaling pathway | 4 | 4.04040404 | 0.01903863 | 1.72 |
| 6 | mmu05213:Endometrial cancer | 3 | 3.03030303 | 0.019575313 | 1.71 |
| 7 | mmu04010:MARK signaling pathway | 5 | 5.050505051 | 0.022772811 | 1.64 |
| 8 | mmu03018:RNA degradation | 3 | 3.03030303 | 0.025605774 | 1.59 |
| 9 | mmu04510:Focal adhesion | 4 | 4.04040404 | 0.048078613 | 1.32 |
| 10 | mmu05210:Colorectal cancer | 3 | 3.03030303 | 0.049516956 | 1.31 |
| 11 | mmu04350:TGF-beta signaling | 3 | 3.03030303 | 0.050554685 | 1.3 |
| | pathway | | | | |

Ku KEGG pathways (b):

| ID | Genes |
|---|---|
| 1 | IPI00453999,IPI00125319,IPI00453603,IPI00230719,IPI00318938,IPI00668709,IPI00131999 |
| 2 | IPI00453603,IPI00230719,IPI00318938,IPI00668709,IPI00221581 |
| 3 | IPI00453603,IPI00230719,IPI00318938,IPI00668709,IPI00131999 |
| 4 | IPI00125319,IPI00124717,IPI00137864,IPI00131999 |
| 5 | IPI00125319,IPI00453603,IPI00230719,IPI00318938,IPI00668709 |
| 6 | IPI00125319,IPI00230719,IPI00668709,IPI00131999 |
| 7 | IPI00230719,IPI00117088,IPI00123474,IPI00668709,IPI00664670,IPI00131999 |
| 8 | IPI00309059,IPI00119442,IPI00330066 |
| 9 | IPI00125319,IPI00230719,IPI00405227,IPI00668709,IPI00664670 |
| 10 | IPI00125319,IPI00230719,IPI00668709,IPI00131999 |
| 11 | IPI00453603,IPI00137864,IPI00131999 |

Ku KEGG pathways (c):

| ID | List Total | Pop Hits | Pop Total | Fold Enrichment | Bonferroni | Benjamini | FDR |
|---|---|---|---|---|---|---|---|
| 1 | 25 | 87 | 5738 | 15.82896552 | 1.38E−03 | 1.38E−03 | 0.023962829 |
| 2 | 25 | 54 | 5738 | 17.00148148 | 0.076050732 | 0.038777202 | 1.366086094 |
| 3 | 25 | 57 | 5738 | 16.10666667 | 0.088400206 | 0.030380355 | 1.596618843 |
| 4 | 25 | 128 | 5738 | 7.1725 | 0.591555799 | 0.200564953 | 14.41914204 |
| 5 | 25 | 138 | 5738 | 6.652753623 | 0.6656829 | 0.196785171 | 17.34826426 |
| 6 | 25 | 52 | 5738 | 13.24153846 | 0.675950335 | 0.171227632 | 17.79538958 |
| 7 | 25 | 265 | 5738 | 4.330566038 | 0.731003969 | 0.171037042 | 20.41453368 |
| 8 | 25 | 60 | 5738 | 11.476 | 0.772029847 | 0.168744085 | 22.67212753 |
| 9 | 25 | 198 | 5738 | 4.636767677 | 0.939707705 | 0.268063281 | 38.63940264 |
| 10 | 25 | 86 | 5738 | 8.006511628 | 0.944686732 | 0.251342928 | 39.55225136 |
| 11 | 25 | 87 | 5738 | 7.914482759 | 0.948025839 | 0.235718393 | 40.20324697 |

TABLE 11

| | |
|---|---|
| Gene Symbol | Annotation |

Rapa replicate 1

| | |
|---|---|
| 1600027N09Rik | RIKEN cDNA 1600027N09 gene |
| Ahctf1 | AT-hook-containing transcription factor 1 |
| Ahnak | AHNAK nucleoprotein isoform 1 |
| Akt1s1 | Proline-rich AKT1 substrate 1 |
| Bat2l | Isoform 1 of Protein BAT2-like |
| Bclaf1 | Isoform 2 of Bcl-2-associated transcription factor 1 |
| Bmp2k | Isoform 1 of BMP-2-inducible protein kinase |
| C130092O11Rik | Isoform 1 of Uncharacterized protein KIAA1680 |
| Carhsp1 | Calcium-regulated heat stable protein 1 |
| Ccn/l | Isoform 1 of Cyclin-L1 |
| Chd1 | Chromodomain-helicase-DNA-binding protein 1 |
| Cttn | Src substrate cortactin |
| D6Wsu116e | Isoform 1 of Protein FAM21 |
| Dab2 | Isoform p93 of Disabled homolog 2 |
| Dock11 | Dedicator of cytokinesis protein 11 |
| Edc4 | Isoform 1 of Enhancer of mRNA-decapping protein 4 |
| Eif3a | Eukaryotic translation initiation factor 3 subunit A |
| Eif4ebp1 | Eukaryotic translation initiation factor 4E-binding protein 1 |
| Eif4ebp2 | Eukaryotic translation initiation factor 4E-binding protein 2 |
| Eif4g1 | Isoform 1 of Eukaryotic translation initiation factor 4 gamma 1 |
| Eif5; LOC100047658 | Eukaryotic translation initiation factor 5 |
| Epyc | Epiphycan |
| Fkbp15 | Isoform B of FK506-binding protein 15 |
| Foxk1 | Forkhead box protein K1 |
| Gm13697 | Novel protein containing MIF4G and MA3 domains |
| Grb10 | Isoform 3 of Growth factor receptor-bound protein 10 |
| Gtf2f1 | General transcription factor IIF subunit 1 |
| Hdgfrp2 | Isoform 3 of Hepatoma-derived growth factor-related protein 2 |
| Hisppd1 | 140 kDa protein |
| Ibtk | Isoform 2 of Inhibitor of Bruton tyrosine kinase |
| Inf2 | Isoform 1 of Inverted formin-2 |
| Iqsec1 | IQ motif and Sec7 domain 1 isoform b |
| Irs2 | Insulin receptor substrate 2 |
| Iws1 | Isoform 1 of Protein IWS1 homolog |
| Kdm3b | Isoform 2 of Lysine-specific demethylase 3B |
| Larp1 | Isoform 1 of La-related protein 1 |
| Larp4 | Putative uncharacterized protein |
| Ldhd | 22 kDa protein |
| Luc7l2 | Isoform 1 of Putative RNA-binding protein Luc7-like 2 |
| Macf1 | Isoform 3 of Microtubule-actin cross-linking factor 1 |
| Mett10d | Isoform 1 of Putative methyltransferase METT10D |
| Mtap1b | Microtubule-associated protein 1B |
| Myef2 | Isoform 2 of Myelin expression factor 2 |
| Myo5a | Myosin-Va |
| Ndrg3 | Protein NDRG3 |
| Nfic | Isoform 1 of Nuclear factor 1 C-type |
| Npm1 | Nucleophosmin |
| Numa1 | Nuclear mitotic apparatus protein 1 |
| Patl1 | Protein PAT1 homolog 1 |
| Pcbp2 | Isoform 1 of Poly(rC)-binding protein 2 |
| Pds5b | Isoform 1 of Sister chromatid cohesion protein PDS5 homolog B |
| Peg3 | Isoform 1 of Paternally-expressed gene 3 protein |
| Phf3 | PHD finger protein 3 |
| Phldb1 | Isoform 2 of Pleckstrin homology-like domain family B member 1 |
| Phldb2 | Isoform 1 of Pleckstrin homology-like domain family B member 2 |
| Pi4k2a | Phosphatidylinositol 4-kinase type 2-alpha |
| Pkn2 | Isoform 1 of Serine/threonine-protein kinase N2 |
| Pla2g4a | Cytosolic phospholipase A2 |
| Plekhm1 | Pleckstrin homology domain-containing family M member 1 |
| Ppp1r12a | MCG122391 isoform CRA_e |
| Rab1 | Ras-related protein Rab-1A |
| Rps6 | 29 kDa protein |
| Rps6kb1 | Isoform Alpha I of Ribosomal protein S6 kinase beta-1 |
| Rsl1d1 | Putative uncharacterized protein |
| Sap30 | Histone deacetylase complex subunit SAP30 |
| Serbp1 | Isoform 1 of Plasminogen activator inhibitor 1 RNA-binding protein |
| Sfrs8 | splicing factor arginine/serine-rich 8 |
| Sgta | Isoform 1 of Small glutamine-rich tetratricopeptide repeat-containing protein alpha |
| Smarca4 | Putative uncharacterized protein |
| Snx17 | Sorting nexin-17 |
| Srpk2 | serine/arginine-rich protein-specific kinase 2 |
| Srrm2 | Isoform 3 of Serine/arginine repetitive matrix protein 2 |
| Syap1 | Synapse-associated protein 1 |
| Tbc1d4 | 140 kDa protein |
| Tcfeb | Transcription factor EB |
| Tjp2 | Tight junction protein ZO-2 |
| Tmem106b | Transmembrane protein 106B isoform CRA_b |
| Tmpo | Isoform Beta of Lamina-associated polypeptide 2 isoforms beta/delta/epsilon/gamma |
| Trim28 | Isoform 1 of Transcription intermediary factor 1-beta |
| Ubxn7 | UBX domain-containing protein 7 |
| Usp24 | Isoform 1 of Ubiquitin carboxyl-terminal hydrolase 24 |
| Uvrag | UV radiation resistance associated |
| Wnk1 | Serine/threonine-protein kinase WNK1 |
| Zc3hc1 | Isoform 1 of Nuclear-interacting partner of ALK |
| Zfp516 | Zinc finger protein 516 |

Rapa Replicate 2

| | |
|---|---|
| Mib1 | E3 ubiquitin-protein ligase MIB1 |
| rsp6 | 29 kDa protein |
| Srrm2 | Isoform 3 of Serine/arginine repetitive matrix protein 2 |
| Gm13099 | Novel protein similar to preferentially expressed antigen in melanoma-like family |
| Rnf19b | IBR domain containing 3 |
| Rps6 | 29 kDa protein |
| Carhsp1 | Calcium-regulated heat stable protein 1 |
| Pgrmc2 | Membrane-associated progesterone receptor component 2 |
| FLNA | Filamin-A |
| Ext1 | Exostosin-1 |
| Gm14085 | Novel protein similar to solute carrier family 28 (Sodium-coupled nucleoside |
| | transporter) member 2 |
| Akt1s1 | Proline-rich AKT1 substrate 1 |
| Gm9757 | Putative uncharacterized protein |
| Eif4ebp2 | Eukaryotic translation initiation factor 4E-binding protein 2 |
| Gon4l | RIKEN cDNA 5830417l10 gene |
| Mogat1 | 2-acylglycerol O-acyltransferase 1 |
| Eef2k | Elongation factor 2 kinase |
| Dhx15 | Putative pre-mRNA-splicing factor ATP-dependent RNA helicase DHX15 |
| Srrm1 | Isoform 2 of Serine/arginine repetitive matrix protein 1 |
| Setd1a | SET domain containing 1A |
| D830015G02Rik | Putative uncharacterized protein |
| Vwa5b1 | von Willebrand factor A domain-containing protein 5B1 |
| Ibtk | Isoform 2 of Inhibitor of Bruton tyrosine kinase |
| Erc1; LOC100048600 | Isoform 1 of ELKS/RAB6-interacting/CAST family member 1 |
| Adamts2 | A disintegrin and metalloproteinase with thrombospondin motifs 2 |
| Grb10 | Isoform 3 of Growth factor receptor-bound protein 10 |
| Foxk1 | Forkhead box protein K1 |
| Gm6988 | similar to hCG1640785 |
| Tnc | Isoform 1 of Tenascin |
| Larp1 | Isoform 1 of La-related protein 1 |
| Alkbh6 | 24 kDa protein |
| Larp7 | Isoform 1 of La-related protein 7 |
| Ahnak | AHNAK nucleoprotein isoform 1 |
| Qsox2 | Isoform 3 of Sulfhydryl oxidase 2 |
| Wnk1 | Serine/threonine-protein kinase WNK1 |
| Megf11 | Isoform 4 of Multiple epidermal growth factor-like domains 11 |
| Ahctf1 | AT-hook-containing transcription factor 1 |
| Fxr1 | Isoform E of Fragile X mental retardation syndrome-related protein 1 |
| Ddx21 | Nucleolar RNA helicase 2 |
| Lin9 | Isoform 2 of Lin-9 homolog |
| D6Wsu116e | Isoform 1 of Protein FAM21 |
| Sfrs8 | splicing factor arginine/serine-rich 8 |
| Ppfibp2 | Isoform 4 of Liprin-beta-2 |
| Hdgfrp2 | Isoform 3 of Hepatoma-derived growth factor-related protein 2 |
| Irs2 | Insulin receptor substrate 2 |
| Tinf2 | Putative uncharacterized protein |
| Hisppd1 | 140 kDa protein |
| Edc3 | Enhancer of mRNA-decapping protein 3 |
| Aim1l | Absent in melanoma 1-like |
| Herc1 | hect (homologous to the E6-AP (UBE3A) carboxyl terminus) domain and RCC1 |
| | (CHC1)-like domain (RLD) 1 |
| Fbxw9 | F-box and WD-40 domain protein 9 |
| Sfrs18 | splicing factor arginine/serine-rich 18 |
| Dnajc2 | DnaJ homolog subfamily C member 2 |
| Cux1 | cut-like homeobox 1 isoform a |
| Ranbp10 | Ran-binding protein 10 |
| Foxk2 | Isoform 1 of Forkhead box protein K2 |
| Bod1l | biorientation of chromosomes in cell division 1-like |
| Zeb2 | Zinc finger E-box-binding homeobox 2 |
| Bcr | Breakpoint cluster region protein |
| Pdcd11 | Protein RRP5 homolog |
| Hn1l | Hematological and neurological expressed 1-like protein |
| Palm | Isoform 1 of Paralemmin |
| Phf3 | PHD finger protein 3 |
| Ptk2 | Isoform 1 of Focal adhesion kinase 1 |
| 1110013L07Rik | Putative uncharacterized protein |
| Tcfeb | Transcription factor EB |
| Ehmt2 | Isoform 1 of Histone-lysine N-methyltransferase H3 lysine-9 specific 3 |
| Zc3h4 | Isoform 2 of Zinc finger CCCH domain-containing protein 4 |
| Ktn1 | Isoform 1 of Kinectin |
| Pbx2 | Pre-B-cell leukemia transcription factor 2 |
| Phldb1 | Isoform 2 of Pleckstrin homology-like domain family B member 1 |
| Cdgap | Cdc42 GTPase-activating protein |
| Eif3a | Eukaryotic translation initiation factor 3 subunit A |
| Tbc1d4 | 140 kDa protein |
| Dennd4a | hypothetical protein LOC102442 |
| Wdr91 | WD repeat-containing protein 91 |
| Junb | Transcription factor jun-B |
| Srpk2 | serine/arginine-rich protein-specific kinase 2 |
| Eif4b | Eukaryotic translation initiation factor 4B |
| Gbf1 | Golgi-specific brefeldin A-resistance factor 1 |
| Cd2ap | CD2-associated protein |
| LOC100048559; Sfrs1 | Isoform 1 of Splicing factor arginine/serine-rich 1 |
| Ccdc6 | coiled-coil domain containing 6 |
| Ndrg3 | Protein NDRG3 |
| Ulk1 | Putative uncharacterized protein |
| Arhgap17 | Isoform 1 of Rho GTPase-activating protein 17 |
| Spna2 | Isoform 2 of Spectrin alpha chain brain |
| Patl1 | Protein PAT1 homolog 1 |
| Bcas3 | Isoform 1 of Breast carcinoma-amplified sequence 3 homolog |
| Atg2a | Autophagy-related protein 2 homolog A |
| Ccnl1 | Isoform 1 of Cyclin-L1 |
| Znrf2 | E3 ubiquitin-protein ligase ZNRF2 |
| Larp4 | Putative uncharacterized protein |
| Mtor | Isoform 1 of FKBP12-rapamycin complex-associated protein |
| Gsk3b | Glycogen synthase kinase-3 beta |
| Ranbp9 | RAN binding protein 9 |
| Kdm6a | Isoform 1 of Lysine-specific demethylase 6A |
| C130092O11Rik | Isoform 1 of Uncharacterized protein KIAA1680 |
| Mtap1b | Microtubule-associated protein 1B |
| Prkd2 | Serine/threonine-protein kinase D2 |
| Usp36 | Ubiquitin specific peptidase 36 |
| Slc4a1ap | solute carrier family 4 (anion exchanger) member 1 adaptor protein |
| Llgl1 | lethal giant larvae homolog 1 isoform 1 |
| Cabin1 | calcineurin binding protein 1 |
| Pcbp1 | Poly(rC)-binding protein 1 |
| Eif4ebp1 | Eukaryotic translation initiation factor 4E-binding protein 1 |
| Rfc1 | Rfc1 protein |
| Pds5b | Isoform 1 of Sister chromatid cohesion protein PDS5 homolog B |
| St5 | Isoform 1 of Suppression of tumorigenicity 5 |
| Eif4g1 | Isoform 1 of Eukaryotic translation initiation factor 4 gamma 1 |
| Pom121 | Nuclear envelope pore membrane protein POM 121 |
| Smarcc2 | Isoform 2 of SWI/SNF complex subunit SMARCC2 |
| Eps8l2 | Isoform 1 of Epidermal growth factor receptor kinase substrate 8-like protein 2 |
| Plekhm1 | Pleckstrin homology domain-containing family M member 1 |
| B230208H17Rik | Putative GTP-binding protein Parf |
| Mll2 | similar to myeloid/lymphoid or mixed-lineage leukemia 2 |
| Snx30 | Sorting nexin-30 |
| Rps6kb1 | Isoform Alpha I of Ribosomal protein S6 kinase beta-1 |
| Usp24 | Isoform 1 of Ubiquitin carboxyl-terminal hydrolase 24 |
| Micall1 | Isoform 1 of MICAL-like protein 1 |
| Med1 | Isoform 4 of Mediator of RNA polymerase II transcription subunit 1 |
| Sorbs3 | Vinexin |
| Phldb2 | Isoform 1 of Pleckstrin homology-like domain family B member 2 |
| Smarcad1 | Isoform 1 of SWI/SNF-related matrix-associated actin-dependent regulator of |
| | chromatin subfamily A containing DEAD/H box 1 |
| Crkrs | Isoform 2 of Cell division cycle 2-related protein kinase 7 |
| Gtf2f1 | General transcription factor IIF subunit 1 |
| Scrib | Isoform 1 of Protein LAP4 |
| 2610110G12Rik | Isoform 1 of UPF0635 protein C6orf134 homolog |
| Mllt4 | Isoform 3 of Afadin |
| Nacc1 | Nucleus accumbens-associated protein 1 |
| Zfp106 | Isoform 1 of Zinc finger protein 106 |
| Rictor | Isoform 1 of Rapamycin-insensitive companion of mTOR |
| Tns1 | tensin 1 |
| Cttn | Src substrate cortactin |
| Sbno1 | Isoform 2 of Protein strawberry notch homolog 1 |
| Fmnl3 | Isoform 1 of Formin-like protein 3 |
| Syap1 | Synapse-associated protein 1 |
| Strn3 | Striatin-3 |
| Rb1 | Retinoblastoma-associated protein |
| Ehbp1 | Isoform 2 of EH domain-binding protein 1 |
| Sqstm1 | Isoform 1 of Sequestosome-1 |
| Zc3hc1 | Isoform 1 of Nuclear-interacting partner of ALK |
| Tox4 | TOX high mobility group box family member 4 |
| Emg1 | Probable ribosome biogenesis protein NEP1 |
| Pcm1 | Isoform 1 of Pericentriolar material 1 protein |
| Trip10 | Isoform 3 of Cdc42-interacting protein 4 |
| LOC100048123; Akt2 | RAC-beta serine/threonine-protein kinase |
| Aak1 | Isoform 2 of AP2-associated protein kinase 1 |
| Srrm2 | Isoform 3 of Serine/arginine repetitive matrix protein 2 |
| Phldb2 | Isoform 1 of Pleckstrin homology-like domain family B member 2 |
| Phldb1 | Isoform 2 of Pleckstrin homology-like domain family B member 1 |
| Ahctf1 | AT-hook-containing transcription factor 1 |
| Ahctf1 | AT-hook-containing transcription factor 1 |
| Ahctf1 | AT-hook-containing transcription factor 1 |
| Arhgap17 | Isoform 1 of Rho GTPase-activating protein 17 |
| Rb1 | Retinoblastoma-associated protein |
| Rb1 | Retinoblastoma-associated protein |
| Tox4 | TOX high mobility group box family member 4 |
| Tox4 | TOX high mobility group box family member 4 |
| Hells | Isoform 1 of Lymphocyte-specific helicase |
| Hells | Isoform 1 of Lymphocyte-specific helicase |
| Hells | Isoform 1 of Lymphocyte-specific helicase |
| Erbb2ip | Isoform 1 of Protein LAP2 |
| Eif4ebp2 | Eukaryotic translation initiation factor 4E-binding protein 2 |
| Eif4ebp2 | Eukaryotic translation initiation factor 4E-binding protein 2 |
| Pds5b | Isoform 1 of Sister chromatid cohesion protein PDS5 homolog B |
| Pds5b | Isoform 1 of Sister chromatid cohesion protein PDS5 homolog B |
| Pds5b | Isoform 1 of Sister chromatid cohesion protein PDS5 homolog B |
| Fam129a | Protein Niban |
| Eif4ebp1 | Eukaryotic translation initiation factor 4E-binding protein 1 |
| Ahnak | AHNAK nucleoprotein isoform 1 |
| Ahnak | AHNAK nucleoprotein isoform 1 |
| Ahnak | AHNAK nucleoprotein isoform 1 |
| Eif4ebp1 | Eukaryotic translation initiation factor 4E-binding protein 1 |
| Arid1a | AT rich interactive domain 1A |
| Fam129a | Protein Niban |
| Fam129a | Protein Niban |
| Atxn2 | ataxin 2 |
| Atxn2 | ataxin 2 |
| Ranbp10 | Ran-binding protein 10 |
| Ranbp10 | Ran-binding protein 10 |
| Ranbp10 | Ran-binding protein 10 |
| Ranbp10 | Ran-binding protein 10 |
| Ranbp10 | Ran-binding protein 10 |
| Tox4 | TOX high mobility group box family member 4 |
| Tox4 | TOX high mobility group box family member 4 |

Ku

| | |
|---|---|
| Myo18a | Isoform 4 of Myosin-XVIIIa |
| Brd2 | Isoform 2 of Bromodomain-containing protein 2 |
| 6330577E15Rik | Uncharacterized protein C10orf78 homolog |
| Best3 | Bestrophin-3 |
| Larp1 | Isoform 1 of La-related protein 1 |
| Fam62c | Isoform 1 of Extended synaptotagmin-3 |
| Mdn1 | Midasin homolog |
| Rtn4 | Isoform 2 of Reticulon-4 |
| Pcdh24 | Pcdh24 protein |
| Synj1 | similar to mKIAA0910 protein |
| Zfp318 | zinc finger protein 318 isoform 1 |
| Akt1s1 | Proline-rich AKT1 substrate 1 |
| Alpk3 | myocyte induction differentiation originator |
| Zc3h14 | Isoform 2 of Zinc finger CCCH domain-containing protein 14 |
| Edc4 | Isoform 1 of Enhancer of mRNA-decapping protein 4 |
| Srrm2 | Isoform 3 of Serine/arginine repetitive matrix protein 2 |
| Kif20b | Isoform 1 of M-phase phosphoprotein 1 |
| 1110007A13Rik | UPF0557 protein C10orf119 homolog |
| Pi4k2a | Phosphatidylinositol 4-kinase type 2-alpha |
| Top2b | DNA topoisomerase 2-beta |
| BC021381 | Isoform 2 of Uncharacterized protein KIAA1931 |
| Bbx | Isoform 1 of HMG box transcription factor BBX |
| Vcl | Vinculin |
| Eif4ebp1 | Eukaryotic translation initiation factor 4E-binding protein 1 |
| Rrp15 | RRP15-like protein |
| D10Wsu102e | Uncharacterized protein C12orf45 homolog |
| Braf | Isoform 1 of B-Raf proto-oncogene serine/threonine-protein kinase |
| Atg2b | Isoform 1 of Autophagy-related protein 2 homolog B |
| Npm1 | Nucleophosmin |
| Pcsk5 | proprotein convertase subtilisin/kexin type 5 |
| Mtap1b | Microtubule-associated protein 1B |
| Ercc6l | DNA excision repair protein ERCC-6-like |
| Serinc1 | Serine incorporator 1 |
| Klf3; LOC100046855 | Krueppel-like factor 3 |
| Smarca4 | Putative uncharacterized protein |
| Aak1 | Uncharacterized protein FLJ45252 homolog |
| Eif4ebp2 | Eukaryotic translation initiation factor 4E-binding protein 2 |
| Ndrg1 | Protein NDRG1 |
| Melk | Maternal embryonic leucine zipper kinase |
| Arhgef17 | Isoform 1 of Rho guanine nucleotide exchange factor 17 |
| Grit | Isoform 2 of Rho/Cdc42/Rac GTPase-activating protein RICS |
| Mdc1 | mediator of DNA damage checkpoint 1 |
| Nfkb2 | NF-kB2 splice variant 4 |
| Pcm1 | Isoform 1 of Pericentriolar material 1 protein |
| Mybbp1a | Myb-binding protein 1A |
| Sf3b1 | Splicing factor 3B subunit 1 |
| Atrx | Transcriptional regulator ATRX |
| Ccdc88a | Isoform 2 of Girdin |
| Baz1b | Isoform 1 of Tyrosine-protein kinase BAZ1B |
| Nedd4l | Isoform 3 of E3 ubiquitin-protein ligase NEDD4-like |
| Orc6l | Origin recognition complex subunit 6 |
| Trp53bp1 | Transformation related protein 53 binding protein 1 |
| Map3k2 | Mitogen-activated protein kinase kinase kinase 2 |
| Hectd2 | Hectd2 protein |
| Usp10 | Ubiquitin carboxyl-terminal hydrolase 10 |
| D830031N03Rik | similar to mKIAA0754 protein |
| Nck1 | non-catalytic region of tyrosine kinase adaptor protein 1 |
| Exosc9 | Exosome complex exonuclease RRP45 |
| Dap | Death-associated protein 1 |
| Rps6kb1 | Isoform Alpha I of Ribosomal protein S6 kinase beta-1 |
| Lmna | Isoform C2 of Lamin-A/C |
| Sltm | Isoform 1 of SAFB-like transcription modulator |
| Sh3pxd2a | Isoform 1 of SH3 and PX domain-containing protein 2A |
| Flnc | Isoform 1 of Filamin-C |
| Oxr1 | Isoform 2 of Oxidation resistance protein 1 |
| Rin2 | Isoform 1 of Ras and Rab interactor 2 |
| Nek9 | Serine/threonine-protein kinase Nek9 |
| Pebp1 | Phosphatidylethanolamine-binding protein 1 |
| Pop1 | Processing of 1 ribonuclease P/MRP family |
| Serhl | Serine hydrolase-like protein |
| Epb4.1l3 | Isoform 1 of Band 4.1-like protein 3 |
| Hnrpll | Isoform 1 of Heterogeneous nuclear ribonucleoprotein L-like |
| Samhd1 | SAM domain and HD domain-containing protein 1 |
| Zfp828 | Zinc finger protein 828 |
| Larp7 | Isoform 1 of La-related protein 7 |
| Myc | myc proto-oncogene protein |
| Myo9a | Isoform 2 of Myosin-IXa |
| Gsk3b | Glycogen synthase kinase-3 beta |
| Zfp395 | zinc finger protein 395 |
| Bend3 | BEN domain-containing protein 3 |
| Akap12 | Isoform 1 of A-kinase anchor protein 12 |
| Eif4g1 | Isoform 1 of Eukaryotic translation initiation factor 4 gamma 1 |
| Eif4b | Eukaryotic translation initiation factor 4B |
| Dock7 | Isoform 2 of Dedicator of cytokinesis protein 7 |
| Patl1 | Protein PAT1 homolog 1 |
| Slc7a11 | Cystine/glutamate transporter |
| Myo9b | Isoform 1 of Myosin-IXb |
| Setd2 | SET domain containing 2 |
| Gphn | Gephyrin |
| Erf | ETS domain-containing transcription factor ERF |
| Spnb2 | Isoform 2 of Spectrin beta chain brain 1 |
| Phip | PH-interacting protein |
| Sdpr | Serum deprivation-response protein |
| Tcof1 | Treacle protein |
| Pwp1 | Periodic tryptophan protein 1 homolog |
| Rb/1 | Isoform Long of Retinoblastoma-like protein 1 |
| Eef1b2 | Elongation factor 1-beta |
| Phactr4 | Isoform 1 of Phosphatase and actin regulator 4 |
| C230081A13Rik | Tyrosine-protein kinase-protein kinase SgK269 |
| Ahnak2 | Putative uncharacterized protein |

Table 11. Proteins with downregulated phosphorylation identified in the rapamycin and Ku-0063794 screen.

1. A method for determining mTOR kinase activity in a cancer cell, the method comprising
(a) obtaining a cancer cell from a subject diagnosed to have a cancer,
(b) determining the level of Grb10 phosphorylation in the cell, and
(c) comparing the level of Grb10 phosphorylation to a reference level, wherein if the level of Grb10 phosphorylation in the cell is higher than the reference level, then the cell is determined to exhibit an elevated level of mTOR kinase activity.

2. The method of claim 1, further comprising selecting a method of treatment based on the cell exhibiting an elevated level of mTOR kinase activity.

3. The method of claim 2, wherein if the cell is determined to exhibit an elevated level of mTOR kinase activity, then the selected method of treatment comprises administering an effective amount of an mTOR kinase inhibitor to the subject.

4. The method of claim 3, wherein the method of treatment further comprises administering an effective amount of a compound that stabilizes Grb10 or that inhibits the degradation of Grb10 and/or an effective amount of a PI3K inhibitor to the subject.

5. The method of claim 3, wherein the mTOR inhibitor is an allosteric mTOR kinase inhibitor or a catalytic mTOR kinase inhibitor.

6. The method of claim 5, wherein the allosteric mTOR kinase inhibitor is rapamycin or a rapamycin analog, or wherein the catalytic mTOR kinase inhibitor is an ATP-competitive mTOR kinase inhibitor.

7. The method of claim 3, wherein the mTOR inhibitor is an mTORC1 inhibitor or an mTORC1/2 inhibitor.

8-10. (canceled)

11. The method of claim 3, wherein the mTOR inhibitor is a dual PI3K/mTOR kinase inhibitor.

12-13. (canceled)

14. The method of claim 4, wherein the PI3K inhibitor is a dual PI3K/mTOR kinase inhibitor.

15. (canceled)

16. The method of claim 3, wherein the method of treatment further comprises administering an effective amount of an Akt inhibitor to the subject.

17. (canceled)

18. The method of claim 4, wherein the compound that inhibits the degradation of Grb10 is a ubiquitin ligase inhibitor.

19. The method of claim 18, wherein the ubiquitin ligase inhibitor is an E3 ubiquitin ligase inhibitor.

20. The method of claim 2, further comprising carrying out the selected method of treatment.

21. A method for selecting a treatment of a cancer, the method comprising,
(a) obtaining a cancer cell from a subject diagnosed to have a cancer exhibiting an elevated level of mTOR activity,
(b) determining the level of Grb10 expression in the cell, and
(c) comparing the level of Grb10 expression to a reference level, wherein if the level of Grb10 expression in the cell is higher than the reference level, then the cell is determined to exhibit a high risk of expressing an elevated level of PI3K, Akt, and/or MAPK activity upon being contacted with an mTORC1 inhibitor.

22. The method of claim 21, the method further comprising selecting a method of treatment based on the cell exhibiting a high risk of expressing an elevated level of PI3K, Akt, and/or MAPK activity upon being contacted with an mTORC1 inhibitor.

23. The method of claim 22, wherein the method of treatment comprises
administering (i) an effective amount of an mTOR kinase inhibitor and/or (ii) an effective amount of an IGF1R, EGFR, PI3K, Akt, MEK, and/or RSK inhibitors and/or of a compound stabilizing Grb10 to the subject, or
administering an effective amount of a dual or multi-target inhibitor that inhibits mTOR and inhibits IGFR, EGFR, PI3K, Akt, MEK, and/or RSK and/or of a compound stabilizing Grb10 to the subject.

24-40. (canceled)

41. A method for determining mTOR kinase activity in a cell, the method comprising
(a) determining the level of phosphorylation of a plurality of phosphorylation sites disclosed in Table 1, 2, 3, 7, or 8, or 11 in the cell; and
(b) comparing the level of phosphorylation determined in step (a) to a reference level, wherein
(i) if the level of phosphorylation determined in step (b) is higher than the reference level, then the cell is determined to exhibit an elevated level of mTOR kinase activity, or
(ii) if the level of phosphorylation determined in step (b) is equal or lower than the reference level, then the cell is determined to not exhibit an elevated level of mTOR kinase activity.

42-78. (canceled)

79. A method for determining mTOR kinase activity in a cell, the method comprising
(a) determining the level of phosphorylation of a phosphorylation site disclosed in Table 1, 2, 3, 7, 8, or 11 in the cell; and
(b) comparing the level of phosphorylation determined in step (a) to a reference level, and
(i) if the level of phosphorylation determined in step (b) is higher than the reference level, then the cell is determined to exhibit an elevated level of mTOR kinase activity, or
(ii) if the level of phosphorylation determined in step (b) is equal or lower than the reference level, then the cell is determined to not exhibit an elevated level of mTOR kinase activity.

80-126. (canceled)

127. A method of identifying an mTOR kinase inhibitor, the method comprising
(a) contacting an mTOR kinase with a polypeptide comprising a phosphorylation site disclosed in Table 1, 2, 3, 7, 8, or 11 under conditions suitable for the mTOR kinase to phosphorylate the phosphorylation site in the presence of a candidate agent,
(b) determining the level of phosphorylation at the phosphorylation site,
(c) comparing the level of phosphorylation determined in step (b) to a reference level, wherein if the level determined in step (b) is lower than the reference level, then the candidate agent is identified as an mTOR kinase inhibitor.

128-139. (canceled)

140. A method for determining mTOR kinase activity in a cell, the method comprising
(a) obtaining a cell from a subject,
(b) determining the level of phosphorylation of a protein selected from the group consisting of Grb10, FOXK1, ZEB2, NDRG3, LARP1, SRPK2, CDK12, MIB1, and IBTK in the cell, and
(c) comparing the level of phosphorylation of the protein to a reference level, wherein if the level of phosphorylation of the protein in the cell is higher than the reference level, then the cell is determined to exhibit an elevated level of mTOR kinase activity.

141-148. (canceled)
